(19) United States
(12) Patent Application Publication
(10) Pub. No.: US 2009/0010884 A1
(43) Pub. Date: Jan. 8, 2009

(54) PHARMACEUTICAL COMPOSITIONS AND METHODS OF PREVENTING, TREATING, OR INHIBITING INFLAMMATORY DISEASES, DISORDERS, OR CONDITIONS OF THE SKIN, AND DISEASES, DISORDERS, OR CONDITIONS ASSOCIATED WITH COLLAGEN DEPLETION

(75) Inventors: Chien-Neng CHANG, Collegeville, PA (US); Yihe WANG, Westfield, NJ (US); Sunil NAGPAL, Collegeville, PA (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 12/167,850

(22) Filed: Jul. 3, 2008

Related U.S. Application Data
(60) Provisional application No. 60/948,248, filed on Jul. 6, 2007.
(60) Provisional application No. 60/950,951, filed on Jul. 20, 2007.

(51) Int. Cl. A61K 38/21; A61K 31/423; A61K 31/055; A61K 31/343; A61K 31/47; A61P 17/06; A61P 17/00; A61K 31/381; A61K 31/70; A61K 38/02; A61K 38/13

(52) U.S. Cl. 424/85.5; 514/375; 514/732; 514/468; 514/311; 514/444; 514/54; 514/2; 514/11

(57) ABSTRACT

The present invention provides compositions and methods for preventing, treating, or inhibiting inflammatory diseases, disorders, or conditions of the skin, and diseases, disorders, or conditions associated with collagen depletion using one or more estrogenic agents.

### CROSS-REFERENCE TO RELATED APPLICATIONS

The present application claims benefit of priority from U.S. Provisional Application Ser. No. 60/950,951, filed Jul. 20, 2007, and from U.S. Provisional Application Ser. No. 60/948,248, filed Jul. 6, 2007, each of which is incorporated by reference in its entirety for any purpose.

### FIELD OF THE INVENTION

The present invention relates to pharmaceutical compositions and methods for the prevention, treatment, or inhibition of inflammatory diseases, disorders, or conditions of the skin, and diseases, disorders, or conditions associated with collagen depletion. In some embodiments, the methods include the use of one or more estrogenic agents.

### BACKGROUND OF THE INVENTION

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

As mentioned above, estrogens affect a panoply of biological processes. In addition, where gender differences have been described (e.g. disease frequencies, responses to challenge, etc), it is possible that the explanation involves the difference in estrogen levels between males and females.

It is known that estrogen receptors, both ERα and ERβ, are expressed in skin, and human skin in particular. There is a need for improved treatment of skin diseases, disorders, or conditions, particularly inflammatory diseases, disorders, or conditions of the skin and diseases, disorders, or conditions associated with collagen depletion. Accordingly, the invention described herein provides compounds, compositions, and methods for their use in preventing, treating, or inhibiting such skin diseases, disorders, or conditions.

### SUMMARY OF THE INVENTION

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of at least one compound of Formula I, having the structure:

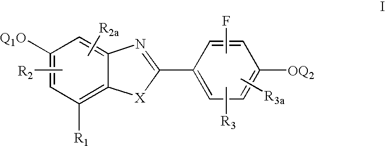

wherein:
Q₁ and Q₂ are independently H, a sugar residue selected from an unmodified or modified hexose residue, or S(O)ₜ—OH, provided that Q₁ and Q₂ are not both H;
t is 0, 1 or 2;
R₁ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₅ and R₆ are each, independently hydrogen, alkyl of 1-6 carbon atoms, or aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅; a pharmaceutically acceptable salt thereof; or

a gluocuronide, sulfate, or glucuronide-sulfate derivative thereof.

In some embodiments, said at least one compound of Formula I is selected from: 2-(3′-fluoro-4′-glucuronide phenyl)-7-vinyl-1,3-benzoxazol-5-ol; 2-(3′-fluoro-4′-sulfate phenyl)-7-vinyl-1,3-benzoxazol-5-ol; 2-(3′-fluoro-4′-hydroxy phenyl)-7-vinyl-1,3-benzoxazol-5-glucuronide; 2-(3′-fluoro-4′-hydroxy phenyl)-7-vinyl-1,3-benzoxazol-5-sulfate; 2-(3′-fluoro-4′-glucuroride phenyl)-7-vinyl-1,3-benzoxazol-5-glucuronide; 2-(3′-fluoro-4′-glucuronide phenyl)-7-vinyl-1,3-benzoxazol-5-sulfate; 2-(3′-fluoro-4′-sulfate phenyl)-7-vinyl-1,3-benzoxazol-5-glucuronide; 2-(3′-fluoro-4′-sulfate phenyl)-7-vinyl-1,3-benzoxazol-5-sulfate; 2-(2′-fluoro-4′-glucuronide phenyl)-7-vinyl-1,3-benzoxazol-5-ol; 2-(2′-fluoro-4′-sulfate phenyl)-7-vinyl-1,3-benzoxazol-5-ol, 2-(2′-fluoro-4′-hydroxy phenyl)-7-vinyl-1,3-benzoxazol-5-glucuronide; 2-(2′-fluoro-4′-hydroxy phenyl)-7-vinyl-1,3-benzoxazol-5-sulfate; 2-(2′-fluoro-4′-glucuronide phenyl)-7-vinyl-1,3-benzoxazol-5-glucuronide; 2-(2′-fluoro-4′-glucuronide phenyl)-7-vinyl-1,3-benzoxazol-5-sulfate; 2-(2′-fluoro-4′-sulfate phenyl)-7-vinyl-1,3-benzoxazol-5-glucuronide; 2-(2′-fluoro-4′-sulfate phenyl)-7-vinyl-1,3-benzoxazol-5-sulfate; 2-(2′,3′-difluoro-4′-glucuronide phenyl)-7-vinyl-1,3-benzoxazol-5-ol; 2-(2′,3′-difluoro-4′-sulfate phenyl)-7-vinyl-1,3-benzoxazol-5-ol; 2-(2′,3′-difluoro-4′-hydroxyphenyl)-7-vinyl-1,3-benzoxazol-5-glucuronide; 2-(2′,3′-difluoro-4′-hydroxyphenyl)-7-vinyl-1,3-benzoxazol-5-sulfate; 2-(2′,3′-difluoro-4′-glucuronide phenyl)-7-vinyl-1,3-benzoxazol-5-glucuronide; 2-(2′,3′-difluoro-4′-glucuronide phenyl)-7-vinyl-1,3-benzoxazol-5-sulfate; 2-(2′,3′-difluoro-4′-sulfate phenyl)-7-vinyl-1,3-benzoxazol-5-glucuronide; 2-(2′,3′-difluoro-4′-sulfate phenyl)-7-vinyl-1,3-benzoxazol-5-sulfate; 4-bromo-2-(3′-fluoro-4′-glucuronide phenyl)-7-vinyl-1,3-benzoxazol-5-ol; 4-bromo-2-(3′-fluoro-4′-sulfate phenyl)-7-vinyl-1,3-benzoxazol-5-ol; 4-bromo-2-(3′-fluoro-4′-hydroxyphenyl)-7-vinyl-1,3-benzoxazol-5-glucuronide; 4-bromo-2-(3′-fluoro-4′-hydroxyphenyl)-7-vinyl-1,3-benzoxazol-5-sulfate; 4-bromo-2-(3′-fluoro-4′-glucuronide phenyl)-7-vinyl-1,3-benzoxazol-5-glucuronide; 4-bromo-2-(3′-fluoro-4′-glucuronide phenyl)-7-vinyl-1,3-benzoxazol-5-sulfate; 4-bromo-2-(3′-fluoro-4′-sulfate phenyl)-7-vinyl-1,3-benzoxazol-5-glucuronide; 4-bromo-2-(3′-fluoro-4′-sulfate phenyl)-7-vinyl-1,3-benzoxazol-5-sulfate; 4,6-dibromo-2-(3′-fluoro-4′-glucuronide phenyl)-7-vinyl-1,3-benzoxazol-5-ol; 4,6-dibromo-2-(3′-fluoro-4′-sulfate phenyl)-7-vinyl-1,3-benzoxazol-5-ol; 4,6-di bromo-2-(3′-fluoro-4′-hydroxyphenyl)-7-vinyl-1,3-benzoxazol-5-glucuronide; 4,6-di bromo-2-(3′-fluoro-4′-hydroxyphenyl)-7-vinyl-1,3-benzoxazol-5-sulfate; 4,6-dibromo-2-(3′-fluoro-4′-glucuronide phenyl)-7-vinyl-1,3-benzoxazol-5-glucuronide; 4,6-dibromo-2-(3′-fluoro-4′-glucuronide phenyl)-7-vinyl-1,3-benzoxazol-5-sulfate; 4,6-dibromo-2-(3′-fluoro-4′-sulfate phenyl)-7-vinyl-1,3-benzoxazol-5-glucuronide; 4,6-dibromo-2-(3′-fluoro-4′-sulfate phenyl)-7-vinyl-1,3-benzoxazol-5-sulfate; 7-(1-bromovinyl)-2-(2′-fluoro-4′-glucuronide phenyl)-1,3-benzoxazol-5-ol; 7-(1-bromovinyl)-2-(2′-fluoro-4′-sulfate phenyl)-1,3-benzoxazol-5-ol; 7-(1-bromovinyl)-2-(2′-fluoro-4′-hydroxyphenyl)-1,3-benzoxazol-5-glucuronide; 7-(1-bromovinyl)-2-(2′-fluoro-4′-hydroxyphenyl)-1,3-benzoxazol-5-sulfate; 7-(1-bromovinyl)-2-(2′-fluoro-4′-glucuronide phenyl)-1,3-benzoxazol-5-glucuronide; 7-(1-bromovinyl)-2-(2′-fluoro-4′-glucuronide phenyl)-1,3-benzoxazol-5-sulfate; 7-(1-bromovinyl)-2-(2′-fluoro-4′-sulfate phenyl)-1,3-benzoxazol-5-glucuronide; 7-(1-bromovinyl)-2-(2′-fluoro-4′-sulfate phenyl)-1,3-benzoxazol-5-sulfate; 7-(1-bromovinyl)-2-(2′,3′-difluoro-4′-glucuronide phenyl)-1,3-benzoxazol-5-ol; 7-(1-bromovinyl)-2-(2′,3′-difluoro-4′-sulfate phenyl)-1,3-benzoxazol-5-ol; 7-(1-bromovinyl)-2-(2′,3′-difluoro-4′-hydroxyphenyl)-1,3-benzoxazol-5-glucuronide; 7-(1-bromovinyl)-2-(2′,3′-difluoro-4′-hydroxyphenyl)-1,3-benzoxazol-5-sulfate; 7-(1-bromovinyl)-2-(2′,3′-difluoro-4′-glucuronide phenyl)-1,3-benzoxazol-5-glucuronide; 7-(1-bromovinyl)-2-(2′,3′-difluoro-4′-glucuronide phenyl)-1,3-benzoxazol-5-sulfate; 7-(1-bromovinyl)-2-(2′,3′-difluoro-4′-sulfate phenyl)-1,3-benzoxazol-5-glucuronide; 7-(1-bromovinyl)-2-(2′,3′-difluoro-4′-sulfate phenyl)-1,3-benzoxazol-5-sulfate; 7-allyl-2-(3′-fluoro-4′-glucuroinide phenyl)-1,3-benzoxazol-5-ol; 7-allyl-2-(3′-fluoro-4′-sulfate phenyl)-1,3-benzoxazol-5-ol; 7-allyl-2-(3′-fluoro-4′-hydroxyphenyl)-1,3-benzoxazol-5-glucuronide; 7-allyl-2-(3′-fluoro-4′-hydroxyphenyl)-1,3-benzoxazol-5-sulfate; 7-allyl-2-(3′-fluoro-4′-glucuroinide phenyl)-1,3-benzoxazol-5-glucuronide; 7-allyl-2-(3′-fluoro-4′-glucuroinide phenyl)-1,3-benzoxazol-5-sulfate; 7-allyl-2-(3′-fluoro-4′-sulfate phenyl)-1,3-benzoxazol-5-glucuronide; 7-allyl-2-(3′-fluoro-4′-sulfate phenyl)-1,3-benzoxazol-5-sulfate; 2-(3′,5′-difluoro-4′-glucuronide phenyl)-7-vinyl-1,3-benzoxazol-5-ol; 2-(3′,5′-difluoro-4′-sulfate phenyl)-7-vinyl-1,3-benzoxazol-5-ol; 2-(3′,5′-difluoro-4′-hydroxy phenyl)-7-vinyl-1,3-benzoxazol-5-glucuronide; 2-(3′,5′-difluoro-4′-hydroxyphenyl)-7-vinyl-1,3-benzoxazol-5-sulfate; 2-(3′,5′-difluoro-4′-glucuronide phenyl)-7-vinyl-1,3-benzoxazol-5-glucuronide; 2-(3′,5′-difluoro-4′-glucuronide phenyl)-7-vinyl-1,3-benzoxazol-5-sulfate; 2-(3′,5′-difluoro-4′-sulfate phenyl)-7-vinyl-1,3-benzoxazol-5-glucuronide; 2-(3′,5′-difluoro-4′-sulfate phenyl)-7-vinyl-1,3-benzoxazol-5-sulfate; 2-(3′-fluoro-4′-glucuronide phenyl)-7-(1-fluorovinyl)-1,3-benzoxazol-5-ol; 2-(3′-fluoro-4′-sulfate phenyl)-7-(1-fluorovinyl)-1,3-benzoxazol-5-ol; 2-(3′-fluoro-4′-hydroxyphenyl)-7-(1-fluorovinyl)-1,3-benzoxazol-5-glucuronide; 2-(3′-fluoro-4′-hydroxyphenyl)-7-(1-fluorovinyl)-1,3-benzoxazol-5-sulfate; 2-(3′-fluoro-4′-glucuronide phenyl)-7-(1-fluorovinyl)-1,3-benzoxazol-5-glucuronide; 2-(3′-fluoro-4′-glucuronide phenyl)-7-(1-fluorovinyl)-1,3-benzoxazol-5-sulfate; 2-(3′-fluoro-4′-sulfate phenyl)-7-(1-fluorovinyl)-1,3-benzoxazol-5-glucuronide; 2-(3′-fluoro-4′-sulfate phenyl)-7-(1-fluorovinyl)-1,3-benzoxazol-5-sulfate; and a pharmaceutically acceptable salt thereof.

Some further embodiments of the present invention provide methods of preventing, treating, or inhibiting an inflammatory disease, disorder, or condition of the skin, or a disease, disorder, or condition associated with collagen depletion, comprising: providing to a mammal in need thereof, a therapeutically effective amount of one or more estrogenic agents, wherein at least one of said one or more estrogenic agents is selected from a gluocuronide derivative, a sulfate derivative, or a glucuronide-sulfate derivative of: 2-(5-hydroxy-1,3-benzoxazol-2-yl) benzene-1,4-diol; 3-(5-hydroxy-1,3-benzoxazol-2-yl)benzene-1,2-diol; 2-(3-fluoro-4-hydroxyphenyl)-1,3-benzoxazol-5-ol; 2-(3-chloro-4-hydroxyphenyl)-1,3-benzoxazol-5-ol; 2-(2-chloro-4-hydroxyphenyl)-1,3-benzoxazol-5-ol; 2-(3-fluoro-4-hydroxyphenyl)-1,3-benzoxazol-6-ol; 2-(3-tert-butyl-4-hydroxyphenyl)-1,3-benzoxazol-6-ol; 2-(6-hydroxy-1,3-benzoxazol-2-yl)benzene-1,4-diol; 3-(6-hydroxy-1,3-benzoxazol-2-yl)benzene-1,2-diol; 4-(6-hydroxy-1,3-benzoxazol-2-yl)benzene-1,2-diol; 2-(3-chloro-4-hydroxyphenyl)-1,3-benzoxazol-6-ol; 4-(5-hydroxy-1,3-benzoxazol-2-yl)benzene-1,3-diol; 4-(6-hydroxy-1,3-benzoxazol-2-yl)benzene-1,3-diol; 6-chloro-2-(3-fluoro-4-hydroxyphenyl)-1,3-benzoxazol-5-ol; 6-bromo-2-(3-fluoro-4-hydroxyphenyl)-1,3-benzoxazol-5-ol; 6-chloro-2-(4-hydroxyphenyl)-1,3-benzoxazol-5-ol; 5-chloro-2-(4-hydroxyphenyl)-1,3-benzoxazol-6-ol; 7-bromo-2-(3-fluoro-4-hydroxyphenyl)-1,3-benzoxazol-5-ol; 7-bromo-2-(2-fluoro-4-hydroxyphenyl)-1,3-benzoxazol-5-ol; 7-bromo-2-(2,3-difluoro-4-hydroxyphenyl)-1,3-benzoxazol-5-ol; 2-(4-hydroxyphenyl)-7-vinyl-1,3-benzoxazol-5-ol; 7-(1,2-dibromoethyl)-2-(4-hydroxyphenyl)-1,3-benzoxazol-5-ol; 7-(1-bromovinyl)-2-(4-hydroxyphenyl)-1,3-benzoxazol-5-ol; 7-ethynyl-2-(4-hydroxyphenyl)-1,3-benzoxazol-5-ol; 2-(4-hydroxyphenyl)-7-propyl-1,3-benzoxazol-5-ol; 7-butyl-2-(4-hydroxyphenyl)-1,3-benzoxazol-5-ol; 7-cyclopentyl-2-(4-hydroxyphenyl)-1,3-benzoxazol-5-ol; ethyl 5-hydroxy-2-(4-hydroxyphenyl)-1,3-benzoxazole-7-carboxylate; 2-(4-hydroxyphenyl)-7-phenyl-1,3-benzoxazol-5-ol; 2-(4-hydroxyphenyl)-7-methoxy-1,3-benzoxazol-5-ol; 7-ethyl-2-(4-hydroxyphenyl)-1,3-benzoxazol-5-ol; 7-ethyl-2-(2-ethyl-4-hydroxyphenyl)-1,3-benzoxazol-5-ol; 5-hydroxy-2-(4-hydroxyphenyl)-1,3-benzoxazole-7-carbaldehyde; 7-(hydroxymethyl)-2-(4-hydroxyphenyl)-1,3-benzoxazol-5-ol; 7-(bromomethyl)-2-(4-hydroxyphenyl)-1,3-benzoxazol-5-ol; [5-hydroxy-2-(4-hydroxyphenyl)-1,3-benzoxazol-7-yl]acetonitrile; 7-(1-hydroxy-1-methylethyl)-2-(4-hydroxyphenyl)-1,3-benzoxazol-5-ol; 2-(4-hydroxyphenyl)-7-isopropenyl-1,3-benzoxazol-5-ol; 2-(4-hydroxyphenyl)-7-isopropyl-1,3-benzoxazol-5-ol; 7-bromo-2-(4-hydroxy-3-(trifluoromethyl)phenyl)-1,3-benzoxazol-5-ol; 7-(2-furyl)-2-(4-hydroxyphenyl)-1,3-benzoxazol-5-ol; 2-(3-fluoro-4-hydroxyphenyl)-7-(2-furyl)-1,3-benzoxazol-5-ol; 2-(4-hydroxyphenyl)-7-thien-2-yl-1,3-benzoxazol-5-ol; 2-(4-hydroxyphenyl)-7-(1,3-thiazol-2-yl)-1,3-benzoxazol-5-ol; 2-(3-fluoro-4-hydroxyphenyl)-5-hydroxy-1,3-benzoxazole-7-carbonitrile; 4-bromo-2-(4-hydroxyphenyl)-7-methoxy-1,3-benzoxazol-5-ol; 4,6-dibromo-2-(4-hydroxyphenyl)-7-methoxy-1,3-benzoxazol-5-ol; 7-bromo-2-(3,5-difluoro-4-hydroxyphenyl)-1,3-benzoxazol-5-ol; and a pharmaceutically acceptable salt thereof.

Some further embodiments of the present invention provide methods of preventing, treating, or inhibiting an inflammatory disease, disorder, or condition of the skin, or a disease, disorder, or condition associated with collagen depletion comprising: providing to a mammal in need thereof, a therapeutically effective amount of one or more estrogenic agents. In some embodiments, at least one of said one or more estrogenic agents is:

a. a compound of Formula II, having the structure:

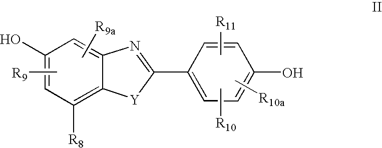

wherein

R₈ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, CONR₁₂R₁₃, NR₁₂R₁₃ or N(R₁₂)COR₁₃;

R₉ and R₉ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;

R₁₀, R₁₀ₐ and R₁₁ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;

R₁₂ and R₁₃ are each, independently hydrogen, alkyl of 1-6 carbon atoms, aryl of 6-10 carbon atoms;

### Y is O, S, or NR₁₄;

R₁₄ is hydrogen, alkyl of 1-6 carbon atoms, aryl of 6-10 carbon atoms, —COR₁₂, —CO₂R₁₂ or —SO₂R₁₂;

or a pharmaceutically acceptable salt thereof;

b. a compound of Formula III, having the structure:

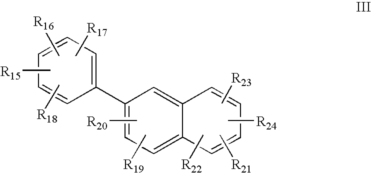

wherein
R₁₅, R₁₆, R₁₇, and R₁₈ are each, independently, selected from hydrogen, hydroxyl, alkyl of 1-6 carbon atoms, alkoxy of 1-6 carbon atoms, or halogen;
R₁₉, R₂₀, R₂₁, R₂₂, R₂₃, and R₂₄, are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₉, R₂₀, R₂₁, R₂₂, R₂₃, or R₂₄ may be optionally substituted with hydroxyl, —CN, halogen, trifluoroalkyl, trifluoroalkoxy, —NO₂, or phenyl; wherein the phenyl moiety of R₁₉, R₂₀, R₂₁, R₂₂, R₂₃, or R₂₄ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
with the proviso that at least one of R₁₅, R₁₆, R₁₇, R₁₈, R₂₁, R₂₂, R₂₃, or R₂₄ is hydroxyl, or a pharmaceutically acceptable salt thereof;
   c. a compound of Formula IV, having the structure:

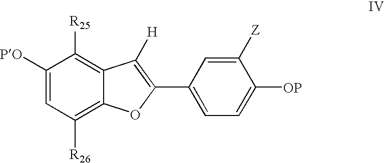

wherein
P and P′ are each, independently, hydrogen, alkyl of 1-6 carbon atoms, or acyl of 2-7 carbon atoms;
Z is hydrogen or halogen;
R₂₅ is hydrogen, alkyl of 1-6 carbon atoms, halogen, —CN, or —CHO;
R₂₆ is alkoxy of 1-6 carbon atoms, or cyanoalkyl having 1-6 carbon atoms in the alkyl moiety; or a pharmaceutically acceptable salt thereof;
   d. a compound of Formula V, having the structure:

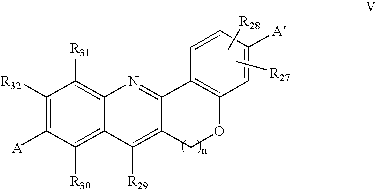

wherein:

A and A′ are each independently OH, H or OU;

each U is independently selected from the group consisting of C₁-C₆ alkyl, alkenyl, benzyl, acyl of 2-7 carbon atoms, aroyl, alkoxycarbonyl, sulfonyl and phosphoryl;

R₂₇ and R₂₈ are independently selected from the group consisting of H, halogen, C₁₋₆ alkyl, C₁₋₆ perhaloalkyl, —CF₃, C₂-C₇ alkenyl and C₁-C₆ alkoxy;

R₂₉, R₃₀, R₃₁ and R₃₂ are each independently selected from the group consisting of H, halogen, —CF₃, C₁₋₆ perhaloalkyl, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl and heteroaryl;

wherein each alkyl or alkenyl moiety of R₂₉, R₃₀, R₃₁ and R₃₂ can be optionally substituted with up to three substituents independently selected from halogen, OH, —CN, trifluoroalkyl of 1-6 carbon atoms, trifluoroalkoxy of 1-6 carbon atoms, —NO₂ and phenyl, wherein said phenyl is optionally substituted with up to three independently selected R₃₃ groups;

wherein each alkynyl moiety of R₂₉, R₃₀, R₃₁ and R₃₂ can be optionally substituted with up to three substituents selected from halogen, —CN, —CHO, acyl, trifluoroalkyl of 1-6 carbon atoms, trialkylsilyl and phenyl, wherein said phenyl is optionally substituted with up to three independently selected R₃₃ groups;

each R₃₃ is independently selected from the group consisting of halogen, C₁-C₆ alkyl, C₂-C₇ alkenyl, —OH, C₁-C₆ alkoxy, —CN, —CHO, —NO₂, amino, C₁-C₆ alkylamino, di-(C₁-C₆)alkylamino, thiol, and C₁-C₆ alkylthio; and

n is 0, 1, 2, or 3;

provided that:

at least one of A and A′ is not H;

if n is 0, then R₂₉ is not halogen; and

at least one of R₂₉, R₃₀ and R₃₁ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;

or a pharmaceutically acceptable salt thereof;

e. a compound of Formula VI, having the structure:

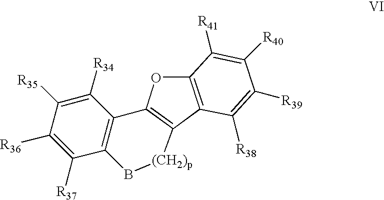

wherein:
B has the structure (i), (ii) or (iii):

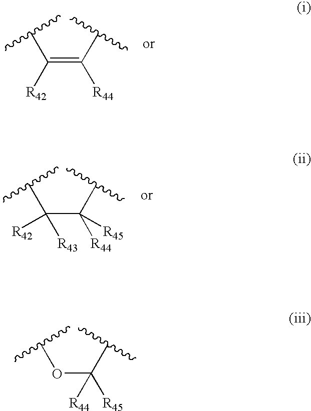

R₃₄, R₃₇, R₃₈, R₄₁, R₄₂, R₄₃, R₄₄, and R₄₅ are each independently selected from the group consisting of hydrogen, C₁-C₆ alkyl, —OR₄₆, halogen, —CF₃, —CF₂CF₃, —CH₂CF₃, —SR₄₆, —NR₄₆R₄₇, —CN, —CH₂CN, —CH₂CH₂CN, —CH═CHCN, —NO₂, —CH₂NO₂, —CH₂CH₂NO₂, —CH═CHNO₂ and —COR₄₆;

p=0 or 1;

each R₄₆ and R₄₇ is independently selected from the group consisting of hydrogen, C₁-C₆ alkyl, —CF₃, benzyl, —CO₂(C₁-C₆ alkyl) and —CO(C₁-C₆ alkyl);

provided that:

a) one of R₃₅ or R₃₆ must be —OR₄₆;

b) one of R₃₉ or R₄₀ must be —OR₄₆;

c) when R₃₅ is —OR₄₆, then R₃₄ and R₃₆ are independently selected from the group consisting of hydrogen, halogen, C₁-C₆ alkyl, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;

d) when R₃₆ is —OR₄₆, then R₃₅ and R₃₇ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;

e) when R₃₉ is —OR₄₆, then R₃₈ and R₄₀ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₃;

f) when R₄₀ is —OR₄₆, then R₃₉ and R₄₁ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆; and

g) when B has the structure (iii), and R₃₈, R₃₉, R₄₁, R₄₄, and R₄₅ are each H, and p=0, then R₄₀ is not —OR₄₆;

f. a compound of Formula VII, having the structure:

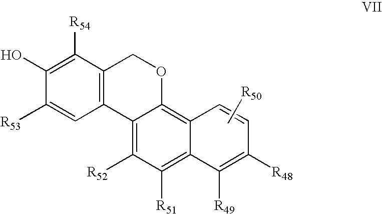

wherein
R₄₈ and R₄₉ are each, independently, selected from hydrogen, hydroxyl, alkyl of 1-6 carbon atoms, alkenyl of 2-7 carbon atoms, alkynyl of 2-7 carbon atoms, alkoxy of 1-6 carbon atoms, or halogen; wherein each alkyl or alkenyl moiety of R₄₈ or R₄₉ may be optionally and independently substituted with hydroxyl, —CN, halogen, trifluoroalkyl of 1-6 carbon atoms, trifluoroalkoxy of 1-6 carbon atoms, —NO₂ or phenyl; and provided that at least one of R₄₈ or R₄₉ is hydroxyl;
R₅₀, R₅₁, R₅₂, R₅₃, and R₅₄ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein each alkyl or alkenyl moiety of R₅₁, R₅₂, R₅₃ or R₅₄ may be optionally and independently substituted with hydroxyl, —CN, halogen, trifluoroalkyl of 1-6 carbon atoms, trifluoroalkoxy of 1-6 carbon atoms, —NO₂ or phenyl; wherein the phenyl moiety of R₅₁ or R₅₂ may be optionally mono-, di-, or tri-substituted with a substituent, the same or different, selected from alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

or a pharmaceutically acceptable salt thereof;

g. a compound of Formula VIII, having the structure:

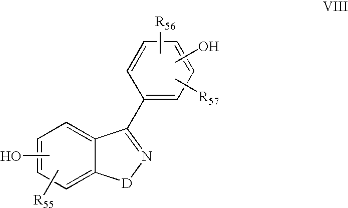

wherein,
R₅₅ and R₅₆ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, trifluoroalkyl of 1-6 carbon atoms, trifluoroalkoxy of 1-6 carbon atoms, alkylthio of 1-6 carbon atoms, or aryl of 6-10 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₇ is hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, alkylthio of 1-6 carbon atoms, trifluoroalkyl of 1-6 carbon atoms, or trifluoroalkoxy of 1-6 carbon atoms, aryl of 6-10 carbon atoms, —NO₂, —CN, —NR₅₈R₅₉ or —N(R₅₈)COR₅₉, —CHFCN, —CF₂CN, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₈ and R₅₉ are each, independently, hydrogen, alkyl of 1-6 carbon atoms, or aryl of 6-10 carbon atoms;
D is O, S, or NR₆₀;
R₆₀ is hydrogen, alkyl of 1-6 carbon atoms, aryl of 6-10 carbon atoms, —COR₅₈, —CO₂R₅₈, or —SO₂R₅₈;
   h. a compound of Formula IX, having the structure:

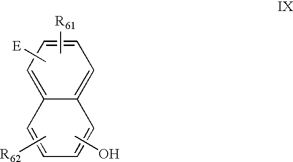

wherein,

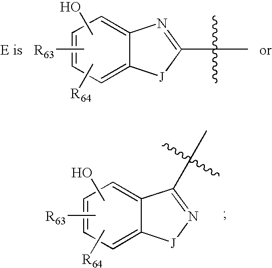

R₆₁, R₆₃, and R₆₄ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₂ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₆₅R₆₆, —N(R₆₅)COR₆₆, —CN, —CHFCN, —CF₂CN, alkynyl of 2-7 carbon atoms, or alkenyl of 2-7 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₅ and R₆₆ are each, independently, hydrogen, alkyl of 1-6 carbon atoms, alkenyl of 2-7 carbon atoms, aryl of 6-10 carbon atoms, —CN, —CHFCN, or —CF₂CN; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₆₇, —CO₂R₆₈, —NO₂, —CONR₆₇R₆₈, —NR₆₇R₆₈ or —N(R₆₇)COR₆₈;
R₆₇ and R₆₈ are each, independently, hydrogen, alkyl of 1-6 carbon atoms, or aryl of 6-10 carbon atoms;
J is O, S, or NR₆₉;
R₆₉ is hydrogen, alkyl of 1-6 carbon atoms, aryl of 6-10 carbon atoms, —COR₆₅, —CO₂R₆₅, or —SO₂R₆₅;

or a pharmaceutically acceptable salt thereof;

i. a compound of Formula X, having the structure:

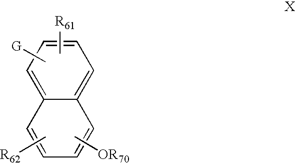

wherein,

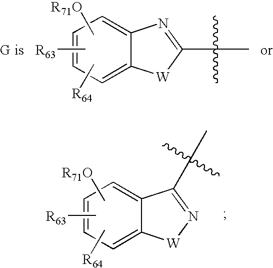

wherein
R₆₁, R₆₂, R₆₃, and R₆₄ are as defined above;
W is O, S, or NR₆₉;
R₆₉ is as defined above; and
R₇₀ and R₇₁ are alkyl of 1-6 carbon atoms;

or a pharmaceutically acceptable salt thereof;

j. a compound of Formula XI, having the structure:

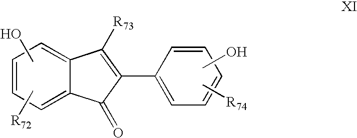

wherein,
R₇₂ is hydrogen, hydroxyl, halogen, trifluoroalkyl of 1-6 carbon atoms, alkyl of 1-6 carbon atoms, cycloalkyl of 3-8 carbon atoms, alkenyl of 2-7 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, —CN, —NO₂, —CHFCN, —CF₂CN, aryl of 6-10 carbon atoms, —NR₇₅R₇₆, —NCOR₇₅, —SR₇₅, —SO₂R₇₅, —SO₂R₇₅, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₇₅, —CO₂R₇₅, —CONR₇₅R₇₆, or —NR₇₅R₇₆;
R₇₃ is hydrogen, hydroxyl, alkyl of 1-6 carbon atoms, halogen, phenyl substituted with R₁, alkylthio of 1-6 carbon atoms, thioalkyl of 1-6 carbon atoms, amino, aminoalkyl of 1-6 carbon atoms, alkylamino of 1-6 carbon atoms, alkoxy of 1-6 carbon atoms, or alkenyl of 2-7 carbon atoms;
R₇₄ is hydrogen, halogen, hydroxyl, alkyl of 1-6 carbon atoms, alkoxy of 1-6 carbon atoms, trifluoroalkyl of 1-6 carbon atoms, or trifluoroalkoxy of 1-6 carbon atoms;
R₇₅ and R₇₆ are each, independently, hydrogen, alkyl of 1-6 carbon atoms, or aryl of 6-10 carbon atoms;

or a pharmaceutically acceptable salt thereof;

k. a compound of Formula XII, having the structure:

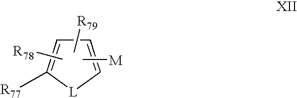

wherein,
R₇₇ is phenyl optionally substituted with 1-4 T groups;
R₇₈ is phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
R₇₉ is hydrogen, phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
L is O, —CH═CH—, or S;
T is —OH, —OR₈₀, halogen, —CN, —CO₂H, —CO₂R₈₀, alkyl of 1-6 carbon atoms, alkenyl of 2-7 carbon atoms, alkynyl of 2-7 carbon atoms, perfluoroalkyl of 1-6 carbon atoms, or —COR₈₀;
M is —CHO, —CN, —CO₂H, —CO₂R₈₀, —CONR₈OR₈₁, —NO₂, —CH═NR₈₀, —CH═NOH, or —CH═NOR₈₀;
R₈₀ and R₈₁ are each, independently, alkyl of 1-6 carbon atoms, alkenyl of 2-7 carbon atoms, alkynyl of 2-7 carbon atoms, or cycloalkyl of 3-8 carbon atoms;
with the proviso that at least one of R₇₈ or R₇₉ is phenyl or phenyl substituted with 1-4 T groups;

or a pharmaceutically acceptable salt thereof;

l. a compound of Formula XIII, having the structure:

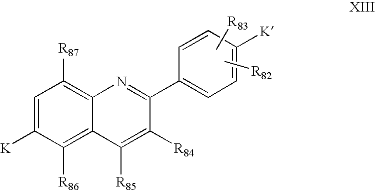

wherein:

K and K′ are each, independently, OH or OV;
V is alkyl, alkenyl, benzyl, acyl of 2-7 carbon atoms, aroyl, alkoxycarbonyl, sulfonyl or phosphoryl;

R₈₂ and R₈₃ are each, independently, H, halogen, C₁-C₆ alkyl, C₂-C₇ alkenyl, or C₁-C₆ alkoxy;

R₈₄ is H, halogen, or C₁-C₆ alkyl;

R₈₅ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, or heteroaryl;

R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₅, R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;

wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;

wherein each alkynyl moiety of R₈₅, R₈₆ or R₈₇ may be optionally substituted with halogen, —CN, —CHO, acyl, trifluoroalkyl of 1-6 carbon atoms, trialkylsilyl, or optionally substituted phenyl;

wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;

provided that when each of R₈₅, R₈₆ and R₈₇ are H, C₁-C₆ alkyl, C₂-C₇ alkenyl, or C₁-C₆ alkoxy, then at least one of R₈₂ and R₈₃ is halogen, C₁-C₆ alkyl, C₂-C₇ alkenyl, or C₁-C₆ alkoxy;

provided that at least one of R₈₅ and R₈₇ is other than H;

or a pharmaceutically acceptable salt thereof;
m. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;

R₈₂ and R₈₃ are each H;

R₈₄ is H, halogen, or C₁-C₆ alkyl;

R₈₅, R₈₆ and R₈₇ are each, independently, H, C₁-C₆ alkyl, C₂-C₇ alkenyl, or C₁-C₆ alkoxy,

provided that at least one of R₈₅, R₈₆ and R₈₇ is C₁-C₆ alkyl, C₂-C₇ alkenyl, or C₁-C₆ alkoxy;

wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;

or a pharmaceutically acceptable salt thereof; or

n. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;

R₈₂ and R₈₃ are each, independently, H, halogen, C₁-C₆ alkyl, C₂-C₇ alkenyl, or C₁-C₆ alkoxy;

R₈₄ is H;

R₈₅ is H, aryl, or substituted aryl;

R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;

wherein each alkyl or alkenyl moiety of R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;

wherein each alkynyl moiety of R₈₆ or R₈₇ may be optionally substituted with halogen, —CN, —CHO, acyl of 2-7 carbon atoms, trifluoroalkyl of 1-6 carbon atoms, trialkylsilyl, or optionally substituted phenyl;

wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;

or a N-oxide thereof or a pharmaceutically acceptable salt thereof or a prodrug thereof.

In some embodiments, at least one of said one or more estrogenic agents is a compound of Formula II, wherein Y is O. In some embodiments, at least one of said one or more estrogenic agents is a compound of Formula II, wherein Y is O, and R₈ is halogen or alkenyl of 2-3 carbon atoms, which is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃, or —N(R₁₂)COR₁₃.

In some embodiments, at least one of said one or more estrogenic agents is a compound of Formula II, wherein R₁₅, R₁₆, R₁₇, and R₁₈ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen; R₁₉, R₂₀, R₂₁, R₂₂, R₂₃, and R₂₄ are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₉, R₂₀, R₂₁, R₂₂, R₂₃, or R₂₄ may be optionally substituted with hydroxyl, —CN, halogen, trifluoroalkyl, trifluoroalkoxy, —NO₂, or phenyl; wherein the phenyl moiety of R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, halogen, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; with the proviso that at least one of R₁₉ or R₂₃ is not hydrogen; and R₂₄ is hydroxyl.

In some further embodiments, at least one of said one or more estrogenic agents is a compound of Formula II, wherein the 5 or 6-membered heterocyclic ring having 1 to 4 heteroatoms selected from O, N or S is furan, thiophene, or pyridine or a pharmaceutically acceptable salt thereof. In yet further embodiments, at least one of said one or more estrogenic agents is a compound of Formula III wherein the 5 or 6-membered heterocyclic ring having 1 to 4 heteroatoms selected from O, N or S is furan, thiophene, or pyridine; and wherein one of R₁₅, R₁₆, R₁₇, and R₁₈ is hydroxyl, wherein R₁₉, R₂₀, R₂₁, R₂₂ and R₂₃ are each, independently, hydrogen, halogen, —CN, or alkynyl of 2-7 carbon atoms, and wherein R₂₄ is hydroxyl; or a pharmaceutically acceptable salt thereof.

In some embodiments, at least one of said one or more estrogenic agents is selected from: 2-(3-fluoro-4-hydroxyphenyl)-7-vinyl-1,3-benzoxazol-5-ol; 2-(2-fluoro-4-hydroxyphenyl)-7-vinyl-1,3-benzoxazol-5-ol; 2-(2,3-difluoro-4-hydroxyphenyl)-7-vinyl-1,3-benzoxazol-5-ol; 4-bromo-2-(3-fluoro-4-hydroxyphenyl)-7-vinyl-1,3-benzoxazol-5-ol; 4,6-dibromo-2-(3-fluoro-4-hydroxyphenyl)-7-vinyl-1,3-benzoxazol-5-ol; 7-(1-bromovinyl)-2-(2-fluoro-4-hydroxyphenyl)-1,3-benzoxazol-5-ol; 7-(1-bromovinyl)-2-(2,3-difluoro-4-hydroxyphenyl)-1,3-benzoxazol-5-ol; 7-allyl-2-(3-fluoro-4-hydroxyphenyl)-1,3-benzoxazol-5-ol; 2-(3,5-difluoro-4-hydroxyphenyl)-7-vinyl-1,3-benzoxazol-5-ol; 2-(3-fluoro-4-hydroxyphenyl)-7-(1-fluorovinyl)-1,3-benzoxazol-5-ol; and a pharmaceutically acceptable salt thereof.

In some embodiments, at least one of said one or more estrogenic agents is selected from: 2-(5-hydroxy-1,3-benzoxazol-2-yl)benzene-1,4-diol; 3-(5-hydroxy-1,3-benzoxazol-2-yl)benzene-1,2-diol; 2-(3-fluoro-4-hydroxyphenyl)-1,3-benzoxazol-5-ol; 2-(3-chloro-4-hydroxyphenyl)-1,3-benzoxazol-5-ol; 2-(2-chloro-4-hydroxyphenyl)-1,3-benzoxazol-5-ol; 2-(3-fluoro-4-hydroxyphenyl)-1,3-benzoxazol-6-ol; 2-(3-tert-butyl-4-hydroxyphenyl)-1,3-benzoxazol-6-ol; 2-(6-hydroxy-1,3-benzoxazol-2-yl)benzene-1,4-diol; 3-(6-hydroxy-1,3-benzoxazol-2-yl)benzene-1,2-diol; 4-(6-hydroxy-1,3-benzoxazol-2-yl)benzene-1,2-diol; 2-(3-chloro-4-hydroxyphenyl)-1,3-benzoxazol-6-ol; 4-(5-hydroxy-1,3-benzoxazol-2-yl)benzene-1,3-diol; 4-(6-hydroxy-1,3-benzoxazol-2-yl)benzene-1,3-diol; 6-chloro-2-(3-fluoro-4-hydroxyphenyl)-1,3-benzoxazol-5-ol; 6-bromo-2-(3-fluoro-4-hydroxyphenyl)-1,3-benzoxazol-5-ol; 6-chloro-2-(4-hydroxyphenyl)-1,3-benzoxazol-5-ol; 5-chloro-2-(4-hydroxyphenyl)-1,3-benzoxazol-6-ol; 7-bromo-2-(3-fluoro-4-hydroxyphenyl)-1,3-benzoxazol-5-ol; 7-bromo-2-(2-fluoro-4-hydroxyphenyl)-1,3-benzoxazol-5-ol; 7-bromo-2-(2,3-difluoro-4-hydroxyphenyl)-1,3-benzoxazol-5-ol; 2-(4-hydroxyphenyl)-7-vinyl-1,3-benzoxazol-5-ol; 7-(1,2-dibromoethyl)-2-(4-hydroxyphenyl)-1,3-benzoxazol-5-ol; 7-(1-bromovinyl)-2-(4-hydroxyphenyl)-1,3-benzoxazol-5-ol; 7-ethynyl-2-(4-hydroxyphenyl)-1,3-benzoxazol-5-ol; 2-(4-hydroxyphenyl)-7-propyl-1,3-benzoxazol-5-ol; 7-butyl-2-(4-hydroxyphenyl)-1,3-benzoxazol-5-ol; 7-cyclopentyl-2-(4-hydroxyphenyl)-1,3-benzoxazol-5-ol; ethyl 5-hydroxy-2-(4-hydroxyphenyl)-1,3-benzoxazole-7-carboxylate; 2-(4-hydroxyphenyl)-7-phenyl-1,3-benzoxazol-5-ol; 2-(4-hydroxyphenyl)-7-methoxy-1,3-benzoxazol-5-ol; 7-ethyl-2-(4-hydroxyphenyl)-1,3-benzoxazol-5-ol; 7-ethyl-2-(2-ethyl-4-hydroxyphenyl)-1,3-benzoxazol-5-ol; 5-hydroxy-2-(4-hydroxyphenyl)-1,3-benzoxazole-7-carbaldehyde; 7-(hydroxymethyl)-2-(4-hydroxyphenyl)-1,3-benzoxazol-5-ol; 7-(bromomethyl)-2-(4-hydroxyphenyl)-1,3-benzoxazol-5-ol; [5-hydroxy-2-(4-hydroxyphenyl)-1,3-benzoxazol-7-yl]acetonitrile; 7-(1-hydroxy-1-methylethyl)-2-(4-hydroxyphenyl)-1,3-benzoxazol-5-ol; 2-(4-hydroxyphenyl)-7-isopropenyl-1,3-benzoxazol-5-ol; 2-(4-hydroxyphenyl)-7-isopropyl-1,3-benzoxazol-5-ol; 7-bromo-2-(4-hydroxy-3-(trifluoromethyl)phenyl)-1,3-benzoxazol-5-ol; 7-(2-furyl)-2-(4-hydroxyphenyl)-1,3-benzoxazol-5-ol; 2-(3-fluoro-4-hydroxyphenyl)-7-(2-furyl)-1,3-benzoxazol-5-ol; 2-(4-hydroxyphenyl)-7-thien-2-yl-1,3-benzoxazol-5-ol; 2-(4-hydroxyphenyl)-7-(1,3-thiazol-2-yl)-1,3-benzoxazol-5-ol; 2-(3-fluoro-4-hydroxyphenyl)-5-hydroxy-1,3-benzoxazole-7-carbonitrile; 4-bromo-2-(4-hydroxyphenyl)-7-methoxy-1,3-benzoxazol-5-ol; 4,6-bi bromo-2-(4-hydroxyphenyl)-7-methoxy-1,3-benzoxazol-5-ol; 7-bromo-2-(3,5-difluoro-4-hydroxyphenyl)-1,3-benzoxazol-5-ol; and a pharmaceutically acceptable salt thereof.

In some embodiments, at least one of said one or more estrogenic agents is selected from: 8-fluoro-6-(3-fluoro-4-hydroxyphenyl)-2-naphthol; 1-chloro-8-fluoro-6-(3-fluoro-4-hydroxyphenyl)-2-naphthol; 3-(3-fluoro-4-hydroxyphenyl)-7-hydroxy-1-naphthonitrile; 3-(3,5-difluoro-4-hydroxyphenyl)-7-hydroxy-1-naphthonitrile; and a pharmaceutically acceptable salt thereof.

In some further embodiments, at least one of said one or more estrogenic agents is selected from: 7-(4-hydroxyphenyl)-2-naphthol; 7-(3-hydroxyphenyl)-2-naphthol; 6-(4-hydroxyphenyl)-1-naphthol; 6-phenyl-2-naphthol; 6-(3-hydroxyphenyl)-2-naphthol; 6-(3-chlorophenyl)-2-naphthol; 2-fluoro-4-(2-naphthyl)phenol; 6-(3-fluoro-4-hydroxyphenyl)-2-naphthol; 6-(3-chloro-4-hydroxyphenol)-2-naphthol; 1-chloro-6-phenyl-2-naphthol; 1-bromo-6-(4-hydroxyphenyl)-2-naphthol; 1-chloro-6-(4-hydroxyphenyl)-2-naphthol; 1-fluoro-6-(4-hydroxyphenyl)-2-naphthol; 2-hydroxy-6-(4-hydroxyphenyl)-1-naphthonitrile; 6-(4-hydroxyphenyl)-1-phenyl-2-naphthol; 6-(4-hydroxyphenyl)-1-methyl-2-naphthol; 1-chloro-6-(3-fluoro-4-hydroxyphenyl)-2-naphthol; 1-chloro-6-(3-chloro-4-hydroxyphenyl)-2-naphthol; 6-(4-hydroxyphenyl)-1-nitro-2-naphthol; 1-chloro-6-(4-hydroxy-2-methylphenyl)-2-naphthol; 6-(4-hydroxy-2-methylphenyl)-2-naphthol; 6-(4-hydroxy-2-methoxyphenyl)-2-naphthol; 6-(2-chloro-4-hydroxyphenyl)-2-naphthol; 1-chloro-6-(2-chloro-4-hydroxyphenyl)-2-naphthol; 6-(2-fluoro-4-hydroxyphenyl)-2-naphthol; 6-(2,5-difluoro-4-hydroxyphenyl)-2-naphthol; 6-(2,6-difluoro-4-hydroxyphenyl)-2-naphthol; 1-chloro-6-(2-fluoro-4-hydroxyphenyl)-2-naphthol; 1-chloro-6-(2,5-difluoro-4-hydroxyphenyl)-2-naphthol; 1-chloro-6-(2,6-difluoro-4-hydroxyphenyl)-2-naphthol; 8-fluoro-6-(4-hydroxyphenyl)-2-naphthol; 1-chloro-8-fluoro-6-(4-hydroxyphenyl)-2-naphthol; 8-chloro-6-(4-hydroxyphenyl)-2-naphthol; 1,5-dichloro-8-fluoro-6-(4-hydroxyphenyl)-2-naphthol; 2-chloro-4-(2-naphthyl)phenol; 3-bromo-8-chloro-6-(4-hydroxyphenyl)-2-naphthol; 1,8-dichloro-6-(4-hydroxyphenyl)-2-naphthol; 3-bromo-1,8-dichloro-6-(4-hydroxyphenyl)-2-naphthol; 7-hydroxy-3-(4-hydroxyphenyl)-1-naphthonitrile; 8-chloro-3-(4-hydroxyphenyl)-7-hydroxy-1-naphthonitrile; 8-chloro-3-(3-fluoro-4-hydroxyphenyl)-7-hydroxy-1-naphthonitrile; 6-(3,5-difluoro-4-hydroxyphenyl)-2-naphthol; 1-chloro-6-(3,5-difluoro-4-hydroxyphenyl)-2-naphthol; 8-bromo-7-hydroxy-3-(4-hydroxyphenyl)-1-naphthonitrile; and a pharmaceutically acceptable salt thereof.

In some further embodiments, at least one of said one or more estrogenic agents is selected from: [5-Hydroxy-2-(4-hydroxy-phenyl)-benzofuran-7-yl]-acetonitrile; [5-Methoxy-2-(4-methoxy-phenyl)-benzofuran-7-yl]-acetonitrile; 2-[5-Methoxy-2-(4-methoxy-phenyl)-benzofuran-7-yl]-propionitrile; 2-[5-Methoxy-2-(4-methoxy-phenyl)-benzofuran-7-yl]-2-methyl-propionitrile; 2-[5-Hydroxy-2-(4-hydroxy-phenyl)-benzofuran-7-yl]-propionitrile; 2-[5-Hydroxy-2-(4-hydroxyphenyl)-benzofuran-7-yl]-2-methyl-propionitrile; [2-(3-Fluoro-4-hydroxy-phenyl)-5-hydroxybenzofuran-7-yl]-acetonitrile; 3-[5-Hydroxy-2-(4-hydroxy-phenyl)-benzofuran-7-yl]-propionitrile; 2-(4-Hydroxy-phenyl)-7-methoxy-benzofuran-5-ol; 4-Chloro-2-(4-hydroxy-phenyl)-7-methoxy-benzofuran-5-ol; 4-Bromo-2-(4-hydroxy-phenyl)-7-methoxy-benzofuran-5-ol; 5-Hydroxy-2-(4-hydroxy-phenyl)-7-methoxy-benzofuran-4-carbaldehyde; 5-Hydroxy-2-(4-hydroxy-phenyl)-7-methoxy-benzofuran-4-carbonitrile; 2-(3-Fluoro-4-hydroxy-phenyl)-5-hydroxy-7-methoxy-benzofuran-4-carbonitrile; and a pharmaceutically acceptable salt thereof.

In some further embodiments, at least one of said one or more estrogenic agents is selected from: 2-Hydroxy-3-iodo-5-methoxy-benzaldehyde; 5-Methoxy-2-(4-methoxy-phenyl)-benzofuran-7-carbaldehyde; [5-Methoxy-2-(4-methoxy-phenyl)-benzofuran-7-yl]-methanol; 7-Bromomethyl-2-(4-hydroxy-phenyl)-benzofuran-5-ol; 2-(4-Hydroxy-phenyl)-7-methoxymethyl-benzofuran-5-ol; 7-Ethoxymethyl-2-(4-hydroxy-phenyl)-benzofuran-5-ol; 2-(4-Hydroxy-phenyl)-7-isopropoxymethyl benzofuran-5-ol; 2-(4-Hydroxy-phenyl)-7-methyl-benzofuran-5-ol; 2-(4-Hydroxy-phenyl)-7-methylsulfanylmethyl-benzofuran-5-ol; 7-Ethylsulfanylmethyl-2-(4-hydroxyphenyl)-benzofuran-5-ol; 2-(4-Hydroxy-phenyl)-7-phenylsulfanylmethyl-benzofuran-5-ol; 2-(4-Hydroxy-phenyl)-7-methanesulfinylmethyl-benzofuran-5-ol; 2-(4-Hydroxy-phenyl)-7-methanesulfonylmethyl-benzofuran-5-ol; 2-(4-Hydroxy-phenyl)-7-thiocyanatomethyl-benzofuran-5-ol; 2-(4-Hydroxy-phenyl)-7-imidazol-1-ylmethyl-benzofuran-5-ol; 7-Bromomethyl-5-methoxy-2-(4-methoxy-phenyl)-benzofuran; 5-Methoxy-2-(4-methoxy-phenyl)-benzofuran-7-carboxylic acid; 5-Hydroxy-2-(4-hydroxy-phenyl)-benzofuran-7-carboxylic acid; 7-Hydroxymethyl-2-(4-hydroxyphenyl)-benzofuran-5-ol; 5-Methoxy-2-(4-methoxy-phenyl)-benzofuran-7-carbaldehyde oxime; 5-Methoxy-2-(4-methoxy-phenyl)-7-vinyl-benzofuran; 7-Ethyl-5-methoxy-2-(4-methoxy-phenyl)benzofuran; 7-Ethyl-2-(4-hydroxy-phenyl)-benzofuran-5-ol; 7-(2,2-Dichloro-vinyl)-5-methoxy-2-(4-methoxy-phenyl)-benzofuran; 7-(2,2-Dichlorovinyl)-5-hydroxy-2-(4-hydroxyphenyl)benzofuran-5-ol; 5-Methoxy-2-(4-methoxy-phenyl)-7-propenyl-benzofuran; 5-Methoxy-2-(4-methoxy-phenyl)-7-propyl-benzofuran; 2-(4-Hydroxy-phenyl)-7-propyl-benzofuran-5-ol; 5-Hydroxy-2-(4-hydroxy-phenyl)-benzofuran-7-carboxylic acid isopropyl ester; 5-Hydroxy-2-(4-hydroxy-phenyl)-benzofuran-7-carboxylic acid propyl ester; 5-Hydroxy-2-(4-hydroxy-phenyl)-benzofuran-7-carboxylic acid ethyl ester; 2-(3-Fluoro-4-methoxy-phenyl)-5-methoxy-benzofuran-7-carboxylic acid methyl ester; [2-(3-Fluoro-4-methoxy-phenyl)-5-methoxy-benzofuran-7-yl]-methanol; 7-Bromomethyl-2-(3-fluoro-4-hydroxy-phenyl)-benzofuran-5-ol; 2-(3-Fluoro-4-methoxy-phenyl)-5-methoxy-benzofuran-7-carboxylic acid; 2-(3-Fluoro-4-methoxyphenyl)-5-methoxy-benzofuran-7-carboxylic acid methoxy-methyl-amide; 2-(3-Fluoro-4-methoxy-phenyl)-5-methoxy-benzofuran-7-carbaldehyde; 2-(3-Fluoro-4-methoxy-phenyl)-5-methoxy-benzofuran-7-carbaldehyde oxime; 2-(3-Fluoro-4-hydroxy-phenyl)-5-hydroxybenzofuran-7-carbonitrile; 2-(3-Fluoro-4-hydroxy-phenyl)-7-methyl-benzofuran-5-ol; 3-Bromo-2-hydroxy-5-methoxy-benzaldehyde; 3-Bromo-2,5-dimethoxy-benzaldehyde; (3-Bromo-2,5-dimethoxy-phenyl)-methanol; 1-Bromo-3-chloromethyl-2,5-dimethoxy-benzene; (3-Bromo-2,5-dimethoxy-phenyl)-acetonitrile; (3-Bromo-2,5-dimethoxy-phenyl)-acetic acid; 2-(3-Bromo-2,5-dimethoxy-phenyl)-1-(4-methoxy-phenyl)-ethanone; 7-Chloro-2-(4-hydroxy-phenyl)-benzofuran-5-ol; 7-Bromo-2-(4-hydroxy-phenyl)-benzofuran-5-ol; 3-[5-Hydroxy-2-(4-hydroxyphenyl)benzofuran-7-yl]-acrylic acid methyl ester; 3-[5-Hydroxy-2-(4-hydroxy-phenyl)benzofuran-7-yl]-propionic acid methyl ester; 3-[5-Hydroxy-2-(4-hydroxyphenyl)-benzofuran-7-yl]-acrylamide; 4,7-Dibromo-2-(4-hydroxy-phenyl)-benzofuran-5-ol; 3-[5-Hydroxy-2-(4-hydroxyphenyl)-benzofuran-7-yl]-acrylonitrile; 7-Bromo-5-(tert-butyl-dimethyl-silanyloxy)-2-[4-(tert-butyldimethyl-silanyloxy)-phenyl]-benzofuran; 2-(4-Hydroxy-phenyl)-7-vinyl-benzofuran-5-ol; 5-Hydroxy-2-(4-hydroxy-phenyl)-7-methoxy-benzofuran-4-carbaldehyde oxime; 2-(2,5-Dimethoxyphenyl)-1-(4-methoxy-phenyl)-ethanone; 2-(4-Hydroxy-phenyl)-benzofuran-5-ol; 2-(2,5-Dimethoxy-phenyl)-1-(2-fluoro-4-methoxy-phenyl)-ethanone; 2-(2-Fluoro-4-hydroxy-phenyl)-benzofuran-5-ol; 5-OMe-Benzofuran 2-boronic acid; 4-(5-Methoxy-benzofuran-2-yl)-3-methyl-phenol; 2-(4-Hydroxy-2-methyl-phenyl)-benzofuran-5-ol; 5-Bromo-2-(4-methoxy-phenyl)benzofuran; 5-Chloro-2-(4-methoxy-phenyl)-benzofuran; 5-Fluoro-2-(4-methoxy-phenyl)benzofuran; 5-tert-Butyl-2-(4-methoxy-phenyl)-benzofuran; 5,7-Dichloro-2-(4-methoxy-phenyl)benzofuran; 5,7-Difluoro-2-(4-methoxy-phenyl)-benzofuran; 5,7-Dibromo-2-(4-methoxy-phenyl)benzofuran; 2-(4-Methoxy-phenyl)-5-trifluoromethyl-benzofuran; 4-(5-Bromo-benzofuran-2-yl)phenol; 4-(5-Chloro-benzofuran-2-yl)-phenol; 4-(5-Fluoro-benzofuran-2-yl)-phenol; 4-(5-tert-Butyl-benzofuran-2-yl)-phenol; 4-(5,7-Dichloro-benzofuran-2-yl)-phenol; 4-(5,7-Difluoro-benzofuran-2-yl)-phenol; 4-(5,7-Dibromo-benzofuran-2-yl)-phenol; 4-(5-Trifluoromethyl-benzofuran-2-yl)-phenol; 2-Iodo-4-methoxy-6-nitro-phenol; 5-Methoxy-2-(4-methoxy-phenyl)-7-nitro-benzofuran; 2-(4-Hydroxy-phenyl)-7-nitro-benzofuran-5-ol; 7-Amino-2-(4-hydroxy-phenyl)benzofuran-5-ol; 1-(2-Bromo-4-methoxyphenyl)-2-(2,5-dimethoxy-phenyl)-ethanone; 2-(2-Bromo-4-hydroxy-phenyl)-benzofuran-5-ol; 2-(5-Hydroxy-biphenyl-2-yl)-benzofuran-5-ol; 2-(4′-Benzyloxy-5-hydroxy-biphenyl-2-yl)-benzofuran-5-ol; 6-(5-Hydroxy-benzofuran-2-yl)-biphenyl-3,4′-diol; 2-[5-Hydroxy-4′-(2-pyrrolidin-1-yl-ethoxy)-biphenyl-2-yl]-benzofuran-5-ol; 2,2-Dimethyl-propionic acid 2-[4-(2,2-dimethyl-propionyloxy)-phenyl]-benzofuran-5-yl ester; 1-[5-Hydroxy-2-(4-hydroxy-phenyl)-benzofuran-3-yl]-ethanone; 1-[5-Hydroxy-2-(4-hydroxy-phenyl)-benzofuran-3-yl]-ethanone oxime; 3-(1-Hydroxy-ethyl)-2-(4-hydroxy-phenyl)-benzofuran-5-ol; 2-[5-Methoxy-2-(4-methoxy-phenyl)-benzofuran-7-yl]-propan-2-ol; 7-Isopropenyl-5-methoxy-2-(4-methoxyphenyl)-benzofuran; 7-Isopropyl-5-methoxy-2-(4-methoxy-phenyl)-benzofuran; 2-(4-Hydroxyphenyl)-7-isopropyl-benzofuran-5-ol; 5-Hydroxy-2-(4-hydroxy-phenyl)-benzofuran-7-carboxylic acid methoxy-methyl-amide; 5-Hydroxy-2-(4-hydroxy-phenyl)-benzofuran-7-carbaldehyde; 5-Methoxy-2-(4-methoxy-phenyl)-benzofuran-7-carboxylic acid methoxy-methyl-amide; 1-[5-Methoxy-2-(4-methoxy-phenyl)-benzofuran-7-yl]-ethanone; 1-[5-Hydroxy-2-(4-hydroxy-phenyl)benzofuran-7-yl]-ethanone; 1-[5-Hydroxy-2-(4-hydroxy-phenyl)-benzofuran-7-yl]-propan-1-one; 2-(2,5-Dimethoxy-phenyl)-1-(3-fluoro-4-methoxy-phenyl)-ethanone; 2-(3-Fluoro-4-hydroxyphenyl)-benzofuran-5-ol; 4-(5-Methoxy-benzofuran-2-yl)-benzoic acid methyl ester; 4-(5-Hydroxy-benzofuran-2-yl)-benzoic acid; 2-(4-Hydroxymethyl-phenyl)-benzofuran-5-ol; 4-Bromo-2-(4-hydroxy-phenyl)-benzofuran-5-ol; 4-Chloro-2-(4-hydroxy-phenyl)-benzofuran-5-ol; 2-(4-Hydroxy-phenyl)-4-methoxy-benzofuran-5-ol; 4-Bromo-5-methoxy-2-(4-methoxy-phenyl)benzofuran; 5-Methoxy-2-(4-methoxy-phenyl)-benzofuran-4-carbonitrile; 5-Hydroxy-2-(4-hydroxy-phenyl)-benzofuran-4-carbonitrile; 5-Methoxy-2-(4-methoxy-phenyl)-4-methyl-benzofuran; 2-(4-Hydroxy-phenyl)-4-methyl-benzofuran-5-ol; 2-(4-Hydroxy-phenyl)-7-[1,3,4]oxadiazol-2-yl-benzofuran-5-ol; 2,2,2-Trifluoro-1-[5-methoxy-2-(4-methoxy-phenyl)benzofuran-7-yl]-ethanol; 5-Methoxy-2-(4-methoxyphenyl)-7-(2,2,2-trifluoroethyl)-benzofuran; 2-(4-Hydroxy-phenyl)-7-(2,2,2-trifluoro-ethyl)-benzofuran-5-ol; 2-(4-Methoxy-phenyl)-benzofuran-5-carboxylic acid methyl ester; 2-(4-Hydroxy-phenyl)-benzofuran-5-carboxylic acid; 4-(5-Hydroxymethyl-benzofuran-2-yl)-phenol; and a pharmaceutically acceptable salt thereof.

In some further embodiments, at least one of said one or more estrogenic agents is selected from: 3,9-Dimethoxy-6H-chromeno[4,3-b]quinolin-7-ol; 7-Chloro-3,9-dimethoxy-6H-chromeno[4,3-b]quinoline; 7-Bromo-3,9-dimethoxy-6H-chromeno[4,3-b]quinoline; 7-Chloro-3,9-dihydroxy-6H-chromeno[4,3-b]quinoline; 7-Bromo-3,9-dihydroxy-6H-chromeno[4,3-b]quinoline; 3,9-Dihydroxy-7-vinyl-6H-chromeno[4,3-b]quinoline; 3,9-Dihydroxy-7-[(trimethylsilyl)ethynyl]-6H-chromeno[4,3-b]quinoline; 3,9-Dihydroxy-7-ethynyl-6H-chromeno[4,3-b]quinoline; 3,9-Dihydroxy-7-ethyl-6H-chromeno[4,3-b]quinoline; 7-Cyano-3,9-dihydroxy-6H-chromeno[4,3-b]quinoline; 7-(4-Chlorophenyl)-3,9-dihydroxy-6H-chromeno[4,3-b]quinoline; 7-(4-Cyanophenyl)-3,9-dihydroxy-6H-chromeno[4,3-b]quinoline; 3,9-Dihydroxy-7-(4-methoxyphenyl)-6H-chromeno[4,3-b]quinoline; 3,9-Dihydroxy-7-[4-(trifluoromethyl)phenyl]-6H-chromeno[4,3-b]quinoline; 7-(3-Chlorophenyl)-3,9-dihydroxy-6H-chromeno[4,3-b]quinoline; 3,9-Dihydroxy-7-(3-methoxyphenyl)-6H-chromeno[4,3-b]quinoline; 7-(3-Cyanophenyl)-3,9-dihydroxy-6H-chromeno[4,3-b]quinoline; and a pharmaceutically acceptable salt, chelate, complex, or prodrug thereof.

In some further embodiments, at least one of said one or more estrogenic agents is selected from: 5,6-Dihydro-benzo[b]naphtho[2,1-d]furan-3,9-diol; Benzo[b]naphtho[2,1-d]furan-3,9-diol; 5-Bromo-benzo[b]naphtho[2,1-d]furan-3,9-diol; 3,8-Dihydroxy-5,6-dihydro-benzo[b]naphtho[2,1-d]furan-10-carbonitrile; 3,9-Dihydroxy-6,7-dihydro-5H-12-oxa-dibenzo[a,e]azulen-11-carbonitrile; 3,9-Dihydroxy-5,6-dihydro-benzo[b]naphtho[2,1-d]furan-10-carbonitrile; 3,9-Dihydroxy-benzo[b]naphtho[2,1-d]furan-10-carbonitrile; 3,8-Dihydroxy-5,5-dimethyl-5,6-dihydro-benzo[b]naphtho[2,1-d]furan-10-carbonitrile; 6H-Benzo[4,5]furo[3,2-c]chromen-3,8-diol; 3,8-Dihydroxy-6H-Benzo[4,5]furo[3,2-c]chromene-10-carbonitrile; 10-Bromo-6H-benzo[4,5]furo[3,2-c]chromene-3,8-diol; 2,9-Dihydroxy-5,6-dihydro-benzo[b]naphtho[2,1-d]furan-10-benzonitrile; 2,9-Dihydroxy-benzo[b]naphtho[2,1-d]furan-10-carbonitrile; and a pharmaceutically acceptable salt thereof.

In some further embodiments, at least one of said one or more estrogenic agents is selected from:

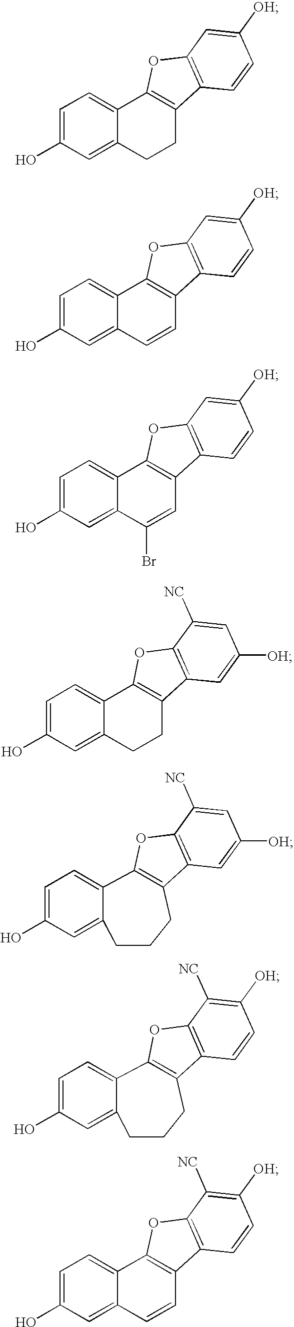

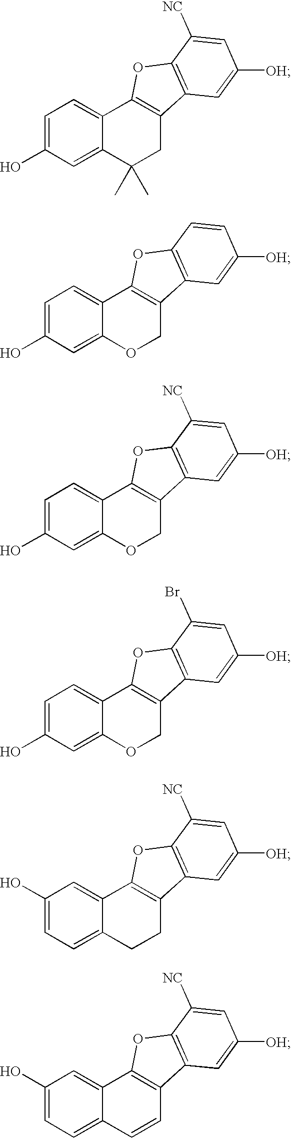

and a pharmaceutically acceptable salt thereof.

In yet further embodiments, at least one of said one or more estrogenic agents is selected from: 6H-Dibenzo[c,h]chromene-2,8-diol; 9-Fluoro-6H-dibenzo[c,h]chromene-2,8-diol; 2,8-Dihydroxy-6H-dibenzo[c,h]chromene-12-carbonitrile; 9-fluoro-2,8-dihydroxy-6H-dibenzo[c,h]chromene-12-carbonitrile; 1-Chloro-2,8-dihydroxy-6H-dibenzo[c,h]chromene-12-carbonitrile; 1-Chloro-6H-dibenzo[c,h]chromene-2,8-diol; 12-Bromo-6H-dibenzo[c,h]chromene-2,8-diol; 12-Chloro-6H-dibenzo[c,h]chromene-2,8-diol; 12-Bromo-9-fluoro-6H-dibenzo[c,h]chromene-2,8-diol; 12-Chloro-9-fluoro-6H-dibenzo[c,h]chromene-2,8-diol; 12-Methoxy-6H-dibenzo[c,h]chromene-2,8-diol; 11-Chloro-12-methoxy-6H-dibenzo[c,h]chromene-2,8-diol; 12-Methyl-6H-dibenzo[c,h]chromene-2,8-diol; 12-Vinyl-6H-dibenzo[c,h]chromene-2,8-diol; 12-Ethyl-6H-dibenzo[c,h]chromene-2,8-diol; 12-Thien-3-yl-6H-dibenzo[c,h]chromene-2,8-diol; 12-Thien-2-yl-6H-dibenzo[c,h]chromene-2,8-diol; 12-Butyl-6H-dibenzo[c,h]chromene-2,8-diol; 12-Propyl-6H-dibenzo[c,h]chromene-2,8-diol; 6H-dibenzo[c,h]chromene-1,8-diol; 12-Chloro-6H-dibenzo[c,h]chromene-1,8-diol; 12-Bromo-6H-dibenzo[c,h]chromene-1,8-diol; 1,8-Dihydroxy-6H-dibenzo[c,h]chromene-12-carbonitrile; 2-Chloro-6H-dibenzo[c,h]chromene-1,8-diol; 4-chloro-6H-dibenzo[c,h]chromene-1,8-diol; 6H-Dibenzo[c,h]chromen-8-ol; and a pharmaceutically acceptable salt thereof.

In some further embodiments, at least one of said one or more estrogenic agents is selected from: 3-(4-Hydroxy-2-methylphenyl)-1,2-benzisoxazol-6-ol; 3-(4-Hydroxy-2,5-dipropylphenyl)-1,2-benzisoxazol-6-ol; 3-(2-Ethyl-4-hydroxy-5-propylphenyl)-1,2-benzisoxazol-6-ol; 3-(4-Hydroxy-2-isobutyl-5-propylphenyl)-1,2-benzisoxazol-6-ol; 3-(4-Hydroxy-3-propylphenyl)-1,2-benzisoxazol-6-ol; 3-(4-Hydroxy-2-propylphenyl)-1,2-benzisoxazol-6-ol; 3-(4-Hydroxy-3-methylphenyl)-1,2-benzisoxazol-6-ol; 3-(4-Hydroxyphenyl)-1,2-benzisoxazol-6-ol; 3-(2-Hydroxyphenyl)-1,2-benzisoxazol-6-ol; 2-(6-Hydroxy-1,2-benzisoxazol-3-yl)benzene-1,4-diol; 4-(6-Hydroxy-1,2-benzisoxazol-3-yl)benzene-1,3-diol; 3-(4-Hydroxyphenyl)-1,2-benzisoxazol-5-ol; 7-Bromo-3-(4-hydroxyphenyl)-1,2-benzisoxazol-5-ol; 3-(3-Bromo-4-hydroxyphenyl)-1,2-benzisoxazol-6-ol; 3-(3-Chloro-4-hydroxyphenyl)-1,2-benzisoxazol-6-ol; 3-(3-Fluoro-4-hydroxyphenyl)-1,2-benzisoxazol-6-ol; 4-Bromo-6-(6-hydroxy-1,2-benzisoxazol-3-yl)benzene-1,3-diol; 4-Chloro-6-(6-hydroxy-1,2-benzisoxazol-3-yl)benzene-1,3-diol; 4-(6-Hydroxy-1,2-benzisoxazol-3-yl)-6-methylbenzene-1,3-diol; 4-(6-Hydroxy-1,2-benzisoxazol-3-yl)-2-methylbenzene-1,3-diol; and a pharmaceutically acceptable salt thereof.

In some embodiments, at least one of said one or more estrogenic agents is selected from: 2-(6-Hydroxy-1-naphthyl)-1,3-benzoxazol-5-ol; 2-(6-Hydroxy-1-naphthyl)-1,3-benzoxazol-6-ol; 2-(6-Hydroxy-2-naphthyl)-1,3-benzoxazol-5-ol; 2-(6-Hydroxy-2-naphthyl)-1,3-benzoxazol-6-ol; 2-(5-Hydroxy-1-naphthyl)-1,3-benzoxazol-6-ol; 2-(4-Hydroxy-1-naphthyl)-1,3-benzoxazol-5-ol; 2-(4-Hydroxy-1-naphthyl)-1,3-benzoxazol-6-ol; 2-(5-Bromo-6-hydroxy-1-naphthyl)-1,3-benzoxazol-6-ol; 3-(6-Hydroxy-2-naphthyl)-1,2-benzisoxazol-6-ol; 3-(6-Hydroxy-2-naphthyl)-1,2-benzisoxazol-5-ol; 3-(5-Hydroxy-2-naphthyl)-1,2-benzisoxazol-6-ol; 3-(5-Hydroxy-1-naphthyl)-1,2-benzisoxazol-6-ol; 3-(6-Hydroxy-1-naphthyl)-1,2-benzisoxazol-6-ol; 3-(7-Hydroxy-1-naphthyl)-1,2-benzisoxazol-6-ol; 3-(5-Bromo-6-hydroxy-2-naphthyl)-1,2-benzisoxazol-6-ol; 3-(6-Hydroxy-2-naphthyl)-1-methyl-1H-indazol-6-ol; 1-Ethyl-3-(6-hydroxy-2-naphthyl)-1H-indazol-6-ol; and a pharmaceutically acceptable salt thereof.

In some embodiments, at least one of said one or more estrogenic agents is selected from: 3-amino-5-hydroxy-2-(4-hydroxyphenyl)-1H-inden-1-one; 5-hydroxy-2-(4-hydroxyphenyl-1H-indene-1,3(2H)-dione; 3-bromo-6-hydroxy-2-(4-hydroxyphenyl)-1H-inden-1-one; 3-bromo-5-hydroxy-2-(4-hydroxyphenyl)-1H-inden-1-one; 3-(ethylamino)-6-hydroxy-2-(4-hydroxyphenyl)-1H-inden-1-one; 3-(ethylthio)-6-hydroxy-2-(4-hydroxyphenyl)-1H-inden-1-one; 3-(ethylthio)-5-hydroxy-2-(4-hydroxyphenyl)-1H-inden-1-one; 6-hydroxy-2-(4-hydroxyphenyl)-3-(methylthio)-1H-inden-1-one; 5-hydroxy-2-(4-hydroxyphenyl)-3-(methylthio)-1H-inden-1-one; 6-hydroxy-2-(4-hydroxyphenyl)-3-phenyl-1H-inden-1-one; 5-hydroxy-2-(4-hydroxyphenyl)-3-phenyl-1H-inden-1-one; 6-hydroxy-2-(4-hydroxyphenyl)-3-methyl-1H-inden-1-one; 5-hydroxy-2-(4-hydroxyphenyl)-3-methyl-1H-inden-1-one; 4,6-dihydroxy-2-(4-hydroxyphenyl)-1H-inden-1,3(2H)-dione; 3-bromo-4,6-dihydroxy-2-(4-hydroxyphenyl)-1H-inden-1-one; 3-bromo-6-hydroxy-2-(4-hydroxyphenyl)-4-methoxy-1H-inden-1-one; 5,7-dihydroxy-2-(4-hydroxyphenyl)-3-methyl-1H-inden-1-one; 4,6-dihydroxy-2-(4-hydroxyphenyl)-3-(methylthio)-1H-inden-1-one; 3-(ethylthio)-4,6-Dihydroxy-2-(4-hydroxyphenyl)-1H-inden-1-one; 4,6-dihydroxy-2-(4-hydroxyphenyl)-3-phenyl-1H-inden-1-one; 5,7-dihydroxy-2-(4-hydroxyphenyl)-3-phenyl-1H-inden-1-one; 5,6-dihydroxy-2-(4-hydroxyphenyl)-3-methyl-1H-inden-1-one; and a pharmaceutically acceptable salt thereof.

In some embodiments, at least one of said one or more estrogenic agents is selected from: 5-(3-hydroxyphenyl)-3-(4-hydroxyphenyl)-4-methylthiophene-2-carbaldehyde; 3-(3-hydroxyphenyl)-5-(4-hydroxyphenyl)-4-methylthiophene-2-carbaldehyde; 3-(4-hydroxy-2-methylphenyl)-5-(4-hydroxyphenyl)-4-methyl-2-thiophenecarbaldehyde; 3-(5-hydroxy-2-methylphenyl)-5-(4-hydroxyphenyl)-4-methyl-2-thiophenecarbaldehyde; 3-(3-fluoro-4-hydroxyphenyl)-5-(4-hydroxyphenyl)-4-methyl-2-thiophenecarbaldehyde; 3-(3-formyl-4-hydroxyphenyl)-5-(4-hydroxyphenyl)-4-methyl-2-thiophenecarbaldehyde; 3-(3-chloro-4-hydroxyphenyl)-5-(4-hydroxyphenyl)-4-methylthiophene-2-carbaldehyde; 5-(3-fluoro-4-hydroxyphenyl)-3-(4-hydroxyphenyl)-4-methyl-2-thiophenecarbaldehyde; 5-(3-fluoro-4-hydroxyphenyl)-3-(4-hydroxy-2-methylphenyl)-4-methylthiophene-2-carbaldehyde; 3-(3-chloro-4-hydroxyphenyl)-5-(3-fluoro-4-hydroxyphenyl)-4-methylthiophene-2-carbaldehyde; 5-(4-hydroxy-2-methylphenyl)-3-(4-hydroxyphenyl)-4-methyl-2-thiophenecarbaldehyde; 3-(3-fluoro-4-hydroxyphenyl)-5-(4-hydroxy-2-methylphenyl)-4-methylthiophene-2-carbaldehyde; 5-(3-chloro-4-hydroxyphenyl)-3-(4-hydroxyphenyl)-4-methylthiophene-2-carbaldehyde; 5-(3-chloro-4-hydroxyphenyl)-3-(3-fluoro-4-hydroxyphenyl)-4-methylthiophene-2-carbaldehyde; 3,5-bis(4-hydroxyphenyl)-4-methylthiophene-2-carbaldehyde; 3-(3-hydroxyphenyl)-5-(4-hydroxyphenyl)-4-methylthiophene-2-carbaldehyde oxime; 3-(3-hydroxyphenyl)-5-(4-hydroxyphenyl)-4-methylthiophene-2-carbonitrile; 3,5-bis(4-hydroxyphenyl)-4-methylthiophene-2-carbaldehyde oxime; 3,5-bis(4-hydroxyphenyl)-4-methylthiophene-2-carbonitrile; 3,5-bis(4-hydroxyphenyl)-4-methylthiophene-2-carbaldehyde O-methyloxime; 3-(4-hydroxyphenyl)-5-(3-hydroxyphenyl)-4-methylthiophene-2-carbaldehyde oxime; 3-(4-hydroxyphenyl)-5-(3-hydroxyphenyl)-4-methylthiophene-2-carbonitrile; 3-(3-fluoro-4-hydroxyphenyl)-5-(4-hydroxy-2-methylphenyl)-4-methylthiophene-2-carbaldehyde O-methyloxime; and a pharmaceutically acceptable salt thereof.

In some further embodiments, at least one of said one or more estrogenic agents is selected from: 4-Chloro-2-(4-hydroxyphenyl)quinolin-6-ol; 4-Chloro-2-(3-fluoro-4-hydroxyphenyl)quinolin-6-ol; 8-Bromo-4-chloro-2-(3-fluoro-4-hydroxyphenyl)-quinolin-6-ol; 4-Bromo-2-(4-hydroxyphenyl)quinolin-6-ol; 4-Bromo-2-(3-fluoro-4-hydroxyphenyl)quinolin-6-ol; 4-Bromo-2-(3,5-difluoro-4-hydroxyphenyl)quinolin-6-ol; 4-Cyano-2-(4-hydroxyphenyl)quinolin-6-ol; 4-Cyano-2-(3-fluoro-4-hydroxyphenyl)quinolin-6-ol; 6-Acetoxy-4-bromo-2-(3-fluoro-4-acetoxyphenyl)quinoline; 2-(4-Hydroxyphenyl)-4-iodoquinolin-6-ol; 2-(3-Fluoro-4-hydroxyphenyl)-4-iodoquinolin-6-ol; 2-(3-Fluoro-4-hydroxyphenyl)-4-vinylquinolin-6-ol; 4-Ethyl-2-(3-fluoro-4-hydroxyphenyl)quinolin-6-ol; 2-(4-Hydroxyphenyl)-4-[(trimethylsilyl)ethynyl]quinolin-6-ol; 2-(3-Fluoro-4-hydroxyphenyl)-4-[(trimethylsilyl)ethynyl]quinolin-6-ol; 4-Ethynyl-2-(4-hydroxyphenyl)quinolin-6-ol; 4-Ethynyl-2-(3-fluoro-4-hydroxyphenyl)quinolin-6-ol; 2-(3,5-Difluoro-4-hydroxyphenyl)-4-ethynylquinolin-6-ol; 2-(3-Fluoro-4-hydroxyphenyl)-4-(phenylethynyl)quinolin-6-ol; 4-Acetyl-2-(4-hydroxyphenyl)quinolin-6-ol; 4-Acetyl-2-(3-fluoro-4-hydroxyphenyl)quinolin-6-ol; 4-(1-Hydroxyethyl)-2-(4-hydroxyphenyl)quinolin-6-ol; 2-(3-Fluoro-4-hydroxyphenyl)-4-(1-hydroxyethyl)quinolin-6-ol; 2-(3-Fluoro-4-hydroxyphenyl)-4-thiazol-2-yl-quinolin-6-ol; 2-(3-Fluoro-4-hydroxyphenyl)-4-thiophen-3-yl-quinolin-6-ol; 2-(3-Fluoro-4-hydroxyphenyl)-4-(4-pyridyl)quinolin-6-ol; 2-(3-Fluoro-4-hydroxyphenyl)-4-(5-pyrimidyl)quinolin-6-ol; 5-Chloro-2-(4-hydroxyphenyl)quinolin-6-ol; 5-Bromo-2-(4-hydroxyphenyl)quinolin-6-ol; 4-Bromo-3-chloro-2-(4-hydroxyphenyl)quinolin-6-ol; 4-Bromo-3-chloro-2-(3-fluoro-4-hydroxyphenyl)quinolin-6-ol; 3-Chloro-4-(4-fluorophenyl)-2-(4-hydroxyphenyl)quinolin-6-ol; 3-Chloro-4-Cyano-2-(3-fluoro-4-hydroxyphenyl)quinolin-6-ol; 4-Bromo-8-chloro-2-(3-fluoro-4-hydroxyphenyl)quinolin-6-ol; 4,8-Dibromo-2-(3-fluoro-4-hydroxyphenyl)quinolin-6-ol; 8-Chloro-4-cyano-2-(3-fluoro-4-hydroxyphenyl)quinolin-6-ol; 8-Chloro-4-ethynyl-2-(3-fluoro-4-hydroxyphenyl)quinolin-6-ol; 8-Chloro-2-(3-fluoro-4-hydroxyphenyl)-4-hex-1-ynylquinolin-6-ol; 8-Chloro-2-(3-fluoro-4-hydroxyphenyl)-4-vinylquinolin-6-ol; 8-Chloro-4-(4-cyanophenyl)-2-(3-fluoro-4-hydroxyphenyl)quinolin-6-ol; 8-Chloro-2-(3-fluoro-4-hydroxyphenyl)-4-(4-methoxyphenyl)quinolin-6-ol; 4-Chloro-8-cyano-2-(3-fluoro-4-hydroxyphenyl)quinolin-6-ol; 4-Bromo-8-cyano-2-(3-fluoro-4-hydroxyphenyl)quinolin-6-ol; 8-Cyano-4-ethynyl-2-(3-fluoro-4-hydroxyphenyl)quinolin-6-ol; 4-Chloro-8-(4-cyanophenyl)-2-(3-fluoro-4-hydroxyphenyl)quinolin-6-ol; 8-Cyano-2-(3-fluoro-4-hydroxyphenyl)quinolin-6-ol; 2-(4-Hydroxyphenyl)-4-methoxyquinolin-6-ol; 2-(4-Hydroxyphenyl)-4-vinylquinolin-6-ol; 4-Ethyl-2-(4-hydroxyphenyl)quinolin-6-ol; 2-(3-Fluoro-4-hydroxyphenyl)-4-phenylquinolin-6-ol; 2-(3-Fluoro-4-hydroxylphenyl)-4-(methylphenyl)quinolin-6-ol; 2-(3-Fluoro-4-hydroxyphenyl)-4-(4-fluorophenyl)quinolin-6-ol; 4-(4-Chlorophenyl)-2-(3-fluoro-4-hydroxyphenyl)quinolin-6-ol; 4-(4-Cyanophenyl)-2-(3-fluoro-4-hydroxyphenyl)quinolin-6-ol; 2-(3-Fluoro-4-hydroxyphenyl)-4-(4-trifluoromethylphenyl)quinolin-6-ol; and a pharmaceutically acceptable salt thereof.

In some embodiments, at least one of said one or more estrogenic agents is provided in a transdermal dosage form. In some embodiments, the transdermal dosage form is selected from: a lotion, a cream, an ointment, a foam, a patch, a suspension, a solution, a pledget, a mask, and a suppository.

In some embodiments, the methods of the present invention further comprise: providing to a mammal in need thereof, a therapeutically effective amount of one or more estrogenic agents in combination with one or more therapeutic agents useful in the treatment of inflammatory diseases, disorders, or conditions of the skin, or diseases, disorders, or conditions associated with collagen depletion. In some embodiments, at least one of said one or more therapeutic agents useful in the treatment of inflammatory diseases, disorders, or conditions of the skin, or diseases, disorders, or conditions associated with collagen depletion is tretinoin, retinol, retinaldehyde, alpha tocopherol, monolaurin, alpha hydroxy acids, hyaluronic acid, one or more hyaluronic acid fragments, botulinum toxin (Botox® Cosmetic), an antibiotic, an antihistamine, a barrier-repair moisturizer, coal tar, a corticosteroid, cyclosporine, interferon gamma, mycophenolate mofetil, a topical calcineurin inhibitor, or a dietary supplement.

In some embodiments, said combination of one or more estrogenic agents and one or more therapeutic agents useful in the treatment of inflammatory diseases, disorders, or conditions of the skin, or diseases, disorders, or conditions associated with collagen depletion, is provided in a transdermal dosage form. In some embodiments, said transdermal dosage form is selected from: a lotion, a cream, an ointment, a foam, a patch, a suspension, a solution, a pledget, a mask, and a suppository.

In some further embodiments, the present invention provides a pharmaceutical composition comprising a therapeutically effective amount of one or more estrogenic agents in combination with one or more therapeutic agents useful in the treatment of inflammatory diseases, disorders, or conditions of the skin, or diseases, disorders, or conditions associated with collagen depletion and at least one pharmaceutically acceptable diluent or carrier. In some embodiments, said pharmaceutical composition is provided in a transdermal dosage form. In some embodiments, said transdermal dosage form is selected from: a lotion, a cream, an ointment, a foam, a patch, a suspension, a solution, a pledget, a mask, and a suppository.

Some further embodiments, provide pharmaceutical compositions comprising a therapeutically effective amount of one or more estrogenic agents, wherein at least one of said one or more estrogenic agents is selected from the compounds of Formulae I through XIII described herein; and

one or more therapeutic agents useful in the treatment of inflammatory diseases, disorders, or conditions of the skin, or diseases, disorders, or conditions associated with collagen depletion; and

at least one pharmaceutically acceptable diluent or carrier.

In some embodiments, the disease, disorder, or condition associated with collagen depletion is photo aging. In some embodiments, the disease, disorder, or condition associated with collagen depletion is chronological skin aging. In some embodiments, the disease, disorder, or condition associated with collagen depletion is smoking induced skin aging. In some embodiments, the inflammatory disease, disorder, or condition of the skin is eczema. In some embodiments, inflammatory disease, disorder, or condition of the skin is a type of dermatitis. In some embodiments, the inflammatory disease, disorder, or condition of the skin is psoriasis. In some embodiments, the dermatitis is selected from: cercarial dermatitis, dermatitis herpetiformis, atopic dermatitis, seborrhoeic dermatitis, contact dermatitis, chronic dermatitis, irritant dermatitis, urushiol-induced contact dermatitis, nummular dermatitis, and dyshidrotic dermatitis.

Some embodiments of the present invention provide methods of suppressing MMP1, IL1B, and/or IL8 activity in mammalian epidermal and dermal skin comprising providing to a mammal in need thereof, a therapeutically effective amount of one or more estrogenic agents. In some embodiments, at least one of said one or more estrogenic agents provided to suppress MMP1, IL1B, and/or IL8 activity in mammalian epidermal skin is selected from the compounds of Formulae I-XIII, described herein.

The present invention further provides a compound of any one of Formulae I-XIII as described herein, or a composition as described herein, for use as a medicament for preventing, treating, or inhibiting an inflammatory disease, disorder, or condition of the skin, or a disease, disorder, or condition associated with collagen depletion.

The present invention further provides the use of a compound of any one of Formulae I-XIII as described herein, or a composition as described herein, in the manufacture of a medicament for preventing, treating, or inhibiting an inflammatory disease, disorder, or condition of the skin, or a disease, disorder, or condition associated with collagen depletion.

Some embodiments of the present invention provide uses of a therapeutically effective amount of one or more estrogenic agents, a prodrug thereof, or a pharmaceutically acceptable salt thereof; for the manufacture of a medicament for preventing, treating, or inhibiting an inflammatory disease, disorder, or condition of the skin, or a disease, disorder, or condition associated with collagen depletion. Some further embodiments of the present invention provide uses of a therapeutically effective amount of one or more estrogenic agents, a prodrug thereof, or a pharmaceutically acceptable salt thereof; for the manufacture of a medicament for preventing, treating, or inhibiting photo aging, chronological skin aging, smoking induced skin aging, psoriasis, eczema, or dermatitis.

Further embodiments of the present invention provide uses of a therapeutically effective amount of one or more estrogenic agents, a prodrug thereof, or a pharmaceutically acceptable salt thereof; in combination with one or more therapeutic agents useful in the treatment of inflammatory diseases, disorders, or conditions of the skin, or diseases, disorders, or conditions associated with collagen depletion; for the manufacture of a medicament for preventing, treating, or inhibiting an inflammatory disease, disorder, or condition of the skin, or a disease, disorder, or condition associated with collagen depletion. Some embodiments provide uses of a therapeutically effective amount of one or more estrogenic agents, a prodrug thereof, or a pharmaceutically acceptable salt thereof; in combination with one or more therapeutic agents useful in the treatment of inflammatory diseases, disorders, or conditions of the skin, or diseases, disorders, or conditions associated with collagen depletion; for the manufacture of a medicament for preventing, treating, or inhibiting photo aging, chronological skin aging, smoking induced skin aging, psoriasis, eczema, or dermatitis.

In some embodiments, the present invention provides a method of preventing, treating, or inhibiting an inflammatory disease, disorder, or condition of the skin, or a disease, disorder, or condition associated with collagen depletion, comprising providing to a mammal in need thereof, a therapeutically effective amount of one or more estrogenic agents, wherein at least one of said one or more estrogenic agents is:

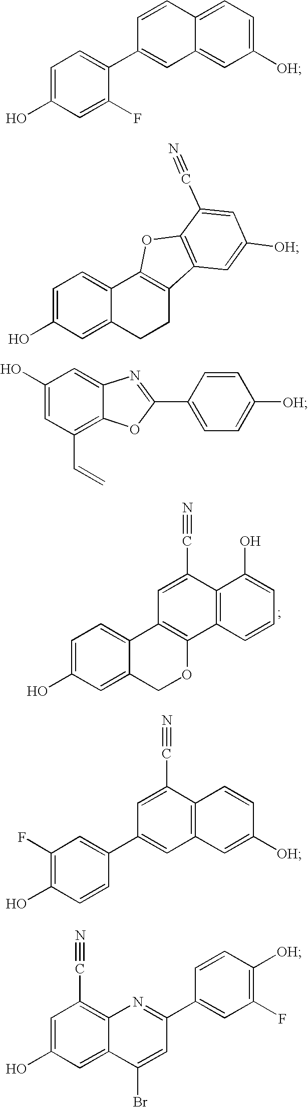

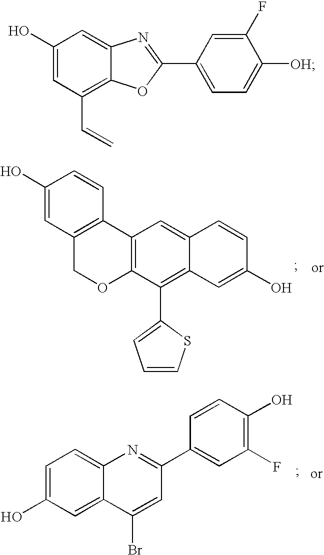

a N-oxide thereof or a pharmaceutically acceptable salt thereof or a prodrug thereof.

In some further embodiments, the present invention provides a pharmaceutical composition comprising a therapeutically effective amount of one or more estrogenic agents, wherein at least one of said one or more estrogenic agents is:

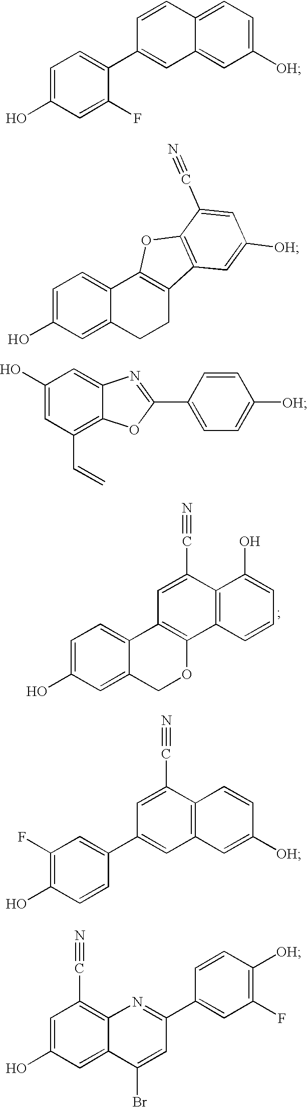

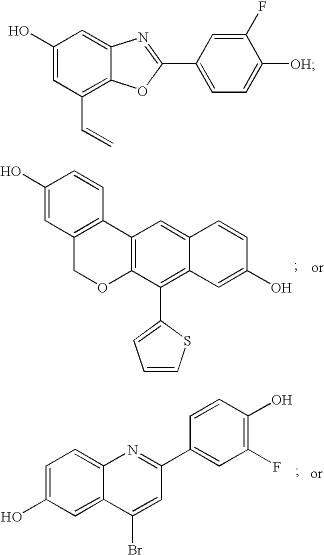

a N-oxide thereof or a pharmaceutically acceptable salt thereof or a prodrug thereof; and at least one pharmaceutically acceptable carrier or diluent.

In yet further embodiments, the present invention provides a pharmaceutical composition comprising a therapeutically effective amount of one or more estrogenic agents, wherein at least one of said one or more estrogenic agents is:

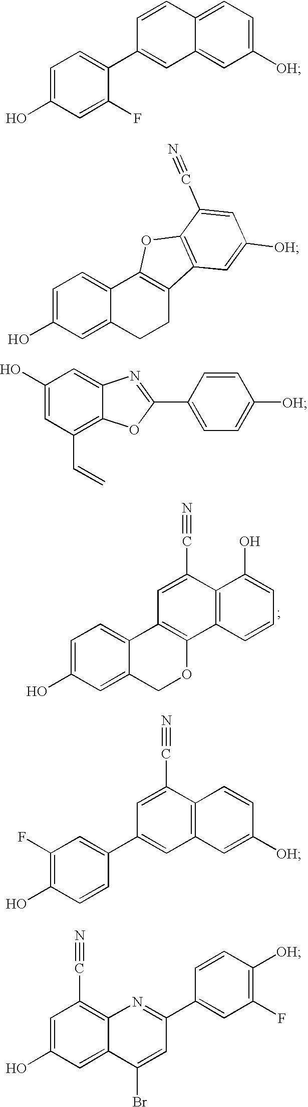

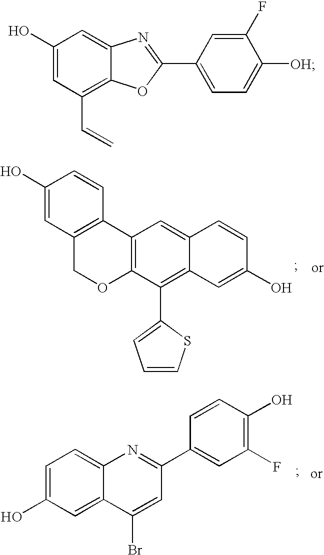

a N-oxide thereof or a pharmaceutically acceptable salt thereof or a prodrug thereof; and one or more therapeutic agents useful in the treatment of inflammatory diseases, disorders, or conditions of the skin, or diseases disorders, or conditions associated with collagen depletion; and at least one pharmaceutically acceptable diluent or carrier.

### DESCRIPTION OF THE FIGURES

FIG. 1 is a graph which depicts the effects an estrogen receptor β ligand on cytokine and matrix metalloproteinase (MMP) expression in a human keratinocyte cell line (KERTr).

FIG. 2 is a graph which depicts dose responses of cytokine and MMP expression in KERTr cells contacted with an estrogen receptor β ligand.

FIG. 3 is a graph which depicts the effects of an estrogen receptor β ligand on cytokine and MMP expression in Normal Human Epidermal Keratinocytes (NHEKs) activated with 60 mJ of UV radiation.

FIG. 4 is a graph which depicts the effects of an estrogen receptor β ligand on cytokine and MMP expression in NHEKs activated with 8 mJ of UV radiation.

FIGS. 5 and 6 are graphs which depict a comparison of the effects of two estrogen receptor β ligands on cytokine and MMP expression in KERTr cells.

FIG. 7 is a graph which depicts the effects an estrogen receptor β ligand on cytokine, collagen type 1-alpha 1 (COL1A1), and tissue inhibitor of metalloproteinase 1 (TIMP1) expression in KERTr cells.

FIGS. 8 and 9 are graphs which depict the effects of a pan-estrogen receptor antagonist (ICI) on cytokines and MMPs in KERTr cells.

FIG. 10 is a graph which depicts the effects of a pan-estrogen receptor antagonist on MMP1 expression in KERTr cells treated with an estrogen receptor β ligand.

FIG. 11 is a graph which depicts the effects of a pan-estrogen receptor antagonist on IL1B expression in KERTr cells treated with an estrogen receptor β ligand.

FIG. 12 is a graph which depicts the relative expression levels of estrogen receptor a in human skin cells.

FIG. 13 is a graph which depicts the relative expression levels of estrogen receptor β in human skin cells.

FIG. 14 is a graph which depicts a comparison of the effects of three estrogen receptor β ligands on cytokines, MMP, and tumor necrosis factor (TNF) expression in KERTr cells.

FIG. 15 is a graph which depicts the effects an estrogen receptor β ligand on MMP1, prostaglandin endperoxide-synthase 2 (PTGS2), COL1A1, and TIMP1 expression in KERTr cells.

FIG. 16 is a graph which depicts the effects an estrogen receptor β ligand on MMPs, TIMP1, and TNF expression in KERTr cells.

FIG. 17 depicts treatment paradigm 1, from which KERTr cell data for an embodiment of the present invention were obtained.

FIG. 18 depicts treatment paradigm 2, from which the NHEK cell data for an embodiment of the present invention were obtained.

FIGS. 19 and 20 depict the results of gene expression studies in a mouse model.

FIG. 21 depicts the results of a murine model of photoaging.

FIG. 22 depicts the effect of an estrogenic agent on UV-induced skin thickness.

FIG. 23 depicts the effects of an estrogenic agent on wrinkle score in an in vivo model.

### DETAILED DESCRIPTION OF THE INVENTION

In vitro epidermal and dermal human skin cell culture systems show that several ER-β selective agonists were capable of effectively suppressing cytokines, chemokines and matrix metallo-proteinases (MMPS) in either UV or TNF-alpha activated skin cells. These in vitro human skin cell culture systems mimic skin photoaging and dermatitis responses, and the treatments of these ER-β agonists counteract many of these responses. The most significant suppressions by these ER-β agonists were found with MMP1, IL1B, and IL8, where more than 5 fold suppressions could be detected in many cases. In proper formulations these compounds could enter the skin topically and provide therapeutic value for inflammatory diseases, disorders, or conditions of the skin, and diseases, disorders, or conditions associated with collagen depletion.

Accordingly, one or more estrogenic agents, particularly ER-β agonists of Formulae II-XIII as described herein, may be used to prevent, treat, or inhibit such diseases, disorders, or conditions in a patient in need of such treatment. In addition, one or more estrogenic agents may be used in combination with one or more therapeutic agents useful in the treatment of inflammatory diseases, disorders, or conditions of the skin, or diseases, disorders, or conditions associated with collagen depletion.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of at least one compound of Formula I, having the structure:

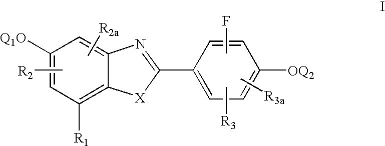

wherein:
Q₁ and Q₂ are independently H, a sugar residue selected from an unmodified or modified hexose residue, or S(O)ₜ—OH, provided that Q₁ and Q₂ are not both H;
t is 0, 1 or 2;
R₁ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₅ and R₆ are each, independently hydrogen, alkyl of 1-6 carbon atoms, or aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅; a pharmaceutically acceptable salt thereof; or
   a gluocuronide, sulfate, or glucuronide-sulfate derivative thereof.

In some embodiments, said at least one compound of Formula I is selected from: 2-(3′-fluoro-4′-glucuronide phenyl)-7-vinyl-1,3-benzoxazol-5-ol; 2-(3′-fluoro-4′-sulfate phenyl)-7-vinyl-1,3-benzoxazol-5-ol; 2-(3′-fluoro-4′-hydroxy phenyl)-7-vinyl-1,3-benzoxazol-5-glucuronide; 2-(3′-fluoro-4′-hydroxy phenyl)-7-vinyl-1,3-benzoxazol-5-sulfate; 2-(3′-fluoro-4′-glucuroride phenyl)-7-vinyl-1,3-benzoxazol-5-glucuronide; 2-(3′-fluoro-4′-glucuronide phenyl)-7-vinyl-1,3-benzoxazol-5-sulfate; 2-(3′-fluoro-4′-sulfate phenyl)-7-vinyl-1,3-benzoxazol-5-glucuronide; 2-(3′-fluoro-4′-sulfate phenyl)-7-vinyl-1,3-benzoxazol-5-sulfate; 2-(2′-fluoro-4′-glucuronide phenyl)-7-vinyl-1,3-benzoxazol-5-ol; 2-(2′-fluoro-4′-sulfate phenyl)-7-vinyl-1,3-benzoxazol-5-ol, 2-(2′-fluoro-4′-hydroxy phenyl)-7-vinyl-1,3-benzoxazol-5-glucuronide; 2-(2′-fluoro-4′-hydroxy phenyl)-7-vinyl-1,3-benzoxazol-5-sulfate; 2-(2′-fluoro-4′-glucuronide phenyl)-7-vinyl-1,3-benzoxazol-5-glucuronide; 2-(2′-fluoro-4′-glucuronide phenyl)-7-vinyl-1,3-benzoxazol-5-sulfate; 2-(2′-fluoro-4′-sulfate phenyl)-7-vinyl-1,3-benzoxazol-5-glucuronide; 2-(2′-fluoro-4′-sulfate phenyl)-7-vinyl-1,3-benzoxazol-5-sulfate; 2-(2′,3′-difluoro-4′-glucuronide phenyl)-7-vinyl-1,3-benzoxazol-5-ol; 2-(2′,3′-difluoro-4′-sulfate phenyl)-7-vinyl-1,3-benzoxazol-5-ol; 2-(2′,3′-difluoro-4′-hydroxyphenyl)-7-vinyl-1,3-benzoxazol-5-glucuronide; 2-(2′,3′-difluoro-4′-hydroxyphenyl)-7-vinyl-1,3-benzoxazol-5-sulfate; 2-(2′,3′-difluoro-4′-glucuronide phenyl)-7-vinyl-1,3-benzoxazol-5-glucuronide; 2-(2′,3′-difluoro-4′-glucuronide phenyl)-7-vinyl-1,3-benzoxazol-5-sulfate; 2-(2′,3′-difluoro-4′-sulfate phenyl)-7-vinyl-1,3-benzoxazol-5-glucuronide; 2-(2′,3′-difluoro-4′-sulfate phenyl)-7-vinyl-1,3-benzoxazol-5-sulfate; 4-bromo-2-(3′-fluoro-4′-glucuronide phenyl)-7-vinyl-1,3-benzoxazol-5-ol; 4-bromo-2-(3′-fluoro-4′-sulfate phenyl)-7-vinyl-1,3-benzoxazol-5-ol; 4-bromo-2-(3′-fluoro-4′-hydroxyphenyl)-7-vinyl-1,3-benzoxazol-5-glucoronide; 4-bromo-2-(3′-fluoro-4′-hydroxyphenyl)-7-vinyl-1,3-benzoxazol-5-sulfate; 4-bromo-2-(3′-fluoro-4′-glucuronide phenyl)-7-vinyl-1,3-benzoxazol-5-glucuronide; 4-bromo-2-(3′-fluoro-4′-glucuronide phenyl)-7-vinyl-1,3-benzoxazol-5-sulfate; 4-bromo-2-(3′-fluoro-4′-sulfate phenyl)-7-vinyl-1,3-benzoxazol-5-glucuronide; 4-bromo-2-(3′-fluoro-4′-sulfate phenyl)-7-vinyl-1,3-benzoxazol-5-sulfate; 4,6-dibromo-2-(3′-fluoro-4′-glucuronide phenyl)-7-vinyl-1,3-benzoxazol-5-ol; 4,6-dibromo-2-(3′-fluoro-4′-sulfate phenyl)-7-vinyl-1,3-benzoxazol-5-ol; 4,6-di bromo-2-(3′-fluoro-4′-hydroxyphenyl)-7-vinyl-1,3-benzoxazol-5-glucuronide; 4,6-dibromo-2-(3′-fluoro-4′-hydroxyphenyl)-7-vinyl-1,3-benzoxazol-5-sulfate; 4,6-dibromo-2-(3′-fluoro-4′-glucuronide phenyl)-7-vinyl-1,3-benzoxazol-5-glucuronide; 4,6-dibromo-2-(3′-fluoro-4′-glucuronide phenyl)-7-vinyl-1,3-benzoxazol-5-sulfate; 4,6-dibromo-2-(3′-fluoro-4′-sulfate phenyl)-7-vinyl-1,3-benzoxazol-5-glucuronide; 4,6-dibromo-2-(3′-fluoro-4′-sulfate phenyl)-7-vinyl-1,3-benzoxazol-5-sulfate; 7-(1-bromovinyl)-2-(2′-fluoro-4′-glucuronide phenyl)-1,3-benzoxazol-5-ol; 7-(1-bromovinyl)-2-(2′-fluoro-4′-sulfate phenyl)-1,3-benzoxazol-5-ol; 7-(1-bromovinyl)-2-(2′-fluoro-4′-hydroxyphenyl)-1,3-benzoxazol-5-glucuronide; 7-(1-bromovinyl)-2-(2′-fluoro-4′-hydroxyphenyl)-1,3-benzoxazol-5-sulfate; 7-(1-bromovinyl)-2-(2′-fluoro-4′-glucuronide phenyl)-1,3-benzoxazol-5-glucuronide; 7-(1-bromovinyl)-2-(2′-fluoro-4′-glucuronide phenyl)-1,3-benzoxazol-5-sulfate; 7-(1-bromovinyl)-2-(2′-fluoro-4′-sulfate phenyl)-1,3-benzoxazol-5-glucuronide; 7-(1-bromovinyl)-2-(2′-fluoro-4′-sulfate phenyl)-1,3-benzoxazol-5-sulfate; 7-(1-bromovinyl)-2-(2′,3′-difluoro-4′-glucuronide phenyl)-1,3-benzoxazol-5-ol; 7-(1-bromovinyl)-2-(2′,3′-difluoro-4′-sulfate phenyl)-1,3-benzoxazol-5-ol; 7-(1-bromovinyl)-2-(2′,3′-difluoro-4′-hydroxyphenyl)-1,3-benzoxazol-5-glucuronide; 7-(1-bromovinyl)-2-(2′,3′-difluoro-4′-hydroxyphenyl)-1,3-benzoxazol-5-sulfate; 7-(1-bromovinyl)-2-(2′,3′-difluoro-4′-glucuronide phenyl)-1,3-benzoxazol-5-glucuronide; 7-(1-bromovinyl)-2-(2′,3′-difluoro-4′-glucuronide phenyl)-1,3-benzoxazol-5-sulfate; 7-(1-bromovinyl)-2-(2′,3′-difluoro-4′-sulfate phenyl)-1,3-benzoxazol-5-glucuronide; 7-(1-bromovinyl)-2-(2′,3′-difluoro-4′-sulfate phenyl)-1,3-benzoxazol-5-sulfate; 7-allyl-2-(3′-fluoro-4′-glucuroinide phenyl)-1,3-benzoxazol-5-ol; 7-allyl-2-(3′-fluoro-4′-sulfate phenyl)-1,3-benzoxazol-5-ol; 7-allyl-2-(3′-fluoro-4′-hydroxyphenyl)-1,3-benzoxazol-5-glucuronide; 7-allyl-2-(3′-fluoro-4′-hydroxyphenyl)-1,3-benzoxazol-5-sulfate; 7-allyl-2-(3′-fluoro-4′-glucuroinide phenyl)-1,3-benzoxazol-5-glucuronide; 7-allyl-2-(3′-fluoro-4′-glucuroinide phenyl)-1,3-benzoxazol-5-sulfate; 7-allyl-2-(3′-fluoro-4′-sulfate phenyl)-1,3-benzoxazol-5-glucuronide; 7-allyl-2-(3′-fluoro-4′-sulfate phenyl)-1,3-benzoxazol-5-sulfate; 2-(3′,5′-difluoro-4′-glucuronide phenyl)-7-vinyl-1,3-benzoxazol-5-ol; 2-(3′,5′-difluoro-4′-sulfate phenyl)-7-vinyl-1,3-benzoxazol-5-ol; 2-(3′,5′-difluoro-4′-hydroxy phenyl)-7-vinyl-1,3-benzoxazol-5-glucuronide; 2-(3′,5′-difluoro-4′-hydroxyphenyl)-7-vinyl-1,3-benzoxazol-5-sulfate; 2-(3′,5′-difluoro-4′-glucuronide phenyl)-7-vinyl-1,3-benzoxazol-5-glucuronide; 2-(3′,5′-difluoro-4′-glucuronide phenyl)-7-vinyl-1,3-benzoxazol-5-sulfate; 2-(3′,5′-difluoro-4′-sulfate phenyl)-7-vinyl-1,3-benzoxazol-5-glucuronide; 2-(3′,5′-difluoro-4′-sulfate phenyl)-7-vinyl-1,3-benzoxazol-5-sulfate; 2-(3′-fluoro-4′-glucuronide phenyl)-7-(1-fluorovinyl)-1,3-benzoxazol-5-ol; 2-(3′-fluoro-4′-sulfate phenyl)-7-(1-fluorovinyl)-1,3-benzoxazol-5-ol; 2-(3′-fluoro-4′-hydroxyphenyl)-7-(1-fluorovinyl)-1,3-benzoxazol-5-glucuronide; 2-(3′-fluoro-4′-hydroxyphenyl)-7-(1-fluorovinyl)-1,3-benzoxazol-5-sulfate; 2-(3′-fluoro-4′-glucuronide phenyl)-7-(1-fluorovinyl)-1,3-benzoxazol-5-glucuronide; 2-(3′-fluoro-4′-glucuronide phenyl)-7-(1-fluorovinyl)-1,3-benzoxazol-5-sulfate; 2-(3′-fluoro-4′-sulfate phenyl)-7-(1-fluorovinyl)-1,3-benzoxazol-5-glucuronide; 2-(3′-fluoro-4′-sulfate phenyl)-7-(1-fluorovinyl)-1,3-benzoxazol-5-sulfate; and a pharmaceutically acceptable salt thereof.

Some further embodiments of the present invention provide methods of preventing, treating, or inhibiting an inflammatory disease, disorder, or condition of the skin, or a disease, disorder, or condition associated with collagen depletion, comprising: providing to a mammal in need thereof, a therapeutically effective amount of one or more one estrogenic agents, wherein at least one of said one or more estrogenic agents is selected from a gluocuronide derivative, a sulfate derivative, or a glucuronide-sulfate derivative of: 2-(5-hydroxy-1,3-benzoxazol-2-yl)benzene-1,4-diol; 3-(5-hydroxy-1,3-benzoxazol-2-yl)benzene-1,2-diol; 2-(3-fluoro-4-hydroxyphenyl)-1,3-benzoxazol-5-ol; 2-(3-chloro-4-hydroxyphenyl)-1,3-benzoxazol-5-ol; 2-(2-chloro-4-hydroxyphenyl)-1,3-benzoxazol-5-ol; 2-(3-fluoro-4-hydroxyphenyl)-1,3-benzoxazol-6-ol; 2-(3-tert-butyl-4-hydroxyphenyl)-1,3-benzoxazol-6-ol; 2-(6-hydroxy-1,3-benzoxazol-2-yl)benzene-1,4-diol; 3-(6-hydroxy-1,3-benzoxazol-2-yl)benzene-1,2-diol; 4-(6-hydroxy-1,3-benzoxazol-2-yl)benzene-1,2-diol; 2-(3-chloro-4-hydroxyphenyl)-1,3-benzoxazol-6-ol; 4-(5-hydroxy-1,3-benzoxazol-2-yl)benzene-1,3-diol; 4-(6-hydroxy-1,3-benzoxazol-2-yl)benzene-1,3-diol; 6-chloro-2-(3-fluoro-4-hydroxyphenyl)-1,3-benzoxazol-5-ol; 6-bromo-2-(3-fluoro-4-hydroxyphenyl)-1,3-benzoxazol-5-ol; 6-chloro-2-(4-hydroxyphenyl)-1,3-benzoxazol-5-ol; 5-chloro-2-(4-hydroxyphenyl)-1,3-benzoxazol-6-ol; 7-bromo-2-(3-fluoro-4-hydroxyphenyl)-1,3-benzoxazol-5-ol; 7-bromo-2-(2-fluoro-4-hydroxyphenyl)-1,3-benzoxazol-5-ol; 7-bromo-2-(2,3-difluoro-4-hydroxyphenyl)-1,3-benzoxazol-5-ol; 2-(4-hydroxyphenyl)-7-vinyl-1,3-benzoxazol-5-ol; 7-(1,2-dibromoethyl)-2-(4-hydroxyphenyl)-1,3-benzoxazol-5-ol; 7-(1-bromovinyl)-2-(4-hydroxyphenyl)-1,3-benzoxazol-5-ol; 7-ethynyl-2-(4-hydroxyphenyl)-1,3-benzoxazol-5-ol; 2-(4-hydroxyphenyl)-7-propyl-1,3-benzoxazol-5-ol; 7-butyl-2-(4-hydroxyphenyl)-1,3-benzoxazol-5-ol; 7-cyclopentyl-2-(4-hydroxyphenyl)-1,3-benzoxazol-5-ol; ethyl 5-hydroxy-2-(4-hydroxyphenyl)-1,3-benzoxazole-7-carboxylate; 2-(4-hydroxyphenyl)-7-phenyl-1,3-benzoxazol-5-ol; 2-(4-hydroxyphenyl)-7-methoxy-1,3-benzoxazol-5-ol; 7-ethyl-2-(4-hydroxyphenyl)-1,3-benzoxazol-5-ol; 7-ethyl-2-(2-ethyl-4-hydroxyphenyl)-1,3-benzoxazol-5-ol; 5-hydroxy-2-(4-hydroxyphenyl)-1,3-benzoxazole-7-carbaldehyde; 7-(hydroxymethyl)-2-(4-hydroxyphenyl)-1,3-benzoxazol-5-ol; 7-(bromomethyl)-2-(4-hydroxyphenyl)-1,3-benzoxazol-5-ol; [5-hydroxy-2-(4-hydroxyphenyl)-1,3-benzoxazol-7-yl]acetonitrile; 7-(1-hydroxy-1-methylethyl)-2-(4-hydroxyphenyl)-1,3-benzoxazol-5-ol; 2-(4-hydroxyphenyl)-7-isopropenyl-1,3-benzoxazol-5-ol; 2-(4-hydroxyphenyl)-7-isopropyl-1,3-benzoxazol-5-ol; 7-bromo-2-(4-hydroxy-3-(trifluoromethyl)phenyl)-1,3-benzoxazol-5-ol; 7-(2-furyl)-2-(4-hydroxyphenyl)-1,3-benzoxazol-5-ol; 2-(3-fluoro-4-hydroxyphenyl)-7-(2-furyl)-1,3-benzoxazol-5-ol; 2-(4-hydroxyphenyl)-7-thien-2-yl-1,3-benzoxazol-5-ol; 2-(4-hydroxyphenyl)-7-(1,3-thiazol-2-yl)-1,3-benzoxazol-5-ol; 2-(3-fluoro-4-hydroxyphenyl)-5-hydroxy-1,3-benzoxazole-7-carbonitrile; 4-bromo-2-(4-hydroxyphenyl)-7-methoxy-1,3-benzoxazol-5-ol; 4,6-dibromo-2-(4-hydroxyphenyl)-7-methoxy-1,3-benzoxazol-5-ol; 7-bromo-2-(3,5-difluoro-4-hydroxyphenyl)-1,3-benzoxazol-5-ol; and a pharmaceutically acceptable salt thereof.

Some further embodiments of the present invention provide methods of preventing, treating, or inhibiting an inflammatory disease, disorder, or condition of the skin, or a disease, disorder, or condition associated with collagen depletion, comprising: providing to a mammal in need thereof, a therapeutically effective amount of one or more estrogenic agents. In some embodiments, at least one of said one or more estrogenic agents is:

a. a compound of Formula II, having the structure:

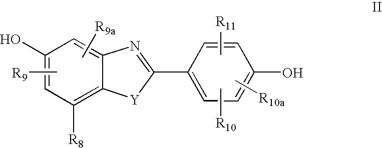

wherein

R₈ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, CONR₁₂R₁₃, NR₁₂R₁₃ or N(R₁₂)COR₁₃;

R₉ and R₉ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₁₂, —CO₂R₁₂, —NO₂, —CONR₁₂R₁₃, —NR₁₂R₁₃ or —N(R₁₂)COR₁₃;

R₁₀, R₁₀ₐ and R₁₁ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;

R₁₂ and R₁₃ are each, independently hydrogen, alkyl of 1-6 carbon atoms, aryl of 6-10 carbon atoms;

### Y is O, S, or NR₁₄;

R₁₄ is hydrogen, alkyl of 1-6 carbon atoms, aryl of 6-10 carbon atoms, —COR₁₂, —CO₂R₁₂ or —SO₂R₁₂;

or a pharmaceutically acceptable salt thereof;

b. a compound of Formula III, having the structure:

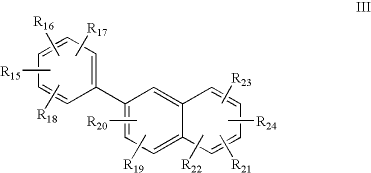

wherein
R₁₅, R₁₆, R₁₇, and R₁₈ are each, independently, selected from hydrogen, hydroxyl, alkyl of 1-6 carbon atoms, alkoxy of 1-6 carbon atoms, or halogen;
R₁₉, R₂₀, R₂₁, R₂₂, R₂₃, and R₂₄, are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₉, R₂₀, R₂₁, R₂₂, R₂₃, or R₂₄ may be optionally substituted with hydroxyl, —CN, halogen, trifluoroalkyl, trifluoroalkoxy, —NO₂, or phenyl; wherein the phenyl moiety of R₁₉, R₂₀, R₂₁, R₂₂, R₂₃, or R₂₄ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
with the proviso that at least one of R₁₅, R₁₆, R₁₇, R₁₈, R₂₁, R₂₂, R₂₃, or R₂₄ is hydroxyl, or a pharmaceutically acceptable salt thereof;
   c. a compound of Formula IV, having the structure:

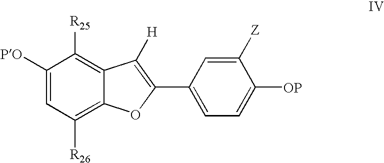

wherein
P and P′ are each, independently, hydrogen, alkyl of 1-6 carbon atoms, or acyl of 2-7 carbon atoms;
Z is hydrogen or halogen;
R₂₅ is hydrogen, alkyl of 1-6 carbon atoms, halogen, —CN, or —CHO;
R₂₆ is alkoxy of 1-6 carbon atoms, or cyanoalkyl having 1-6 carbon atoms in the alkyl moiety; or a pharmaceutically acceptable salt thereof;
   d. a compound of Formula V, having the structure:

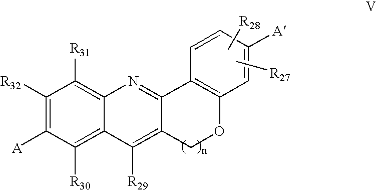

wherein:

A and A′ are each independently OH, H or OU;

each U is independently selected from the group consisting of C₁-C₆ alkyl, alkenyl, benzyl, acyl of 2-7 carbon atoms, aroyl, alkoxycarbonyl, sulfonyl and phosphoryl;

R₂₇ and R₂₈ are independently selected from the group consisting of H, halogen, C₁₋₆ alkyl, C₁₋₆ perhaloalkyl, —CF₃, C₂-C₇ alkenyl and C₁-C₆ alkoxy;

R₂₉, R₃₀, R₃₁ and R₃₂ are each independently selected from the group consisting of H, halogen, —CF₃, C₁₋₆ perhaloalkyl, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl and heteroaryl;

wherein each alkyl or alkenyl moiety of R₂₉, R₃₀, R₃₁ and R₃₂ can be optionally substituted with up to three substituents independently selected from halogen, OH, —CN, trifluoroalkyl of 1-6 carbon atoms, trifluoroalkoxy of 1-6 carbon atoms, —NO₂ and phenyl, wherein said phenyl is optionally substituted with up to three independently selected R₃₃ groups;

wherein each alkynyl moiety of R₂₉, R₃₀, R₃₁ and R₃₂ can be optionally substituted with up to three substituents selected from halogen, —CN, —CHO, acyl, trifluoroalkyl of 1-6 carbon atoms, trialkylsilyl and phenyl, wherein said phenyl is optionally substituted with up to three independently selected R₃₃ groups;

each R₃₃ is independently selected from the group consisting of halogen, C₁-C₆ alkyl, C₂-C₇ alkenyl, —OH, C₁-C₆ alkoxy, —CN, —CHO, —NO₂, amino, C₁-C₆ alkylamino, di-(C₁-C₆)alkylamino, thiol, and C₁-C₆ alkylthio; and

n is 0, 1, 2, or 3;

provided that:

at least one of A and A′ is not H;

if n is 0, then R₂₉ is not halogen; and

at least one of R₂₉, R₃₀ and R₃₁ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;

or a pharmaceutically acceptable salt thereof;

e. a compound of Formula VI, having the structure:

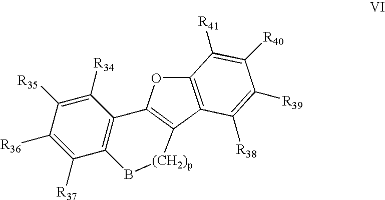

wherein:

B has the structure (i), (ii) or (iii):

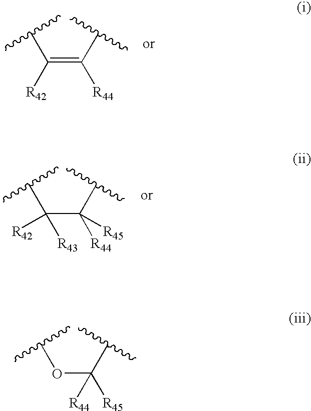

R₃₄, R₃₇, R₃₈, R₄₁, R₄₂, R₄₃, R₄₄, and R₄₅ are each independently selected from the group consisting of hydrogen, C₁-C₆ alkyl, —OR₄₆, halogen, —CF₃, —CF₂CF₃, —CH₂CF₃, —SR₄₆, —NR₄₆R₄₇, —CN, —CH₂CN, —CH₂CH₂CN, —CH═CHCN, —NO₂, —CH₂NO₂, —CH₂CH₂NO₂, —CH═CHNO₂ and —COR₄₆;

p=0 or 1;

each R₄₆ and R₄₇ is independently selected from the group consisting of hydrogen, C₁-C₆ alkyl, —CF₃, benzyl, —CO₂(C₁-C₆ alkyl) and —CO(C₁-C₆ alkyl);

provided that:

a) one of R₃₅ or R₃₆ must be —OR₄₆;

b) one of R₃₉ or R₄₀ must be —OR₄₆;

c) when R₃₅ is —OR₄₆, then R₃₄ and R₃₆ are independently selected from the group consisting of hydrogen, halogen, C₁-C₆ alkyl, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;

d) when R₃₆ is —OR₄₆, then R₃₅ and R₃₇ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;

e) when R₃₉ is —OR₄₆, then R₃₈ and R₄₀ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₃;

f) when R₄₀ is —OR₄₆, then R₃₉ and R₄₁ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆; and

g) when B has the structure (iii), and R₃₈, R₃₉, R₄₁, R₄₄, and R₄₅ are each H, and p=0, then R₄₀ is not —OR₄₆;

f. a compound of Formula VII, having the structure:

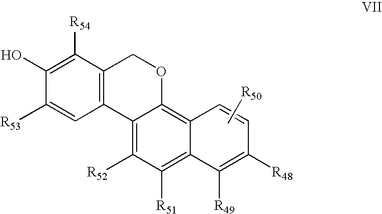

wherein
R₄₈ and R₄₉ are each, independently, selected from hydrogen, hydroxyl, alkyl of 1-6 carbon atoms, alkenyl of 2-7 carbon atoms, alkynyl of 2-7 carbon atoms, alkoxy of 1-6 carbon atoms, or halogen; wherein each alkyl or alkenyl moiety of R₄₈ or R₄₉ may be optionally and independently substituted with hydroxyl, —CN, halogen, trifluoroalkyl of 1-6 carbon atoms, trifluoroalkoxy of 1-6 carbon atoms, —NO₂ or phenyl; and provided that at least one of R₄₈ or R₄₉ is hydroxyl;
R₅₀, R₅₁, R₅₂, R₅₃, and R₅₄ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein each alkyl or alkenyl moiety of R₅₁, R₅₂, R₅₃ or R₅₄ may be optionally and independently substituted with hydroxyl, —CN, halogen, trifluoroalkyl of 1-6 carbon atoms, trifluoroalkoxy of 1-6 carbon atoms, —NO₂ or phenyl; wherein the phenyl moiety of R₅₁ or R₅₂ may be optionally mono-, di-, or tri-substituted with a substituent, the same or different, selected from alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

or a pharmaceutically acceptable salt thereof;

g. a compound of Formula VIII, having the structure:

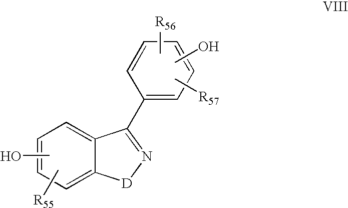

wherein,
R₅₅ and R₅₆ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, trifluoroalkyl of 1-6 carbon atoms, trifluoroalkoxy of 1-6 carbon atoms, alkylthio of 1-6 carbon atoms, or aryl of 6-10 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₇ is hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, alkylthio of 1-6 carbon atoms, trifluoroalkyl of 1-6 carbon atoms, or trifluoroalkoxy of 1-6 carbon atoms, aryl of 6-10 carbon atoms, —NO₂, —CN, —NR₅₈R₅₉ or —N(R₅₈)COR₅₉, —CHFCN, —CF₂CN, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₈ and R₅₉ are each, independently, hydrogen, alkyl of 1-6 carbon atoms, or aryl of 6-10 carbon atoms;
D is O, S, or NR₆₀;
R₆₀ is hydrogen, alkyl of 1-6 carbon atoms, aryl of 6-10 carbon atoms, —COR₅₈, —CO₂R₅₈, or —SO₂R₅₈;
   h. a compound of Formula IX, having the structure:

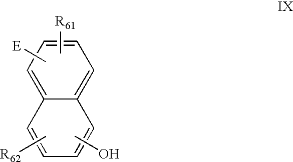

wherein,

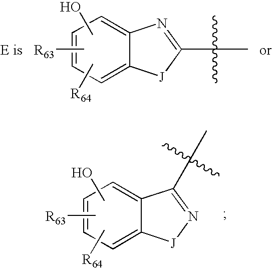

R₆₁, R₆₃, and R₆₄ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₂ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₆₅R₆₆, —N(R₆₅)COR₆₆, —CN, —CHFCN, —CF₂CN, alkynyl of 2-7 carbon atoms, or alkenyl of 2-7 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₅ and R₆₆ are each, independently, hydrogen, alkyl of 1-6 carbon atoms, alkenyl of 2-7 carbon atoms, aryl of 6-10 carbon atoms, —CN, —CHFCN, or —CF₂CN; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₆₇, —CO₂R₆₈, —NO₂, CONR₆₇R₆₈, —NR₆₇R₆₈ or —N(R₆₇)COR₆₈;
R₆₇ and R₆₈ are each, independently, hydrogen, alkyl of 1-6 carbon atoms, or aryl of 6-10 carbon atoms;
J is O, S, or NR₆₉;

R₆₉ is hydrogen, alkyl of 1-6 carbon atoms, aryl of 6-10 carbon atoms, —COR₆₅, —CO₂R₆₅, or —SO₂R₆₅;

or a pharmaceutically acceptable salt thereof;

i. a compound of Formula X, having the structure:

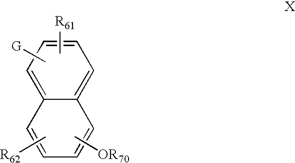

wherein,

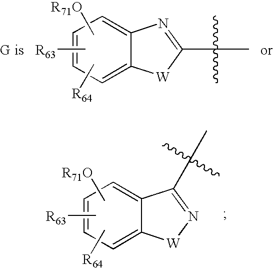

wherein
R₆₁, R₆₂, R₆₃, and R₆₄ are as defined above;
W is O, S, or NR₆₉;
R₆₉ is as defined above; and
R₇₀ and R₇₁ are alkyl of 1-6 carbon atoms;

or a pharmaceutically acceptable salt thereof;

j. a compound of Formula XI, having the structure:

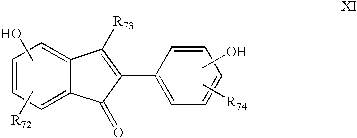

wherein,
R₇₂ is hydrogen, hydroxyl, halogen, trifluoroalkyl of 1-6 carbon atoms, alkyl of 1-6 carbon atoms, cycloalkyl of 3-8 carbon atoms, alkenyl of 2-7 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, —CN, —NO₂, —CHFCN, —CF₂CN, aryl of 6-10 carbon atoms, —NR₇₅R₇₆, —NCOR₇₅, —SR₇₅, —SOR₇₅, —SO₂R₇₅, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₇₅, —CO₂R₇₅, —CONR₇₅R₇₆, or —NR₇₅R₇₆;
R₇₃ is hydrogen, hydroxyl, alkyl of 1-6 carbon atoms, halogen, phenyl substituted with R₁, alkylthio of 1-6 carbon atoms, thioalkyl of 1-6 carbon atoms, amino, aminoalkyl of 1-6 carbon atoms, alkylamino of 1-6 carbon atoms, alkoxy of 1-6 carbon atoms, or alkenyl of 2-7 carbon atoms;
R₇₄ is hydrogen, halogen, hydroxyl, alkyl of 1-6 carbon atoms, alkoxy of 1-6 carbon atoms, trifluoroalkyl of 1-6 carbon atoms, or trifluoroalkoxy of 1-6 carbon atoms;
R₇₅ and R₇₆ are each, independently, hydrogen, alkyl of 1-6 carbon atoms, or aryl of 6-10 carbon atoms;

or a pharmaceutically acceptable salt thereof;

k. a compound of Formula XII, having the structure:

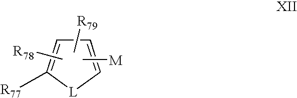

wherein,
R₇₇ is phenyl optionally substituted with 1-4 T groups;
R₇₈ is phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
R₇₉ is hydrogen, phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
L is O, —CH═CH—, or S;
T is —OH, —OR₈₀, halogen, —CN, —CO₂H, —CO₂R₈₀, alkyl of 1-6 carbon atoms, alkenyl of 2-7 carbon atoms, alkynyl of 2-7 carbon atoms, perfluoroalkyl of 1-6 carbon atoms, or —COR₈₀;
M is —CHO, —CN, —CO₂H, —CO₂R₈₀, —CONR₈OR₈₁, —NO₂, —CH═NR₈₀, —CH═NOH, or —CH═NOR₈₀;
R₈₀ and R₈₁ are each, independently, alkyl of 1-6 carbon atoms, alkenyl of 2-7 carbon atoms, alkynyl of 2-7 carbon atoms, or cycloalkyl of 3-8 carbon atoms;
with the proviso that at least one of R₇₈ or R₇₉ is phenyl or phenyl substituted with 1-4 T groups;

or a pharmaceutically acceptable salt thereof;

l. a compound of Formula XIII, having the structure:

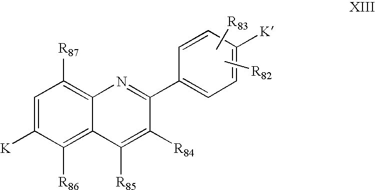

wherein:

K and K′ are each, independently, OH or OV;
V is alkyl, alkenyl, benzyl, acyl of 2-7 carbon atoms, aroyl, alkoxycarbonyl, sulfonyl or phosphoryl;

R₈₂ and R₈₃ are each, independently, H, halogen, C₁-C₆ alkyl, C₂-C₇ alkenyl, or C₁-C₆ alkoxy;

R₈₄ is H, halogen, or C₁-C₆ alkyl;

R₈₅ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, or heteroaryl;

R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₅, R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;

wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;

wherein each alkynyl moiety of R₈₅, R₈₆ or R₈₇ may be optionally substituted with halogen, —CN, —CHO, acyl, trifluoroalkyl of 1-6 carbon atoms, trialkylsilyl, or optionally substituted phenyl;

wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;

provided that when each of R₈₅, R₈₆ and R₈₇ are H, C₁-C₆ alkyl, C₂-C₇ alkenyl, or C₁-C₆ alkoxy, then at least one of R₈₂ and R₈₃ is halogen, C₁-C₆ alkyl, C₂-C₇ alkenyl, or C₁-C₆ alkoxy;

provided that at least one of R₈₅ and R₈₇ is other than H;

or a pharmaceutically acceptable salt thereof;

m. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;

R₈₂ and R₈₃ are each H;

R₈₄ is H, halogen, or C₁-C₆ alkyl;

R₈₅, R₈₆ and R₈₇ are each, independently, H, C₁-C₆ alkyl, C₂-C₇ alkenyl, or C₁-C₆ alkoxy,

provided that at least one of R₈₅, R₈₆ and R₈₇ is C₁-C₆ alkyl, C₂-C₇ alkenyl, or C₁-C₆ alkoxy;

wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;

or a pharmaceutically acceptable salt thereof; or

n. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;

R₈₂ and R₈₃ are each, independently, H, halogen, C₁-C₆ alkyl, C₂-C₇ alkenyl, or C₁-C₆ alkoxy;

R₈₄ is H;

R₈₅ is H, aryl, or substituted aryl;

R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;

wherein each alkyl or alkenyl moiety of R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;

wherein each alkynyl moiety of R₈₆ or R₈₇ may be optionally substituted with halogen, —CN, —CHO, acyl of 2-7 carbon atoms, trifluoroalkyl of 1-6 carbon atoms, trialkylsilyl, or optionally substituted phenyl;

wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;

or a N-oxide thereof or a pharmaceutically acceptable salt thereof or a prodrug thereof.

In some embodiments, at least one of said one or more estrogenic agents is a compound of Formula IIa, having the structure:

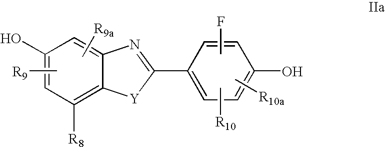

wherein

R₈′ is alkenyl of 2-7 carbon atoms; wherein the alkenyl moiety is optionally substituted with hydroxyl, —CN, halogen, trifluoroalkyl, trifluoroalkoxy, —COR₁₁′, —CO₂R₁₁′, —NO₂, CONR₁₁′R₁₂′, NR₁₁′R₁₂′ or N(R₁₁′)COR₁₂′;

R₉′ and R₉ₐ′ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₁₁′, —CO₂R₁₁′, —NO₂, —CONR₁₁′R₁₂′, —NR₁₁′R₁₂′ or —N(R₁₁′)COR₁₂′;

R₁₀′ and R₁₀ₐ′ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₁₁′, —CO₂R₁₁′, —NO₂, —CONR₁₁′R₁₂′, —NR₁₁′R₁₂′ or —N(R₁₁′)COR₁₂′;

R₁₁′ and R₁₂′ are each, independently hydrogen, alkyl of 1-6 carbon atoms, aryl of 6-10 carbon atoms;

### Y is O, S, or NR₁₃′;

R₁₃′ is hydrogen, alkyl of 1-6 carbon atoms, aryl of 6-10 carbon atoms, —COR₁₁′, —CO₂R₁₁′ or —SO₂R₁₁′;

or a pharmaceutically acceptable salt thereof.

In some embodiments, at least one of said one or more estrogenic agents is a compound of Formula IIIa, having the structure:

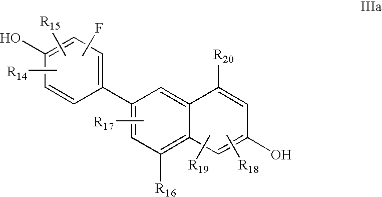

wherein

R₁₄′ and R₁₅′ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₆′, R₁₇′, R₁₈′, R₁₉′, and R₂₀′ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₆′, R₁₇′, R₁₈′, R₁₉′, or R₂₀′ may be optionally substituted with hydroxyl, —CN, halogen, trifluoroalkyl, trifluoroalkoxy, —NO₂, or phenyl; wherein the phenyl moiety of R₁₆′, R₁₇′, R₁₈′, R₁₉′, or R₂₀′ may be optionally mono-, di-, or tri-substituted with alkyl of 1-6 carbon atoms, alkenyl of 2-7 carbon atoms, halogen, hydroxyl, alkoxy of 1-6 carbon atoms, halogen, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

with the proviso that at least one of R₁₆′ or R₂₀′ is not hydrogen;

or a pharmaceutically acceptable salt thereof;

In some embodiments at least one of said one or more estrogenic agents is a compound of Formula II, wherein Y is O. In further embodiments at least one of said one or more estrogenic agents is a compound of Formula II, wherein Y is O; and R₈ is halogen or alkenyl of 2-3 carbon atoms, which is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃, or —N(R₁₂)COR₁₃.

In some embodiments, at least one of said one or more estrogenic agents is a compound of Formula II, wherein R₁₅, R₁₆, R₁₇, and R₁₈ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen; R₁₉, R₂₀, R₂₁, R₂₂, R₂₃, and R₂₄ are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₉, R₂₀, R₂₁, R₂₂, R₂₃, and R₂₄ may be optionally substituted with hydroxyl, —CN, halogen, trifluoroalkyl, trifluoroalkoxy, —NO₂, or phenyl; wherein the phenyl moiety of R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, halogen, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; with the proviso that at least one of R₁₉ or R₂₃ is not hydrogen; and R₂₄ is hydroxyl.

In some further embodiments, at least one of said one or more estrogenic agents is a compound of Formula II, wherein the 5 or 6-membered heterocyclic ring having 1 to 4 heteroatoms selected from O, N or S is furan, thiophene, or pyridine or a pharmaceutically acceptable salt thereof. In other embodiments, at least one of said one or more estrogenic agents is a compound of Formula III wherein the 5 or 6-membered heterocyclic ring having 1 to 4 heteroatoms selected from O, N or S is furan, thiophene, or pyridine; and wherein one of R₁₅, R₁₆, R₁₇, and R₁₈ is hydroxyl, wherein R₁₉, R₂₀, R₂₁, R₂₂ and R₂₃ are each, independently, hydrogen, halogen, —CN, or alkynyl of 2-7 carbon atoms, and wherein R₂₄ is hydroxyl; or a pharmaceutically acceptable salt thereof.

In some embodiments, at least one of said one or more estrogenic agents is selected from: 2-(3-fluoro-4-hydroxyphenyl)-7-vinyl-1,3-benzoxazol-5-ol; 2-(2-fluoro-4-hydroxyphenyl)-7-vinyl-1,3-benzoxazol-5-ol; 2-(2,3-difluoro-4-hydroxyphenyl)-7-vinyl-1,3-benzoxazol-5-ol; 4-bromo-2-(3-fluoro-4-hydroxyphenyl)-7-vinyl-1,3-benzoxazol-5-ol; 4,6-di bromo-2-(3-fluoro-4-hydroxyphenyl)-7-vinyl-1,3-benzoxazol-5-ol; 7-(1-bromovinyl)-2-(2-fluoro-4-hydroxyphenyl)-1,3-benzoxazol-5-ol; 7-(1-bromovinyl)-2-(2,3-difluoro-4-hydroxyphenyl)-1,3-benzoxazol-5-ol; 7-allyl-2-(3-fluoro-4-hydroxyphenyl)-1,3-benzoxazol-5-ol; 2-(3,5-difluoro-4-hydroxyphenyl)-7-vinyl-1,3-benzoxazol-5-ol; 2-(3-fluoro-4-hydroxyphenyl)-7-(1-fluorovinyl)-1,3-benzoxazol-5-ol; and a pharmaceutically acceptable salt thereof.

In some embodiments, at least one of said one or more estrogenic agents is selected from: 2-(5-hydroxy-1,3-benzoxazol-2-yl)benzene-1,4-diol; 3-(5-hydroxy-1,3-benzoxazol-2-yl)benzene-1,2-diol; 2-(3-fluoro-4-hydroxyphenyl)-1,3-benzoxazol-5-ol; 2-(3-chloro-4-hydroxyphenyl)-1,3-benzoxazol-5-ol; 2-(2-chloro-4-hydroxyphenyl)-1,3-benzoxazol-5-ol; 2-(3-fluoro-4-hydroxyphenyl)-1,3-benzoxazol-6-ol; 2-(3-tert-butyl-4-hydroxyphenyl)-1,3-benzoxazol-6-ol; 2-(6-hydroxy-1,3-benzoxazol-2-yl)benzene-1,4-diol; 3-(6-hydroxy-1,3-benzoxazol-2-yl)benzene-1,2-diol; 4-(6-hydroxy-1,3-benzoxazol-2-yl)benzene-1,2-diol; 2-(3-chloro-4-hydroxyphenyl)-1,3-benzoxazol-6-ol; 4-(5-hydroxy-1,3-benzoxazol-2-yl)benzene-1,3-diol; 4-(6-hydroxy-1,3-benzoxazol-2-yl)benzene-1,3-diol; 6-chloro-2-(3-fluoro-4-hydroxyphenyl)-1,3-benzoxazol-5-ol; 6-bromo-2-(3-fluoro-4-hydroxyphenyl)-1,3-benzoxazol-5-ol; 6-chloro-2-(4-hydroxyphenyl)-1,3-benzoxazol-5-ol; 5-chloro-2-(4-hydroxyphenyl)-1,3-benzoxazol-6-ol; 7-bromo-2-(3-fluoro-4-hydroxyphenyl)-1,3-benzoxazol-5-ol; 7-bromo-2-(2-fluoro-4-hydroxyphenyl)-1,3-benzoxazol-5-ol; 7-bromo-2-(2,3-difluoro-4-hydroxyphenyl)-1,3-benzoxazol-5-ol; 2-(4-hydroxyphenyl)-7-vinyl-1,3-benzoxazol-5-ol; 7-(1,2-dibromoethyl)-2-(4-hydroxyphenyl)-1,3-benzoxazol-5-ol; 7-(1-bromovinyl)-2-(4-hydroxyphenyl)-1,3-benzoxazol-5-ol; 7-ethynyl-2-(4-hydroxyphenyl)-1,3-benzoxazol-5-ol; 2-(4-hydroxyphenyl)-7-propyl-1,3-benzoxazol-5-ol; 7-butyl-2-(4-hydroxyphenyl)-1,3-benzoxazol-5-ol; 7-cyclopentyl-2-(4-hydroxyphenyl)-1,3-benzoxazol-5-ol; ethyl 5-hydroxy-2-(4-hydroxyphenyl)-1,3-benzoxazole-7-carboxylate; 2-(4-hydroxyphenyl)-7-phenyl-1,3-benzoxazol-5-ol; 2-(4-hydroxyphenyl)-7-methoxy-1,3-benzoxazol-5-ol; 7-ethyl-2-(4-hydroxyphenyl)-1,3-benzoxazol-5-ol; 7-ethyl-2-(2-ethyl-4-hydroxyphenyl)-1,3-benzoxazol-5-ol; 5-hydroxy-2-(4-hydroxyphenyl)-1,3-benzoxazole-7-carbaldehyde; 7-(hydroxymethyl)-2-(4-hydroxyphenyl)-1,3-benzoxazol-5-ol; 7-(bromomethyl)-2-(4-hydroxyphenyl)-1,3-benzoxazol-5-ol; [5-hydroxy-2-(4-hydroxyphenyl)-1,3-benzoxazol-7-yl]acetonitrile; 7-(1-hydroxy-1-methylethyl)-2-(4-hydroxyphenyl)-1,3-benzoxazol-5-ol; 2-(4-hydroxyphenyl)-7-isopropenyl-1,3-benzoxazol-5-ol; 2-(4-hydroxyphenyl)-7-isopropyl-1,3-benzoxazol-5-ol; 7-bromo-2-(4-hydroxy-3-(trifluoromethyl)phenyl)-1,3-benzoxazol-5-ol; 7-(2-furyl)-2-(4-hydroxyphenyl)-1,3-benzoxazol-5-ol; 2-(3-fluoro-4-hydroxyphenyl)-7-(2-furyl)-1,3-benzoxazol-5-ol; 2-(4-hydroxyphenyl)-7-thien-2-yl-1,3-benzoxazol-5-ol; 2-(4-hydroxyphenyl)-7-(1,3-thiazol-2-yl)-1,3-benzoxazol-5-ol; 2-(3-fluoro-4-hydroxyphenyl)-5-hydroxy-1,3-benzoxazole-7-carbonitrile; 4-bromo-2-(4-hydroxyphenyl)-7-methoxy-1,3-benzoxazol-5-ol; 4,6-dibromo-2-(4-hydroxyphenyl)-7-methoxy-1,3-benzoxazol-5-ol; 7-bromo-2-(3,5-difluoro-4-hydroxyphenyl)-1,3-benzoxazol-5-ol; and a pharmaceutically acceptable salt thereof.

In some embodiments, at least one of said one or more estrogenic agents is selected from: 8-fluoro-6-(3-fluoro-4-hydroxyphenyl)-2-naphthol; 1-chloro-8-fluoro-6-(3-fluoro-4-hydroxyphenyl)-2-naphthol; 3-(3-fluoro-4-hydroxyphenyl)-7-hydroxy-1-naphthonitrile; 3-(3,5-difluoro-4-hydroxyphenyl)-7-hydroxy-1-naphthonitrile; and a pharmaceutically acceptable salt thereof.

In some embodiments, at least one of said one or more estrogenic agents is selected from: 7-(4-hydroxyphenyl)-2-naphthol; 7-(3-hydroxyphenyl)-2-naphthol; 6-(4-hydroxyphenyl)-1-naphthol; 6-phenyl-2-naphthol; 6-(3-hydroxyphenyl)-2-naphthol; 6-(3-chlorophenyl)-2-naphthol; 2-fluoro-4-(2-naphthyl)phenol; 6-(3-fluoro-4-hydroxyphenyl)-2-naphthol; 6-(3-chloro-4-hydroxyphenol)-2-naphthol; 1-chloro-6-phenyl-2-naphthol; 1-bromo-6-(4-hydroxyphenyl)-2-naphthol; 1-chloro-6-(4-hydroxyphenyl)-2-naphthol; 1-fluoro-6-(4-hydroxyphenyl)-2-naphthol; 2-hydroxy-6-(4-hydroxyphenyl)-1-naphthonitrile; 6-(4-hydroxyphenyl)-1-phenyl-2-naphthol; 6-(4-hydroxyphenyl)-1-methyl-2-naphthol; 1-chloro-6-(3-fluoro-4-hydroxyphenyl)-2-naphthol; 1-chloro-6-(3-chloro-4-hydroxyphenyl)-2-naphthol; 6-(4-hydroxyphenyl)-1-nitro-2-naphthol; 1-chloro-6-(4-hydroxy-2-methylphenyl)-2-naphthol; 6-(4-hydroxy-2-methylphenyl)-2-naphthol; 6-(4-hydroxy-2-methoxyphenyl)-2-naphthol; 6-(2-chloro-4-hydroxyphenyl)-2-naphthol; 1-chloro-6-(2-chloro-4-hydroxyphenyl)-2-naphthol; 6-(2-fluoro-4-hydroxyphenyl)-2-naphthol; 6-(2,5-difluoro-4-hydroxyphenyl)-2-naphthol; 6-(2,6-difluoro-4-hydroxyphenyl)-2-naphthol; 1-chloro-6-(2-fluoro-4-hydroxyphenyl)-2-naphthol; 1-chloro-6-(2,5-difluoro-4-hydroxyphenyl)-2-naphthol; 1-chloro-6-(2,6-difluoro-4-hydroxyphenyl)-2-naphthol; 8-fluoro-6-(4-hydroxyphenyl)-2-naphthol; 1-chloro-8-fluoro-6-(4-hydroxyphenyl)-2-naphthol; 8-chloro-6-(4-hydroxyphenyl)-2-naphthol; 1,5-dichloro-8-fluoro-6-(4-hydroxyphenyl)-2-naphthol; 2-chloro-4-(2-naphthyl)phenol; 3-bromo-8-chloro-6-(4-hydroxyphenyl)-2-naphthol; 1,8-dichloro-6-(4-hydroxyphenyl)-2-naphthol; 3-bromo-1,8-dichloro-6-(4-hydroxyphenyl)-2-naphthol; 7-hydroxy-3-(4-hydroxyphenyl)-1-naphthonitrile; 8-chloro-3-(4-hydroxyphenyl)-7-hydroxy-1-naphthonitrile; 8-chloro-3-(3-fluoro-4-hydroxyphenyl)-7-hydroxy-1-naphthonitrile; 6-(3,5-difluoro-4-hydroxyphenyl)-2-naphthol; 1-chloro-6-(3,5-difluoro-4-hydroxyphenyl)-2-naphthol; 8-bromo-7-hydroxy-3-(4-hydroxyphenyl)-1-naphthonitrile; and a pharmaceutically acceptable salt thereof.

In some further embodiments, at least one of said one or more estrogenic agents is selected from: [5-Hydroxy-2-(4-hydroxy-phenyl)-benzofuran-7-yl]-acetonitrile; [5-Methoxy-2-(4-methoxy-phenyl)-benzofuran-7-yl]-acetonitrile; 2-[5-Methoxy-2-(4-methoxy-phenyl)-benzofuran-7-yl]-propionitrile; 2-[5-Methoxy-2-(4-methoxy-phenyl)-benzofuran-7-yl]-2-methyl-propionitrile; 2-[5-Hydroxy-2-(4-hydroxy-phenyl)-benzofuran-7-yl]-propionitrile; 2-[5-Hydroxy-2-(4-hydroxyphenyl)-benzofuran-7-yl]-2-methyl-propionitrile; [2-(3-Fluoro-4-hydroxy-phenyl)-5-hydroxybenzofuran-7-yl]-acetonitrile; 3-[5-Hydroxy-2-(4-hydroxy-phenyl)-benzofuran-7-yl]-propionitrile; 2-(4-Hydroxy-phenyl)-7-methoxy-benzofuran-5-ol; 4-Chloro-2-(4-hydroxy-phenyl)-7-methoxy-benzofuran-5-ol; 4-Bromo-2-(4-hydroxy-phenyl)-7-methoxy-benzofuran-5-ol; 5-Hydroxy-2-(4-hydroxy-phenyl)-7-methoxy-benzofuran-4-carbaldehyde; 5-Hydroxy-2-(4-hydroxy-phenyl)-7-methoxy-benzofuran-4-carbonitrile; 2-(3-Fluoro-4-hydroxy-phenyl)-5-hydroxy-7-methoxy-benzofuran-4-carbonitrile; and a pharmaceutically acceptable salt thereof.

In some further embodiments, at least one of said one or more estrogenic agents is selected from: 2-Hydroxy-3-iodo-5-methoxy-benzaldehyde; 5-Methoxy-2-(4-methoxy-phenyl)benzofuran-7-carbaldehyde; [5-Methoxy-2-(4-methoxy-phenyl)-benzofuran-7-yl]-methanol; 7-Bromomethyl-2-(4-hydroxy-phenyl)-benzofuran-5-ol; 2-(4-Hydroxy-phenyl)-7-methoxymethyl-benzofuran-5-ol; 7-Ethoxymethyl-2-(4-hydroxy-phenyl)-benzofuran-5-ol; 2-(4-Hydroxy-phenyl)-7-isopropoxymethyl benzofuran-5-ol; 2-(4-Hydroxy-phenyl)-7-methyl-benzofuran-5-ol; 2-(4-Hydroxy-phenyl)-7-methylsulfanylmethyl-benzofuran-5-ol; 7-Ethylsulfanylmethyl-2-(4-hydroxyphenyl)-benzofuran-5-ol; 2-(4-Hydroxy-phenyl)-7-phenylsulfanylmethyl-benzofuran-5-ol; 2-(4-Hydroxy-phenyl)-7-methanesulfinylmethyl-benzofuran-5-ol; 2-(4-Hydroxy-phenyl)-7-methanesulfonylmethyl-benzofuran-5-ol; 2-(4-Hydroxy-phenyl)-7-thiocyanatomethyl-benzofuran-5-ol; 2-(4-Hydroxy-phenyl)-7-imidazol-1-ylmethyl-benzofuran-5-ol; 7-Bromomethyl-5-methoxy-2-(4-methoxy-phenyl)-benzofuran; 5-Methoxy-2-(4-methoxy-phenyl)-benzofuran-7-carboxylic acid; 5-Hydroxy-2-(4-hydroxy-phenyl)-benzofuran-7-carboxylic acid; 7-Hydroxymethyl-2-(4-hydroxyphenyl)-benzofuran-5-ol; 5-Methoxy-2-(4-methoxy-phenyl)-benzofuran-7-carbaldehyde oxime; 5-Methoxy-2-(4-methoxy-phenyl)-7-vinyl-benzofuran; 7-Ethyl-5-methoxy-2-(4-methoxy-phenyl)benzofuran; 7-Ethyl-2-(4-hydroxy-phenyl)-benzofuran-5-ol; 7-(2,2-Dichloro-vinyl)-5-methoxy-2-(4-methoxy-phenyl)-benzofuran; 7-(2,2-Dichlorovinyl)-5-hydroxy-2-(4-hydroxyphenyl)benzofuran-5-ol; 5-Methoxy-2-(4-methoxy-phenyl)-7-propenyl-benzofuran; 5-Methoxy-2-(4-methoxy-phenyl)-7-propyl-benzofuran; 2-(4-Hydroxy-phenyl)-7-propyl-benzofuran-5-ol; 5-Hydroxy-2-(4-hydroxy-phenyl)-benzofuran-7-carboxylic acid isopropyl ester; 5-Hydroxy-2-(4-hydroxy-phenyl)-benzofuran-7-carboxylic acid propyl ester; 5-Hydroxy-2-(4-hydroxy-phenyl)-benzofuran-7-carboxylic acid ethyl ester; 2-(3-Fluoro-4-methoxy-phenyl)-5-methoxy-benzofuran-7-carboxylic acid methyl ester; [2-(3-Fluoro-4-methoxy-phenyl)-5-methoxy-benzofuran-7-yl]-methanol; 7-Bromomethyl-2-(3-fluoro-4-hydroxy-phenyl)-benzofuran-5-ol; 2-(3-Fluoro-4-methoxy-phenyl)-5-methoxy-benzofuran-7-carboxylic acid; 2-(3-Fluoro-4-methoxyphenyl)-5-methoxy-benzofuran-7-carboxylic acid methoxy-methyl-amide; 2-(3-Fluoro-4-methoxy-phenyl)-5-methoxy-benzofuran-7-carbaldehyde; 2-(3-Fluoro-4-methoxy-phenyl)-5-methoxy-benzofuran-7-carbaldehyde oxime; 2-(3-Fluoro-4-hydroxy-phenyl)-5-hydroxybenzofuran-7-carbonitrile; 2-(3-Fluoro-4-hydroxy-phenyl)-7-methyl-benzofuran-5-ol; 3-Bromo-2-hydroxy-5-methoxy-benzaldehyde; 3-Bromo-2,5-dimethoxy-benzaldehyde; (3-Bromo-2,5-dimethoxy-phenyl)-methanol; 1-Bromo-3-chloromethyl-2,5-dimethoxy-benzene; (3-Bromo-2,5-dimethoxy-phenyl)-acetonitrile; (3-Bromo-2,5-dimethoxy-phenyl)-acetic acid; 2-(3-Bromo-2,5-dimethoxy-phenyl)-1-(4-methoxy-phenyl)-ethanone; 7-Chloro-2-(4-hydroxy-phenyl)-benzofuran-5-ol; 7-Bromo-2-(4-hydroxy-phenyl)-benzofuran-5-ol; 3-[5-Hydroxy-2-(4-hydroxyphenyl)benzofuran-7-yl]-acrylic acid methyl ester; 3-[5-Hydroxy-2-(4-hydroxy-phenyl)benzofuran-7-yl]-propionic acid methyl ester; 3-[5-Hydroxy-2-(4-hydroxyphenyl)-benzofuran-7-yl]-acrylamide; 4,7-Dibromo-2-(4-hydroxy-phenyl)-benzofuran-5-ol; 3-[5-Hydroxy-2-(4-hydroxyphenyl)-benzofuran-7-yl]-acrylonitrile; 7-Bromo-5-(tert-butyl-dimethyl-silanyloxy)-2-[4-(tert-butyldimethyl-silanyloxy)-phenyl]-benzofuran; 2-(4-Hydroxy-phenyl)-7-vinyl-benzofuran-5-ol; 5-Hydroxy-2-(4-hydroxy-phenyl)-7-methoxy-benzofuran-4-carbaldehyde oxime; 2-(2,5-Dimethoxyphenyl)-1-(4-methoxy-phenyl)-ethanone; 2-(4-Hydroxy-phenyl)-benzofuran-5-ol; 2-(2,5-Dimethoxy-phenyl)-1-(2-fluoro-4-methoxy-phenyl)-ethanone; 2-(2-Fluoro-4-hydroxy-phenyl)benzofuran-5-ol; 5-OMe-Benzofuran 2-boronic acid; 4-(5-Methoxy-benzofuran-2-yl)-3-methyl-phenol; 2-(4-Hydroxy-2-methyl-phenyl)-benzofuran-5-ol; 5-Bromo-2-(4-methoxy-phenyl)benzofuran; 5-Chloro-2-(4-methoxy-phenyl)-benzofuran; 5-Fluoro-2-(4-methoxy-phenyl)benzofuran; 5-tert-Butyl-2-(4-methoxy-phenyl)-benzofuran; 5,7-Dichloro-2-(4-methoxy-phenyl)benzofuran; 5,7-Difluoro-2-(4-methoxy-phenyl)-benzofuran; 5,7-Dibromo-2-(4-methoxy-phenyl)benzofuran; 2-(4-Methoxy-phenyl)-5-trifluoromethyl-benzofuran; 4-(5-Bromo-benzofuran-2-yl)phenol; 4-(5-Chloro-benzofuran-2-yl)-phenol; 4-(5-Fluoro-benzofuran-2-yl)-phenol; 4-(5-tert-Butyl-benzofuran-2-yl)-phenol; 4-(5,7-Dichloro-benzofuran-2-yl)-phenol; 4-(5,7-Difluoro-benzofuran-2-yl)-phenol; 4-(5,7-Dibromo-benzofuran-2-yl)-phenol; 4-(5-Trifluoromethyl-benzofuran-2-yl)-phenol; 2-Iodo-4-methoxy-6-nitro-phenol; 5-Methoxy-2-(4-methoxy-phenyl)-7-nitro-benzofuran; 2-(4-Hydroxy-phenyl)-7-nitro-benzofuran-5-ol; 7-Amino-2-(4-hydroxy-phenyl)benzofuran-5-ol; 1-(2-Bromo-4-methoxyphenyl)-2-(2,5-dimethoxy-phenyl)-ethanone; 2-(2-Bromo-4-hydroxy-phenyl)-benzofuran-5-ol; 2-(5-Hydroxy-biphenyl-2-yl)-benzofuran-5-ol; 2-(4′-Benzyloxy-5-hydroxy-biphenyl-2-yl)-benzofuran-5-ol; 6-(5-Hydroxy-benzofuran-2-yl)-biphenyl-3,4′-diol; 2-[5-Hydroxy-4′-(2-pyrrolidin-1-yl-ethoxy)-biphenyl-2-yl]-benzofuran-5-ol; 2,2-Dimethyl-propionic acid 2-[4-(2,2-dimethyl-propionyloxy)-phenyl]-benzofuran-5-yl ester; 1-[5-Hydroxy-2-(4-hydroxy-phenyl)-benzofuran-3-yl]-ethanone; 1-[5-Hydroxy-2-(4-hydroxy-phenyl)-benzofuran-3-yl]-ethanone oxime; 3-(1-Hydroxy-ethyl)-2-(4-hydroxy-phenyl)-benzofuran-5-ol; 2-[5-Methoxy-2-(4-methoxy-phenyl)-benzofuran-7-yl]-propan-2-ol; 7-Isopropenyl-5-methoxy-2-(4-methoxyphenyl)-benzofuran; 7-Isopropyl-5-methoxy-2-(4-methoxy-phenyl)-benzofuran; 2-(4-Hydroxyphenyl)-7-isopropyl-benzofuran-5-ol; 5-Hydroxy-2-(4-hydroxy-phenyl)-benzofuran-7-carboxylic acid methoxy-methyl-amide; 5-Hydroxy-2-(4-hydroxy-phenyl)-benzofuran-7-carbaldehyde; 5-Methoxy-2-(4-methoxy-phenyl)-benzofuran-7-carboxylic acid methoxy-methyl-amide; 1-[5-Methoxy-2-(4-methoxy-phenyl)-benzofuran-7-yl]-ethanone; 1-[5-Hydroxy-2-(4-hydroxy-phenyl)benzofuran-7-yl]-ethanone; 1-[5-Hydroxy-2-(4-hydroxy-phenyl)-benzofuran-7-yl]-propan-1-one; 2-(2,5-Dimethoxy-phenyl)-1-(3-fluoro-4-methoxy-phenyl)-ethanone; 2-(3-Fluoro-4-hydroxyphenyl)-benzofuran-5-ol; 4-(5-Methoxy-benzofuran-2-yl)-benzoic acid methyl ester; 4-(5-Hydroxy-benzofuran-2-yl)-benzoic acid; 2-(4-Hydroxymethyl-phenyl)-benzofuran-5-ol; 4-Bromo-2-(4-hydroxy-phenyl)-benzofuran-5-ol; 4-Chloro-2-(4-hydroxy-phenyl)-benzofuran-5-ol; 2-(4-Hydroxy-phenyl)-4-methoxy-benzofuran-5-ol; 4-Bromo-5-methoxy-2-(4-methoxy-phenyl)benzofuran; 5-Methoxy-2-(4-methoxy-phenyl)-benzofuran-4-carbonitrile; 5-Hydroxy-2-(4-hydroxy-phenyl)-benzofuran-4-carbonitrile; 5-Methoxy-2-(4-methoxy-phenyl)-4-methyl-benzofuran; 2-(4-Hydroxy-phenyl)-4-methyl-benzofuran-5-ol; 2-(4-Hydroxy-phenyl)-7-[1,3,4]oxadiazol-2-yl-benzofuran-5-ol; 2,2,2-Trifluoro-1-[5-methoxy-2-(4-methoxy-phenyl)benzofuran-7-yl]-ethanol; 5-Methoxy-2-(4-methoxyphenyl)-7-(2,2,2-trifluoroethyl)-benzofuran; 2-(4-Hydroxy-phenyl)-7-(2,2,2-trifluoro-ethyl)-benzofuran-5-ol; 2-(4-Methoxy-phenyl)-benzofuran-5-carboxylic acid methyl ester; 2-(4-Hydroxy-phenyl)-benzofuran-5-carboxylic acid; 4-(5-Hydroxymethyl-benzofuran-2-yl)-phenol; and a pharmaceutically acceptable salt thereof.

In some further embodiments, at least one of said one or more estrogenic agents is selected from: 3,9-Dimethoxy-6H-chromeno[4,3-b]quinolin-7-ol; 7-Chloro-3,9-dimethoxy-6H-chromeno[4,3-b]quinoline; 7-Bromo-3,9-dimethoxy-6H-chromeno[4,3-b]quinoline; 7-Chloro-3,9-dihydroxy-6H-chromeno[4,3-b]quinoline; 7-Bromo-3,9-dihydroxy-6H-chromeno[4,3-b]quinoline; 3,9-Dihydroxy-7-vinyl-6H-chromeno[4,3-b]quinoline; 3,9-Dihydroxy-7-[(trimethylsilyl)ethynyl]-6H-chromeno[4,3-b]quinoline; 3,9-Dihydroxy-7-ethynyl-6H-chromeno[4,3-b]quinoline; 3,9-Dihydroxy-7-ethyl-6H-chromeno[4,3-b]quinoline; 7-Cyano-3,9-dihydroxy-6H-chromeno[4,3-b]quinoline; 7-(4-Chlorophenyl)-3,9-dihydroxy-6H-chromeno[4,3-b]quinoline; 7-(4-Cyanophenyl)-3,9-dihydroxy-6H-chromeno[4,3-b]quinoline; 3,9-Dihydroxy-7-(4-methoxyphenyl)-6H-chromeno[4,3-b]quinoline; 3,9-Dihydroxy-7-[4-(trifluoromethyl)phenyl]-6H-chromeno[4,3-b]quinoline; 7-(3-Chlorophenyl)-3,9-dihydroxy-6H-chromeno[4,3-b]quinoline; 3,9-Dihydroxy-7-(3-methoxyphenyl)-6H-chromeno[4,3-b]quinoline; 7-(3-Cyanophenyl)-3,9-dihydroxy-6H-chromeno[4,3-b]quinoline; and a pharmaceutically acceptable salt, chelate, complex or prodrug thereof.

In some further embodiments, at least one of said one or more estrogenic agents is selected from: 5,6-Dihydro-benzo[b]naphtho[2,1-d]furan-3,9-diol; Benzo[b]naphtho[2,1-d]furan-3,9-diol; 5-Bromo-benzo[b]naphtho[2,1-d]furan-3,9-diol; 3,8-Dihydroxy-5,6-dihydro-benzo[b]naphtho[2,1-d]furan-10-carbonitrile; 3,9-Dihydroxy-6,7-dihydro-5H-12-oxa-dibenzo[a,e]azulen-11-carbonitrile; 3,9-Dihydroxy-5,6-dihydro-benzo[b]naphtho[2,1-d]furan-10-carbonitrile; 3,9-Dihydroxy-benzo[b]naphtho[2,1-d]furan-10-carbonitrile; 3,8-Dihydroxy-5,5-dimethyl-5,6-dihydro-benzo[b]naphtho[2,1-d]furan-10-carbonitrile; 6H-Benzo[4,5]furo[3,2-c]chromen-3,8-diol; 3,8-Dihydroxy-6H-Benzo[4,5]furo[3,2-c]chromene-10-carbonitrile; 10-Bromo-6H-benzo[4,5]furo[3,2-c]chromene-3,8-diol; 2,9-Dihydroxy-5,6-dihydro-benzo[b]naphtho[2,1-d]furan-10-benzonitrile; 2,9-Dihydroxy-benzo[b]naphtho[2,1-d]furan-10-carbonitrile; and a pharmaceutically acceptable salt thereof.

In some further embodiments, at least one of said one or more estrogenic agents is selected from:

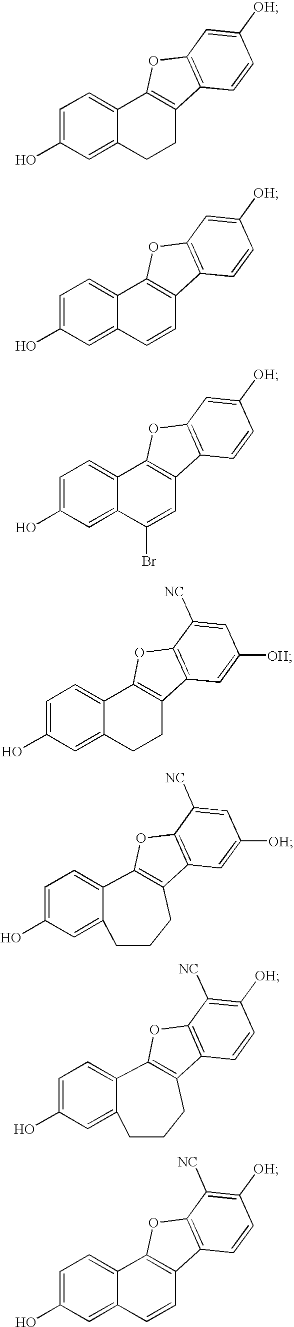

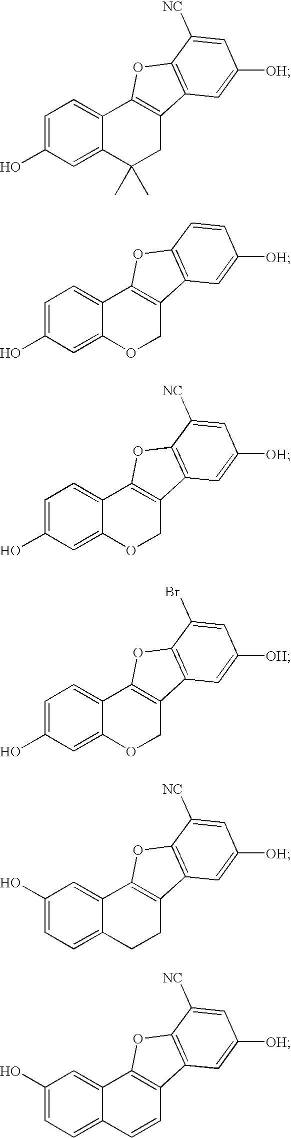

and a pharmaceutically acceptable salt thereof.

In yet further embodiments, at least one of said one or more estrogenic agents is selected from: 6H-Dibenzo[c,h]chromene-2,8-diol; 9-Fluoro-6H-dibenzo[c,h]chromene-2,8-diol; 2,8-Dihydroxy-6H-dibenzo[c,h]chromene-12-carbonitrile; 9-fluoro-2,8-dihydroxy-6H-dibenzo[c,h]chromene-12-carbonitrile; 1-Chloro-2,8-dihydroxy-6H-dibenzo[c,h]chromene-12-carbonitrile; 1-Chloro-6H-dibenzo[c,h]chromene-2,8-diol; 12-Bromo-6H-dibenzo[c,h]chromene-2,8-diol; 12-Chloro-6H-dibenzo[c,h]chromene-2,8-diol; 12-Bromo-9-fluoro-6H-dibenzo[c,h]chromene-2,8-diol; 12-Chloro-9-fluoro-6H-dibenzo[c,h]chromene-2,8-diol; 12-Methoxy-6H-dibenzo[c,h]chromene-2,8-diol; 11-Chloro-12-methoxy-6H-dibenzo[c,h]chromene-2,8-diol; 12-Methyl-6H-dibenzo[c,h]chromene-2,8-diol; 12-Vinyl-6H-dibenzo[c,h]chromene-2,8-diol; 12-Ethyl-6H-dibenzo[c,h]chromene-2,8-diol; 12-Thien-3-yl-6H-dibenzo[c,h]chromene-2,8-diol; 12-Thien-2-yl-6H-dibenzo[c,h]chromene-2,8-diol; 12-Butyl-6H-dibenzo[c,h]chromene-2,8-diol; 12-Propyl-6H-dibenzo[c,h]chromene-2,8-diol; 6H-dibenzo[c,h]chromene-1,8-diol; 12-Chloro-6H-dibenzo[c,h]chromene-1,8-diol; 12-Bromo-6H-dibenzo[c,h]chromene-1,8-diol; 1,8-Dihydroxy-6H-dibenzo[c,h]chromene-12-carbonitrile; 2-Chloro-6H-dibenzo[c,h]chromene-1,8-diol; 4-chloro-6H-dibenzo[c,h]chromene-1,8-diol; 6H-Dibenzo[c,h]chromen-8-ol; and a pharmaceutically acceptable salt thereof.

In some further embodiments, at least one of said one or more estrogenic agents is selected from: 3-(4-Hydroxy-2-methylphenyl)-1,2-benzisoxazol-6-ol; 3-(4-Hydroxy-2,5-dipropylphenyl)-1,2-benzisoxazol-6-ol; 3-(2-Ethyl-4-hydroxy-5-propylphenyl)-1,2-benzisoxazol-6-ol; 3-(4-Hydroxy-2-isobutyl-5-propylphenyl)-1,2-benzisoxazol-6-ol; 3-(4-Hydroxy-3-propylphenyl)-1,2-benzisoxazol-6-ol; 3-(4-Hydroxy-2-propylphenyl)-1,2-benzisoxazol-6-ol; 3-(4-Hydroxy-3-methylphenyl)-1,2-benzisoxazol-6-ol; 3-(4-Hydroxyphenyl)-1,2-benzisoxazol-6-ol; 3-(2-Hydroxyphenyl)-1,2-benzisoxazol-6-ol; 2-(6-Hydroxy-1,2-benzisoxazol-3-yl)benzene-1,4-diol; 4-(6-Hydroxy-1,2-benzisoxazol-3-yl)benzene-1,3-diol; 3-(4-Hydroxyphenyl)-1,2-benzisoxazol-5-ol; 7-Bromo-3-(4-hydroxyphenyl)-1,2-benzisoxazol-5-ol; 3-(3-Bromo-4-hydroxyphenyl)-1,2-benzisoxazol-6-ol; 3-(3-Chloro-4-hydroxyphenyl)-1,2-benzisoxazol-6-ol; 3-(3-Fluoro-4-hydroxyphenyl)-1,2-benzisoxazol-6-ol; 4-Bromo-6-(6-hydroxy-1,2-benzisoxazol-3-yl)benzene-1,3-diol; 4-Chloro-6-(6-hydroxy-1,2-benzisoxazol-3-yl)benzene-1,3-diol; 4-(6-Hydroxy-1,2-benzisoxazol-3-yl)-6-methylbenzene-1,3-diol; 4-(6-Hydroxy-1,2-benzisoxazol-3-yl)-2-methylbenzene-1,3-diol; and a pharmaceutically acceptable salt thereof.

In some embodiments, at least one of said one or more estrogenic agents is selected from: 2-(6-Hydroxy-1-naphthyl)-1,3-benzoxazol-5-ol; 2-(6-Hydroxy-1-naphthyl)-1,3-benzoxazol-6-ol; 2-(6-Hydroxy-2-naphthyl)-1,3-benzoxazol-5-ol; 2-(6-Hydroxy-2-naphthyl)-1,3-benzoxazol-6-ol; 2-(5-Hydroxy-1-naphthyl)-1,3-benzoxazol-6-ol; 2-(4-Hydroxy-1-naphthyl)-1,3-benzoxazol-5-ol; 2-(4-Hydroxy-1-naphthyl)-1,3-benzoxazol-6-ol; 2-(5-Bromo-6-hydroxy-1-naphthyl)-1,3-benzoxazol-6-ol; 3-(6-Hydroxy-2-naphthyl)-1,2-benzisoxazol-6-ol; 3-(6-Hydroxy-2-naphthyl)-1,2-benzisoxazol-5-ol; 3-(5-Hydroxy-2-naphthyl)-1,2-benzisoxazol-6-ol; 3-(5-Hydroxy-1-naphthyl)-1,2-benzisoxazol-6-ol; 3-(6-Hydroxy-1-naphthyl)-1,2-benzisoxazol-6-ol; 3-(7-Hydroxy-1-naphthyl)-1,2-benzisoxazol-6-ol; 3-(5-Bromo-6-hydroxy-2-naphthyl)-1,2-benzisoxazol-6-ol; 3-(6-Hydroxy-2-naphthyl)-1-methyl-1H-indazol-6-ol; 1-Ethyl-3-(6-hydroxy-2-naphthyl)-1H-indazol-6-ol; and a pharmaceutically acceptable salt thereof.

In some embodiments, at least one of said one or more estrogenic agents is selected from: 3-amino-5-hydroxy-2-(4-hydroxyphenyl)-1H-inden-1-one; 5-hydroxy-2-(4-hydroxyphenyl-1H-indene-1,3(2H)-dione; 3-bromo-6-hydroxy-2-(4-hydroxyphenyl)-1H-inden-1-one; 3-bromo-5-hydroxy-2-(4-hydroxyphenyl)-1H-inden-1-one; 3-(ethylamino)-6-hydroxy-2-(4-hydroxyphenyl)-1H-inden-1-one; 3-(ethylthio)-6-hydroxy-2-(4-hydroxyphenyl)-1H-inden-1-one; 3-(ethylthio)-5-hydroxy-2-(4-hydroxyphenyl)-1H-inden-1-one; 6-hydroxy-2-(4-hydroxyphenyl)-3-(methylthio)-1H-inden-1-one; 5-hydroxy-2-(4-hydroxyphenyl)-3-(methylthio)-1H-inden-1-one; 6-hydroxy-2-(4-hydroxyphenyl)-3-phenyl-1H-inden-1-one; 5-hydroxy-2-(4-hydroxyphenyl)-3-phenyl-1H-inden-1-one; 6-hydroxy-2-(4-hydroxyphenyl)-3-methyl-1H-inden-1-one; 5-hydroxy-2-(4-hydroxyphenyl)-3-methyl-1H-inden-1-one; 4,6-dihydroxy-2-(4-hydroxyphenyl)-1H-inden-1,3(2H)-dione; 3-bromo-4,6-dihydroxy-2-(4-hydroxyphenyl)-1H-inden-1-one; 3-bromo-6-hydroxy-2-(4-hydroxyphenyl)-4-methoxy-1H-inden-1-one; 5,7-dihydroxy-2-(4-hydroxyphenyl)-3-methyl-1H-inden-1-one; 4,6-dihydroxy-2-(4-hydroxyphenyl)-3-(methylthio)-1H-inden-1-one; 3-(ethylthio)-4,6-Dihydroxy-2-(4-hydroxyphenyl)-1H-inden-1-one; 4,6-dihydroxy-2-(4-hydroxyphenyl)-3-phenyl-1H-inden-1-one; 5,7-dihydroxy-2-(4-hydroxyphenyl)-3-phenyl-1H-inden-1-one; 5,6-dihydroxy-2-(4-hydroxyphenyl)-3-methyl-1H-inden-1-one; and a pharmaceutically acceptable salt thereof.

In some embodiments, at least one of said one or more estrogenic agents is selected from: 5-(3-hydroxyphenyl)-3-(4-hydroxyphenyl)-4-methylthiophene-2-carbaldehyde; 3-(3-hydroxyphenyl)-5-(4-hydroxyphenyl)-4-methylthiophene-2-carbaldehyde; 3-(4-hydroxy-2-methylphenyl)-5-(4-hydroxyphenyl)-4-methyl-2-thiophenecarbaldehyde; 3-(5-hydroxy-2-methylphenyl)-5-(4-hydroxyphenyl)-4-methyl-2-thiophenecarbaldehyde; 3-(3-fluoro-4-hydroxyphenyl)-5-(4-hydroxyphenyl)-4-methyl-2-thiophenecarbaldehyde; 3-(3-formyl-4-hydroxyphenyl)-5-(4-hydroxyphenyl)-4-methyl-2-thiophenecarbaldehyde; 3-(3-chloro-4-hydroxyphenyl)-5-(4-hydroxyphenyl)-4-methylthiophene-2-carbaldehyde; 5-(3-fluoro-4-hydroxyphenyl)-3-(4-hydroxyphenyl)-4-methyl-2-thiophenecarbaldehyde; 5-(3-fluoro-4-hydroxyphenyl)-3-(4-hydroxy-2-methylphenyl)-4-methylthiophene-2-carbaldehyde; 3-(3-chloro-4-hydroxyphenyl)-5-(3-fluoro-4-hydroxyphenyl)-4-methylthiophene-2-carbaldehyde; 5-(4-hydroxy-2-methylphenyl)-3-(4-hydroxyphenyl)-4-methyl-2-thiophenecarbaldehyde; 3-(3-fluoro-4-hydroxyphenyl)-5-(4-hydroxy-2-methyl phenyl)-4-methylthiophene-2-carbaldehyde; 5-(3-chloro-4-hydroxyphenyl)-3-(4-hydroxyphenyl)-4-methylthiophene-2-carbaldehyde; 5-(3-chloro-4-hydroxyphenyl)-3-(3-fluoro-4-hydroxyphenyl)-4-methylthiophene-2-carbaldehyde; 3,5-bis(4-hydroxyphenyl)-4-methylthiophene-2-carbaldehyde; 3-(3-hydroxyphenyl)-5-(4-hydroxyphenyl)-4-methylthiophene-2-carbaldehyde oxime; 3-(3-hydroxyphenyl)-5-(4-hydroxyphenyl)-4-methylthiophene-2-carbonitrile; 3,5-bis(4-hydroxyphenyl)-4-methylthiophene-2-carbaldehyde oxime; 3,5-bis(4-hydroxyphenyl)-4-methylthiophene-2-carbonitrile; 3,5-bis(4-hydroxyphenyl)-4-methylthiophene-2-carbaldehyde O-methyloxime; 3-(4-hydroxyphenyl)-5-(3-hydroxyphenyl)-4-methylthiophene-2-carbaldehyde oxime; 3-(4-hydroxyphenyl)-5-(3-hydroxyphenyl)-4-methylthiophene-2-carbonitrile; 3-(3-fluoro-4-hydroxyphenyl)-5-(4-hydroxy-2-methyl phenyl)-4-methylthiophene-2-carbaldehyde O-methyloxime; and a pharmaceutically acceptable salt thereof.

In some further embodiments, at least one of said one or more estrogenic agents is selected from: 4-Chloro-2-(4-hydroxyphenyl)quinolin-6-ol; 4-Chloro-2-(3-fluoro-4-hydroxyphenyl)quinolin-6-ol; 8-Bromo-4-chloro-2-(3-fluoro-4-hydroxyphenyl)-quinolin-6-ol; 4-Bromo-2-(4-hydroxyphenyl)quinolin-6-ol; 4-Bromo-2-(3-fluoro-4-hydroxyphenyl)quinolin-6-ol; 4-Bromo-2-(3,5-difluoro-4-hydroxyphenyl)quinolin-6-ol; 4-Cyano-2-(4-hydroxyphenyl)quinolin-6-ol; 4-Cyano-2-(3-fluoro-4-hydroxyphenyl)quinolin-6-ol; 6-Acetoxy-4-bromo-2-(3-fluoro-4-acetoxyphenyl)quinoline; 2-(4-Hydroxyphenyl)-4-iodoquinolin-6-ol; 2-(3-Fluoro-4-hydroxyphenyl)-4-iodoquinolin-6-ol; 2-(3-Fluoro-4-hydroxyphenyl)-4-vinylquinolin-6-ol; 4-Ethyl-2-(3-fluoro-4-hydroxyphenyl)quinolin-6-ol; 2-(4-Hydroxyphenyl)-4-[(trimethylsilyl)ethynyl]quinolin-6-ol; 2-(3-Fluoro-4-hydroxyphenyl)-4-[(trimethylsilyl)ethynyl]quinolin-6-ol; 4-Ethynyl-2-(4-hydroxyphenyl)quinolin-6-ol; 4-Ethynyl-2-(3-fluoro-4-hydroxyphenyl)quinolin-6-ol; 2-(3,5-Difluoro-4-hydroxyphenyl)-4-ethynylquinolin-6-ol; 2-(3-Fluoro-4-hydroxyphenyl)-4-(phenylethynyl)quinolin-6-ol; 4-Acetyl-2-(4-hydroxyphenyl)quinolin-6-ol; 4-Acetyl-2-(3-fluoro-4-hydroxyphenyl)quinolin-6-ol; 4-(1-Hydroxyethyl)-2-(4-hydroxyphenyl)quinolin-6-ol; 2-(3-Fluoro-4-hydroxyphenyl)-4-(1-hydroxyethyl)quinolin-6-ol; 2-(3-Fluoro-4-hydroxyphenyl)-4-thiazol-2-yl-quinolin-6-ol; 2-(3-Fluoro-4-hydroxyphenyl)-4-thiophen-3-yl-quinolin-6-ol; 2-(3-Fluoro-4-hydroxyphenyl)-4-(4-pyridyl)quinolin-6-ol; 2-(3-Fluoro-4-hydroxyphenyl)-4-(5-pyrimidyl)quinolin-6-ol; 5-Chloro-2-(4-hydroxyphenyl)quinolin-6-ol; 5-Bromo-2-(4-hydroxyphenyl)quinolin-6-ol; 4-Bromo-3-chloro-2-(4-hydroxyphenyl)quinolin-6-ol; 4-Bromo-3-chloro-2-(3-fluoro-4-hydroxyphenyl)quinolin-6-ol; 3-Chloro-4-(4-fluorophenyl)-2-(4-hydroxyphenyl)quinolin-6-ol; 3-Chloro-4-Cyano-2-(3-fluoro-4-hydroxyphenyl)quinolin-6-ol; 4-Bromo-8-chloro-2-(3-fluoro-4-hydroxyphenyl)quinolin-6-ol; 4,8-Dibromo-2-(3-fluoro-4-hydroxyphenyl)quinolin-6-ol; 8-Chloro-4-cyano-2-(3-fluoro-4-hydroxyphenyl)quinolin-6-ol; 8-Chloro-4-ethynyl-2-(3-fluoro-4-hydroxyphenyl)quinolin-6-ol; 8-Chloro-2-(3-fluoro-4-hydroxyphenyl)-4-hex-1-ynylquinolin-6-ol; 8-Chloro-2-(3-fluoro-4-hydroxyphenyl)-4-vinylquinolin-6-ol; 8-Chloro-4-(4-cyanophenyl)-2-(3-fluoro-4-hydroxyphenyl)quinolin-6-ol; 8-Chloro-2-(3-fluoro-4-hydroxyphenyl)-4-(4-methoxyphenyl)quinolin-6-ol; 4-Chloro-8-cyano-2-(3-fluoro-4-hydroxyphenyl)quinolin-6-ol; 4-Bromo-8-cyano-2-(3-fluoro-4-hydroxyphenyl)quinolin-6-ol; 8-Cyano-4-ethynyl-2-(3-fluoro-4-hydroxyphenyl)quinolin-6-ol; 4-Chloro-8-(4-cyanophenyl)-2-(3-fluoro-4-hydroxyphenyl)quinolin-6-ol; 8-Cyano-2-(3-fluoro-4-hydroxyphenyl)quinolin-6-ol; 2-(4-Hydroxyphenyl)-4-methoxyquinolin-6-ol; 2-(4-Hydroxyphenyl)-4-vinylquinolin-6-ol; 4-Ethyl-2-(4-hydroxyphenyl)quinolin-6-ol; 2-(3-Fluoro-4-hydroxyphenyl)-4-phenylquinolin-6-ol; 2-(3-Fluoro-4-hydroxylphenyl)-4-(methylphenyl)quinolin-6-ol; 2-(3-Fluoro-4-hydroxyphenyl)-4-(4-fluorophenyl)quinolin-6-ol; 4-(4-Chlorophenyl)-2-(3-fluoro-4-hydroxyphenyl)quinolin-6-ol; 4-(4-Cyanophenyl)-2-(3-fluoro-4-hydroxyphenyl)quinolin-6-ol; 2-(3-Fluoro-4-hydroxyphenyl)-4-(4-trifluoromethylphenyl)quinolin-6-ol; and a pharmaceutically acceptable salt thereof.

In some embodiments, at least one of said one or more estrogenic agents is provided in a transdermal dosage form. In some embodiments, the transdermal dosage form is selected from: a lotion, a cream, an ointment, a foam, a patch, a suspension, a solution, a pledget, a mask, and a suppository.

In some further embodiments, the methods of the present invention further comprise: providing to a mammal in need thereof, a therapeutically effective amount of one or more estrogenic agents in combination with one or more therapeutic agents useful in the treatment of inflammatory diseases, disorders, or conditions of the skin, or diseases, disorders, or conditions associated with collagen depletion. In some embodiments, at least one of said one or more therapeutic agents useful in the treatment of diseases, disorders, or conditions associated with collagen depletion is tretinoin, retinol, retinaldehyde, alpha tocopherol, monolaurin, alpha hydroxy acids, hyaluronic acid, one or more hyaluronic acid fragments, botulinum toxin (Botox® Cosmetic), an antibiotic, an antihistamine, a barrier-repair moisturizer, coal tar, a corticosteroid, cyclosporine, interferon gamma, mycophenolate mofetil, a topical calcineurin inhibitor, or a dietary supplement.

In some embodiments, said combination of one or more estrogenic agents and one or more therapeutic agents useful in the treatment of inflammatory diseases, disorders, or conditions of the skin, or diseases, disorders, or conditions associated with collagen depletion, is provided in a transdermal dosage form. In some embodiments, said transdermal dosage form is selected from: a lotion, a cream, an ointment, a foam, a patch, a suspension, a solution, a pledget, a mask, and a suppository.

In some further embodiments, the present invention provides a pharmaceutical composition comprising a therapeutically effective amount of one or more estrogenic agents in combination with one or more therapeutic agents useful in the treatment of inflammatory diseases, disorders, or conditions of the skin, or diseases, disorders, or conditions associated with collagen depletion and at least one pharmaceutically acceptable diluent or carrier. In some embodiments, said pharmaceutical composition is provided in a transdermal dosage form. In some embodiments, said transdermal dosage form is selected from: a lotion, a cream, an ointment, a foam, a patch, a suspension, a solution, a pledget, a mask, and a suppository.

Some further embodiments, provide pharmaceutical compositions comprising a therapeutically effective amount of one or more estrogenic agents, wherein at least one of said one or more estrogenic agents is selected from the compounds of Formulae I-XIII, described herein.

In some embodiments, the disease, disorder, or condition associated with collagen depletion is photo aging. In some embodiments, the disease, disorder, or condition associated with collagen depletion is chronological skin aging. In some embodiments, the disease, disorder, or condition associated with collagen depletion is smoking induced skin aging. In some embodiments, the inflammatory disease, disorder, or condition of the skin is eczema. In some embodiments, the inflammatory disease, disorder, or condition of the skin is a type of dermatitis. In some embodiments, the inflammatory disease, disorder, or condition of the skin is psoriasis. In some embodiments, the dermatitis is selected from: cercarial dermatitis, dermatitis herpetiformis, atopic dermatitis, seborrhoeic dermatitis, contact dermatitis, chronic dermatitis, irritant dermatitis, urushiol-induced contact dermatitis, nummular dermatitis, and dyshidrotic dermatitis.

Some embodiments of the present invention provide methods of suppressing MMP1, IL1B, and/or IL8 activity in mammalian epidermal and dermal skin comprising providing to a mammal in need thereof, a therapeutically effective amount of one or more estrogenic agents. In some embodiments, at least one of said one or more estrogenic agents provided to suppress MMP1, IL1B, and/or IL8 activity in mammalian epidermal skin is selected from the compounds of Formulae I-XIII, described herein.

Some further embodiments of the present invention provide uses of a therapeutically effective amount of one or more estrogenic agents, a prodrug thereof, or a pharmaceutically acceptable salt thereof; for the manufacture of a medicament for preventing, treating, or inhibiting photo aging, chronological skin aging, smoking induced skin aging, psoriasis, eczema, or dermatitis. In some embodiments, the invention provides such uses of a therapeutically effective amount of one or more estrogenic agents according to Formulae I-XIII as described herein.

Yet further embodiments of the present invention provide uses of a therapeutically effective amount of one or more estrogenic agents, a prodrug thereof, or a pharmaceutically acceptable salt thereof; in combination with one or more therapeutic agents useful in the treatment of inflammatory diseases, disorders, or conditions of the skin, or diseases, disorders, or conditions associated with collagen depletion; for the manufacture of a medicament for preventing, treating, or inhibiting photo aging, chronological skin aging, smoking induced skin aging, psoriasis, eczema, or dermatitis. In some embodiments, the invention provides such uses of a therapeutically effective amount of one or more estrogenic agents according to Formulae I-XIII as described herein.

In some embodiments, the present invention provides a method of preventing, treating, or inhibiting an inflammatory disease, disorder, or condition of the skin, or a disease, disorder, or condition associated with collagen depletion, comprising providing to a mammal in need thereof, a therapeutically effective amount of one or more estrogenic agents, wherein at least one of said one or more estrogenic agents is:

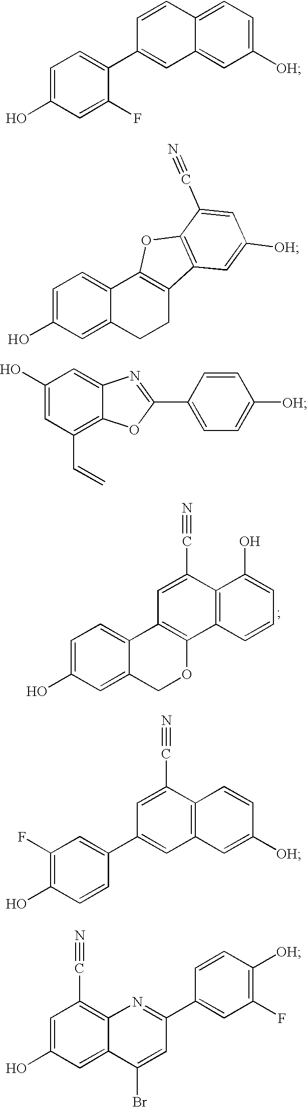

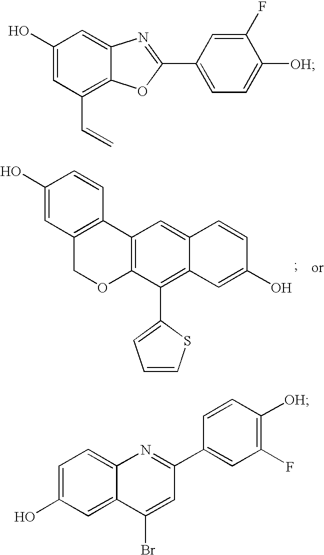

or a N-oxide thereof or a pharmaceutically acceptable salt thereof or a prodrug thereof.

In some further embodiments, the present invention provides a pharmaceutical composition comprising a therapeutically effective amount of one or more estrogenic agents, wherein at least one of said one or more estrogenic agents is:

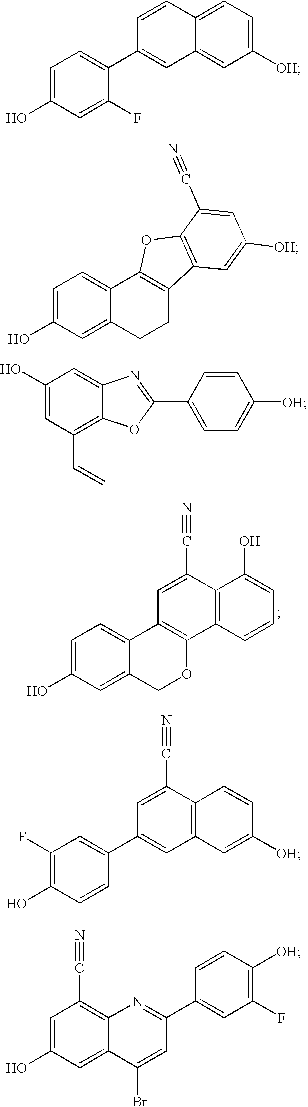

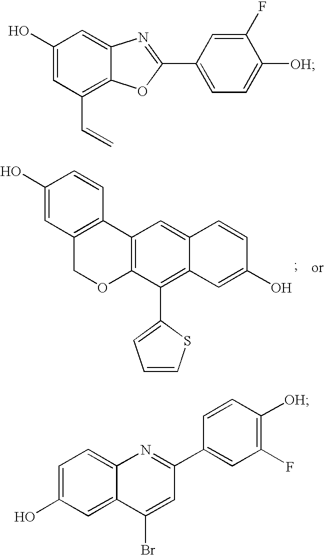

or a N-oxide thereof or a pharmaceutically acceptable salt thereof or a prodrug thereof; and at least one pharmaceutically acceptable carrier or diluent.

In yet further embodiments, the present invention provides a pharmaceutical composition comprising a therapeutically effective amount of one or more estrogenic agents, wherein at least one of said one or more estrogenic agents is:

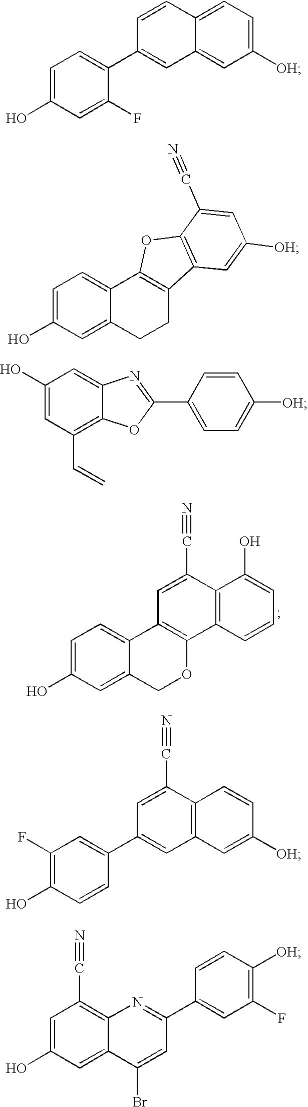

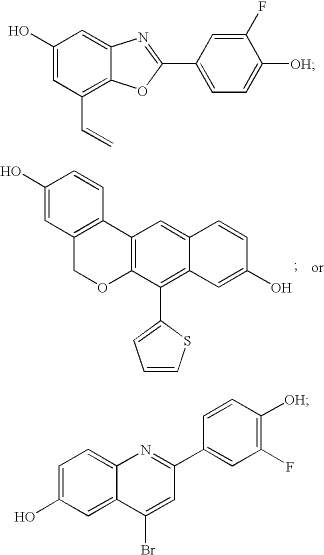

a N-oxide thereof or a pharmaceutically acceptable salt thereof or a prodrug thereof; and one or more therapeutic agents useful in the treatment of inflammatory diseases, disorders, or conditions of the skin, or diseases disorders, or conditions associated with collagen depletion; and at least one pharmaceutically acceptable diluent or carrier.

In some embodiments, the methods of the invention utilize one or more estrogenic agents. In some embodiments, at least one of said one or more estrogenic agents comprises a natural ERβ ligand. In some embodiments, at least one of said one or more estrogenic agents comprises an ERβ selective ligand. In some embodiments, said at least one of said one or more estrogenic agents comprises an ERβ selective ligand selected from the compounds of Formulae I through XIII as described herein.

As used in accordance with this invention, the term “ERβ selective ligand” means that the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERα and ERβ. It is preferred that the ERβ selective ligand will have a binding affinity to ERβ that is at least about 20 times greater than its binding affinity to ERα. It is more preferred that the ERβ selective ligand will have a binding affinity to ERβ that is at least about 50 times greater than its binding affinity to ERα. It is further preferred that the ERβ selective ligand is non-uterotrophic and non-mammotrophic.

As used in accordance with this invention, the term “non-uterotrophic” means producing an increase in wet uterine weight in a standard pharmacological test procedure of less than about 50% of the uterine weight increase observed for a maximally efficacious dose of 17β-estradiol or 17α-ethinyl-17β-estradiol in the same procedure. It is preferred that the increase in wet uterine weight will be less than about 25% of that observed for estradiol, and more preferred that the increase in wet uterine weight will be less than about 10% of that observed for estradiol. It is most preferred that the non-uterotrophic ERβ selective ligand will not increase wet uterine weight significantly (p>0.05) compared with a control that is devoid of uterotrophic activity (e.g. vehicle).

The instant invention also encompasses prodrugs. The prodrugs described herein are convertible in vivo or in vitro into one of the parent compounds. Typically, at least one of the biological activities of a compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound.

As used in accordance with this invention, the term “non-mammotrophic” means producing an increase in casein kinase 11 mRNA in a standard pharmacological test procedure of less than about 50% of the casein kinase 11 mRNA increase observed for a maximally efficacious dose of 17β-estradiol or 17α-ethinyl-17β-estradiol in the same procedure. It is preferred that the increase casein kinase II mRNA will be less than about 25% of that observed for estradiol, and more preferred that the increase in casein kinase II mRNA will be less than about 10% of that observed for estradiol. It is most preferred that the non-mammotrophic ERβ selective ligand will not increase casein kinase II mRNA significantly (p>0.05) compared with a control that is devoid of mammotrophic activity (e.g. vehicle).

As used herein, “therapeutic agents useful in the treatment of inflammatory diseases, disorders, or conditions of the skin, or diseases, disorders, or conditions associated with collagen depletion” include non-estrogenic agents, such as, for example and not limitation, tretinoin, retinol, retinaldehyde, alpha tocopherol, monolaurin, alpha hydroxy acids, hyaluronic acid, one or more hyaluronic acid fragments, botulinum toxin (Botox® Cosmetic), antibiotics, antihistamines, barrier-repair moisturizers, coal tar, corticosteroids, cyclosporine, interferon gamma, mycophenolate mofetil, topical calcineurin inhibitors, dietary supplements, herbs, teas, cold compresses and moisturizers.

As used herein, the term “hexose” means a sugar containing six carbon atoms. Suitable hexoses include but are not limited to glucose, mannose, galactose and fructose, in both their straight chain and pyranose forms. Modified hexoses include naturally occurring derivatives of hexoses, for example, phosphates, and corresponding acid and lactone forms. For example, the term “modified hexose” includes gluconic acid, gluconolactone, glucuronic acid, amino derivates including N-acetyl derivatives, phosphoate derivatives, and the like.

As used herein, the term “glucuronide derivative,” as applied to a specific compound, refers to a derivative of such compound where one or more hydroxyl groups of the compound have been replaced with a moiety of formula XX:

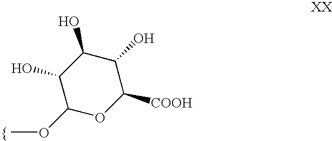

As used herein, the term “sulfate derivative,” as applied to a specific compound, refers to a derivative of such compound where one or more hydroxyl groups of the compound have been replaced with a moiety of formula —O—S(═O)₂—O—H.

As used herein, the term “glucuronide-sulfate derivative,” as applied to a specific compound, refers to a derivative of such compound where at least one hydroxyl group of the compound has been replaced with a moiety of formula XX, and at least one hydroxyl group of the compound has been replaced with a moiety of formula O—S(═O)₂—O—H.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. Alkyl groups can contain from 1 to about 20, 1 to about 10, 1 to about 8, 1 to about 6, 1 to about 4, or 1 to about 3 carbon atoms. In some embodiments, alkyl groups can be substituted with up to four substituent groups, as described below. As used herein, the term “lower alkyl” is intended to mean alkyl groups having up to six carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like. Alkenyl groups can contain from 2 to about 20, 2 to about 10, 2 to about 8, 2 to about 6, 2 to about 4, or 2 to about 3 carbon atoms. In some embodiments, alkenyl groups can be substituted with up to four substituent groups, as described below.

As used herein, “alkynyl” refers to an alkyl group having one or more triple carbon-carbon bonds. Examples of alkynyl groups include ethynyl, propynyl, butynyl, pentynyl, and the like. Alkynyl groups can contain from 2 to about 20, 2 to about 10, 2 to about 8, 2 to about 6, 2 to about 4, or 2 to about 3 carbon atoms. In some embodiments, alkynyl groups can be substituted with up to four substituent groups, as described below.

As used herein, “cycloalkyl” refers to non-aromatic carbocyclic groups including cyclized alkyl, alkenyl, and alkynyl groups. Cycloalkyl groups can be monocyclic (e.g., cyclohexyl) or poly-cyclic (e.g. 2, 3, or 4 fused ring, bridged, or spiro monovalent saturated hydrocarbon moiety), wherein the carbon atoms are located inside or outside of the ring system. Any suitable ring position of the cycloalkyl moiety may be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcarnyl, adamantyl, spiro[4.5]deanyl, homologs, isomers, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like.

As used herein, the term “acyl” refers to alkyl carbonyl groups, e.g., where alkyl is defined as herein. Acyl groups can contain from 2 to about 20, 2 to about 10, 2 to about 7, 2 to about 6, 2 to about 4, or 2 to about 3 carbon atoms.

As used herein, the term “aroyl” refers to any aryl moiety connected through a carbonyl group, such as a benzoyl group.

As used herein, “hydroxy” or “hydroxyl” refers to OH.

As used herein, “halo” or “halogen” includes fluoro, chloro, bromo, and iodo.

As used herein, “cyano” refers to CN.

As used herein, “alkoxy” refers to an —O-alkyl group. Example alkoxy groups include methoxy, ethoxy, propoxy (e.g., n-propoxy and isopropoxy), t-butoxy, and the like. An alkoxy group can contain from 1 to about 20, 1 to about 10, 1 to about 8, 1 to about 6, 1 to about 4, or 1 to about 3 carbon atoms. In some embodiments, alkoxy groups can be substituted with up to four substituent groups, as described below.

As used herein, the term “perfluoroalkoxy” indicates a group of formula —O-perfluoroalkyl.

As used herein, “haloalkyl” refers to an alkyl group, as defined herein, having one or more halogen substituents. Examples of haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.” Examples perhaloalkyl groups include CF₃ and C₂F₅.

As used herein, “haloalkoxy” refers to an —O-haloalkyl group, where alkyl is defined as herein.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “heterocyclic ring” is intended to refer to a monocyclic aromatic or non-aromatic ring system having from 5 to 10 ring atoms and containing 1-3 hetero ring atoms selected from O, N and S. In some embodiments, one or more ring nitrogen atoms can bear a substituent as described herein.

As used herein, “arylalkyl” or “aralkyl” refers to a group of formula -alkyl-aryl. The alkyl portion of the arylalkyl group can contain from 1 to about 20, 1 to about 10, 1 to about 8, 1 to about 6, 1 to about 4, or 1 to about 3 carbon atoms. Preferably, the alkyl portion of the arylalkyl group is a lower alkyl group, i.e., a C₁₋₆ alkyl group, more preferably a C₁₋₃ alkyl group. Examples of aralkyl groups include benzyl and naphthylmethyl groups.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, propyl, isopropyl, n-butyl, sec-butyl, isobutyl, etc.

The phrase “therapeutically effective amount” refers to the amount of a compound of the invention that elicits the biological or medicinal response in a tissue, system, animal, individual, patient, or human that is being sought by a researcher, veterinarian, medical doctor or other clinician. The desired biological or medicinal response may include inhibiting the disorder in a patient.

Compounds containing an amine function may also form N-oxides. A reference herein to a compound that contains an amine function also includes the N-oxide.

Where a compound contains several amine functions, one or more than one nitrogen atom may be oxidized to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of Deady (Syn. Comm., 1977, 7, 509-514) in which the amine compound is reacted with m-chloroperoxybenzoic acid (MCPBA), for example, in an inert solvent such as dichloromethane.

In some embodiments, the present invention provides methods of curbing, retarding, arresting or restraining an inflammatory disease, disorder, or condition of the skin, or a disease, disorder, or condition associated with collagen depletion comprising: administering to a mammal in need thereof, a therapeutically effective amount of one or more estrogenic agents.

In some embodiments, the present invention provides methods of lessening, slowing, stopping, or otherwise ameliorating the pathology, symptomatology, or progression of an inflammatory disease, disorder, or condition of the skin, or a disease, disorder, or condition associated with collagen depletion comprising: administering to a mammal in need thereof, a therapeutically effective amount of one or more estrogenic agents.

In some embodiments, the present invention provides methods which reduce the likelihood of getting, or delay the onset of an inflammatory disease, disorder, or condition of the skin, or a disease, disorder, or condition associated with collagen depletion comprising: administering to a mammal in need thereof, a therapeutically effective amount of one or more estrogenic agents.

In some embodiments, the one or more therapeutic agents useful in the treatment of inflammatory diseases, disorders, or conditions of the skin, or diseases, disorders, or conditions associated with collagen depletion can be provided by any of a variety of routes standard in the art, including for example and not limitation orally, transdermally, via injection, via an implant, intravaginally, rectally and the like. In some embodiments, systemic administration is achieved orally via ingestion of a pill, tablet, capsule or other oral dosage form. In some embodiments, the combination of one or more estrogenic agents and one or more therapeutic agents useful in the treatment of inflammatory diseases, disorders, or conditions of the skin, or diseases, disorders, or conditions associated with collagen depletion, are administered in a single dosage form, for example in cream or ointment.

In some embodiments, the combination of one or more estrogenic agents and one or more therapeutic agents useful in the treatment of inflammatory diseases, disorders, or conditions of the skin, or diseases, disorders, or conditions associated with collagen depletion include combinations of one or more suitable estrogenic agents, such as those described herein, in any combination.

The therapeutically effective amount provided in the prevention, treatment, or inhibition of a specific disease, disorder, or condition may vary according to the specific disease, disorder, or condition being treated, the size, the age and response pattern of the patient, the severity of the disorder, and the judgment of the attending physician or the like. It is projected that a therapeutically effective amount of the compounds of this invention may be given at a daily dose of from about 5 μg/kg to about 100 mg/kg. The projected daily dosages are expected to vary with route of administration, and the nature of the compound administered.

In accordance with the methods of the invention, the one or more estrogenic agents and the one or more therapeutic agents useful in the treatment of inflammatory diseases, disorders, or conditions of the skin, or diseases, disorders, or conditions associated with collagen depletion can each be independently administered in a continuous, intermittent or interrupted dosing regime. The duration of the regime of any or all of the foregoing components can each independently be of any length, from a single administration, to chronic therapy regimes.

In some embodiments, the dosing regime includes or consists of daily dosing of at least one of said one or more estrogenic agents, and daily dosing of at least one of said one or more therapeutic agents useful in the treatment of inflammatory diseases, disorders, or conditions of the skin, or diseases, disorders, or conditions associated with collagen depletion.

In some embodiments, the dosing regime includes or consists of daily dosing of at least one of said one or more estrogenic agents, and intermittent or interrupted dosing of at least one of said one or more therapeutic agents useful in the treatment of inflammatory diseases, disorders, or conditions of the skin, or diseases, disorders, or conditions associated with collagen depletion.

In some further embodiments, the dosing regime includes or consists of intermittent or interrupted dosing of at least one of said one or more estrogenic agents, and daily dosing of at least one of said one or more therapeutic agents useful in the treatment of inflammatory diseases, disorders, or conditions of the skin, or diseases, disorders, or conditions associated with collagen depletion.

In some further embodiments, the dosing regime includes or consists of intermittent or interrupted dosing of at least one of said one or more estrogenic agents, and intermittent or interrupted dosing of at least one of said one or more therapeutic agents useful in the treatment of inflammatory diseases, disorders, or conditions of the skin, or diseases, disorders, or conditions associated with collagen depletion.

As used herein, the term “continuous” as used in connection with a dosing regime of the invention, is intended to mean a regime in which the dose is administered at uniform intervals, up to and including daily administration.

As used herein, the term “intermittent” as used in connection with a dosing regime of the invention, is intended to mean a regime in which the dose is administered at uniform intervals less frequently than daily. Examples of intermittent dosing regimes include alternate days, every third day, every fourth day, every fifth day, every sixth day, weekly, bi-weekly, and the like.

As used herein, the term “interrupted” as used in connection with a dosing regime of the invention, is intended to mean a regime in which the dose is administered at non-sequential or non-uniform intervals. Non-limiting examples of interrupted dosing regimes include a period of continuous administration (e.g., daily) followed by a period of discontinuous or intermittent administration, or a period of non-administration, optionally followed by an additional period of continuous or intermittent administration, or periods in which the various components of the regimen are administered alternately in either a continuous or intermittent fashion.

As used herein, the terms “administering” or “providing” mean either directly administering the one or more estrogenic agents, or administering a prodrug, derivative, or analog of said one or more estrogenic agents that will form an effective amount of the one or more estrogenic agents within the body. The terms include routes of administration that are systemic (e.g., via injection, orally in a tablet, pill, capsule, or other dosage form useful for systemic administration of pharmaceuticals, and the like, such as described herein below), and topical (e.g., creams, solutions, and the like, including solutions such as mouthwashes, for topical oral administration).

The term “in need thereof” and the like as used herein refers to a subject that has been determined to be in need of treatment for an inflammatory disease, disorder, or condition of the skin, or a disease, disorder, or condition associated with collagen depletion such as, for example not limitation, photo aging, chronological skin aging, smoking induced skin aging, psoriasis, eczema, or dermatitis. Such a determination may be a result of a medical diagnosis. Further, subjects “in need” of the methods of the present invention include those known or suspected to be afflicted with an inflammatory disease, disorder, or condition of the skin, or a disease, disorder, or condition associated with collagen depletion. Additionally, subjects “in need thereof” with respect to prophylactic methods include those who are, or are suspected to be, predisposed or susceptible to inflammatory diseases, disorders, or conditions of the skin, or diseases, disorders, or conditions associated with collagen depletion. Predisposition or susceptibility to such diseases, disorders, or conditions can be from any source, genetic, environmental, exposure, etc.

In some embodiments the methods of the present invention comprise administering to the subject escalating doses of one or more estrogenic agents. In some embodiments, at least one of said one or more estrogenic agents are applied topically. In some embodiments, at least one of said one or more estrogenic agents is non-uterotrophic and non-mammotrophic.

In some embodiments, one or more estrogenic agents are applied transdermally to the affected area. In some embodiments, a pharmaceutical composition comprising a combination of one or more estrogenic agents and one or more therapeutic agents useful in the treatment of inflammatory diseases, disorders, or conditions of the skin, or diseases, disorders, or conditions associated with collagen depletion is applied transdermally to the affected area.

For the purposes of this disclosure, “transdermally” or “transdermal” administrations are understood to include all administrations across the surface of the body and the inner linings of bodily passages including epithelial and mucosal tissues.

The transdermally provided estrogenic agents of the present invention can be provided in accordance with the methods of the invention by any of a variety of routes standard in the art, including for example and not limitation, lotions, creams, foams, patches, suspensions, solutions, slurries, pessaries, mechanical carriers, masks, and suppositories (rectal and vaginal). In some embodiments, one or more of said transdermally administered estrogenic agents is administered in a cream. In some embodiments, one or more of said transdermally administered estrogenic agents is administered in an ointment.

In some embodiments, transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Further examples of dosage forms, diluents, carriers, excipients, and the like, that are suitable for use in connection with the embodiments of the invention are known in the art and described in, for example, Remington's Pharmaceutical Sciences, 17th ed., Mack Publishing Company, Easton, Pa., 1985, which is incorporated herein by reference in its entirety.

In some embodiments, compounds of the present invention may be combined with one or more therapeutic agents useful in the treatment of inflammatory diseases, disorders, or conditions of the skin, or diseases, disorders, or conditions associated with collagen depletion such as, for example and not limitation, chronological skin aging, photo aging, dermatitis, psoriasis, and eczema. In some embodiments, the compounds of the present invention may be combined with one or more therapeutic agents which suppress MMP1, IL1B, and/or IL8 activity in mammalian epidermal and dermal skin.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 7-bromo-2-(4-hydroxyphenyl)-1,3-benzoxazol-5-ol. In some further embodiments, the methods of the present invention further comprise: providing to a mammal in need thereof, a therapeutically effective amount of 7-bromo-2-(4-hydroxyphenyl)-1,3-benzoxazol-5-ol, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 7-bromo-2-(4-hydroxyphenyl)-1,3-benzoxazol-5-ol. In some further embodiments, the present invention provides a pharmaceutical composition comprising a therapeutically effective amount of 7-bromo-2-(4-hydroxyphenyl)-1,3-benzoxazol-5-ol, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 7-bromo-2-(3-fluoro-4-hydroxyphenyl)-1,3-benzoxazol-5-ol. In some further embodiments, the methods of the present invention further comprise: providing to a mammal in need thereof, a therapeutically effective amount of 7-bromo-2-(3-fluoro-4-hydroxyphenyl)-1,3-benzoxazol-5-ol, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 7-bromo-2-(3-fluoro-4-hydroxyphenyl)-1,3-benzoxazol-5-ol. In some further embodiments, the present invention provides a pharmaceutical composition comprising a therapeutically effective amount of 7-bromo-2-(3-fluoro-4-hydroxyphenyl)-1,3-benzoxazol-5-ol, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 5-hydroxy-2-(4-hydroxyphenyl)-1,3-benzoxazole-7-carbonitrile. In some further embodiments, the methods of the present invention further comprise: providing to a mammal in need thereof, a therapeutically effective amount of 5-hydroxy-2-(4-hydroxyphenyl)-1,3-benzoxazole-7-carbonitrile, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 5-hydroxy-2-(4-hydroxyphenyl)-1,3-benzoxazole-7-carbonitrile. In some further embodiments, the present invention provides a pharmaceutical composition comprising a therapeutically effective amount of 5-hydroxy-2-(4-hydroxyphenyl)-1,3-benzoxazole-7-carbonitrile, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 2-(3-fluoro-4-hydroxyphenyl)-7-vinyl-1,3-benzoxazol-5-ol. In some further embodiments, the methods of the present invention further comprise: providing to a mammal in need thereof, a therapeutically effective amount of 2-(3-fluoro-4-hydroxyphenyl)-7-vinyl-1,3-benzoxazol-5-ol, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 2-(3-fluoro-4-hydroxyphenyl)-7-vinyl-1,3-benzoxazol-5-ol. In some further embodiments, the present invention provides a pharmaceutical composition comprising a therapeutically effective amount of 2-(3-fluoro-4-hydroxyphenyl)-7-vinyl-1,3-benzoxazol-5-ol, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 3-(3-fluoro-4-hydroxyphenyl)-7-hydroxy-1-naphthonitrile. In some further embodiments, the methods of the present invention further comprise: providing to a mammal in need thereof, a therapeutically effective amount of 3-(3-fluoro-4-hydroxyphenyl)-7-hydroxy-1-naphthonitrile, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 3-(3-fluoro-4-hydroxyphenyl)-7-hydroxy-1-naphthonitrile. In some further embodiments, the present invention provides a pharmaceutical composition comprising a therapeutically effective amount of 3-(3-fluoro-4-hydroxyphenyl)-7-hydroxy-1-naphthonitrile, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 2-(2-fluoro-4-hydroxyphenyl)-7-vinyl-1,3-benzoxazol-5-ol. In some further embodiments, the methods of the present invention further comprise: providing to a mammal in need thereof, a therapeutically effective amount of 2-(2-fluoro-4-hydroxyphenyl)-7-vinyl-1,3-benzoxazol-5-ol, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 2-(2-fluoro-4-hydroxyphenyl)-7-vinyl-1,3-benzoxazol-5-ol. In some further embodiments, the present invention provides a pharmaceutical composition comprising a therapeutically effective amount of 2-(2-fluoro-4-hydroxyphenyl)-7-vinyl-1,3-benzoxazol-5-ol, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 2-(2,3-difluoro-4-hydroxyphenyl)-7-vinyl-1,3-benzoxazol-5-ol. In some further embodiments, the methods of the present invention further comprise: providing to a mammal in need thereof, a therapeutically effective amount of 2-(2,3-difluoro-4-hydroxyphenyl)-7-vinyl-1,3-benzoxazol-5-ol, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 2-(2,3-difluoro-4-hydroxyphenyl)-7-vinyl-1,3-benzoxazol-5-ol. In some further embodiments, the present invention provides a pharmaceutical composition comprising a therapeutically effective amount of 2-(2,3-difluoro-4-hydroxyphenyl)-7-vinyl-1,3-benzoxazol-5-ol, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

In some embodiments, the present invention provides methods of preventing, treating, or inhibiting an inflammatory disease, disorder, or condition of the skin, or a disease, disorder, or condition associated with collagen depletion, comprising: providing to a mammal in need thereof, a therapeutically effective amount of 7-(2-fluoro-4-hydroxyphenyl)-2-naphthol. In some further embodiments, the methods of the present invention further comprise: providing to a mammal in need thereof, a therapeutically effective amount of 7-(2-fluoro-4-hydroxyphenyl)-2-naphthol, in combination with one or more therapeutic agents useful in the treatment of inflammatory diseases, disorders, or conditions of the skin, or diseases, disorders, or conditions associated with collagen depletion. Some embodiments of the present invention provide methods of suppressing MMP1, IL1B, and/or IL8 activity in mammalian epidermal and dermal skin comprising providing to a mammal in need thereof, a therapeutically effective amount of 7-(2-fluoro-4-hydroxyphenyl)-2-naphthol. In some further embodiments, the present invention provides a pharmaceutical composition comprising a therapeutically effective amount of 7-(2-fluoro-4-hydroxyphenyl)-2-naphthol, in combination with one or more therapeutic agents useful in the treatment of inflammatory diseases, disorders, or conditions of the skin, or diseases, disorders, or conditions associated with collagen depletion and at least one pharmaceutically acceptable diluent or carrier.

The present invention further provides a compound of any one of Formulae I-XIII, as described herein, or a composition as described herein, for use as a medicament for preventing, treating, or inhibiting an inflammatory disease, disorder, or condition of the skin, or a disease, disorder, or condition associated with collagen depletion.

The present invention further provides the use of a compound of any one of Formulae I-XIII as described herein, or a composition as described herein, in the manufacture of a medicament for preventing, treating, or inhibiting an inflammatory disease, disorder, or condition of the skin, or a disease, disorder, or condition associated with collagen depletion.

Some further embodiments of the present invention provide a kit comprising:

a) a therapeutically effective amount of one or more estrogenic agents, or a pharmaceutically acceptable salt, solvate, solvate of such a salt, or a prodrug thereof, in a first dosage unit form; and
b) a therapeutically effective amount of one or more therapeutic agents useful in the treatment of inflammatory diseases, disorders, or conditions of the skin, or diseases, disorders, or conditions associated with collagen depletion; or a pharmaceutically acceptable salt, solvate, solvate of such a salt, or a prodrug thereof, in a second dosage unit form.
In some such embodiments, a container means is provided for containing said first and second dosage forms.

Some further embodiments of the present invention provide a kit comprising:

a) a therapeutically effective amount of one or more estrogenic agents wherein said one or more estrogenic agents is a compound of Formulae I-XIII, or a pharmaceutically acceptable salt, solvate, solvate of such a salt, or a prodrug thereof, in a first dosage unit form;
b) a therapeutically effective amount of one or more therapeutic agents useful in the treatment of inflammatory diseases, disorders, or conditions of the skin, or diseases, disorders, or conditions associated with collagen depletion; or a pharmaceutically acceptable salt, solvate, solvate of such a salt, or a prodrug thereof, in a second dosage unit form.
In some such embodiments, a container means is provided for containing said first and second dosage forms.

The materials, methods, and examples presented herein are intended to be illustrative, and are not intended to limit the scope of the invention.

### EXAMPLES

### Materials

In Vitro Epidermal Keratinocyte and Dermal Fibroblast Culture Systems for Dermal Target Compound Evaluation

Normal Human Epidermal Keratinocytes (NHEK, from Cambrex), human keratinocyte cell line KERTr, human dermal fibroblasts cell lines, HFF and BJ-5ta (from ATCC), were used for this study. The culturing procedures were the same as the vendor recommended. In general, cells were trypsinized and seeded on day 0, compound treatments were done on day 1 with and without UV (8 mJ or 60 mJ) or TNF-alpha (0.5 or 1 ng/ml) activations, and cells were harvested on day 2 with lysis buffer directly added to the cultured cells after PBS wash. Cell lysates were either purified through Qiagen RNeasy RNA purification column or directly proceeded to cDNA conversion using “Cell-to-cDNA” lysis buffer from Ambion.

Methods

Taqman Low Density Array and Individual Taqman Assays for Monitoring Gene Regulation of Cytokines, Chemokines, and MMPs

The RNA or cDNA obtained from the compound treated cells were used in Taqman Low Density Array (TLDA, custom made from Applied Biosystems) or individual Taqman assays (Applied Biosystems) by following the vendors standard protocols using ABI 7900HT Real-time PCR machine. The “Cytokine TLDA” designed to evaluate the compounds of the present invention, contained TNF, IL1B, IL6, IL8, MMP1, MMP3, MMP9, TIMP1, DCN, COL1A1, CCL3, CCL4, CCL5, NOS2A, PTSG2, and 18S control. Further, some of these individual gene assays were also used for confirmation or focused assay purposes.

In Vivo Studies

Measurement of Skin Thickness and Wrinkle Formation in the In Vivo Photoaging Model

Dorsal skin of hairless mice was lifted up by pinching gently. Skin-fold thickness was measured using a caliper (Peacock; Ozaki MFG Co. Ltd., Tokyo, Japan). Skin thickness was measured on weeks 0, 2, 4 and 6.

Skin surface impressions (replicas) were prepared by applying silicon rubber to dorsal skin (measured on weeks 0 and 6).

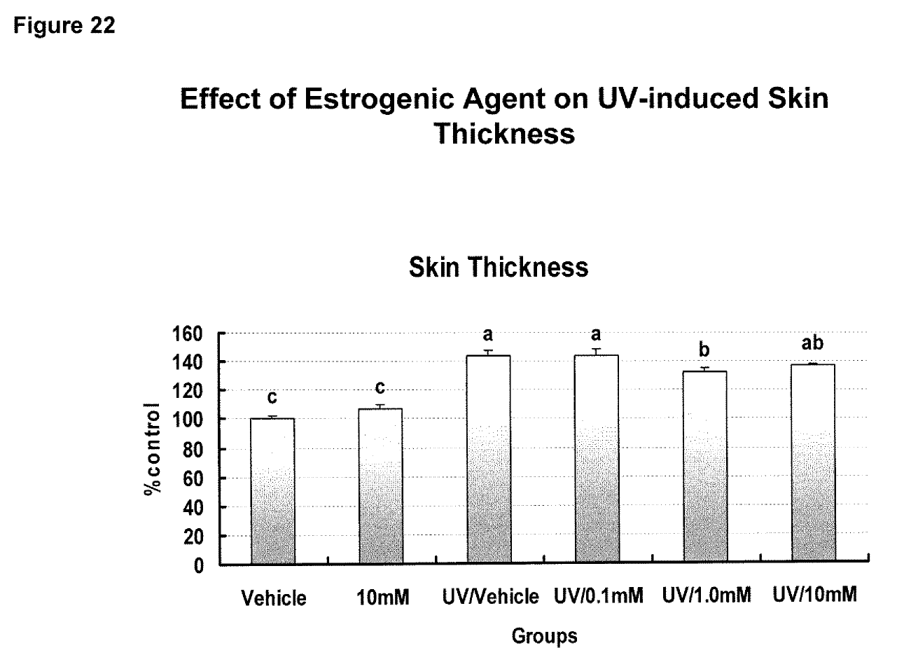

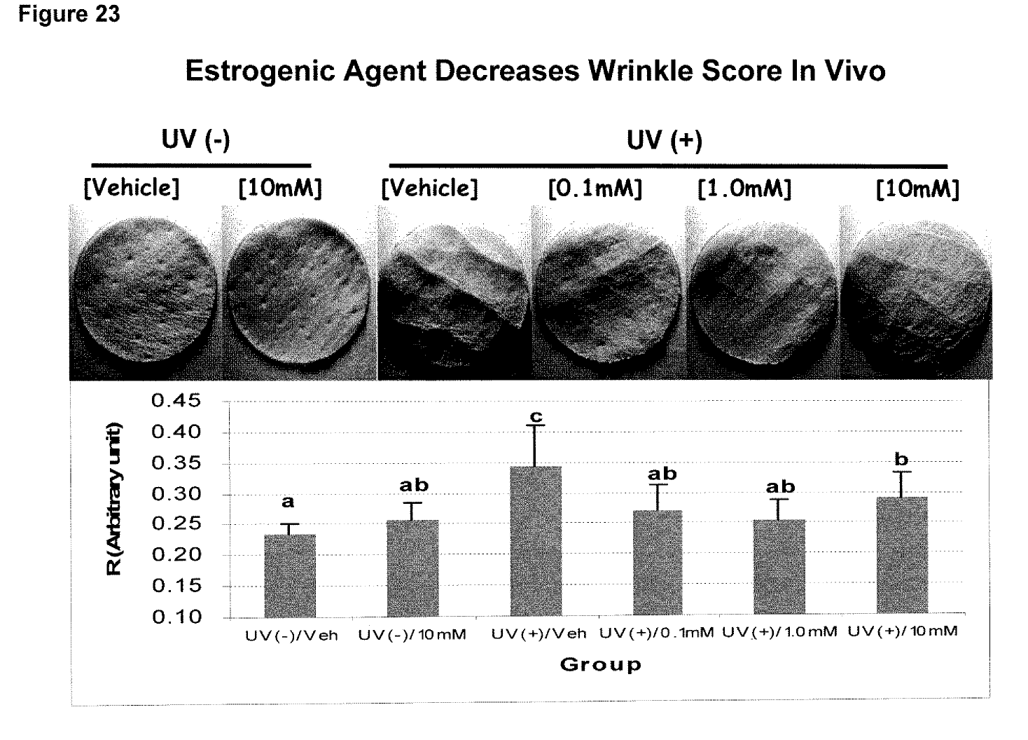

The bars in FIGS. 22 and 23 indicate the mean values±standard error of the mean (sem) of 6 separated experiments with n=8 animals per group. Data were statistically analyzed by one-way ANOVA followed by DUNCAN, and considered significantly different when P<0.05 was obtained.

ERβ KO Mouse Studies

The ERβ KO mouse was developed by Wyeth Research. This KO mouse utilizes a construct that forces translational block after the 19^{th} codon due to the insertion of translational stop codons in all three reading frames. Insertion of the neomycin resistance gene in reverse orientation into the targeting construct results in the truncation of exons 1 and 2 and deletion of intron 1. The detail description and characterization of this line can be found in the publication (Endocrinology 143(5): 1643-1650, 2002).

Skins from newborn mice (2-3 days old) were isolated and floated on 2.5 mg/ml dispase (Invitrogen/Gibco, Carlsbad, Calif.) overnight at 4° C. then carefully separated into epidermal and dermal layers using small forceps. The epidermal and dermal layers were minced and followed by several differential centrifugations, fractionations and filtrations as previously described. These cells were then cultured in Eagle's minimal essential medium containing 8% fetal bovine serum on 24-well culture plate on day 0. Cells were treated with vehicle, T1317 and other compounds on day 2, then isolated and purified RNA on day 3 using RNeasy column (Qiagen). Gene expression profiles were analyzed using Taqman Low Density Array and individual Taqman gene assays (Applied Biosystems, Foster City, Calif.).

In Vivo Photoaging Model

Six-week-old female albino hairless mice (Hos:HR-1) were obtained from SLC (Hizuoka Institute for Laboratory Animals, Inc., Hamamatsu, Shizuoka Prefecture, Japan). Animals were acclimated for 1 week prior to study and had free access to food and water. Eight mice were allocated to each group (total six groups for each test compounds).

UV irradiation device that included TL20W/12RS UV lamp, with an emission spectrum between 275 and 380 nm (peak, 310-315 nm) was used as the UV source¹. A Kodacel filter (TA401/407; Kodak, Rochester, N.Y.) was mounted 2 cm in front of the UV lamps to remove UVC wavelengths≦290 nm. Irradiation intensity on mouse dorsal skin was measured using a UV meter (Model 585100; Waldmann Co., Villingen-Schwenningen, Germany). The irradiation intensity 30 cm from the light source was 1.0 mW cm⁻². The minimal erythemal dose (MED) of mouse dorsal skin was determined. MED is defined as the minimum amount of radiation exposure required to produce erythema with sharp margins after 24 h. Irradiations were performed three times per week (Monday, Wednesday and Friday). The weekly UV dose was increased weekly by 1 MED (1MED=100 mJ cm⁻²) up to 4 MED and then, UV dose was maintained at this level.

ERβ-selective ligand or its vehicle (70% ethanol, 30% polyethylene glycol) was topically applied to the dorsal area (50 ul) after each exposure to UV irradiation (5 times a week—Mon, Tue, Wed, Thu and Fri)

Exemplary Compounds

Preparation of Representative Example Compounds Suitable for Use in Some embodiments of the methods and compositions of the invention, particularly those compounds of Formulae I through XIII, are described in U.S. Pat. Nos. 6,903,238 (Compounds of Formula XI); 6,914,074 (Compounds of Formula III); 6,774,248 (Compounds of Formula IV); 6,960,607 (Compounds of Formula IX); 6,884,814 (Compounds of Formula VIII); 6,794,403 (Compounds of Formula II); 6,723,747 (Compounds of Formula VII); 6,835,745 (Compounds of Formula XII); and 7,084,276 (Compounds of Formula XIII); and pending U.S. patent application Ser. Nos. 11/210,427 (Compounds of Formula I); 11/170,017 (Compounds of Formula VI); 11/219,940 (Compounds of Formula V); and 12/110,728 (Compounds of Formula I); each of which is incorporated herein in its entirety for any purpose.

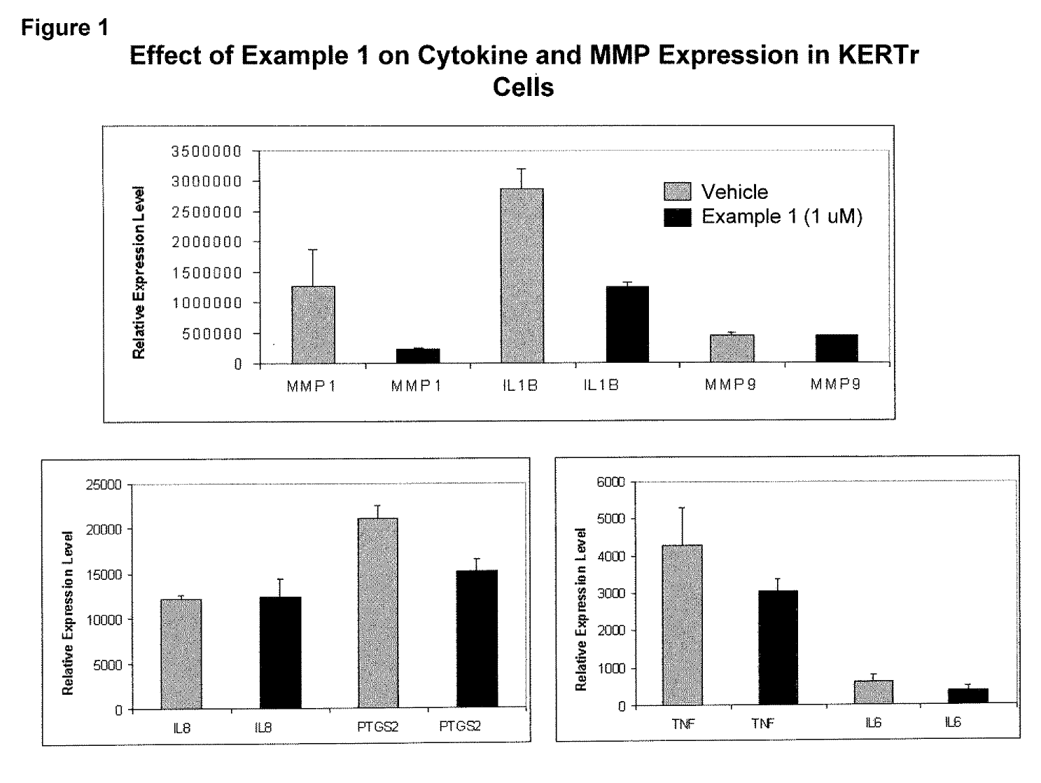

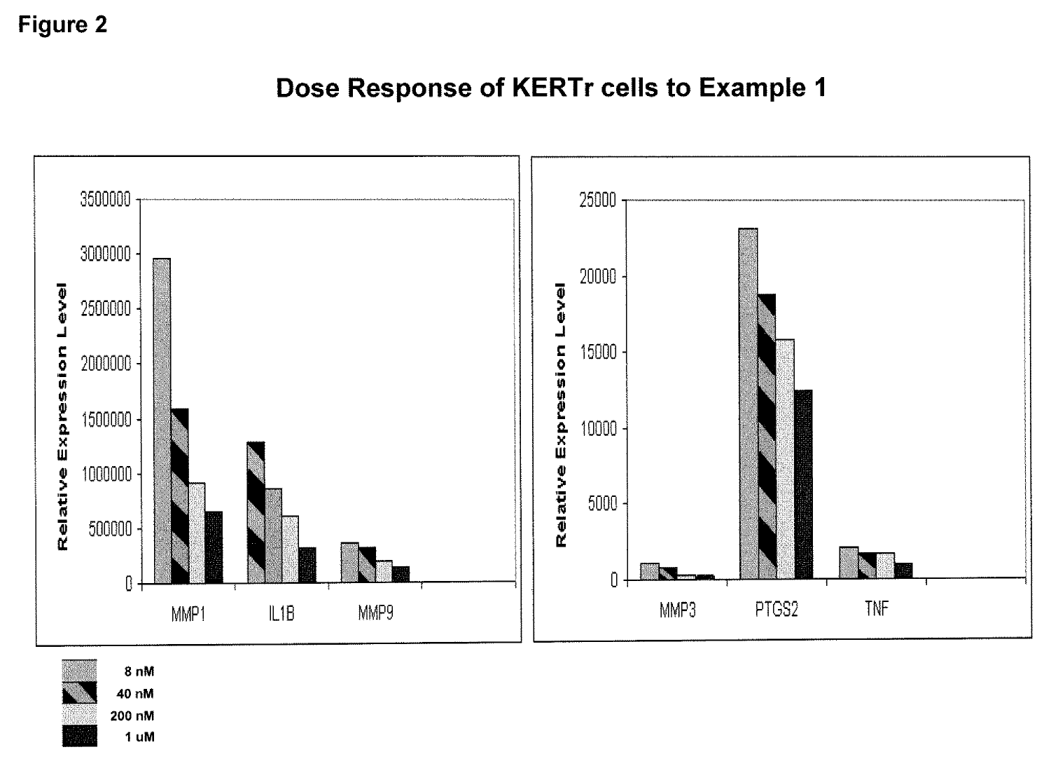

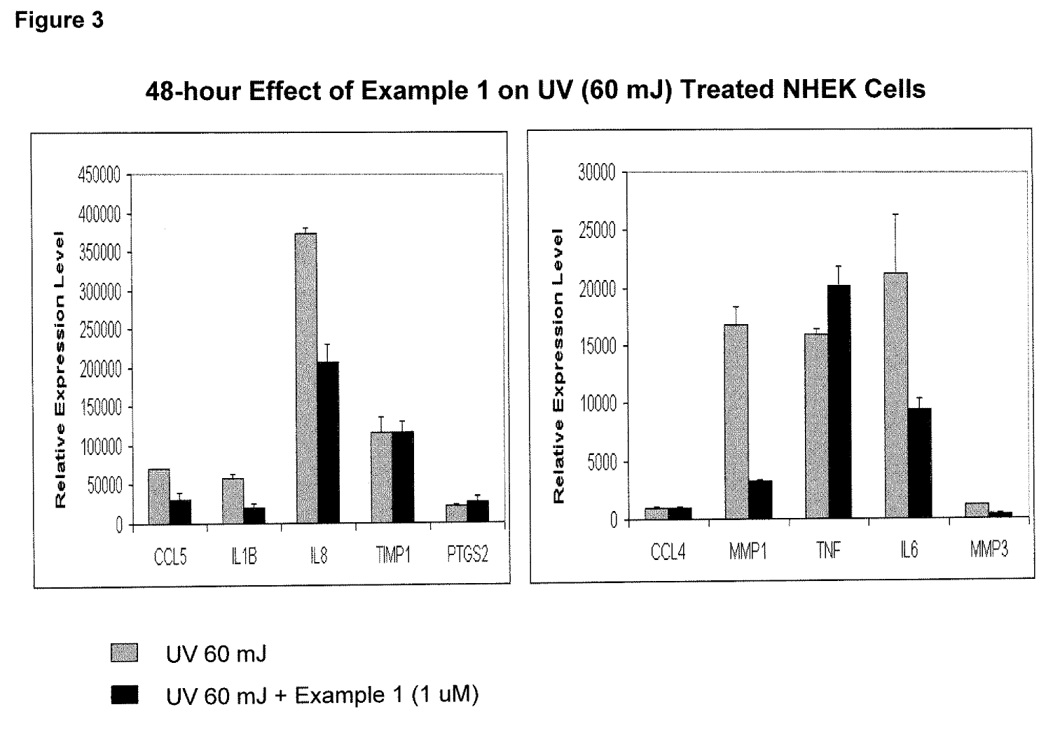

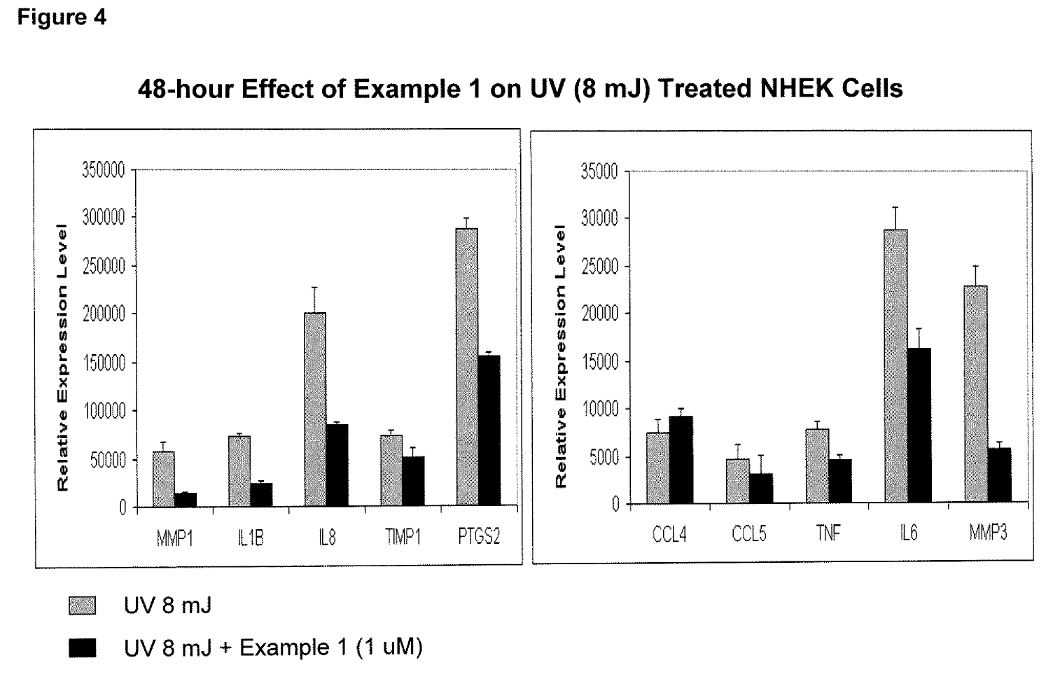

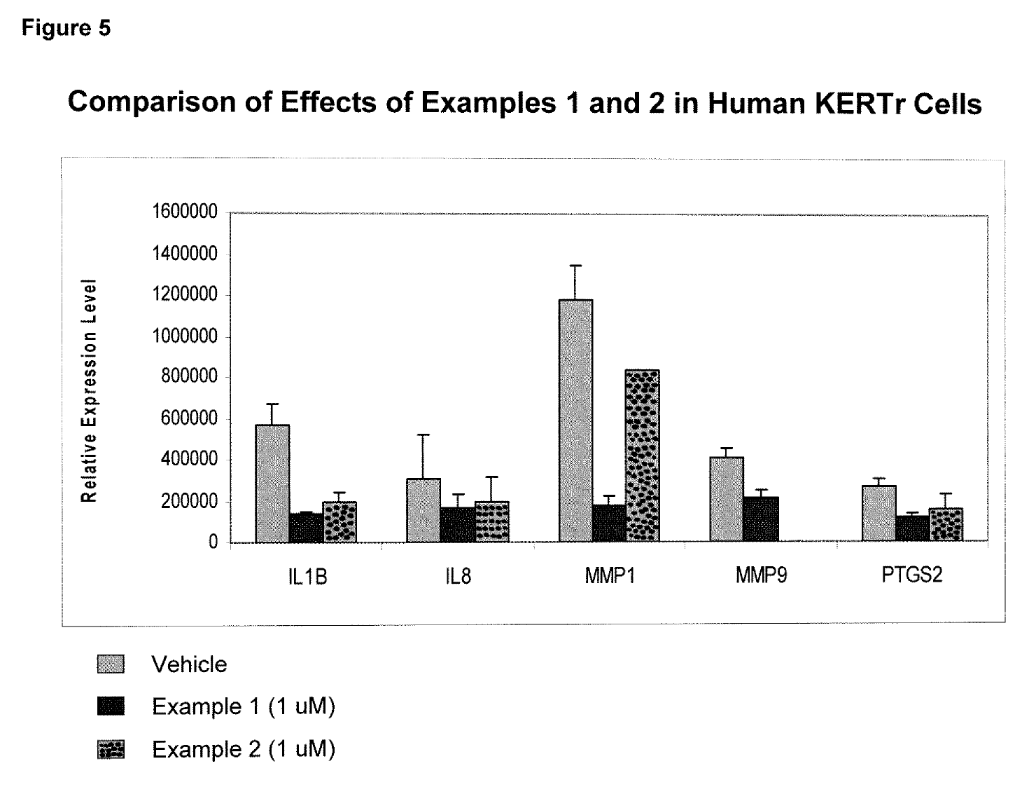

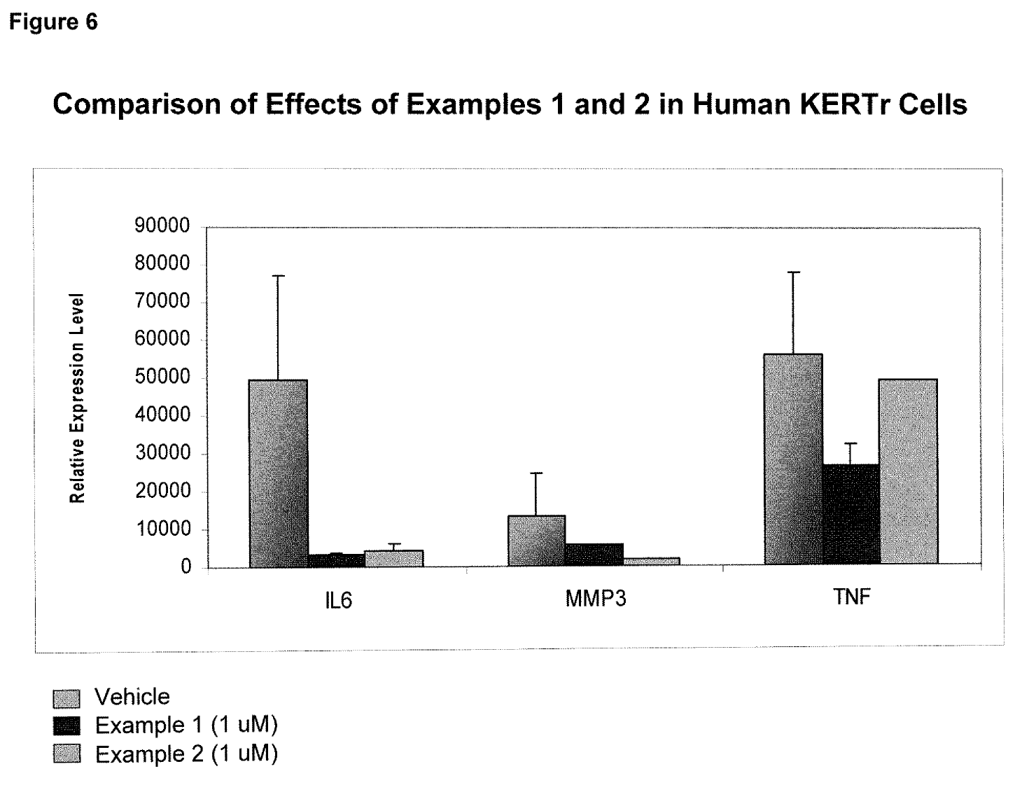

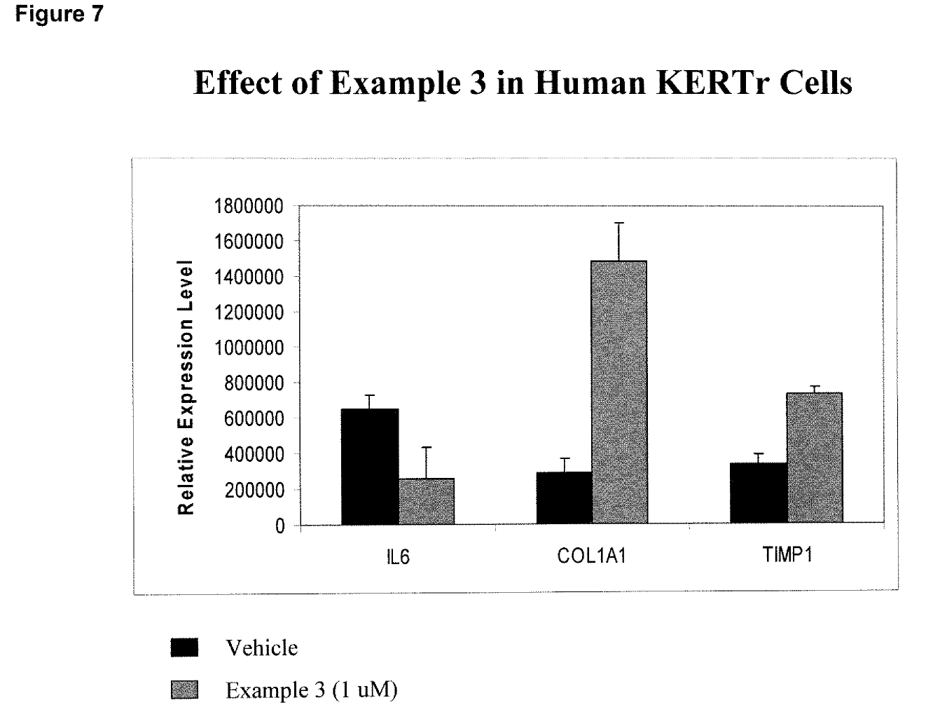

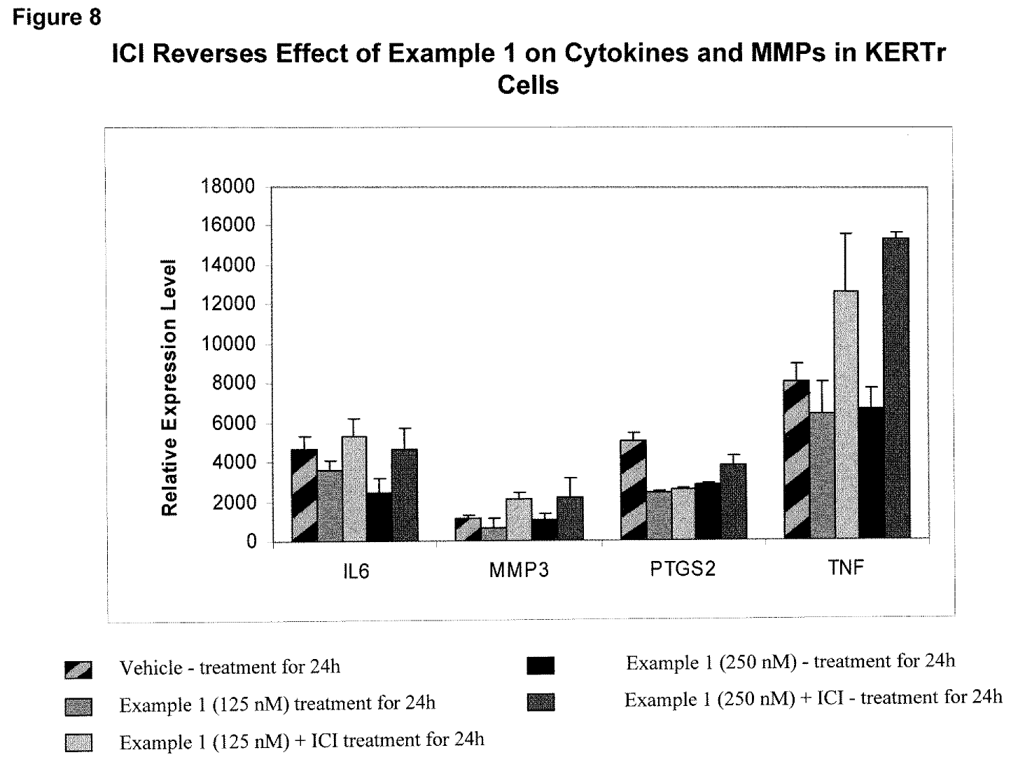

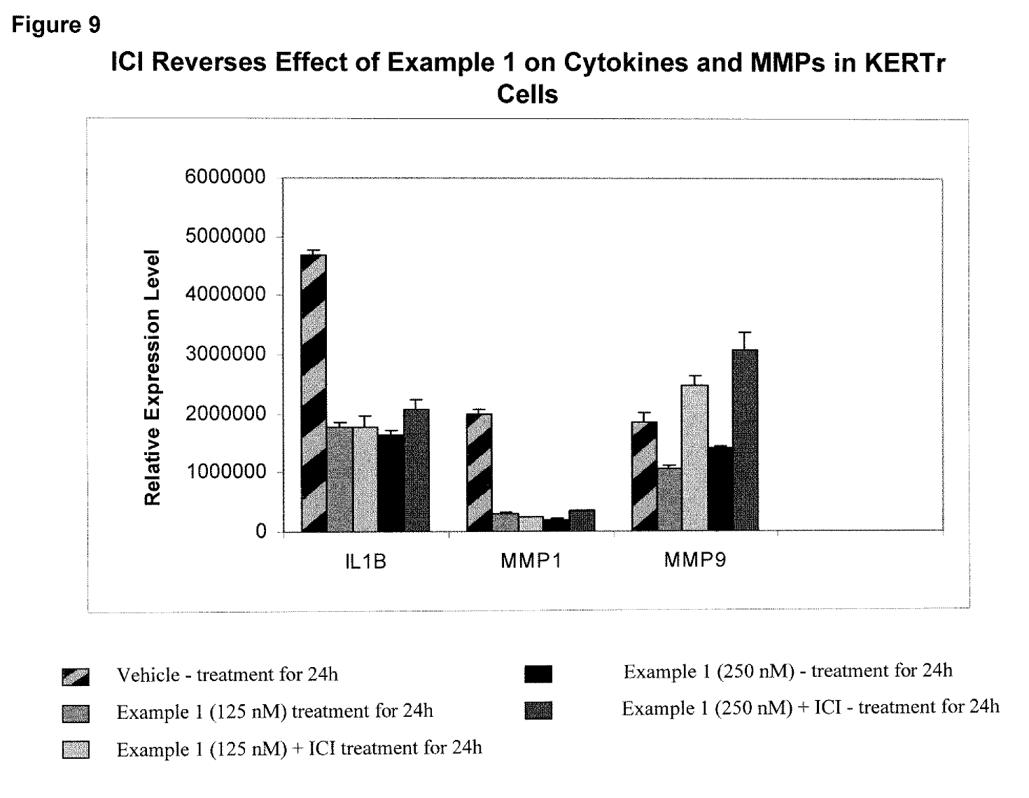

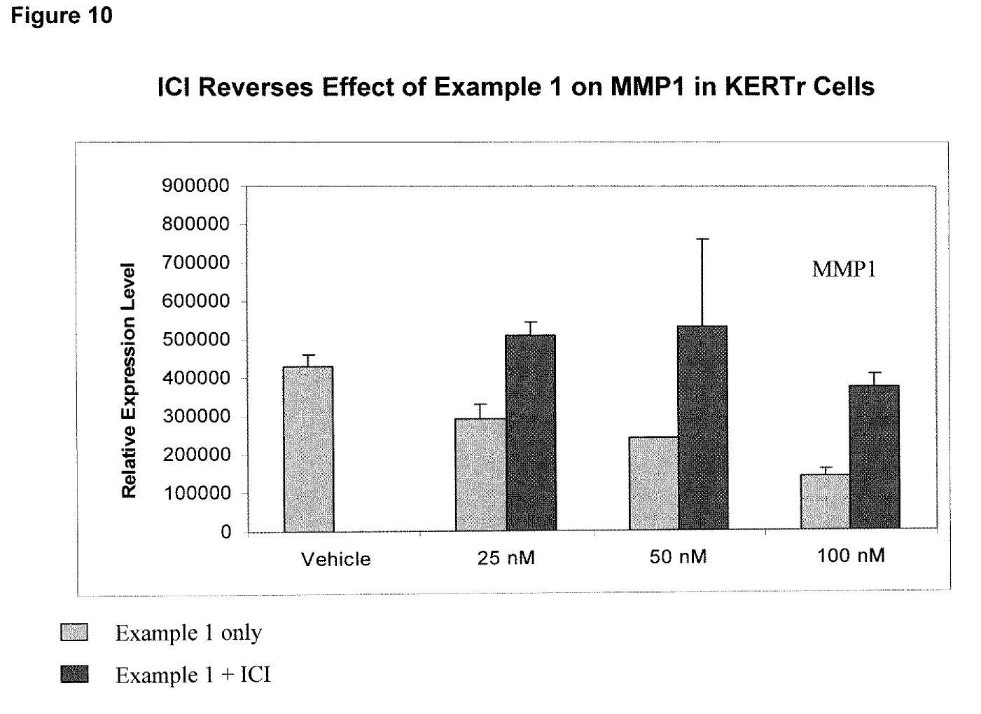

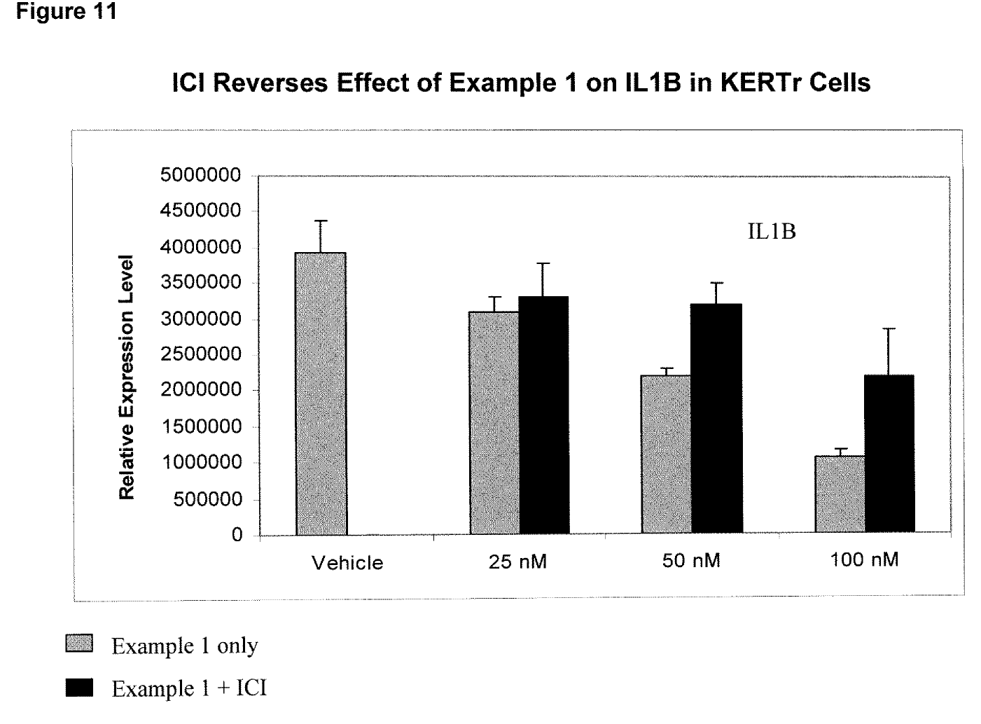

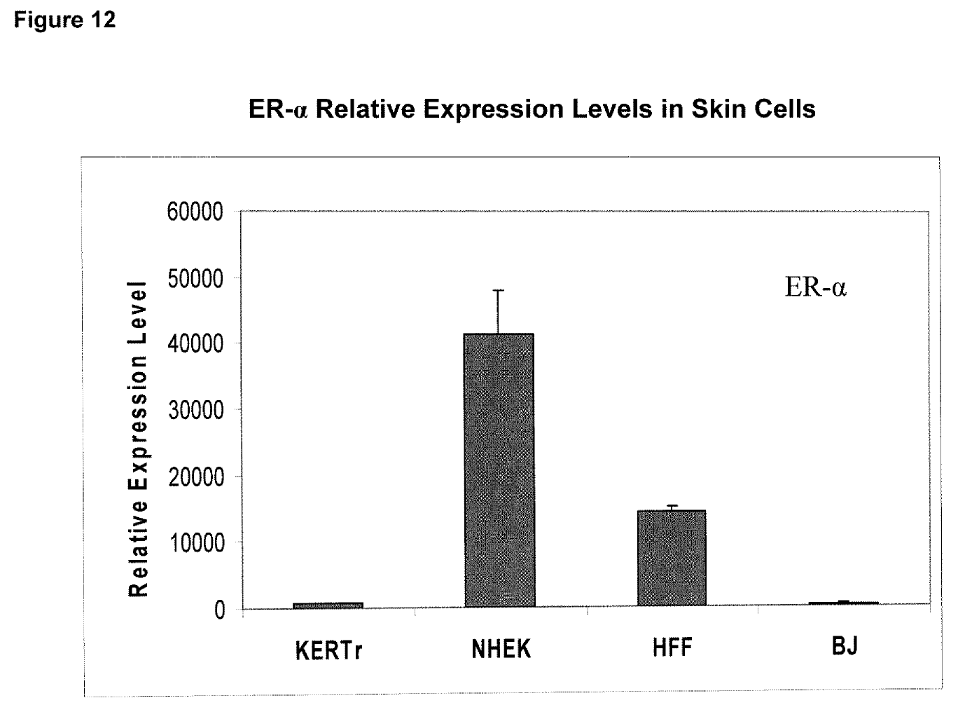

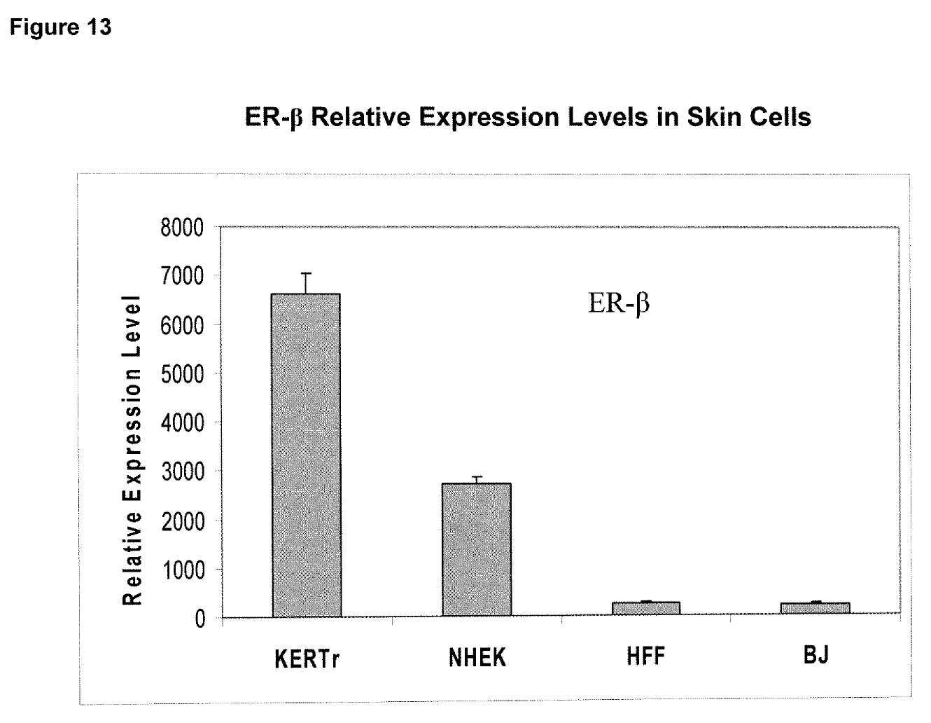

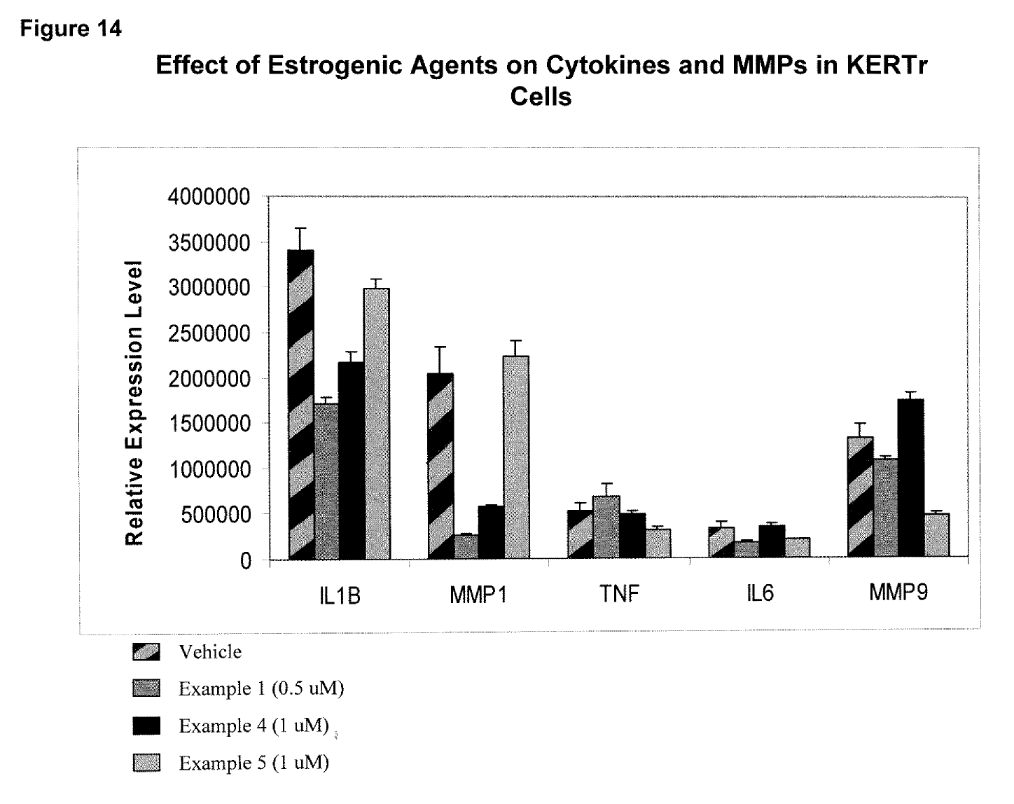

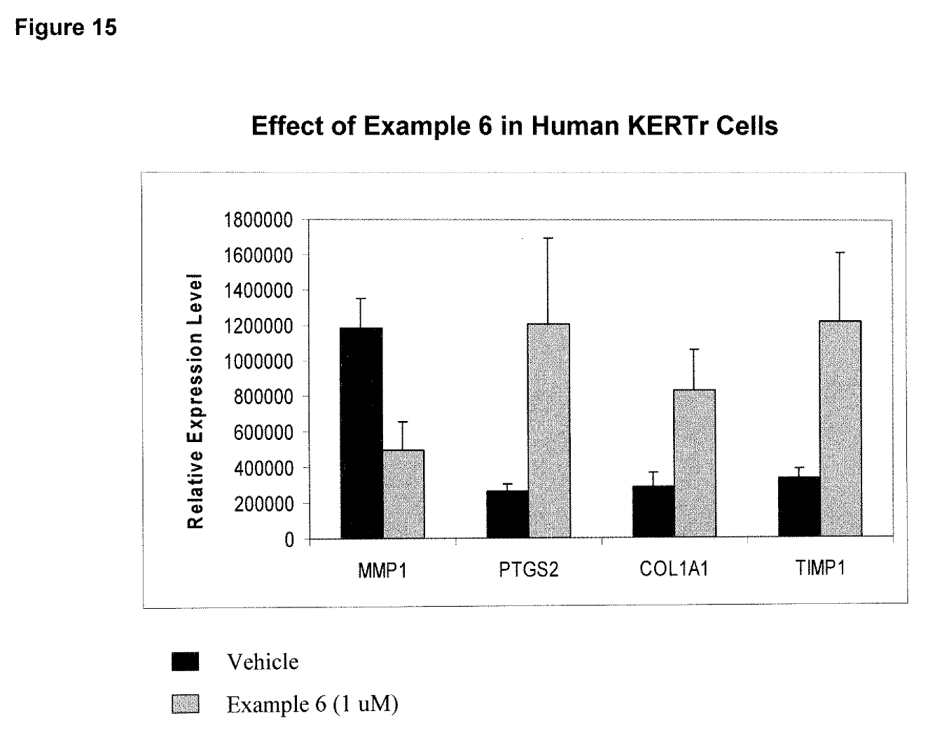

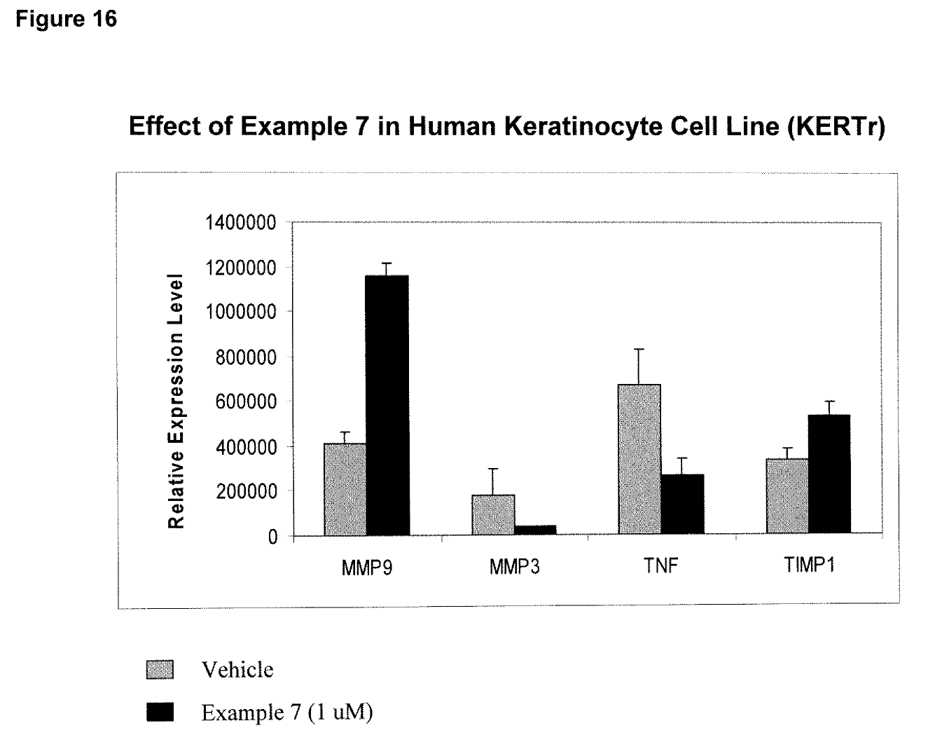

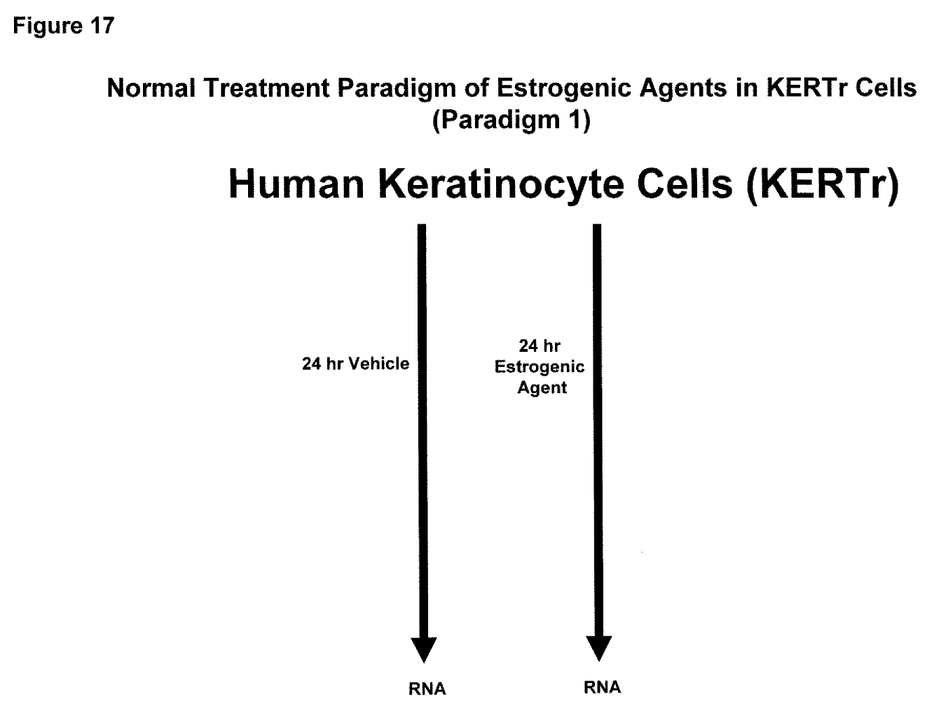

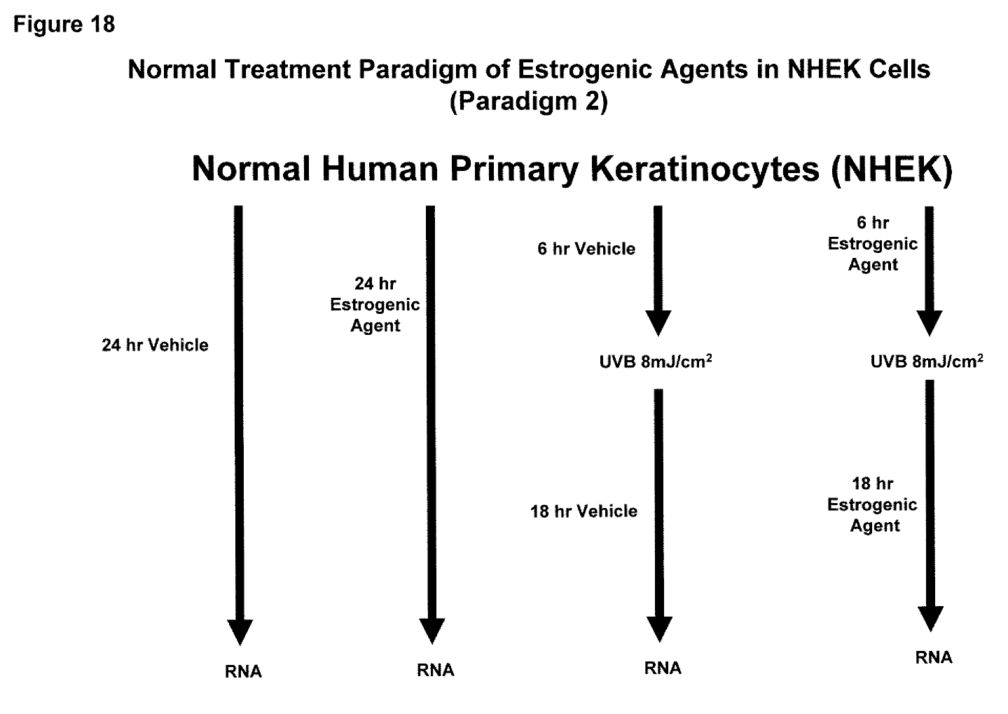

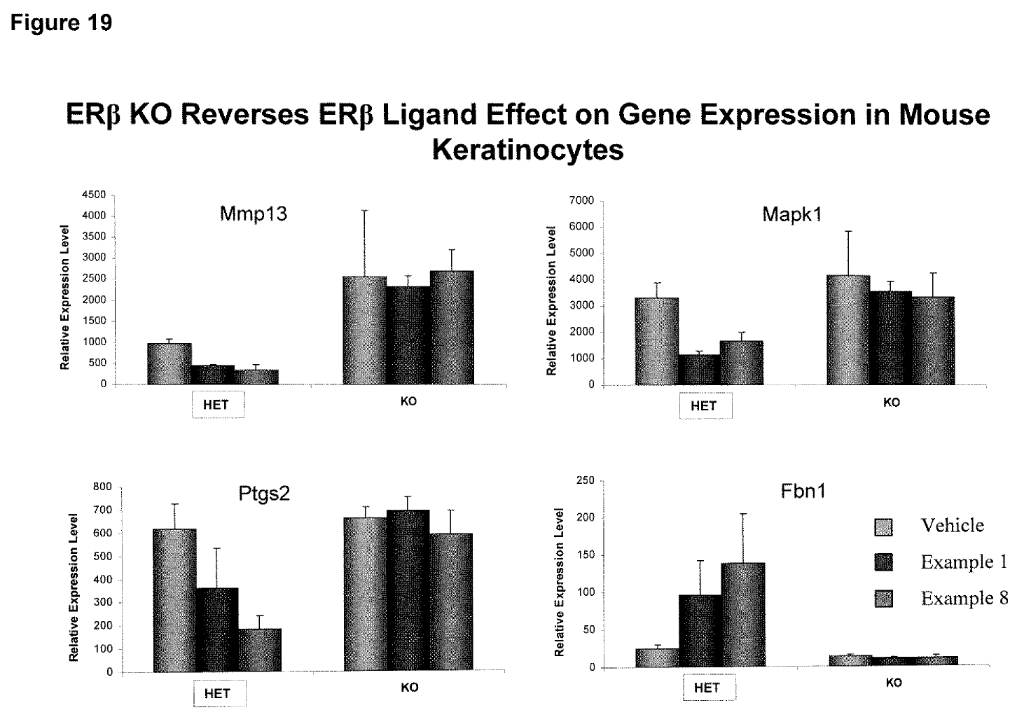

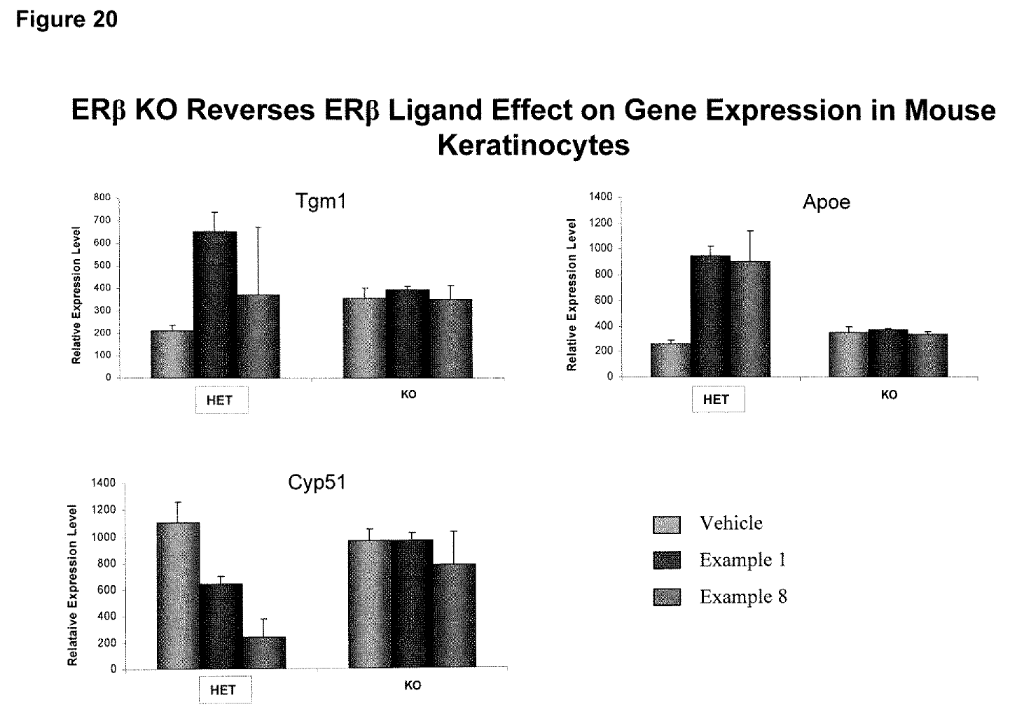

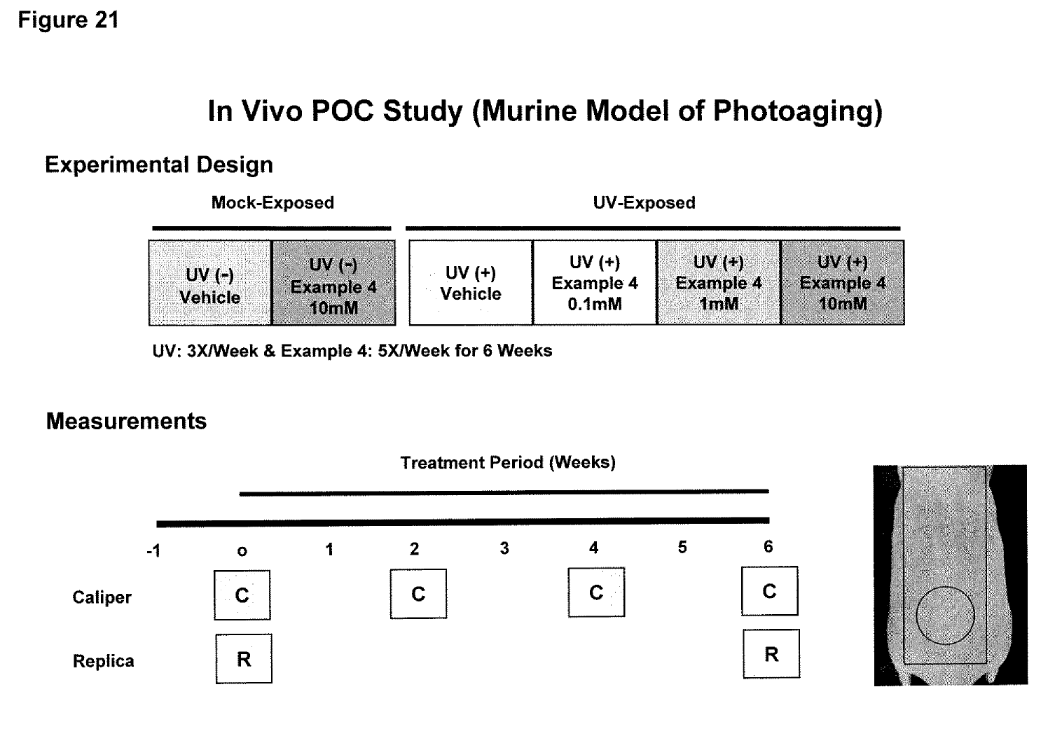

The following exemplary compounds were tested according to the methods described herein. The results obtained are illustrated in FIGS. 1-23.

### Example 1

### 7-bromo-2-(4-hydroxyphenyl)-1,3-benzoxazol-5-ol

### Example 2

### 7-bromo-2-(3-fluoro-4-hydroxyphenyl)-1,3-benzoxazol-5-ol

### Example 3

### 5-hydroxy-2-(4-hydroxyphenyl)-1,3-benzoxazole-7-carbonitrile

### Example 4

### 2-(3-fluoro-4-hydroxyphenyl)-7-vinyl-1,3-benzoxazol-5-ol

### Example 5

### 3-(3-fluoro-4-hydroxyphenyl)-7-hydroxy-1-naphthonitrile

### Example 6

### 2-(2-fluoro-4-hydroxyphenyl)-7-vinyl-1,3-benzoxazol-5-ol

### Example 7

### 2-(2,3-difluoro-4-hydroxyphenyl)-7-vinyl-1,3-benzoxazol-5-ol

### Example 8

### 7-(2-fluoro-4-hydroxyphenyl)-2-naphthol

Evaluation of Binding Affinities to ERα and ERβ

Representative examples of the invention were evaluated for their ability to compete with 17β-estradiol for both ERα and ERβ in a conventional radioligand binding assay. This test procedure provides the methodology for one to determine the relative binding affinities for the ERα or ERβ receptors. The procedure used is briefly described below.

Preparation of Receptor Extracts for Characterization of Binding Selectivity. The ligand binding domains, conveniently defined here as all sequence downstream of the DNA binding domain, were obtained by PCR using full length cDNA as templates and primers that contained appropriate restriction sites for subcloning while maintaining the appropriate reading frame for expression. These templates contained amino acids M₂₅₀-V₅₉₅ of human ERα [Green, et al., Nature 320: 134-9 (1986)] and M₂₁₄-Q₅₃₀ of human ERβ [Ogawa, et al., Biochemical & Biophysical Research Communications 243: 122-6 (1998)]. Human ERβ was cloned into pET15b (Novagen, Madison Wis.) as a Nco1-BamH1 fragment bearing a C-terminal Flag tag. Human ERα was cloned as for human ERβ except that an N-terminal His tag was added. The sequences of all constructs used were verified by complete sequencing of both strands.

BL21 (DE3) cells were used to express the human proteins. Typically a 10 mL overnight culture was used to inoculate a 1 L culture of LB medium containing 100 μg/mL of ampicillin. After incubation overnight at 37° C., IPTG was added to a final concentration of 1 mM and incubation proceeded at 25° C. for 2 hours. Cells were harvested by centrifugation (1500×g), and the pellets washed with and resuspended in 100 mL of 50 mM Tris-Cl (pH 7.4), 150 mM NaCl. Cells were lysed by passing twice through a French press at 12000 psi. The lysate was clarified by centrifugation at 12,000×g for 30 minutes at 4° C. and stored at −70° C.

Evaluation of extracts for specific [³H]-estradiol binding. Dulbecco's phosphate buffered saline (Gibco, 1× final concentration) supplemented with 1 mM EDTA was used as the assay buffer. To optimize the amount of receptor to use in the assay, [³H]-17β-estradiol (New England Nuclear; final concentration=2 nM)±0.6 μM diethlystilbestrol and 100 μL of various dilutions of the E. coli lysate were added to each well of a high binding masked microtiter plate (EG&G Wallac). The final assay volume was 120 μL and the concentration of DMSO was ≦1%. After incubation at room temperature for 5-18 hours, unbound material was aspirated and the plate washed three times with approximately 300 μL of assay buffer. After washing, 135 μL of scintillation cocktail (Optiphase Supermix, EG&G Wallac) was added to the wells, and the plate was sealed and agitated for at least 5 minutes to mix scintillant with residual wash buffer. Bound radioactivity was evaluated by liquid scintillation counting (EG&G Wallac Microbeta Plus).

After determining the dilution of each receptor preparation that provided maximum specific binding, the assay was further optimized by estimating the IC₅₀ of unlabelled 17β-estradiol using various dilutions of the receptor preparation. A final working dilution for each receptor preparation was chosen for which the IC₅₀ of unlabelled 17β-estradiol was 2-4 nM.

Ligand binding competition test procedure Test compounds were initially solubilized in DMSO and the final concentration of DMSO in the binding assay was ≦1%. Eight dilutions of each test compound were used as an unlabelled competitor for [³H]-17β-estradiol. Typically, a set of compound dilutions would be tested simultaneously on human ERα and ERβ. The results were plotted as measured DPM vs. concentration of test compound. For dose-response curve fitting, a four parameter logistic model on the transformed, weighted data was fit and the IC₅₀ was defined as the concentration of compound decreasing maximum [³H]-estradiol binding by 50%.

Binding affinities for ERα and ERβ (as measured by IC₅₀) for representative examples of the invention are described in U.S. Pat. Nos. 6,903,238; 6,914,074; 6,774,248; 6,960,607; 6,884,814; 6,794,403; 6,723,747; 6,835,745; and 7,084,276; and pending U.S. patent application Ser. Nos. 11/210,427; 11/170,017; 11/219,940; and 12/110,728.

The results obtained in the standard pharmacologic test procedure described above demonstrate that the compounds of this invention bind both subtypes of the estrogen receptor. The IC₅₀s are generally lower for ERβ, indicating these compounds are preferentially ERβ selective ligands, but are still considered active at ERα. Compounds of this invention will exhibit a range of activity based, at least partially, on their receptor affinity selectivity profiles. Since the compounds of the invention bind ERβ with higher affinity than ERα, they will be useful in treating or inhibiting diseases than can be modulated via ERβ. Additionally, since each receptor ligand complex is unique and thus its interaction with various coregulatory proteins is unique, compounds of this invention will display different and unpredictable activities depending on cellular context. For example, in some cell-types, it is possible for a compound to behave as an estrogen receptor agonist while in other tissues, an estrogen receptor antagonist. Compounds with such activity have sometimes been referred to as SERMs (Selective Estrogen Receptor Modulators). Unlike many estrogens, however, many of the SERMs do not cause increases in uterine wet weight. These compounds are antiestrogenic in the uterus and can completely antagonize the trophic effects of estrogen receptor agonists in uterine tissue. These compounds, however, act as estrogen receptor agonists in the bone, cardiovascular, and central nervous systems. Due to this tissue selective nature of these compounds, they are useful in treating or inhibiting in a mammal disease states or syndromes which are caused or associated with an estrogen deficiency (in certain tissues such as bone or cardiovascular) or an excess of estrogen (in the uterus or mammary glands). In addition, compounds of this invention also have the potential to behave as estrogen receptor agonists on one receptor type while behaving as estrogen receptor antagonists on the other. For example, it has been demonstrated that compounds can be antagonize the action of 17β-estradiol via ERβ while exhibiting estrogen receptor agonist activity with ERα [Sun, et al., Endocrinology 140: 800-804 (1999)]. Such ERSAA (Estrogen Receptor Selective Agonist Antagonist) activity provides for pharmacologically distinct estrogenic activity within this series of compounds.

What is claimed is:

1. A method of preventing, treating, or inhibiting an inflammatory disease, disorder, or condition of the skin, or a disease, disorder, or condition associated with collagen depletion, comprising: providing to a mammal in need thereof, a therapeutically effective amount of at least one compound of Formula I, having the structure: 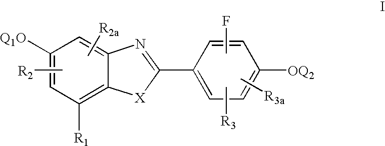
wherein:
Q₁ and Q₂ are independently H, a sugar residue selected from an unmodified or modified hexose residue, or S(O)ₜ—OH, provided that Q₁ and Q₂ are not both H;
t is 0, 1 or 2;
R₁ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, —CONR₅R₆, —NR₅R₆ or —N(R₅)COR₆;
R₅ and R₆ are each, independently hydrogen, alkyl of 1-6 carbon atoms, or aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
a pharmaceutically acceptable salt thereof; or
a gluocuronide, sulfate, or glucuronide-sulfate derivative thereof.

2. The method of claim 1, wherein said at least one compound of Formula I is selected from:
2-(3′-fluoro-4′-glucuronide phenyl)-7-vinyl-1,3-benzoxazol-5-ol;
2-(3′-fluoro-4′-sulfate phenyl)-7-vinyl-1,3-benzoxazol-5-ol;
2-(3′-fluoro-4′-hydroxy phenyl)-7-vinyl-1,3-benzoxazol-5-glucuronide;
2-(3′-fluoro-4′-hydroxy phenyl)-7-vinyl-1,3-benzoxazol-5-sulfate;
2-(3′-fluoro-4′-glucuroride phenyl)-7-vinyl-1,3-benzoxazol-5-glucuronide;
2-(3′-fluoro-4′-glucuronide phenyl)-7-vinyl-1,3-benzoxazol-5-sulfate;
2-(3′-fluoro-4′-sulfate phenyl)-7-vinyl-1,3-benzoxazol-5-glucuronide;
2-(3′-fluoro-4′-sulfate phenyl)-7-vinyl-1,3-benzoxazol-5-sulfate;
2-(2′-fluoro-4′-glucuronide phenyl)-7-vinyl-1,3-benzoxazol-5-ol;
2-(2′-fluoro-4′-sulfate phenyl)-7-vinyl-1,3-benzoxazol-5-ol;
2-(2′-fluoro-4′-hydroxy phenyl)-7-vinyl-1,3-benzoxazol-5-glucuronide;
2-(2′-fluoro-4′-hydroxy phenyl)-7-vinyl-1,3-benzoxazol-5-sulfate;
2-(2′-fluoro-4′-glucuronide phenyl)-7-vinyl-1,3-benzoxazol-5-glucuronide;
2-(2′-fluoro-4′-glucuronide phenyl)-7-vinyl-1,3-benzoxazol-5-sulfate;
2-(2′-fluoro-4′-sulfate phenyl)-7-vinyl-1,3-benzoxazol-5-glucuronide;
2-(2′-fluoro-4′-sulfate phenyl)-7-vinyl-1,3-benzoxazol-5-sulfate;
2-(2′,3′-difluoro-4′-glucuronide phenyl)-7-vinyl-1,3-benzoxazol-5-ol;
2-(2′,3′-difluoro-4′-sulfate phenyl)-7-vinyl-1,3-benzoxazol-5-ol;
2-(2′,3′-difluoro-4′-hydroxyphenyl)-7-vinyl-1,3-benzoxazol-5-glucuronide;
2-(2′,3′-difluoro-4′-hydroxyphenyl)-7-vinyl-1,3-benzoxazol-5-sulfate;
2-(2′,3′-difluoro-4′-glucuronide phenyl)-7-vinyl-1,3-benzoxazol-5-glucuronide;
2-(2′,3′-difluoro-4′-glucuronide phenyl)-7-vinyl-1,3-benzoxazol-5-sulfate;
2-(2′,3′-difluoro-4′-sulfate phenyl)-7-vinyl-1,3-benzoxazol-5-glucuronide;
2-(2′,3′-difluoro-4′-sulfate phenyl)-7-vinyl-1,3-benzoxazol-5-sulfate;
4-bromo-2-(3′-fluoro-4′-glucuronide phenyl)-7-vinyl-1,3-benzoxazol-5-ol;
4-bromo-2-(3′-fluoro-4′-sulfate phenyl)-7-vinyl-1,3-benzoxazol-5-ol;
4-bromo-2-(3′-fluoro-4′-hydroxyphenyl)-7-vinyl-1,3-benzoxazol-5-glucoronide;
4-bromo-2-(3′-fluoro-4′-hydroxyphenyl)-7-vinyl-1,3-benzoxazol-5-sulfate;
4-bromo-2-(3′-fluoro-4′-glucuronide phenyl)-7-vinyl-1,3-benzoxazol-5-glucuronide;
4-bromo-2-(3′-fluoro-4′-glucuronide phenyl)-7-vinyl-1,3-benzoxazol-5-sulfate;
4-bromo-2-(3′-fluoro-4′-sulfate phenyl)-7-vinyl-1,3-benzoxazol-5-glucuronide;
4-bromo-2-(3′-fluoro-4′-sulfate phenyl)-7-vinyl-1,3-benzoxazol-5-sulfate;
4,6-dibromo-2-(3′-fluoro-4′-glucuronide phenyl)-7-vinyl-1,3-benzoxazol-5-ol;
4,6-dibromo-2-(3′-fluoro-4′-sulfate phenyl)-7-vinyl-1,3-benzoxazol-5-ol;
4,6-dibromo-2-(3′-fluoro-4′-hydroxyphenyl)-7-vinyl-1,3-benzoxazol-5-glucuronide;
4,6-dibromo-2-(3′-fluoro-4′-hydroxyphenyl)-7-vinyl-1,3-benzoxazol-5-sulfate;
4,6-dibromo-2-(3′-fluoro-4′-glucuronide phenyl)-7-vinyl-1,3-benzoxazol-5-glucuronide;
4,6-dibromo-2-(3′-fluoro-4′-glucuronide phenyl)-7-vinyl-1,3-benzoxazol-5-sulfate;
4,6-dibromo-2-(3′-fluoro-4′-sulfate phenyl)-7-vinyl-1,3-benzoxazol-5-glucuronide;
4,6-dibromo-2-(3′-fluoro-4′-sulfate phenyl)-7-vinyl-1,3-benzoxazol-5-sulfate;
7-(1-bromovinyl)-2-(2′-fluoro-4′-glucuronide phenyl)-1,3-benzoxazol-5-ol;
7-(1-bromovinyl)-2-(2′-fluoro-4′-sulfate phenyl)-1,3-benzoxazol-5-ol;
7-(1-bromovinyl)-2-(2′-fluoro-4′-hydroxyphenyl)-1,3-benzoxazol-5-glucuronide;
7-(1-bromovinyl)-2-(2′-fluoro-4′-hydroxyphenyl)-1,3-benzoxazol-5-sulfate;
7-(1-bromovinyl)-2-(2′-fluoro-4′-glucuronide phenyl)-1,3-benzoxazol-5-glucuronide;
7-(1-bromovinyl)-2-(2′-fluoro-4′-glucuronide phenyl)-1,3-benzoxazol-5-sulfate;
7-(1-bromovinyl)-2-(2′-fluoro-4′-sulfate phenyl)-1,3-benzoxazol-5-glucuronide;
7-(1-bromovinyl)-2-(2′-fluoro-4′-sulfate phenyl)-1,3-benzoxazol-5-sulfate;
7-(1-bromovinyl)-2-(2′,3′-difluoro-4′-glucuronide phenyl)-1,3-benzoxazol-5-ol;
7-(1-bromovinyl)-2-(2′,3′-difluoro-4′-sulfate phenyl)-1,3-benzoxazol-5-ol;
7-(1-bromovinyl)-2-(2′,3′-difluoro-4′-hydroxyphenyl)-1,3-benzoxazol-5-glucuronide;
7-(1-bromovinyl)-2-(2′,3′-difluoro-4′-hydroxyphenyl)-1,3-benzoxazol-5-sulfate;
7-(1-bromovinyl)-2-(2′,3′-difluoro-4′-glucuronide phenyl)-1,3-benzoxazol-5-glucuronide;
7-(1-bromovinyl)-2-(2′,3′-difluoro-4′-glucuronide phenyl)-1,3-benzoxazol-5-sulfate;
7-(1-bromovinyl)-2-(2′,3′-difluoro-4′-sulfate phenyl)-1,3-benzoxazol-5-glucuronide;
7-(1-bromovinyl)-2-(2′,3′-difluoro-4′-sulfate phenyl)-1,3-benzoxazol-5-sulfate;
7-allyl-2-(3′-fluoro-4′-glucuroinide phenyl)-1,3-benzoxazol-5-ol;
7-allyl-2-(3′-fluoro-4′-sulfate phenyl)-1,3-benzoxazol-5-ol;
7-allyl-2-(3′-fluoro-4′-hydroxyphenyl)-1,3-benzoxazol-5-glucuronide;
7-allyl-2-(3′-fluoro-4′-hydroxyphenyl)-1,3-benzoxazol-5-sulfate;
7-allyl-2-(3′-fluoro-4′-glucuroinide phenyl)-1,3-benzoxazol-5-glucuronide;
7-allyl-2-(3′-fluoro-4′-glucuroinide phenyl)-1,3-benzoxazol-5-sulfate;
7-allyl-2-(3′-fluoro-4′-sulfate phenyl)-1,3-benzoxazol-5-glucuronide;
7-allyl-2-(3′-fluoro-4′-sulfate phenyl)-1,3-benzoxazol-5-sulfate;
2-(3′,5′-difluoro-4′-glucuronide phenyl)-7-vinyl-1,3-benzoxazol-5-ol;
2-(3′,5′-difluoro-4′-sulfate phenyl)-7-vinyl-1,3-benzoxazol-5-ol;
2-(3′,5′-difluoro-4′-hydroxy phenyl)-7-vinyl-1,3-benzoxazol-5-glucuronide;
2-(3′,5′-difluoro-4′-hydroxyphenyl)-7-vinyl-1,3-benzoxazol-5-sulfate;
2-(3′,5′-difluoro-4′-glucuronide phenyl)-7-vinyl-1,3-benzoxazol-5-glucuronide;
2-(3′,5′-difluoro-4′-glucuronide phenyl)-7-vinyl-1,3-benzoxazol-5-sulfate;
2-(3′,5′-difluoro-4′-sulfate phenyl)-7-vinyl-1,3-benzoxazol-5-glucuronide;
2-(3′,5′-difluoro-4′-sulfate phenyl)-7-vinyl-1,3-benzoxazol-5-sulfate;
2-(3′-fluoro-4′-glucuronide phenyl)-7-(1-fluorovinyl)-1,3-benzoxazol-5-ol;
2-(3′-fluoro-4′-sulfate phenyl)-7-(1-fluorovinyl)-1,3-benzoxazol-5-ol;
2-(3′-fluoro-4′-hydroxyphenyl)-7-(1-fluorovinyl)-1,3-benzoxazol-5-glucuronide;
2-(3′-fluoro-4′-hydroxyphenyl)-7-(1-fluorovinyl)-1,3-benzoxazol-5-sulfate;
2-(3′-fluoro-4′-glucuronide phenyl)-7-(1-fluorovinyl)-1,3-benzoxazol-5-glucuronide;
2-(3′-fluoro-4′-glucuronide phenyl)-7-(1-fluorovinyl)-1,3-benzoxazol-5-sulfate;
2-(3′-fluoro-4′-sulfate phenyl)-7-(1-fluorovinyl)-1,3-benzoxazol-5-glucuronide;
2-(3′-fluoro-4′-sulfate phenyl)-7-(1-fluorovinyl)-1,3-benzoxazol-5-sulfate; and
a pharmaceutically acceptable salt thereof.

3. A method of preventing, treating, or inhibiting an inflammatory disease, disorder, or condition of the skin, or a disease, disorder, or condition associated with collagen depletion, comprising: providing to a mammal in need thereof, a therapeutically effective amount of one or more estrogenic agents, wherein at least one of said one or more estrogenic agents is selected from a gluocuronide derivative, a sulfate derivative, or a glucuronide-sulfate derivative of:
2-(5-hydroxy-1,3-benzoxazol-2-yl)benzene-1,4-diol;
2-(3-fluoro-4-hydroxyphenyl)-7-vinyl-1,3-benzoxazol-5-ol;
3-(5-hydroxy-1,3-benzoxazol-2-yl)benzene-1,2-diol;
2-(3-fluoro-4-hydroxyphenyl)-1,3-benzoxazol-5-ol;
2-(3-chloro-4-hydroxyphenyl)-1,3-benzoxazol-5-ol;
2-(2-chloro-4-hydroxyphenyl)-1,3-benzoxazol-5-ol;
2-(3-fluoro-4-hydroxyphenyl)-1,3-benzoxazol-6-ol;
2-(3-tert-butyl-4-hydroxyphenyl)-1,3-benzoxazol-6-ol;
2-(6-hydroxy-1,3-benzoxazol-2-yl)benzene-1,4-diol;
3-(6-hydroxy-1,3-benzoxazol-2-yl)benzene-1,2-diol;
4-(6-hydroxy-1,3-benzoxazol-2-yl)benzene-1,2-diol;
2-(3-chloro-4-hydroxyphenyl)-1,3-benzoxazol-6-ol;
4-(5-hydroxy-1,3-benzoxazol-2-yl)benzene-1,3-diol;
4-(6-hydroxy-1,3-benzoxazol-2-yl)benzene-1,3-diol;
6-chloro-2-(3-fluoro-4-hydroxyphenyl)-1,3-benzoxazol-5-ol;
6-bromo-2-(3-fluoro-4-hydroxyphenyl)-1,3-benzoxazol-5-ol;
6-chloro-2-(4-hydroxyphenyl)-1,3-benzoxazol-5-ol;
5-chloro-2-(4-hydroxyphenyl)-1,3-benzoxazol-6-ol;
7-bromo-2-(3-fluoro-4-hydroxyphenyl)-1,3-benzoxazol-5-ol;
7-bromo-2-(2-fluoro-4-hydroxyphenyl)-1,3-benzoxazol-5-ol;
7-bromo-2-(2,3-difluoro-4-hydroxyphenyl)-1,3-benzoxazol-5-ol;
2-(4-hydroxyphenyl)-7-vinyl-1,3-benzoxazol-5-ol;
7-(1,2-dibromoethyl)-2-(4-hydroxyphenyl)-1,3-benzoxazol-5-ol;
7-(1-bromovinyl)-2-(4-hydroxyphenyl)-1,3-benzoxazol-5-ol;
7-ethynyl-2-(4-hydroxyphenyl)-1,3-benzoxazol-5-ol;
2-(4-hydroxyphenyl)-7-propyl-1,3-benzoxazol-5-ol;
7-butyl-2-(4-hydroxyphenyl)-1,3-benzoxazol-5-ol;
7-cyclopentyl-2-(4-hydroxyphenyl)-1,3-benzoxazol-5-ol;
ethyl 5-hydroxy-2-(4-hydroxyphenyl)-1,3-benzoxazole-7-carboxylate;
2-(4-hydroxyphenyl)-7-phenyl-1,3-benzoxazol-5-ol;
2-(4-hydroxyphenyl)-7-methoxy-1,3-benzoxazol-5-ol;
7-ethyl-2-(4-hydroxyphenyl)-1,3-benzoxazol-5-ol;
7-ethyl-2-(2-ethyl-4-hydroxyphenyl)-1,3-benzoxazol-5-ol;
5-hydroxy-2-(4-hydroxyphenyl)-1,3-benzoxazole-7-carbaldehyde;
7-(hydroxymethyl)-2-(4-hydroxyphenyl)-1,3-benzoxazol-5-ol;
7-(bromomethyl)-2-(4-hydroxyphenyl)-1,3-benzoxazol-5-ol;
[5-hydroxy-2-(4-hydroxyphenyl)-1,3-benzoxazol-7-yl]acetonitrile;
7-(1-hydroxy-1-methylethyl)-2-(4-hydroxyphenyl)-1,3-benzoxazol-5-ol;
2-(4-hydroxyphenyl)-7-isopropenyl-1,3-benzoxazol-5-ol;
2-(4-hydroxyphenyl)-7-isopropyl-1,3-benzoxazol-5-ol;
7-bromo-2-(4-hydroxy-3-(trifluoromethyl)phenyl)-1,3-benzoxazol-5-ol;
7-(2-furyl)-2-(4-hydroxyphenyl)-1,3-benzoxazol-5-ol;
2-(3-fluoro-4-hydroxyphenyl)-7-(2-furyl)-1,3-benzoxazol-5-ol;
2-(4-hydroxyphenyl)-7-thien-2-yl-1,3-benzoxazol-5-ol;
2-(4-hydroxyphenyl)-7-(1,3-thiazol-2-yl)-1,3-benzoxazol-5-ol;
2-(3-fluoro-4-hydroxyphenyl)-5-hydroxy-1,3-benzoxazole-7-carbonitrile;
4-bromo-2-(4-hydroxyphenyl)-7-methoxy-1,3-benzoxazol-5-ol; 4,6-dibromo-2-(4-hydroxyphenyl)-7-methoxy-1,3-benzoxazol-5-ol;
7-bromo-2-(3,5-difluoro-4-hydroxyphenyl)-1,3-benzoxazol-5-ol; and
a pharmaceutically acceptable salt thereof.

4. A method of preventing, treating, or inhibiting an inflammatory disease, disorder, or condition of the skin, or a disease, disorder, or condition associated with collagen depletion, comprising: providing to a mammal in need thereof, a therapeutically effective amount of one or more estrogenic agents, wherein at least one of said one or more estrogenic agents is:
a. a compound of Formula II, having the structure: 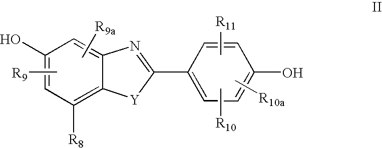
wherein
R₈ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, CONR₁₂R₁₃, NR₁₂R₁₃ or N(R₁₂)COR₁₃;
R₉ and R₉ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;
R₁₀, R₁₀ₐ and R₁₁ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;
R₁₂ and R₁₃ are each, independently hydrogen, alkyl of 1-6 carbon atoms, aryl of 6-10 carbon atoms;
Y is O, S, or NR₁₄;
R₁₄ is hydrogen, alkyl of 1-6 carbon atoms, aryl of 6-10 carbon atoms, —COR₁₂, —CO₂R₁₂ or —SO₂R₁₂;
or a pharmaceutically acceptable salt thereof;
b. a compound of Formula IV, having the structure: 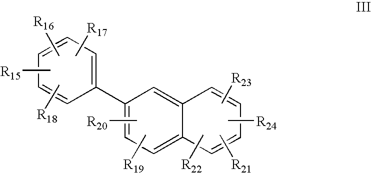
wherein
R₁₅, R₁₆, R₁₇, and R₁₈ are each, independently, selected from hydrogen, hydroxyl, alkyl of 1-6 carbon atoms, alkoxy of 1-6 carbon atoms, or halogen;
R₁₉, R₂₀, R₂₁, R₂₂, R₂₃, and R₂₄ are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₉, R₂₀, R₂₁, R₂₂, R₂₃, or R₂₄ may be optionally substituted with hydroxyl, —CN, halogen, trifluoroalkyl, trifluoroalkoxy, —NO₂, or phenyl; wherein the phenyl moiety of R₁₉, R₂₀, R₂₁, R₂₂, R₂₃, or R₂₄ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
with the proviso that at least one of R₁₅, R₁₆, R₁₇, R₁₈, R₂₁, R₂₂, R₂₃, or R₂₄ is hydroxyl, or a pharmaceutically acceptable salt thereof;
c. a compound of Formula IV, having the structure: 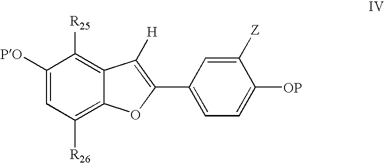
wherein
P and P′ are each, independently, hydrogen, alkyl of 1-6 carbon atoms, or acyl of 2-7 carbon atoms;
Z is hydrogen or halogen;
R₂₅ is hydrogen, alkyl of 1-6 carbon atoms, halogen, —CN, or —CHO;
R₂₆ is alkoxy of 1-6 carbon atoms, or cyanoalkyl having 1-6 carbon atoms in the alkyl moiety;
or a pharmaceutically acceptable salt thereof;
d. a compound of Formula V, having the structure: 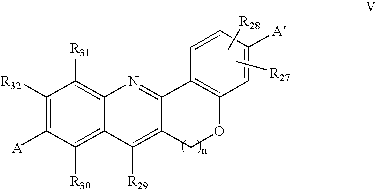
wherein:
A and A′ are each independently OH, H or OU;
each U is independently selected from the group consisting of C₁-C₆ alkyl, alkenyl, benzyl, acyl of 2-7 carbon atoms, aroyl, alkoxycarbonyl, sulfonyl and phosphoryl;
R₂₇ and R₂₈ are independently selected from the group consisting of H, halogen, C₁₋₆ alkyl, C₁₋₆ perhaloalkyl, —CF₃, C₂-C₇ alkenyl and C₁-C₆ alkoxy;
R₂₉, R₃₀, R₃₁ and R₃₂ are each independently selected from the group consisting of H, halogen, —CF₃, C₁₋₆ perhaloalkyl, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl and heteroaryl;
wherein each alkyl or alkenyl moiety of R₂₉, R₃₀, R₃₁ and R₃₂ can be optionally substituted with up to three substituents independently selected from halogen, OH, —CN, trifluoroalkyl of 1-6 carbon atoms, trifluoroalkoxy of 1-6 carbon atoms, —NO₂ and phenyl, wherein said phenyl is optionally substituted with up to three independently selected R₃₃ groups;
wherein each alkynyl moiety of R₂₉, R₃₀, R₃₁ and R₃₂ can be optionally substituted with up to three substituents selected from halogen, —CN, —CHO, acyl, trifluoroalkyl of 1-6 carbon atoms, trialkylsilyl and phenyl, wherein said phenyl is optionally substituted with up to three independently selected R₃₃ groups;
each R₃₃ is independently selected from the group consisting of halogen, C₁-C₆ alkyl, C₂-C₇ alkenyl, —OH, C₁-C₆ alkoxy, —CN, —CHO, —NO₂, amino, C₁-C₆ alkylamino, di-(C₁-C₆)alkylamino, thiol, and C₁-C₆ alkylthio; and
n is 0, 1, 2, or 3;
provided that:
at least one of A and A′ is not H;
if n is 0, then R₂₉ is not halogen; and
at least one of R₂₉, R₃₀ and R₃₁ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
or a pharmaceutically acceptable salt thereof;
e. a compound of Formula VI, having the structure: 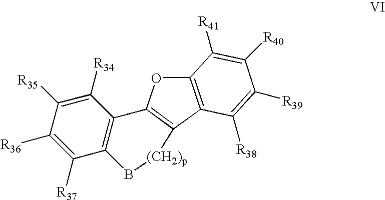
wherein:
B has the structure (i), (ii) or (iii): 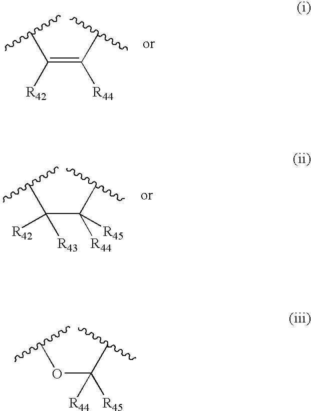
R₃₄, R₃₇, R₃₈, R₄₁, R₄₂, R₄₃, R₄₄, and R₄₅ are each independently selected from the group consisting of hydrogen, C₁-C₆ alkyl, —OR₄₆, halogen, —CF₃, —CF₂CF₃, —CH₂CF₃, —SR₄₆, —NR₄₆R₄₇, —CN, —CH₂CN, —CH₂CH₂CN, —CH═CHCN, —NO₂, —CH₂NO₂, —CH₂CH₂NO₂, —CH═CHNO₂ and —COR₄₆;
p=0 or 1;
each R₄₆ and R₄₇ is independently selected from the group consisting of hydrogen, C₁-C₆ alkyl, —CF₃, benzyl, —CO₂(C₁-C₆ alkyl) and —CO(C₁-C₆ alkyl);
provided that:
a) one of R₃₅ or R₃₆ must be —OR₄₆;
b) one of R₃₉ or R₄₀ must be —OR₄₆;
c) when R₃₅ is —OR₄₆, then R₃₄ and R₃₆ are independently selected from the group consisting of hydrogen, halogen, C₁-C₆ alkyl, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;
d) when R₃₆ is —OR₄₆, then R₃₅ and R₃₇ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;
e) when R₃₉ is —OR₄₆, then R₃₈ and R₄₀ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₃;
f) when R₄₀ is —OR₄₆, then R₃₉ and R₄₁ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆; and
g) when B has the structure (iii), and R₃₈, R₃₉, R₄₁, R₄₄, and R₄₅ are each H, and p=0, then R₄₀ is not —OR₄₆;
f. a compound of Formula VII, having the structure: 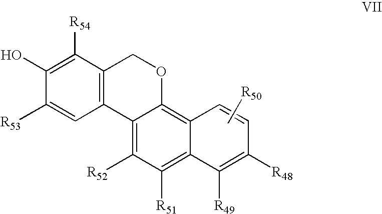
wherein
R₄₈ and R₄₉ are each, independently, selected from hydrogen, hydroxyl, alkyl of 1-6 carbon atoms, alkenyl of 2-7 carbon atoms, alkynyl of 2-7 carbon atoms, alkoxy of 1-6 carbon atoms, or halogen; wherein each alkyl or alkenyl moiety of R₄₈ or R₄₉ may be optionally and independently substituted with hydroxyl, —CN, halogen, trifluoroalkyl of 1-6 carbon atoms, trifluoroalkoxy of 1-6 carbon atoms, —NO₂ or phenyl; and provided that at least one of R₄₈ or R₄₉ is hydroxyl;
R₅₀, R₅₁, R₅₂, R₅₃, and R₅₄ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein each alkyl or alkenyl moiety of R₅₁, R₅₂, R₅₃ or R₅₄ may be optionally and independently substituted with hydroxyl, —CN, halogen, trifluoroalkyl of 1-6 carbon atoms, trifluoroalkoxy of 1-6 carbon atoms, —NO₂ or phenyl; wherein the phenyl moiety of R₅₁ or R₅₂ may be optionally mono-, di-, or tri-substituted with a substituent, the same or different, selected from alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt thereof;
g. a compound of Formula VIII, having the structure: 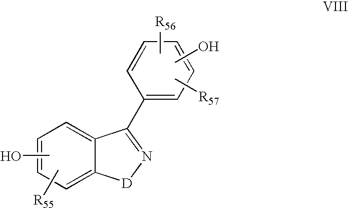
wherein,
R₅₅ and R₅₆ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, trifluoroalkyl of 1-6 carbon atoms, trifluoroalkoxy of 1-6 carbon atoms, alkylthio of 1-6 carbon atoms, or aryl of 6-10 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₇ is hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, alkylthio of 1-6 carbon atoms, trifluoroalkyl of 1-6 carbon atoms, or trifluoroalkoxy of 1-6 carbon atoms, aryl of 6-10 carbon atoms, —NO₂, —CN, —NR₅₈R₅₉ or —N(R₅₈)COR₅₉, —CHFCN, —CF₂CN, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₈ and R₅₉ are each, independently, hydrogen, alkyl of 1-6 carbon atoms, or aryl of 6-10 carbon atoms;
D is O, S, or NR₆₀;
R₆₀ is hydrogen, alkyl of 1-6 carbon atoms, aryl of 6-10 carbon atoms, —COR₅₈, —CO₂R₅₈, or —SO₂R₅₈;
h. a compound of Formula IX, having the structure: 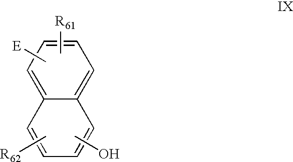
wherein, 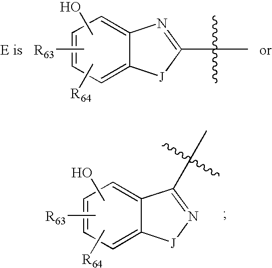
R₆₁, R₆₃, and R₆₄ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₂ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₆₅R₆₆, —N(R₆₅)COR₆₆, —CN, —CHFCN, —CF₂CN, alkynyl of 2-7 carbon atoms, or alkenyl of 2-7 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₅ and R₆₆ are each, independently, hydrogen, alkyl of 1-6 carbon atoms, alkenyl of 2-7 carbon atoms, aryl of 6-10 carbon atoms, —CN, —CHFCN, or —CF₂CN; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₆₇, —CO₂R₆₈, —NO₂, —CONR₆₇R₆₈, —NR₆₇R₆₈ or —N(R₆₇)COR₆₈;
R₆₇ and R₆₈ are each, independently, hydrogen, alkyl of 1-6 carbon atoms, or aryl of 6-10 carbon atoms;
J is O, S, or NR₆₉;
R₆₉ is hydrogen, alkyl of 1-6 carbon atoms, aryl of 6-10 carbon atoms, —COR₆₅, —CO₂R₆₅, or —SO₂R₆₅;
or a pharmaceutically acceptable salt thereof;
i. a compound of Formula X, having the structure: 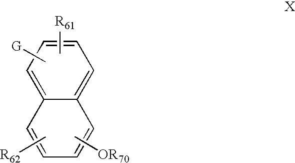
wherein, 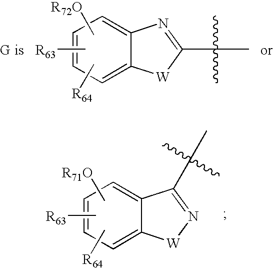
wherein
R₆₁, R₆₂, R₆₃, and R₆₄ are as defined above;
W is O, S, or NR₆₉;
R₆₉ is as defined above; and
R₇₀ and R₇₁ are alkyl of 1-6 carbon atoms;
or a pharmaceutically acceptable salt thereof;
j. a compound of Formula XI, having the structure: 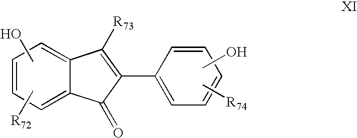
wherein,
R₇₂ is hydrogen, hydroxyl, halogen, trifluoroalkyl of 1-6 carbon atoms, alkyl of 1-6 carbon atoms, cycloalkyl of 3-8 carbon atoms, alkenyl of 2-7 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, —CN, —NO₂, —CHFCN, —CF₂CN, aryl of 6-10 carbon atoms, —NR₇₅R₇₆, —NCOR₇₅, —SR₇₅, —SOR₇₅, —SO₂R₇₅, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₇₅, —CO₂R₇₅, —CONR₇₅R₇₆, or —NR₇₅R₇₆;
R₇₃ is hydrogen, hydroxyl, alkyl of 1-6 carbon atoms, halogen, phenyl substituted with R₁, alkylthio of 1-6 carbon atoms, thioalkyl of 1-6 carbon atoms, amino, aminoalkyl of 1-6 carbon atoms, alkylamino of 1-6 carbon atoms, alkoxy of 1-6 carbon atoms, or alkenyl of 2-7 carbon atoms;
R₇₄ is hydrogen, halogen, hydroxyl, alkyl of 1-6 carbon atoms, alkoxy of 1-6 carbon atoms, trifluoroalkyl of 1-6 carbon atoms, or trifluoroalkoxy of 1-6 carbon atoms;
R₇₅ and R₇₆ are each, independently, hydrogen, alkyl of 1-6 carbon atoms, or aryl of 6-10 carbon atoms;
or a pharmaceutically acceptable salt thereof;
k. a compound of Formula XII, having the structure: 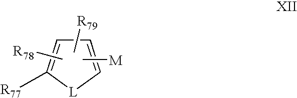
wherein,
R₇₇ is phenyl optionally substituted with 1-4 T groups;
R₇₈ is phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
R₇₉ is hydrogen, phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
L is O, —CH═CH—, or S;
T is —OH, —OR₈₀, halogen, —CN, —CO₂H, —CO₂R₈₀, alkyl of 1-6 carbon atoms, alkenyl of 2-7 carbon atoms, alkynyl of 2-7 carbon atoms, perfluoroalkyl of 1-6 carbon atoms, or —COR₈₀;
M is —CHO, —CN, —CO₂H, —CO₂R₈₀, —CONR₈OR₈₁, —NO₂, —CH═NR₈₀, —CH═NOH, or —CH═NOR₈₀;
R₈₀ and R₈₁ are each, independently, alkyl of 1-6 carbon atoms, alkenyl of 2-7 carbon atoms, alkynyl of 2-7 carbon atoms, or cycloalkyl of 3-8 carbon atoms;
with the proviso that at least one of R₇₈ or R₇₉ is phenyl or phenyl substituted with 1-4 T groups;
or a pharmaceutically acceptable salt thereof;
l. a compound of Formula XIII, having the structure: 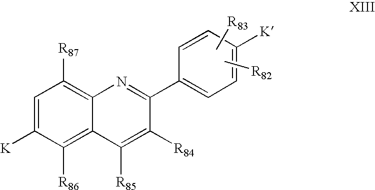
wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each, independently, H, halogen, C₁-C₆ alkyl, C₂-C₇ alkenyl, or C₁-C₆ alkoxy;
R₈₄ is H, halogen, or C₁-C₆ alkyl;
R₈₅ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, or heteroaryl;
R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₅, R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
wherein each alkynyl moiety of R₈₅, R₈₆ or R₈₇ may be optionally substituted with halogen, —CN, —CHO, acyl, trifluoroalkyl of 1-6 carbon atoms, trialkylsilyl, or optionally substituted phenyl;
wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R₈₅, R₈₆ and R₈₇ are H, C₁-C₆ alkyl, C₂-C₇ alkenyl, or C₁-C₆ alkoxy, then at least one of R₈₂ and R₈₃ is halogen, C₁-C₆ alkyl, C₂-C₇ alkenyl, or C₁-C₆ alkoxy; provided that at least one of R₈₅ and R₈₇ is other than H;
or a pharmaceutically acceptable salt thereof;
m. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each H;
R₈₄ is H, halogen, or C₁-C₆ alkyl;
R₈₅, R₈₆ and R₈₇ are each, independently, H, C₁-C₆ alkyl, C₂-C₇ alkenyl, or C₁-C₆ alkoxy, provided that at least one of R₈₅, R₈₆ and R₈₇ is C₁-C₆ alkyl, C₂-C₇ alkenyl, or C₁-C₆ alkoxy;
wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
or a pharmaceutically acceptable salt thereof; or
n. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each, independently, H, halogen, C₁-C₆ alkyl, C₂-C₇ alkenyl, or C₁-C₆ alkoxy;
R₈₄ is H;
R₈₅ is H, aryl, or substituted aryl;
R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
wherein each alkyl or alkenyl moiety of R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
wherein each alkynyl moiety of R₈₆ or R₈₇ may be optionally substituted with halogen, —CN, —CHO, acyl of 2-7 carbon atoms, trifluoroalkyl of 1-6 carbon atoms, trialkylsilyl, or optionally substituted phenyl;
wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
or a N-oxide thereof or a pharmaceutically acceptable salt thereof or a prodrug thereof.

5. The method of claim 4, wherein said one or more estrogenic agents is a compound of Formula II, wherein Y is O.

6. The method of claim 5, wherein said one or more estrogenic agents is a compound of Formula II, wherein R₈ is halogen or alkenyl of 2-3 carbon atoms, which is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃, or —N(R₁₂)COR₁₃.

7. The method of claim 4, wherein said one or more estrogenic agents is a compound of Formula III, wherein the 5 or 6-membered heterocyclic ring having 1 to 4 heteroatoms selected from O, N or S is furan, thiophene, or pyridine.

8. The method of claim 7, wherein said one or more estrogenic agents is a compound of Formula III, wherein one of R₁₅, R₁₆, R₁₇, and R₁₈ is hydroxyl; and R₁₉, R₂₀, R₂₁, R₂₂, R₂₃, and R₂₄ are each, independently, hydrogen, hydroxyl, halogen, —CN, or alkynyl of 2-7 carbon atoms.

9. The method of claim 4, wherein at least one of said one or more estrogenic agents is selected from:
2-(3-fluoro-4-hydroxyphenyl)-7-vinyl-1,3-benzoxazol-5-ol;
2-(2-fluoro-4-hydroxyphenyl)-7-vinyl-1,3-benzoxazol-5-ol;
2-(2,3-difluoro-4-hydroxyphenyl)-7-vinyl-1,3-benzoxazol-5-ol;
5-hydroxy-2-(4-hydroxyphenyl)-1,3-benzoxazole-7-carbonitrile;
7-bromo-2-(4-hydroxyphenyl)-1,3-benzoxazol-5-ol;
4-bromo-2-(3-fluoro-4-hydroxyphenyl)-7-vinyl-1,3-benzoxazol-5-ol;
4,6-dibromo-2-(3-fluoro-4-hydroxyphenyl)-7-vinyl-1,3-benzoxazol-5-ol;
7-(1-bromovinyl)-2-(2-fluoro-4-hydroxyphenyl)-1,3-benzoxazol-5-ol;
7-(1-bromovinyl)-2-(2,3-difluoro-4-hydroxyphenyl)-1,3-benzoxazol-5-ol;
7-allyl-2-(3-fluoro-4-hydroxyphenyl)-1,3-benzoxazol-5-ol;
2-(3,5-difluoro-4-hydroxyphenyl)-7-vinyl-1,3-benzoxazol-5-ol;
2-(3-fluoro-4-hydroxyphenyl)-7-(1-fluorovinyl)-1,3-benzoxazol-5-ol; and
a pharmaceutically acceptable salt thereof.

10. The method of claim 4, wherein at least one of said one or more estrogenic agents is selected from:
2-(5-hydroxy-1,3-benzoxazol-2-yl)benzene-1,4-diol;
3-(5-hydroxy-1,3-benzoxazol-2-yl)benzene-1,2-diol;
2-(3-fluoro-4-hydroxyphenyl)-1,3-benzoxazol-5-ol;
2-(3-chloro-4-hydroxyphenyl)-1,3-benzoxazol-5-ol;
2-(2-chloro-4-hydroxyphenyl)-1,3-benzoxazol-5-ol;
2-(3-fluoro-4-hydroxyphenyl)-1,3-benzoxazol-6-ol;
2-(3-tert-butyl-4-hydroxyphenyl)-1,3-benzoxazol-6-ol;
2-(6-hydroxy-1,3-benzoxazol-2-yl)benzene-1,4-diol;
3-(6-hydroxy-1,3-benzoxazol-2-yl)benzene-1,2-diol;
4-(6-hydroxy-1,3-benzoxazol-2-yl)benzene-1,2-diol;
2-(3-chloro-4-hydroxyphenyl)-1,3-benzoxazol-6-ol;
4-(5-hydroxy-1,3-benzoxazol-2-yl)benzene-1,3-diol;
4-(6-hydroxy-1,3-benzoxazol-2-yl)benzene-1,3-diol;
6-chloro-2-(3-fluoro-4-hydroxyphenyl)-1,3-benzoxazol-5-ol;
6-bromo-2-(3-fluoro-4-hydroxyphenyl)-1,3-benzoxazol-5-ol;
6-chloro-2-(4-hydroxyphenyl)-1,3-benzoxazol-5-ol;
5-chloro-2-(4-hydroxyphenyl)-1,3-benzoxazol-6-ol;
7-bromo-2-(3-fluoro-4-hydroxyphenyl)-1,3-benzoxazol-5-ol;
7-bromo-2-(2-fluoro-4-hydroxyphenyl)-1,3-benzoxazol-5-ol;
7-bromo-2-(2,3-difluoro-4-hydroxyphenyl)-1,3-benzoxazol-5-ol;
2-(4-hydroxyphenyl)-7-vinyl-1,3-benzoxazol-5-ol;
7-(1,2-dibromoethyl)-2-(4-hydroxyphenyl)-1,3-benzoxazol-5-ol;
7-(1-bromovinyl)-2-(4-hydroxyphenyl)-1,3-benzoxazol-5-ol;
7-ethynyl-2-(4-hydroxyphenyl)-1,3-benzoxazol-5-ol;
2-(4-hydroxyphenyl)-7-propyl-1,3-benzoxazol-5-ol;
7-butyl-2-(4-hydroxyphenyl)-1,3-benzoxazol-5-ol;
7-cyclopentyl-2-(4-hydroxyphenyl)-1,3-benzoxazol-5-ol;
ethyl 5-hydroxy-2-(4-hydroxyphenyl)-1,3-benzoxazole-7-carboxylate;
2-(4-hydroxyphenyl)-7-phenyl-1,3-benzoxazol-5-ol;
2-(4-hydroxyphenyl)-7-methoxy-1,3-benzoxazol-5-ol;
7-ethyl-2-(4-hydroxyphenyl)-1,3-benzoxazol-5-ol;
7-ethyl-2-(2-ethyl-4-hydroxyphenyl)-1,3-benzoxazol-5-ol;
5-hydroxy-2-(4-hydroxyphenyl)-1,3-benzoxazole-7-carbaldehyde;
7-(hydroxymethyl)-2-(4-hydroxyphenyl)-1,3-benzoxazol-5-ol;
7-(bromomethyl)-2-(4-hydroxyphenyl)-1,3-benzoxazol-5-ol;
[5-hydroxy-2-(4-hydroxyphenyl)-1,3-benzoxazol-7-yl]acetonitrile;
7-(1-hydroxy-1-methylethyl)-2-(4-hydroxyphenyl)-1,3-benzoxazol-5-ol;
2-(4-hydroxyphenyl)-7-isopropenyl-1,3-benzoxazol-5-ol;
2-(4-hydroxyphenyl)-7-isopropyl-1,3-benzoxazol-5-ol;
7-bromo-2-(4-hydroxy-3-(trifluoromethyl)phenyl)-1,3-benzoxazol-5-ol;
7-(2-furyl)-2-(4-hydroxyphenyl)-1,3-benzoxazol-5-ol;
2-(3-fluoro-4-hydroxyphenyl)-7-(2-furyl)-1,3-benzoxazol-5-ol;
2-(4-hydroxyphenyl)-7-thien-2-yl-1,3-benzoxazol-5-ol;
2-(4-hydroxyphenyl)-7-(1,3-thiazol-2-yl)-1,3-benzoxazol-5-ol;
2-(3-fluoro-4-hydroxyphenyl)-5-hydroxy-1,3-benzoxazole-7-carbonitrile;
4-bromo-2-(4-hydroxyphenyl)-7-methoxy-1,3-benzoxazol-5-ol;
4,6-dibromo-2-(4-hydroxyphenyl)-7-methoxy-1,3-benzoxazol-5-ol;
7-bromo-2-(3,5-difluoro-4-hydroxyphenyl)-1,3-benzoxazol-5-ol; and
a pharmaceutically acceptable salt thereof.

11. The method of claim 4, wherein at least one of said one or more estrogenic agents is selected from:
8-fluoro-6-(3-fluoro-4-hydroxyphenyl)-2-naphthol;
1-chloro-8-fluoro-6-(3-fluoro-4-hydroxyphenyl)-2-naphthol;
3-(3-fluoro-4-hydroxyphenyl)-7-hydroxy-1-naphthonitrile;
3-(3,5-difluoro-4-hydroxyphenyl)-7-hydroxy-1-naphthonitrile; and
a pharmaceutically acceptable salt thereof.

12. The method of claim 4, wherein at least one of said one or more estrogenic agents is selected from:
7-(4-hydroxyphenyl)-2-naphthol;
7-(3-hydroxyphenyl)-2-naphthol;
6-(4-hydroxyphenyl)-1-naphthol;
6-phenyl-2-naphthol;
6-(3-hydroxyphenyl)-2-naphthol;
6-(3-chlorophenyl)-2-naphthol;
2-fluoro-4-(2-naphthyl)phenol;
6-(3-fluoro-4-hydroxyphenyl)-2-naphthol;
6-(3-chloro-4-hydroxyphenol)-2-naphthol;
1-chloro-6-phenyl-2-naphthol;
1-bromo-6-(4-hydroxyphenyl)-2-naphthol;
1-chloro-6-(4-hydroxyphenyl)-2-naphthol;
1-fluoro-6-(4-hydroxyphenyl)-2-naphthol;
2-hydroxy-6-(4-hydroxyphenyl)-1-naphthonitrile;
6-(4-hydroxyphenyl)-1-phenyl-2-naphthol;
6-(4-hydroxyphenyl)-1-methyl-2-naphthol;
1-chloro-6-(3-fluoro-4-hydroxyphenyl)-2-naphthol;
1-chloro-6-(3-chloro-4-hydroxyphenyl)-2-naphthol;
6-(4-hydroxyphenyl)-1-nitro-2-naphthol;
1-chloro-6-(4-hydroxy-2-methylphenyl)-2-naphthol;
6-(4-hydroxy-2-methylphenyl)-2-naphthol;
6-(4-hydroxy-2-methoxyphenyl)-2-naphthol;
6-(2-chloro-4-hydroxyphenyl)-2-naphthol;
1-chloro-6-(2-chloro-4-hydroxyphenyl)-2-naphthol;
6-(2-fluoro-4-hydroxyphenyl)-2-naphthol;
6-(2,5-difluoro-4-hydroxyphenyl)-2-naphthol;
6-(2,6-difluoro-4-hydroxyphenyl)-2-naphthol;
1-chloro-6-(2-fluoro-4-hydroxyphenyl)-2-naphthol;
1-chloro-6-(2,5-difluoro-4-hydroxyphenyl)-2-naphthol;
1-chloro-6-(2,6-difluoro-4-hydroxyphenyl)-2-naphthol;
8-fluoro-6-(4-hydroxyphenyl)-2-naphthol;
1-chloro-8-fluoro-6-(4-hydroxyphenyl)-2-naphthol;
8-chloro-6-(4-hydroxyphenyl)-2-naphthol;
1,5-dichloro-8-fluoro-6-(4-hydroxyphenyl)-2-naphthol;
2-chloro-4-(2-naphthyl)phenol;
3-bromo-8-chloro-6-(4-hydroxyphenyl)-2-naphthol;
1,8-dichloro-6-(4-hydroxyphenyl)-2-naphthol;
3-bromo-1,8-dichloro-6-(4-hydroxyphenyl)-2-naphthol;
7-hydroxy-3-(4-hydroxyphenyl)-1-naphthonitrile;
8-chloro-3-(4-hydroxyphenyl)-7-hydroxy-1-naphthonitrile;
8-chloro-3-(3-fluoro-4-hydroxyphenyl)-7-hydroxy-1-naphthonitrile;
6-(3,5-difluoro-4-hydroxyphenyl)-2-naphthol;
1-chloro-6-(3,5-difluoro-4-hydroxyphenyl)-2-naphthol;
8-bromo-7-hydroxy-3-(4-hydroxyphenyl)-1-naphthonitrile; and
a pharmaceutically acceptable salt thereof.

13. The method of claim 4, wherein at least one of said one or more estrogenic agents is selected from:
[5-Hydroxy-2-(4-hydroxy-phenyl)-benzofuran-7-yl]-acetonitrile;
[5-Methoxy-2-(4-methoxy-phenyl)-benzofuran-7-yl]-acetonitrile;
2-[5-Methoxy-2-(4-methoxy-phenyl)-benzofuran-7-yl]-propionitrile;
2-[5-Methoxy-2-(4-methoxy-phenyl)-benzofuran-7-yl]-2-methyl-propionitrile;
2-[5-Hydroxy-2-(4-hydroxy-phenyl)-benzofuran-7-yl]-propionitrile;
2-[5-Hydroxy-2-(4-hydroxy-phenyl)-benzofuran-7-yl]-2-methyl-propionitrile;
[2-(3-Fluoro-4-hydroxy-phenyl)-5-hydroxy-benzofuran-7-yl]-acetonitrile;
3-[5-Hydroxy-2-(4-hydroxy-phenyl)-benzofuran-7-yl]-propionitrile;
2-(4-Hydroxy-phenyl)-7-methoxy-benzofuran-5-ol;
4-Chloro-2-(4-hydroxy-phenyl)-7-methoxy-benzofuran-5-ol;
4-Bromo-2-(4-hydroxy-phenyl)-7-methoxy-benzofuran-5-ol;
5-Hydroxy-2-(4-hydroxy-phenyl)-7-methoxy-benzofuran-4-carbaldehyde;
5-Hydroxy-2-(4-hydroxy-phenyl)-7-methoxy-benzofuran-4-carbonitrile;
2-(3-Fluoro-4-hydroxy-phenyl)-5-hydroxy-7-methoxy-benzofuran-4-carbonitrile; and
a pharmaceutically acceptable salt thereof.

14. The method of claim 4, wherein at least one of said one or more estrogenic agents is selected from:
2-Hydroxy-3-iodo-5-methoxy-benzaldehyde;
5-Methoxy-2-(4-methoxy-phenyl)-benzofuran-7-carbaldehyde;
[5-Methoxy-2-(4-methoxy-phenyl)-benzofuran-7-yl]-methanol;
7-Bromomethyl-2-(4-hydroxy-phenyl)-benzofuran-5-ol;
2-(4-Hydroxy-phenyl)-7-methoxymethyl-benzofuran-5-ol;
7-Ethoxymethyl-2-(4-hydroxy-phenyl)-benzofuran-5-ol;
2-(4-Hydroxy-phenyl)-7-isopropoxymethyl benzofuran-5-ol;
2-(4-Hydroxy-phenyl)-7-methyl-benzofuran-5-ol;
2-(4-Hydroxy-phenyl)-7-methylsulfanylmethyl-benzofuran-5-ol;
7-Ethylsulfanylmethyl-2-(4-hydroxy-phenyl)-benzofuran-5-ol;
2-(4-Hydroxy-phenyl)-7-phenylsulfanylmethyl-benzofuran-5-ol;
2-(4-Hydroxy-phenyl)-7-methanesulfinylmethyl-benzofuran-5-ol;
2-(4-Hydroxy-phenyl)-7-methanesulfonylmethyl-benzofuran-5-ol;
2-(4-Hydroxy-phenyl)-7-thiocyanatomethyl-benzofuran-5-ol;
2-(4-Hydroxy-phenyl)-7-imidazol-1-ylmethyl-benzofuran-5-ol;
7-Bromomethyl-5-methoxy-2-(4-methoxy-phenyl)-benzofuran;
5-Methoxy-2-(4-methoxy-phenyl)-benzofuran-7-carboxylic acid;
5-Hydroxy-2-(4-hydroxy-phenyl)-benzofuran-7-carboxylic acid;
7-Hydroxymethyl-2-(4-hydroxy-phenyl)-benzofuran-5-ol;
5-Methoxy-2-(4-methoxy-phenyl)-benzofuran-7-carbaldehyde oxime;
5-Methoxy-2-(4-methoxy-phenyl)-7-vinyl-benzofuran;
7-Ethyl-5-methoxy-2-(4-methoxy-phenyl)-benzofuran;
7-Ethyl-2-(4-hydroxy-phenyl)-benzofuran-5-ol;
7-(2,2-Dichloro-vinyl)-5-methoxy-2-(4-methoxy-phenyl)-benzofuran;
7-(2,2-Dichlorovinyl)-5-hydroxy-2-(4-hydroxyphenyl)benzofuran-5-ol;
5-Methoxy-2-(4-methoxy-phenyl)-7-propenyl-benzofuran;
5-Methoxy-2-(4-methoxy-phenyl)-7-propyl-benzofuran;
2-(4-Hydroxy-phenyl)-7-propyl-benzofuran-5-ol;
5-Hydroxy-2-(4-hydroxy-phenyl)-benzofuran-7-carboxylic acid isopropyl ester;
5-Hydroxy-2-(4-hydroxy-phenyl)-benzofuran-7-carboxylic acid propyl ester;
5-Hydroxy-2-(4-hydroxy-phenyl)-benzofuran-7-carboxylic acid ethyl ester;
2-(3-Fluoro-4-methoxy-phenyl)-5-methoxy-benzofuran-7-carboxylic acid methyl ester;
[2-(3-Fluoro-4-methoxy-phenyl)-5-methoxy-benzofuran-7-yl]-methanol;
7-Bromomethyl-2-(3-fluoro-4-hydroxy-phenyl)-benzofuran-5-ol;
2-(3-Fluoro-4-methoxy-phenyl)-5-methoxy-benzofuran-7-carboxylic acid;
2-(3-Fluoro-4-methoxy-phenyl)-5-methoxy-benzofuran-7-carboxylic acid methoxy-methyl-amide;
2-(3-Fluoro-4-methoxy-phenyl)-5-methoxy-benzofuran-7-carbaldehyde;
2-(3-Fluoro-4-methoxy-phenyl)-5-methoxy-benzofuran-7-carbaldehyde oxime;
2-(3-Fluoro-4-hydroxy-phenyl)-5-hydroxy-benzofuran-7-carbonitrile;
2-(3-Fluoro-4-hydroxy-phenyl)-7-methyl-benzofuran-5-ol;
3-Bromo-2-hydroxy-5-methoxy-benzaldehyde;
3-Bromo-2,5-dimethoxy-benzaldehyde;
(3-Bromo-2,5-dimethoxy-phenyl)-methanol;
1-Bromo-3-chloromethyl-2,5-dimethoxy-benzene;
(3-Bromo-2,5-dimethoxy-phenyl)-acetonitrile;
(3-Bromo-2,5-dimethoxy-phenyl)-acetic acid;
2-(3-Bromo-2,5-dimethoxy-phenyl)-1-(4-methoxy-phenyl)-ethanone;
7-Chloro-2-(4-hydroxy-phenyl)-benzofuran-5-ol;
7-Bromo-2-(4-hydroxy-phenyl)-benzofuran-5-ol;
3-[5-Hydroxy-2-(4-hydroxy-phenyl)benzofuran-7-yl]-acrylic acid methyl ester;
3-[5-Hydroxy-2-(4-hydroxy-phenyl)-benzofuran-7-yl]-propionic acid methyl ester;
3-[5-Hydroxy-2-(4-hydroxyphenyl)-benzofuran-7-yl]-acrylamide;
4,7-Dibromo-2-(4-hydroxy-phenyl)-benzofuran-5-ol;
3-[5-Hydroxy-2-(4-hydroxy-phenyl)-benzofuran-7-yl]-acrylonitrile;
7-Bromo-5-(tert-butyl-dimethyl-silanyloxy)-2-[4-(tert-butyl-dimethyl-silanyloxy)-phenyl]-benzofuran;
2-(4-Hydroxy-phenyl)-7-vinyl-benzofuran-5-ol;
5-Hydroxy-2-(4-hydroxy-phenyl)-7-methoxy-benzofuran-4-carbaldehyde oxime;
2-(2,5-Dimethoxy-phenyl)-1-(4-methoxy-phenyl)-ethanone;
2-(4-Hydroxy-phenyl)-benzofuran-5-ol;
2-(2,5-Dimethoxy-phenyl)-1-(2-fluoro-4-methoxy-phenyl)-ethanone;
2-(2-Fluoro-4-hydroxy-phenyl)-benzofuran-5-ol;
5-OMe-Benzofuran 2-boronic acid;
4-(5-Methoxy-benzofuran-2-yl)-3-methyl-phenol;
2-(4-Hydroxy-2-methyl-phenyl)-benzofuran-5-ol;
5-Bromo-2-(4-methoxy-phenyl)-benzofuran;
5-Chloro-2-(4-methoxy-phenyl)-benzofuran;
5-Fluoro-2-(4-methoxy-phenyl)-benzofuran;
5-tert-Butyl-2-(4-methoxy-phenyl)-benzofuran;
5,7-Dichloro-2-(4-methoxy-phenyl)-benzofuran;
5,7-Difluoro-2-(4-methoxy-phenyl)-benzofuran;
5,7-Dibromo-2-(4-methoxy-phenyl)-benzofuran;
2-(4-Methoxy-phenyl)-5-trifluoromethyl-benzofuran;
4-(5-Bromo-benzofuran-2-yl)-phenol;
4-(5-Chloro-benzofuran-2-yl)-phenol;
4-(5-Fluoro-benzofuran-2-yl)-phenol;
4-(5-tert-Butyl-benzofuran-2-yl)-phenol;
4-(5,7-Dichloro-benzofuran-2-yl)-phenol;
4-(5,7-Difluoro-benzofuran-2-yl)-phenol;
4-(5,7-Dibromo-benzofuran-2-yl)-phenol;
4-(5-Trifluoromethyl-benzofuran-2-yl)-phenol;
2-Iodo-4-methoxy-6-nitro-phenol;
5-Methoxy-2-(4-methoxy-phenyl)-7-nitro-benzofuran;
2-(4-Hydroxy-phenyl)-7-nitro-benzofuran-5-ol;
7-Amino-2-(4-hydroxy-phenyl)-benzofuran-5-ol;
1-(2-Bromo-4-methoxyphenyl)-2-(2,5-dimethoxy-phenyl)-ethanone;
2-(2-Bromo-4-hydroxy-phenyl)-benzofuran-5-ol;
2-(5-Hydroxy-biphenyl-2-yl)-benzofuran-5-ol;
2-(4′-Benzyloxy-5-hydroxy-biphenyl-2-yl)-benzofuran-5-ol;
6-(5-Hydroxy-benzofuran-2-yl)-biphenyl-3,4′-diol;
2-[5-Hydroxy-4′-(2-pyrrolidin-1-yl-ethoxy)-biphenyl-2-yl]-benzofuran-5-ol;
2,2-Dimethyl-propionic acid 2-[4-(2,2-dimethyl-propionyloxy)-phenyl]-benzofuran-5-yl ester;
1-[5-Hydroxy-2-(4-hydroxy-phenyl)-benzofuran-3-yl]-ethanone;
1-[5-Hydroxy-2-(4-hydroxy-phenyl)-benzofuran-3-yl]-ethanone oxime;
3-(1-Hydroxy-ethyl)-2-(4-hydroxy-phenyl)-benzofuran-5-ol;
2-[5-Methoxy-2-(4-methoxy-phenyl)-benzofuran-7-yl]-propan-2-ol;
7-Isopropenyl-5-methoxy-2-(4-methoxy-phenyl)-benzofuran;
7-Isopropyl-5-methoxy-2-(4-methoxy-phenyl)-benzofuran;
2-(4-Hydroxy-phenyl)-7-isopropyl-benzofuran-5-ol;
5-Hydroxy-2-(4-hydroxy-phenyl)-benzofuran-7-carboxylic acid methoxy-methyl-amide;
5-Hydroxy-2-(4-hydroxy-phenyl)-benzofuran-7-carbaldehyde;
5-Methoxy-2-(4-methoxy-phenyl)-benzofuran-7-carboxylic acid methoxy-methyl-amide;
1-[5-Methoxy-2-(4-methoxy-phenyl)-benzofuran-7-yl]-ethanone;
1-[5-Hydroxy-2-(4-hydroxy-phenyl)-benzofuran-7-yl]-ethanone;
1-[5-Hydroxy-2-(4-hydroxy-phenyl)-benzofuran-7-yl]-propan-1-one;
2-(2,5-Dimethoxy-phenyl)-1-(3-fluoro-4-methoxy-phenyl)-ethanone;
2-(3-Fluoro-4-hydroxy-phenyl)-benzofuran-5-ol;
4-(5-Methoxy-benzofuran-2-yl)-benzoic acid methyl ester;
4-(5-Hydroxy-benzofuran-2-yl)-benzoic acid;
2-(4-Hydroxymethyl-phenyl)-benzofuran-5-ol;
4-Bromo-2-(4-hydroxy-phenyl)-benzofuran-5-ol;
4-Chloro-2-(4-hydroxy-phenyl)-benzofuran-5-ol;
2-(4-Hydroxy-phenyl)-4-methoxy-benzofuran-5-ol;
4-Bromo-5-methoxy-2-(4-methoxy-phenyl)-benzofuran;
5-Methoxy-2-(4-methoxy-phenyl)-benzofuran-4-carbonitrile;
5-Hydroxy-2-(4-hydroxy-phenyl)-benzofuran-4-carbonitrile;
5-Methoxy-2-(4-methoxy-phenyl)-4-methyl-benzofuran;
2-(4-Hydroxy-phenyl)-4-methyl-benzofuran-5-ol;
2-(4-Hydroxy-phenyl)-7-[1,3,4]oxadiazol-2-yl-benzofuran-5-ol;
2,2,2-Trifluoro-1-[5-methoxy-2-(4-methoxy-phenyl)-benzofuran-7-yl]-ethanol;
5-Methoxy-2-(4-methoxyphenyl)-7-(2,2,2-trifluoroethyl)-benzofuran;
2-(4-Hydroxy-phenyl)-7-(2,2,2-trifluoro-ethyl)-benzofuran-5-ol;
2-(4-Methoxy-phenyl)-benzofuran-5-carboxylic acid methyl ester;
2-(4-Hydroxy-phenyl)-benzofuran-5-carboxylic acid;
4-(5-Hydroxymethyl-benzofuran-2-yl)-phenol; and
a pharmaceutically acceptable salt thereof.

15. The method of claim 4, wherein at least one of said one or more estrogenic agents is selected from:
3,9-Dimethoxy-6H-chromeno[4,3-b]quinolin-7-ol;
7-Chloro-3,9-dimethoxy-6H-chromeno[4,3-b]quinoline;
7-Bromo-3,9-dimethoxy-6H-chromeno[4,3-b]quinoline;
7-Chloro-3,9-dihydroxy-6H-chromeno[4,3-b]quinoline;
7-Bromo-3,9-dihydroxy-6H-chromeno[4,3-b]quinoline;
3,9-Dihydroxy-7-vinyl-6H-chromeno[4,3-b]quinoline;
3,9-Dihydroxy-7-[(trimethylsilyl)ethynyl]-6H-chromeno[4,3-b]quinoline;
3,9-Dihydroxy-7-ethynyl-6H-chromeno[4,3-b]quinoline;
3,9-Dihydroxy-7-ethyl-6H-chromeno[4,3-b]quinoline;
7-Cyano-3,9-dihydroxy-6H-chromeno[4,3-b]quinoline;
7-(4-Chlorophenyl)-3,9-dihydroxy-6H-chromeno[4,3-b]quinoline;
7-(4-Cyanophenyl)-3,9-dihydroxy-6H-chromeno[4,3-b]quinoline;
3,9-Dihydroxy-7-(4-methoxyphenyl)-6H-chromeno[4,3-b]quinoline;
3,9-Dihydroxy-7-[4-(trifluoromethyl)phenyl]-6H-chromeno[4,3-b]quinoline;
7-(3-Chlorophenyl)-3,9-dihydroxy-6H-chromeno[4,3-b]quinoline;
3,9-Dihydroxy-7-(3-methoxyphenyl)-6H-chromeno[4,3-b]quinoline;
7-(3-Cyanophenyl)-3,9-dihydroxy-6H-chromeno[4,3-b]quinoline; and
a pharmaceutically acceptable salt, chelate, complex or prodrug thereof.

16. The method of claim 4, wherein at least one of said one or more estrogenic agents is selected from:
5,6-Dihydro-benzo[b]naphtho[2,1-d]furan-3,9-diol;
Benzo[b]naphtho[2,1-d]furan-3,9-diol;
5-Bromo-benzo[b]naphtho[2,1-d]furan-3,9-diol;
3,8-Dihydroxy-5,6-dihydro-benzo[b]naphtho[2,1-d]furan-10-carbonitrile;
3,9-Dihydroxy-6,7-dihydro-5H-12-oxa-dibenzo[a,e]azulen-11-carbonitrile;
3,9-Dihydroxy-5,6-dihydro-benzo[b]naphtho[2,1-d]furan-10-carbonitrile;
3,9-Dihydroxy-benzo[b]naphtho[2,1-d]furan-10-carbonitrile;
3,8-Dihydroxy-5,5-dimethyl-5,6-dihydro-benzo[b]naphtho[2,1-d]furan-10-carbonitrile;
6H-Benzo[4,5]furo[3,2-c]chromen-3,8-diol;
3,8-Dihydroxy-6H-Benzo[4,5]furo[3,2-c]chromene-10-carbonitrile;
10-Bromo-6H-benzo[4,5]furo[3,2-c]chromene-3,8-diol;
2,9-Dihydroxy-5,6-dihydro-benzo[b]naphtho[2,1-d]furan-10-benzonitrile;
2,9-Dihydroxy-benzo[b]naphtho[2,1-d]furan-10-carbonitrile; and
a pharmaceutically acceptable salt thereof.

17. The method of claim 4, wherein at least one of said one or more estrogenic agents is selected from: 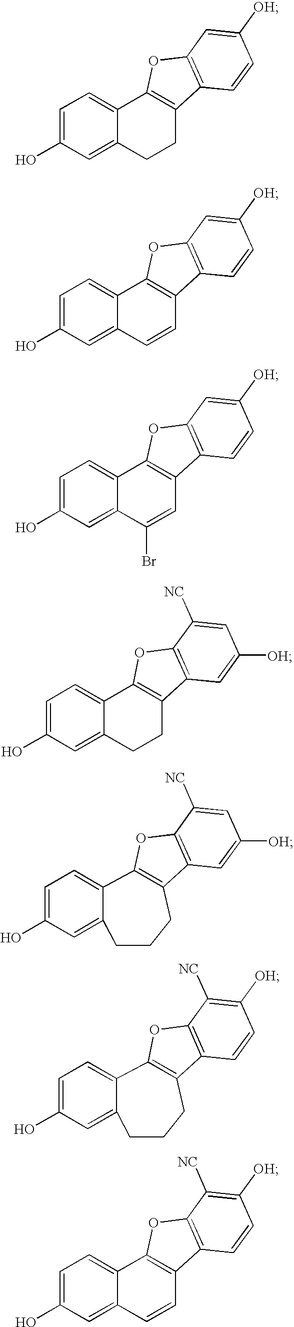 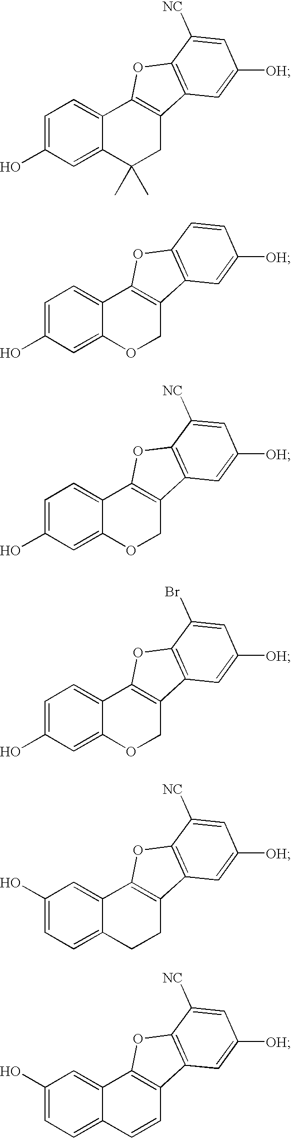
and a pharmaceutically acceptable salt thereof.

18. The method of claim 4, wherein at least one of said one or more estrogenic agents is selected from:
6H-Dibenzo[c,h]chromene-2,8-diol;
9-Fluoro-6H-dibenzo[c,h]chromene-2,8-diol;
2,8-Dihydroxy-6H-dibenzo[c,h]chromene-12-carbonitrile;
9-fluoro-2,8-dihydroxy-6H-dibenzo[c,h]chromene-12-carbonitrile;
1-Chloro-2,8-dihydroxy-6H-dibenzo[c,h]chromene-12-carbonitrile;
1-Chloro-6H-dibenzo[c,h]chromene-2,8-diol;
12-Bromo-6H-dibenzo[c,h]chromene-2,8-diol;
12-Chloro-6H-dibenzo[c,h]chromene-2,8-diol;
12-Bromo-9-fluoro-6H-dibenzo[c,h]chromene-2,8-diol;
12-Chloro-9-fluoro-6H-dibenzo[c,h]chromene-2,8-diol;
12-Methoxy-6H-dibenzo[c,h]chromene-2,8-diol;
11-Chloro-12-methoxy-6H-dibenzo[c,h]chromene-2,8-diol;
12-Methyl-6H-dibenzo[c,h]chromene-2,8-diol;
12-Vinyl-6H-dibenzo[c,h]chromene-2,8-diol;
12-Ethyl-6H-dibenzo[c,h]chromene-2,8-diol;
12-Thien-3-yl-6H-dibenzo[c,h]chromene-2,8-diol;
12-Thien-2-yl-6H-dibenzo[c,h]chromene-2,8-diol;
12-Butyl-6H-dibenzo[c,h]chromene-2,8-diol;
12-Propyl-6H-dibenzo[c,h]chromene-2,8-diol;
6H-dibenzo[c,h]chromene-18-diol;
12-Chloro-6H-dibenzo[c,h]chromene-1,8-diol;
12-Bromo-6H-dibenzo[c,h]chromene-1,8-diol;
1,8-Dihydroxy-6H-dibenzo[c,h]chromene-12-carbonitrile;
2-Chloro-6H-dibenzo[c,h]chromene-1,8-diol;
4-chloro-6H-dibenzo[c,h]chromene-1,8-diol;
6H-Dibenzo[c,h]chromen-8-ol; and
a pharmaceutically acceptable salt thereof.

19. The method of claim 4, wherein at least one of said one or more estrogenic agents is selected from:
3-(4-Hydroxy-2-methylphenyl)-1,2-benzisoxazol-6-ol;
3-(4-Hydroxy-2,5-di propylphenyl)-1,2-benzisoxazol-6-ol;
3-(2-Ethyl-4-hydroxy-5-propylphenyl)-1,2-benzisoxazol-6-ol;
3-(4-Hydroxy-2-isobutyl-5-propylphenyl)-1,2-benzisoxazol-6-ol;
3-(4-Hydroxy-3-propylphenyl)-1,2-benzisoxazol-6-ol;
3-(4-Hydroxy-2-propylphenyl)-1,2-benzisoxazol-6-ol;
3-(4-Hydroxy-3-methylphenyl)-1,2-benzisoxazol-6-ol;
3-(4-Hydroxyphenyl)-1,2-benzisoxazol-6-ol;
3-(2-Hydroxyphenyl)-1,2-benzisoxazol-6-ol;
2-(6-Hydroxy-1,2-benzisoxazol-3-yl)benzene-1,4-diol;
4-(6-Hydroxy-1,2-benzisoxazol-3-yl)benzene-1,3-diol;
3-(4-Hydroxyphenyl)-1,2-benzisoxazol-5-ol;
7-Bromo-3-(4-hydroxyphenyl)-1,2-benzisoxazol-5-ol;
3-(3-Bromo-4-hydroxyphenyl)-1,2-benzisoxazol-6-ol;
3-(3-Chloro-4-hydroxyphenyl)-1,2-benzisoxazol-6-ol;
3-(3-Fluoro-4-hydroxyphenyl)-1,2-benzisoxazol-6-ol;
4-Bromo-6-(6-hydroxy-1,2-benzisoxazol-3-yl)benzene-1,3-diol;
4-Chloro-6-(6-hydroxy-1,2-benzisoxazol-3-yl)benzene-1,3-diol;
4-(6-Hydroxy-1,2-benzisoxazol-3-yl)-6-methylbenzene-1,3-diol;
4-(6-Hydroxy-1,2-benzisoxazol-3-yl)-2-methylbenzene-1,3-diol; and
a pharmaceutically acceptable salt thereof.

20. The method of claim 4, wherein at least one of said one or more estrogenic agents is selected from:
2-(6-Hydroxy-1-naphthyl)-1,3-benzoxazol-5-ol;
2-(6-Hydroxy-1-naphthyl)-1,3-benzoxazol-6-ol;
2-(6-Hydroxy-2-naphthyl)-1,3-benzoxazol-5-ol;
2-(6-Hydroxy-2-naphthyl)-1,3-benzoxazol-6-ol;
2-(5-Hydroxy-1-naphthyl)-1,3-benzoxazol-6-ol;
2-(4-Hydroxy-1-naphthyl)-1,3-benzoxazol-5-ol;
2-(4-Hydroxy-1-naphthyl)-1,3-benzoxazol-6-ol;
2-(5-Bromo-6-hydroxy-1-naphthyl)-1,3-benzoxazol-6-ol;
3-(6-Hydroxy-2-naphthyl)-1,2-benzisoxazol-6-ol;
3-(6-Hydroxy-2-naphthyl)-1,2-benzisoxazol-5-ol;
3-(5-Hydroxy-2-naphthyl)-1,2-benzisoxazol-6-ol;
3-(5-Hydroxy-1-naphthyl)-1,2-benzisoxazol-6-ol;
3-(6-Hydroxy-1-naphthyl)-1,2-benzisoxazol-6-ol;
3-(7-Hydroxy-1-naphthyl)-1,2-benzisoxazol-6-ol;
3-(5-Bromo-6-hydroxy-2-naphthyl)-1,2-benzisoxazol-6-ol;
3-(6-Hydroxy-2-naphthyl)-1-methyl-1H-indazol-6-ol;
1-Ethyl-3-(6-hydroxy-2-naphthyl)-1H-indazol-6-ol; and
a pharmaceutically acceptable salt thereof.

21. The method of claim 4, wherein at least one of said one or more estrogenic agents is selected from:
3-amino-5-hydroxy-2-(4-hydroxyphenyl)-1H-inden-1-one;
5-hydroxy-2-(4-hydroxyphenyl-1H-indene-1,3(2H)-dione;
3-bromo-6-hydroxy-2-(4-hydroxyphenyl)-1H-inden-1-one;
3-bromo-5-hydroxy-2-(4-hydroxyphenyl)-1H-inden-1-one;
3-(ethylamino)-6-hydroxy-2-(4-hydroxyphenyl)-1H-inden-1-one;
3-(ethylthio)-6-hydroxy-2-(4-hydroxyphenyl)-1H-inden-1-one;
3-(ethylthio)-5-hydroxy-2-(4-hydroxyphenyl)-1H-inden-1-one;
6-hydroxy-2-(4-hydroxyphenyl)-3-(methylthio)-1H-inden-1-one;
5-hydroxy-2-(4-hydroxyphenyl)-3-(methylthio)-1H-inden-1-one;
6-hydroxy-2-(4-hydroxyphenyl)-3-phenyl-1H-inden-1-one;
5-hydroxy-2-(4-hydroxyphenyl)-3-phenyl-1H-inden-1-one;
6-hydroxy-2-(4-hydroxyphenyl)-3-methyl-1H-inden-1-one;
5-hydroxy-2-(4-hydroxyphenyl)-3-methyl-1H-inden-1-one;
4,6-dihydroxy-2-(4-hydroxyphenyl)-1H-inden-1,3(2H)-dione;
3-bromo-4,6-dihydroxy-2-(4-hydroxyphenyl)-1H-inden-1-one;
3-bromo-6-hydroxy-2-(4-hydroxyphenyl)-4-methoxy-1H-inden-1-one;
5,7-dihydroxy-2-(4-hydroxyphenyl)-3-methyl-1H-inden-1-one;
4,6-dihydroxy-2-(4-hydroxyphenyl)-3-(methylthio)-1H-inden-1-one;
3-(ethylthio)-4,6-Dihydroxy-2-(4-hydroxyphenyl)-1H-inden-1-one;
4,6-dihydroxy-2-(4-hydroxyphenyl)-3-phenyl-1H-inden-1-one;
5,7-dihydroxy-2-(4-hydroxyphenyl)-3-phenyl-1H-inden-1-one;
5,6-dihydroxy-2-(4-hydroxyphenyl)-3-methyl-1H-inden-1-one; and
a pharmaceutically acceptable salt thereof.

22. The method of claim 4, wherein at least one of said one or more estrogenic agents is selected from:
5-(3-hydroxyphenyl)-3-(4-hydroxyphenyl)-4-methylthiophene-2-carbaldehyde;
3-(3-hydroxyphenyl)-5-(4-hydroxyphenyl)-4-methylthiophene-2-carbaldehyde;
3-(4-hydroxy-2-methylphenyl)-5-(4-hydroxyphenyl)-4-methyl-2-thiophenecarbaldehyde;
3-(5-hydroxy-2-methylphenyl)-5-(4-hydroxyphenyl)-4-methyl-2-thiophenecarbaldehyde;
3-(3-fluoro-4-hydroxyphenyl)-5-(4-hydroxyphenyl)-4-methyl-2-thiophenecarbaldehyde;
3-(3-formyl-4-hydroxyphenyl)-5-(4-hydroxyphenyl)-4-methyl-2-thiophenecarbaldehyde;
3-(3-chloro-4-hydroxyphenyl)-5-(4-hydroxyphenyl)-4-methylthiophene-2-carbaldehyde;
5-(3-fluoro-4-hydroxyphenyl)-3-(4-hydroxyphenyl)-4-methyl-2-thiophenecarbaldehyde;
5-(3-fluoro-4-hydroxyphenyl)-3-(4-hydroxy-2-methyl phenyl)-4-methylthiophene-2-carbaldehyde;
3-(3-chloro-4-hydroxyphenyl)-5-(3-fluoro-4-hydroxyphenyl)-4-methylthiophene-2-carbaldehyde;
5-(4-hydroxy-2-methyl phenyl)-3-(4-hydroxyphenyl)-4-methyl-2-thiophenecarbaldehyde;
3-(3-fluoro-4-hydroxyphenyl)-5-(4-hydroxy-2-methyl phenyl)-4-methylthiophene-2-carbaldehyde;
5-(3-chloro-4-hydroxyphenyl)-3-(4-hydroxyphenyl)-4-methylthiophene-2-carbaldehyde;
5-(3-chloro-4-hydroxyphenyl)-3-(3-fluoro-4-hydroxyphenyl)-4-methylthiophene-2-carbaldehyde;
3,5-bis(4-hydroxyphenyl)-4-methylthiophene-2-carbaldehyde;
3-(3-hydroxyphenyl)-5-(4-hydroxyphenyl)-4-methylthiophene-2-carbaldehyde oxime;
3-(3-hydroxyphenyl)-5-(4-hydroxyphenyl)-4-methylthiophene-2-carbonitrile;
3,5-bis(4-hydroxyphenyl)-4-methylthiophene-2-carbaldehyde oxime;
3,5-bis(4-hydroxyphenyl)-4-methylthiophene-2-carbonitrile;
3,5-bis(4-hydroxyphenyl)-4-methylthiophene-2-carbaldehyde O-methyloxime;
3-(4-hydroxyphenyl)-5-(3-hydroxyphenyl)-4-methylthiophene-2-carbaldehyde oxime;
3-(4-hydroxyphenyl)-5-(3-hydroxyphenyl)-4-methylthiophene-2-carbonitrile;
3-(3-fluoro-4-hydroxyphenyl)-5-(4-hydroxy-2-methylphenyl)-4-methylthiophene-2-carbaldehyde O-methyloxime; and
a pharmaceutically acceptable salt thereof.

23. The method of claim 4, wherein at least one of said one or more estrogenic agents is selected from:
4-Chloro-2-(4-hydroxyphenyl)quinolin-6-ol;
4-Chloro-2-(3-fluoro-4-hydroxyphenyl)quinolin-6-ol;
8-Bromo-4-chloro-2-(3-fluoro-4-hydroxyphenyl)-quinolin-6-ol;
4-Bromo-2-(4-hydroxyphenyl)quinolin-6-ol;
4-Bromo-2-(3-fluoro-4-hydroxyphenyl)quinolin-6-ol;
4-Bromo-2-(3,5-difluoro-4-hydroxyphenyl)quinolin-6-ol;
4-Cyano-2-(4-hydroxyphenyl)quinolin-6-ol;
4-Cyano-2-(3-fluoro-4-hydroxyphenyl)quinolin-6-ol;
6-Acetoxy-4-bromo-2-(3-fluoro-4-acetoxyphenyl)quinoline;
2-(4-Hydroxyphenyl)-4-iodoquinolin-6-ol;
2-(3-Fluoro-4-hydroxyphenyl)-4-iodoquinolin-6-ol;
2-(3-Fluoro-4-hydroxyphenyl)-4-vinylquinolin-6-ol;
4-Ethyl-2-(3-fluoro-4-hydroxyphenyl)quinolin-6-ol;
2-(4-Hydroxyphenyl)-4-[(trimethylsilyl)ethynyl]quinolin-6-ol;
2-(3-Fluoro-4-hydroxyphenyl)-4-[(trimethylsilyl)ethynyl]quinolin-6-ol;
4-Ethynyl-2-(4-hydroxyphenyl)quinolin-6-ol;
4-Ethynyl-2-(3-fluoro-4-hydroxyphenyl)quinolin-6-ol;
2-(3,5-Difluoro-4-hydroxyphenyl)-4-ethynylquinolin-6-ol;
2-(3-Fluoro-4-hydroxyphenyl)-4-(phenylethynyl)quinolin-6-ol;
4-Acetyl-2-(4-hydroxyphenyl)quinolin-6-ol;
4-Acetyl-2-(3-fluoro-4-hydroxyphenyl)quinolin-6-ol;
4-(1-Hydroxyethyl)-2-(4-hydroxyphenyl)quinolin-6-ol;
2-(3-Fluoro-4-hydroxyphenyl)-4-(1-hydroxyethyl)quinolin-6-ol;
2-(3-Fluoro-4-hydroxyphenyl)-4-thiazol-2-yl-quinolin-6-ol;
2-(3-Fluoro-4-hydroxyphenyl)-4-thiophen-3-yl-quinolin-6-ol;
2-(3-Fluoro-4-hydroxyphenyl)-4-(4-pyridyl)quinolin-6-ol;
2-(3-Fluoro-4-hydroxyphenyl)-4-(5-pyrimidyl)quinolin-6-ol;
5-Chloro-2-(4-hydroxyphenyl)quinolin-6-ol;
5-Bromo-2-(4-hydroxyphenyl)quinolin-6-ol;
4-Bromo-3-chloro-2-(4-hydroxyphenyl)quinolin-6-ol;
4-Bromo-3-chloro-2-(3-fluoro-4-hydroxyphenyl)quinolin-6-ol;
3-Chloro-4-(4-fluorophenyl)-2-(4-hydroxyphenyl)quinolin-6-ol;
3-Chloro-4-Cyano-2-(3-fluoro-4-hydroxyphenyl)quinolin-6-ol;
4-Bromo-8-chloro-2-(3-fluoro-4-hydroxyphenyl)quinolin-6-ol;
4,8-Dibromo-2-(3-fluoro-4-hydroxyphenyl)quinolin-6-ol;
8-Chloro-4-cyano-2-(3-fluoro-4-hydroxyphenyl)quinolin-6-ol;
8-Chloro-4-ethynyl-2-(3-fluoro-4-hydroxyphenyl)quinolin-6-ol;
8-Chloro-2-(3-fluoro-4-hydroxyphenyl)-4-hex-1-ynylquinolin-6-ol;
8-Chloro-2-(3-fluoro-4-hydroxyphenyl)-4-vinylquinolin-6-ol;
8-Chloro-4-(4-cyanophenyl)-2-(3-fluoro-4-hydroxyphenyl)quinolin-6-ol;
8-Chloro-2-(3-fluoro-4-hydroxyphenyl)-4-(4-methoxyphenyl)quinolin-6-ol;
4-Chloro-8-cyano-2-(3-fluoro-4-hydroxyphenyl)quinolin-6-ol;
4-Bromo-8-cyano-2-(3-fluoro-4-hydroxyphenyl)quinolin-6-ol;
8-Cyano-4-ethynyl-2-(3-fluoro-4-hydroxyphenyl)quinolin-6-ol;
4-Chloro-8-(4-cyanophenyl)-2-(3-fluoro-4-hydroxyphenyl)quinolin-6-ol;
8-Cyano-2-(3-fluoro-4-hydroxyphenyl)quinolin-6-ol;
2-(4-Hydroxyphenyl)-4-methoxyquinolin-6-ol;
2-(4-Hydroxyphenyl)-4-vinylquinolin-6-ol;
4-Ethyl-2-(4-hydroxyphenyl)quinolin-6-ol;
2-(3-Fluoro-4-hydroxyphenyl)-4-phenylquinolin-6-ol;
2-(3-Fluoro-4-hydroxylphenyl)-4-(methylphenyl)quinolin-6-ol;
2-(3-Fluoro-4-hydroxyphenyl)-4-(4-fluorophenyl)quinolin-6-ol;
4-(4-Chlorophenyl)-2-(3-fluoro-4-hydroxyphenyl)quinolin-6-ol;
4-(4-Cyanophenyl)-2-(3-fluoro-4-hydroxyphenyl)quinolin-6-ol;
2-(3-Fluoro-4-hydroxyphenyl)-4-(4-trifluoromethylphenyl)quinolin-6-ol; and
a pharmaceutically acceptable salt thereof.

24. The method of claim 4, wherein at least one of said one or more estrogenic agents is selected from:
7-bromo-2-(4-hydroxyphenyl)-1,3-benzoxazol-5-ol;
7-bromo-2-(3-fluoro-4-hydroxyphenyl)-1,3-benzoxazol-5-ol;
5-hydroxy-2-(4-hydroxyphenyl)-1,3-benzoxazole-7-carbonitrile;
3-(3-fluoro-4-hydroxyphenyl)-7-hydroxy-1-naphthonitrile;
2-(3-fluoro-4-hydroxyphenyl)-7-vinyl-1,3-benzoxazol-5-ol;
2-(2-fluoro-4-hydroxyphenyl)-7-vinyl-1,3-benzoxazol-5-ol; and
2-(2,3-difluoro-4-hydroxyphenyl)-7-vinyl-1,3-benzoxazol-5-ol.

25. The method of claim 4, wherein at least one of said one or more estrogenic agents is: 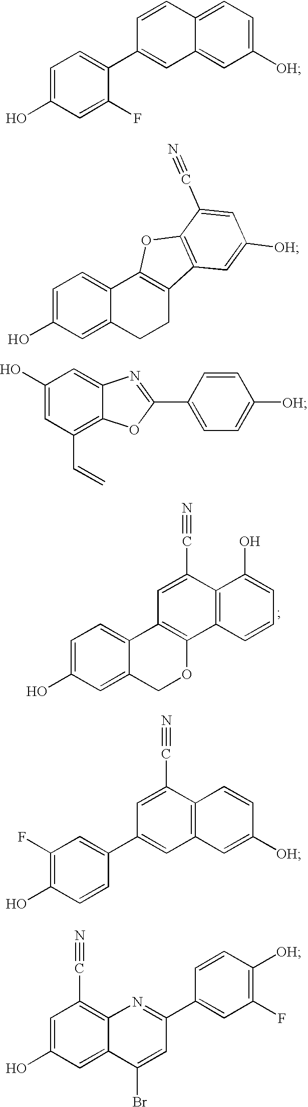 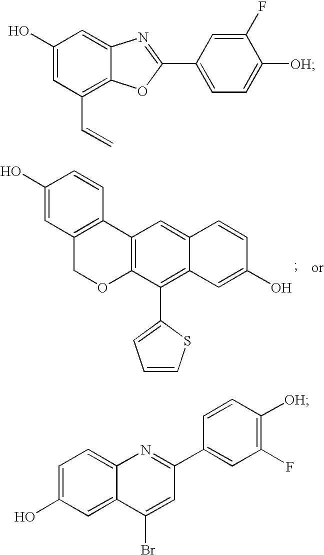
or a pharmaceutically acceptable salt thereof.

26. The method of claim 4, wherein at least one of said one or more estrogenic agents is provided in a transdermal dosage form.

27. The method of claim 26, wherein the transdermal dosage form is selected from: a lotion, a cream, an ointment, a foam, a patch, a suspension, a solution, a pledget, a mask, and a suppository.

28. The method of claim 4, comprising: additionally providing to a mammal in need thereof, one or more therapeutic agents useful in the treatment of inflammatory diseases, disorders, or conditions of the skin, or diseases, disorders, or conditions associated with collagen depletion.

29. The method of claim 28, wherein at least one of said one or more therapeutic agents useful in the treatment of inflammatory diseases, disorders, or conditions of the skin, or diseases, disorders, or conditions associated with collagen depletion is tretinoin, retinol, retinaldehyde, alpha tocopherol, monolaurin, alpha hydroxy acids, hyaluronic acid, one or more hyaluronic acid fragments, botulinum toxin (Botox® Cosmetic), an antibiotic, an antihistamine, a barrier-repair moisturizer, coal tar, a corticosteroid, cyclosporine, interferon gamma, mycophenolate mofetil, a topical calcineurin inhibitor, or a dietary supplement.

30. The method of claim 28, wherein said combination is provided in a transdermal dosage form.

31. The method of claim 30, wherein said transdermal dosage form is selected from: a lotion, a cream, an ointment, a foam, a patch, a suspension, a solution, a pledget, a mask, and a suppository.

32. A pharmaceutical composition comprising a therapeutically effective amount of one or more estrogenic agents, wherein at least one of said one or more estrogenic agents is:
a. a compound of Formula II, having the structure: 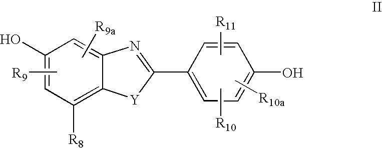
wherein
R₈ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, CONR₁₂R₁₃, NR₁₂R₁₃ or N(R₁₂)COR₁₃;
R₉ and R₉ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;
R₁₀, R₁₀ₐ and R₁₁ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;
R₁₂ and R₁₃ are each, independently hydrogen, alkyl of 1-6 carbon atoms, aryl of 6-10 carbon atoms;
Y is O, S, or NR₁₄;
R₁₄ is hydrogen, alkyl of 1-6 carbon atoms, aryl of 6-10 carbon atoms, —COR₁₂, —CO₂R₁₂ or —SO₂R₁₂;
or a pharmaceutically acceptable salt thereof;
b. a compound of Formula III, having the structure: 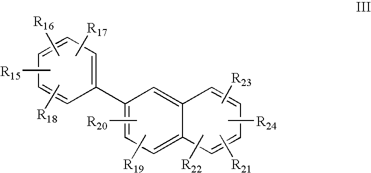
wherein
R₁₅, R₁₆, R₁₇, and R₁₈ are each, independently, selected from hydrogen, hydroxyl, alkyl of 1-6 carbon atoms, alkoxy of 1-6 carbon atoms, or halogen;
R₁₉, R₂₀, R₂₁, R₂₂, R₂₃, and R₂₄ are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₉, R₂₀′ R₂₁, R₂₂, R₂₃, or R₂₄ may be optionally substituted with hydroxyl, —CN, halogen, trifluoroalkyl, trifluoroalkoxy, —NO₂, or phenyl; wherein the phenyl moiety of R₁₉, R₂₀, R₂₁′ R₂₂, R₂₃, or R₂₄ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
with the proviso that at least one of R₁₅, R₁₆, R₁₇, R₁₈, R₂₁, R₂₂, R₂₃, or R₂₄ is hydroxyl, or a pharmaceutically acceptable salt thereof;
c. a compound of Formula IV, having the structure: 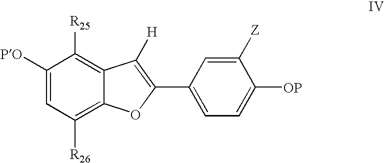
wherein
P and P′ are each, independently, hydrogen, alkyl of 1-6 carbon atoms, or acyl of 2-7 carbon atoms;
Z is hydrogen or halogen;
R₂₅ is hydrogen, alkyl of 1-6 carbon atoms, halogen, —CN, or —CHO;
R₂₆ is alkoxy of 1-6 carbon atoms, or cyanoalkyl having 1-6 carbon atoms in the alkyl moiety;
or a pharmaceutically acceptable salt thereof;
d. a compound of Formula V, having the structure: 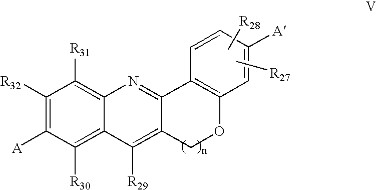
wherein:
A and A′ are each independently OH, H or OU;
each U is independently selected from the group consisting of C₁-C₆ alkyl, alkenyl, benzyl, acyl of 2-7 carbon atoms, aroyl, alkoxycarbonyl, sulfonyl and phosphoryl;
R₂₇ and R₂₈ are independently selected from the group consisting of H, halogen, C₁₋₆ alkyl, C₁₋₆ perhaloalkyl, —CF₃, C₂-C₇ alkenyl and C₁-C₆ alkoxy;
R₂₉, R₃₀, R₃₁ and R₃₂ are each independently selected from the group consisting of H, halogen, —CF₃, C₁₋₆ perhaloalkyl, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl and heteroaryl;
wherein each alkyl or alkenyl moiety of R₂₉, R₃₀, R₃₁ and R₃₂ can be optionally substituted with up to three substituents independently selected from halogen, OH, —CN, trifluoroalkyl of 1-6 carbon atoms, trifluoroalkoxy of 1-6 carbon atoms, —NO₂ and phenyl, wherein said phenyl is optionally substituted with up to three independently selected R₃₃ groups;
wherein each alkynyl moiety of R₂₉, R₃₀, R₃₁ and R₃₂ can be optionally substituted with up to three substituents selected from halogen, —CN, —CHO, acyl, trifluoroalkyl of 1-6 carbon atoms, trialkylsilyl and phenyl, wherein said phenyl is optionally substituted with up to three independently selected R₃₃ groups;
each R₃₃ is independently selected from the group consisting of halogen, C₁-C₆ alkyl, C₂-C₇ alkenyl, —OH, C₁-C₆ alkoxy, —CN, —CHO, —NO₂, amino, C₁-C₆ alkylamino, di-(C₁-C₆)alkylamino, thiol, and C₁-C₆ alkylthio; and
n is 0, 1, 2, or 3;
provided that:
at least one of A and A′ is not H;
if n is 0, then R₂₉ is not halogen; and
at least one of R₂₉, R₃₀ and R₃₁ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
or a pharmaceutically acceptable salt thereof;
e. a compound of Formula VI, having the structure: 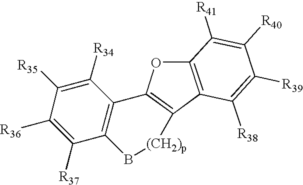
wherein:
B has the structure (i), (ii) or (iii): 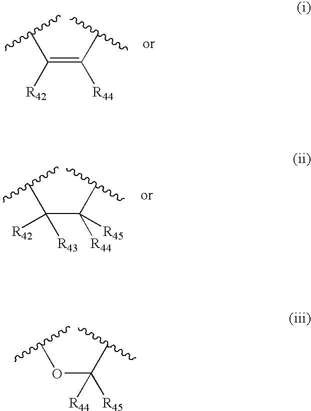
R₃₄, R₃₇, R₃₈, R₄₁, R₄₂, R₄₃, R₄₄, and R₄₅ are each independently selected from the group consisting of hydrogen, C₁-C₆ alkyl, —OR₄₆, halogen, —CF₃, —CF₂CF₃, —CH₂CF₃, —SR₄₆, —NR₄₆R₄₇, —CN, —CH₂CN, —CH₂CH₂CN, —CH═CHCN, —NO₂, —CH₂NO₂, —CH₂CH₂NO₂, —CH═CHNO₂ and —COR₄₆;
p=0 or 1;
each R₄₆ and R₄₇ is independently selected from the group consisting of hydrogen, C₁-C₆ alkyl, —CF₃, benzyl, —CO₂(C₁-C₆ alkyl) and —CO(C₁-C₆ alkyl);
provided that:
a) one of R₃₅ or R₃₆ must be —OR₄₆;
b) one of R₃₉ or R₄₀ must be —OR₄₆;
c) when R₃₅ is —OR₄₆, then R₃₄ and R₃₆ are independently selected from the group consisting of hydrogen, halogen, C₁-C₆ alkyl, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;
d) when R₃₆ is —OR₄₆, then R₃₅ and R₃₇ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;
e) when R₃₉ is —OR₄₆, then R₃₈ and R₄₀ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₃;
f) when R₄₀ is —OR₄₆, then R₃₉ and R₄₁ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆; and
g) when B has the structure (iii), and R₃₈, R₃₉, R₄₁, R₄₄, and R₄₅ are each H, and p=0, then R₄₀ is not —OR₄₆;
f. a compound of Formula VII, having the structure: 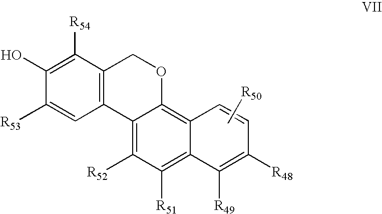
wherein
R₄₈ and R₄₉ are each, independently, selected from hydrogen, hydroxyl, alkyl of 1-6 carbon atoms, alkenyl of 2-7 carbon atoms, alkynyl of 2-7 carbon atoms, alkoxy of 1-6 carbon atoms, or halogen; wherein each alkyl or alkenyl moiety of R₄₈ or R₄₉ may be optionally and independently substituted with hydroxyl, —CN, halogen, trifluoroalkyl of 1-6 carbon atoms, trifluoroalkoxy of 1-6 carbon atoms, —NO₂ or phenyl; and provided that at least one of R₄₈ or R₄₉ is hydroxyl;
R₅₀, R₅₁, R₅₂, R₅₃, and R₅₄ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein each alkyl or alkenyl moiety of R₅₁, R₅₂, R₅₃ or R₅₄ may be optionally and independently substituted with hydroxyl, —CN, halogen, trifluoroalkyl of 1-6 carbon atoms, trifluoroalkoxy of 1-6 carbon atoms, —NO₂ or phenyl; wherein the phenyl moiety of R₅₁ or R₅₂ may be optionally mono-, di-, or tri-substituted with a substituent, the same or different, selected from alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt thereof;
g. a compound of Formula VIII, having the structure: 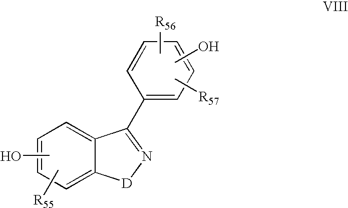
wherein,
R₅₅ and R₅₆ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, trifluoroalkyl of 1-6 carbon atoms, trifluoroalkoxy of 1-6 carbon atoms, alkylthio of 1-6 carbon atoms, or aryl of 6-10 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₇ is hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, alkylthio of 1-6 carbon atoms, trifluoroalkyl of 1-6 carbon atoms, or trifluoroalkoxy of 1-6 carbon atoms, aryl of 6-10 carbon atoms, —NO₂, —CN, —NR₅₈R₅₉ or —N(R₅₈)COR₅₉, —CHFCN, —CF₂CN, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₈ and R₅₉ are each, independently, hydrogen, alkyl of 1-6 carbon atoms, or aryl of 6-10 carbon atoms;
D is O, S, or NR₆₀;
R₆₀ is hydrogen, alkyl of 1-6 carbon atoms, aryl of 6-10 carbon atoms, —COR₅₈, —CO₂R₅₈, or —SO₂R₅₈;
h. a compound of Formula IX, having the structure: 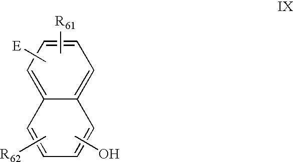
wherein, 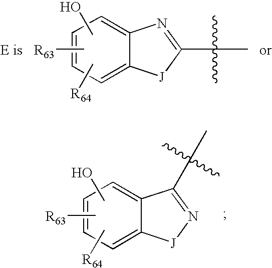
R₆₁, R₆₃, and R₆₄ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₂ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₆₅R₆₆, —N(R₆₅)COR₆₆, —CN, —CHFCN, —CF₂CN, alkynyl of 2-7 carbon atoms, or alkenyl of 2-7 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₅ and R₆₆ are each, independently, hydrogen, alkyl of 1-6 carbon atoms, alkenyl of 2-7 carbon atoms, aryl of 6-10 carbon atoms, —CN, —CHFCN, or —CF₂CN; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₆₇, —CO₂R₆₈, —NO₂, CO NR₆₇R₆₈, —NR₆₇R₆₈ or —N(R₆₇)CO R₆₈;
R₆₇ and R₆₈ are each, independently, hydrogen, alkyl of 1-6 carbon atoms, or aryl of 6-10 carbon atoms;
J is O, S, or NR₆₉;
R₆₉ is hydrogen, alkyl of 1-6 carbon atoms, aryl of 6-10 carbon atoms, —COR₆₅, —CO₂R₆₅, or —SO₂R₆₅;
or a pharmaceutically acceptable salt thereof;
i. a compound of Formula X, having the structure: 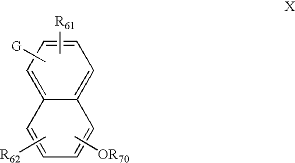
wherein, 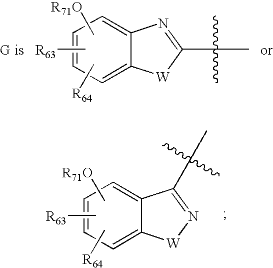
wherein
R₆₁, R₆₂, R₆₃, and R₆₄ are as defined above;
W is O, S, or NR₆₉;
R₆₉ is as defined above; and
R₇₀ and R₇₁ are alkyl of 1-6 carbon atoms;
or a pharmaceutically acceptable salt thereof;
j. a compound of Formula XI, having the structure: 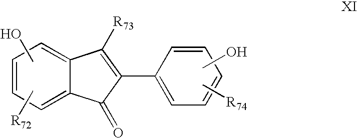
wherein,
R₇₂ is hydrogen, hydroxyl, halogen, trifluoroalkyl of 1-6 carbon atoms, alkyl of 1-6 carbon atoms, cycloalkyl of 3-8 carbon atoms, alkenyl of 2-7 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, —CN, —NO₂, —CHFCN, —CF₂CN, aryl of 6-10 carbon atoms, —NR₇₅R₇₆, —NCOR₇₅, —SR₇₅, —SOR₇₅, —SO₂R₇₅, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₇₅, —CO₂R₇₅, —CONR₇₅R₇₆, or —NR₇₅R₇₆;
R₇₃ is hydrogen, hydroxyl, alkyl of 1-6 carbon atoms, halogen, phenyl substituted with R₁, alkylthio of 1-6 carbon atoms, thioalkyl of 1-6 carbon atoms, amino, aminoalkyl of 1-6 carbon atoms, alkylamino of 1-6 carbon atoms, alkoxy of 1-6 carbon atoms, or alkenyl of 2-7 carbon atoms;
R₇₄ is hydrogen, halogen, hydroxyl, alkyl of 1-6 carbon atoms, alkoxy of 1-6 carbon atoms, trifluoroalkyl of 1-6 carbon atoms, or trifluoroalkoxy of 1-6 carbon atoms;
R₇₅ and R₇₆ are each, independently, hydrogen, alkyl of 1-6 carbon atoms, or aryl of 6-10 carbon atoms;
or a pharmaceutically acceptable salt thereof;
k. a compound of Formula XII, having the structure: 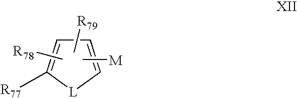
wherein,
R₇₇ is phenyl optionally substituted with 1-4 T groups;
R₇₈ is phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
R₇₉ is hydrogen, phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
L is O, —CH═CH—, or S;
T is —OH, —OR₈₀, halogen, —CN, —CO₂H, —CO₂R₈₀, alkyl of 1-6 carbon atoms, alkenyl of 2-7 carbon atoms, alkynyl of 2-7 carbon atoms, perfluoroalkyl of 1-6 carbon atoms, or —COR₈₀;
M is —CHO, —CN, —CO₂H, —CO₂R₈₀, —CONR₈OR₈₁, —NO₂, —CH═NR₈₀, —CH═NOH, or —CH═NOR₈₀;
R₈₀ and R₈₁ are each, independently, alkyl of 1-6 carbon atoms, alkenyl of 2-7 carbon atoms, alkynyl of 2-7 carbon atoms, or cycloalkyl of 3-8 carbon atoms;
with the proviso that at least one of R₇₈ or R₇₉ is phenyl or phenyl substituted with 1-4 T groups;
or a pharmaceutically acceptable salt thereof;
l. a compound of Formula XIII, having the structure: 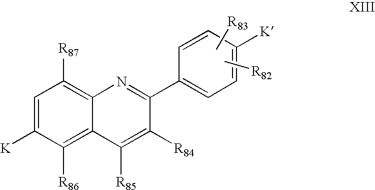
wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each, independently, H, halogen, C₁-C₆ alkyl, C₂-C₇ alkenyl, or C₁-C₆ alkoxy;
R₈₄ is H, halogen, or C₁-C₆ alkyl;
R₈₅ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, or heteroaryl;
R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₅, R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
wherein each alkynyl moiety of R₈₅, R₈₆ or R₈₇ may be optionally substituted with halogen, —CN, —CHO, acyl, trifluoroalkyl of 1-6 carbon atoms, trialkylsilyl, or optionally substituted phenyl;
wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R₈₅, R₈₆ and R₈₇ are H, C₁-C₆ alkyl, C₂-C₇ alkenyl, or C₁-C₆ alkoxy, then at least one of R₈₂ and R₈₃ is halogen, C₁-C₆ alkyl, C₂-C₇ alkenyl, or C₁-C₆ alkoxy;
provided that at least one of R₈₅ and R₈₇ is other than H;
or a pharmaceutically acceptable salt thereof;
m. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each H;
R₈₄ is H, halogen, or C₁-C₆ alkyl;
R₈₅, R₈₆ and R₈₇ are each, independently, H, C₁-C₆ alkyl, C₂-C₇ alkenyl, or C₁-C₆ alkoxy, provided that at least one of R₈₅, R₈₆ and R₈₇ is C₁-C₆ alkyl, C₂-C₇ alkenyl, or C₁-C₆ alkoxy;
wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
or a pharmaceutically acceptable salt thereof; or
n. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each, independently, H, halogen, C₁-C₆ alkyl, C₂-C₇ alkenyl, or C₁-C₆ alkoxy;
R₈₄ is H;
R₈₅ is H, aryl, or substituted aryl;
R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
wherein each alkyl or alkenyl moiety of R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
wherein each alkynyl moiety of R₈₆ or R₈₇ may be optionally substituted with halogen, —CN, —CHO, acyl of 2-7 carbon atoms, trifluoroalkyl of 1-6 carbon atoms, trialkylsilyl, or optionally substituted phenyl;
wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
or a N-oxide thereof or a pharmaceutically acceptable salt thereof or a prodrug thereof;
in combination with one or more therapeutic agents useful in the treatment of inflammatory diseases, disorders, or conditions of the skin, or diseases, disorders, or conditions associated with collagen depletion; and
at least one pharmaceutically acceptable diluent or carrier.

33. The composition of claim 32, wherein at least one of said one or more estrogenic agents is: 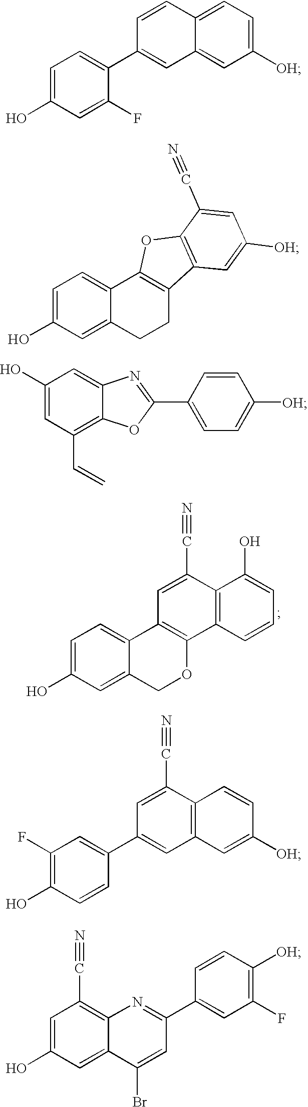 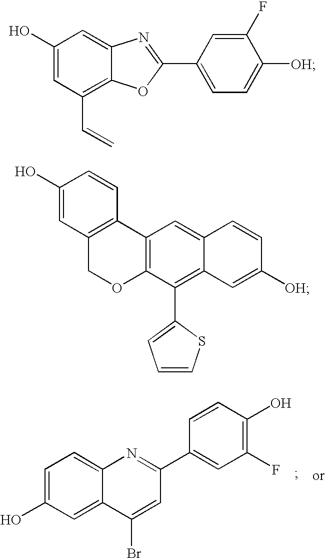
a N-oxide thereof or a pharmaceutically acceptable salt thereof or a prodrug thereof.

34. The composition of claim 32, wherein said combination is provided in a transdermal dosage form.

35. The composition of claim 34, wherein said transdermal dosage form is selected from: a lotion, a cream, an ointment, a foam, a patch, a suspension, a solution, a pledget, a mask, and a suppository.

36. The method of claim 4, wherein the disease, disorder, or condition associated with collagen depletion is photo aging.

37. The method of claim 4, wherein the disease, disorder, or condition associated with collagen depletion is chronological skin aging.

38. The method of claim 4, wherein the disease, disorder, or condition associated with collagen depletion is smoking induced skin aging.

39. The method of claim 4, wherein the inflammatory disease, disorder, or condition of the skin is eczema.

40. The method of claim 4, wherein the inflammatory disease, disorder, or condition of the skin is a type of dermatitis.

41. The method of claim 40, wherein the dermatitis is selected from: cercarial dermatitis, dermatitis herpetiformis, atopic dermatitis, seborrhoeic dermatitis, contact dermatitis, chronic dermatitis, irritant dermatitis, urushiol-induced contact dermatitis, nummular dermatitis, and dyshidrotic dermatitis.

42. The method of claim 4, wherein the inflammatory disease, disorder, or condition of the skin is psoriasis.

43. A method of suppressing MMP1, IL1B, and/or IL8 activity in mammalian epidermal and dermal skin comprising: providing to a mammal in need thereof, a therapeutically effective amount of one or more estrogenic agents, wherein at least one of said one or more estrogenic agents is:
a. a compound of Formula II, having the structure: 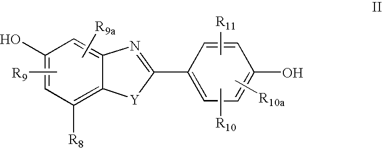
wherein
R₈ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, CONR₁₂R₁₃, NR₁₂R₁₃ or N(R₁₂)COR₁₃;
R₉ and R₉ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;
R₁₀, R₁₀ₐ and R₁₁ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₁₂, —CO₂R₁₂, —NO₂, —CONR₁₂R₁₃, —NR₁₂R₁₃ or —N(R₁₂)COR₁₃;
R₁₂ and R₁₃ are each, independently hydrogen, alkyl of 1-6 carbon atoms, aryl of 6-10 carbon atoms;
Y is O, S, or NR₁₄;
R₁₄ is hydrogen, alkyl of 1-6 carbon atoms, aryl of 6-10 carbon atoms, —COR₁₂, —CO₂R₁₂ or —SO₂R₁₂;
or a pharmaceutically acceptable salt thereof;
b. a compound of Formula III, having the structure: 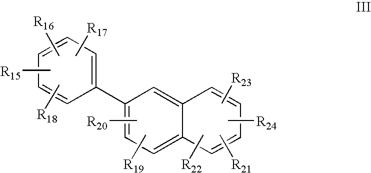
wherein
R₁₅, R₁₆, R₁₇, and R₁₈ are each, independently, selected from hydrogen, hydroxyl, alkyl of 1-6 carbon atoms, alkoxy of 1-6 carbon atoms, or halogen;
R₁₉, R₂₀, R₂₁, R₂₂, R₂₃, and R₂₄ are each, independently, hydrogen, hydroxyl, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₉, R₂₀, R₂₁, R₂₂, R₂₃, or R₂₄ may be optionally substituted with hydroxyl, —CN, halogen, trifluoroalkyl, trifluoroalkoxy, —NO₂, or phenyl; wherein the phenyl moiety of R₁₉, R₂₀, R₂₁, R₂₂, R₂₃, or R₂₄ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
with the proviso that at least one of R₁₅, R₁₆, R₁₇, R₁₈, R₂₁, R₂₂, R₂₃, or R₂₄ is hydroxyl, or a pharmaceutically acceptable salt thereof;
c. a compound of Formula IV, having the structure: 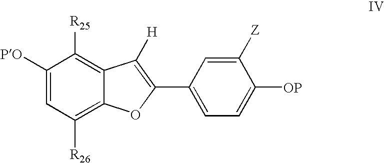
wherein
P and P′ are each, independently, hydrogen, alkyl of 1-6 carbon atoms, or acyl of 2-7 carbon atoms;
Z is hydrogen or halogen;
R₂₅ is hydrogen, alkyl of 1-6 carbon atoms, halogen, —CN, or —CHO;
R₂₆ is alkoxy of 1-6 carbon atoms, or cyanoalkyl having 1-6 carbon atoms in the alkyl moiety; or a pharmaceutically acceptable salt thereof;
d. a compound of Formula V, having the structure: 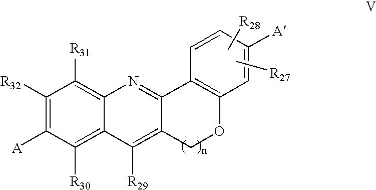
wherein:
A and A′ are each independently OH, H or OU;
each U is independently selected from the group consisting of C₁-C₆ alkyl, alkenyl, benzyl, acyl of 2-7 carbon atoms, aroyl, alkoxycarbonyl, sulfonyl and phosphoryl;
R₂₇ and R₂₈ are independently selected from the group consisting of H, halogen, C₁₋₆ alkyl, C₁₋₆ perhaloalkyl, —CF₃, C₂-C₇ alkenyl and C₁-C₆ alkoxy;
R₂₉, R₃₀, R₃₁ and R₃₂ are each independently selected from the group consisting of H, halogen, —CF₃, C₁₋₆ perhaloalkyl, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl and heteroaryl;
wherein each alkyl or alkenyl moiety of R₂₉, R₃₀, R₃₁ and R₃₂ can be optionally substituted with up to three substituents independently selected from halogen, OH, —CN, trifluoroalkyl of 1-6 carbon atoms, trifluoroalkoxy of 1-6 carbon atoms, —NO₂ and phenyl, wherein said phenyl is optionally substituted with up to three independently selected R₃₃ groups;
wherein each alkynyl moiety of R₂₉, R₃₀, R₃₁ and R₃₂ can be optionally substituted with up to three substituents selected from halogen, —CN, —CHO, acyl, trifluoroalkyl of 1-6 carbon atoms, trialkylsilyl and phenyl, wherein said phenyl is optionally substituted with up to three independently selected R₃₃ groups;
each R₃₃ is independently selected from the group consisting of halogen, C₁-C₆ alkyl, C₂-C₇ alkenyl, —OH, C₁-C₆ alkoxy, —CN, —CHO, —NO₂, amino, C₁-C₆ alkylamino, di-(C₁-C₆)alkylamino, thiol, and C₁-C₆ alkylthio; and
n is 0, 1, 2, or 3;
provided that:
at least one of A and A′ is not H;
if n is 0, then R₂₉ is not halogen; and
at least one of R₂₉, R₃₀ and R₃₁ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
or a pharmaceutically acceptable salt thereof;
e. a compound of Formula VI, having the structure: 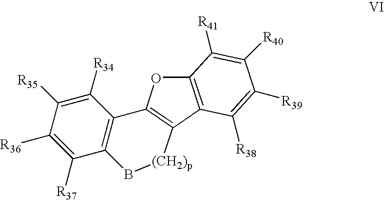
wherein:
B has the structure (i), (ii) or (iii): 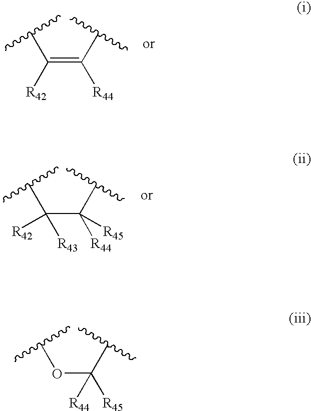
R₃₄, R₃₇, R₃₈, R₄₁, R₄₂, R₄₃, R₄₄, and R₄₅ are each independently selected from the group consisting of hydrogen, C₁-C₆ alkyl, —OR₄₆, halogen, —CF₃, —CF₂CF₃, —CH₂CF₃, —SR₄₆, —NR₄₆R₄₇, —CN, —CH₂CN, —CH₂CH₂CN, —CH═CHCN, —NO₂, —CH₂NO₂, —CH₂CH₂NO₂, —CH═CHNO₂ and —COR₄₆;
p=0 or 1;
each R₄₆ and R₄₇ is independently selected from the group consisting of hydrogen, C₁-C₆ alkyl, —CF₃, benzyl, —CO₂(C₁-C₆ alkyl) and —CO(C₁-C₆ alkyl);
provided that:
a) one of R₃₅ or R₃₆ must be —OR₄₆;
b) one of R₃₉ or R₄₀ must be —OR₄₆;
c) when R₃₅ is —OR₄₆, then R₃₄ and R₃₆ are independently selected from the group consisting of hydrogen, halogen, C₁-C₆ alkyl, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;
d) when R₃₆ is —OR₄₆, then R₃₅ and R₃₇ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆;
e) when R₃₉ is —OR₄₆, then R₃₈ and R₄₀ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₃;
f) when R₄₀ is —OR₄₆, then R₃₉ and R₄₁ are independently selected from the group consisting of hydrogen, C₁-C₆ alkyl, halogen, —CF₃, —CF₂CF₃, —CH₂CF₃, —SR₄₆, —CN, —CH₂CN, —CH₂CH₂CN, —CH═CHCN, —NO₂, —CH₂NO₂, —CH₂CH₂NO₂, —CH═CHNO₂ and —COR₄₆; and
g) when B has the structure (iii), and R₃₈, R₃₉, R₄₁, R₄₄, and R₄₅ are each H, and p=0, then R₄₀ is not —OR₄₆;
f. a compound of Formula VII, having the structure: 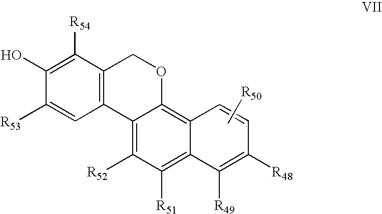
wherein
R₄₈ and R₄₉ are each, independently, selected from hydrogen, hydroxyl, alkyl of 1-6 carbon atoms, alkenyl of 2-7 carbon atoms, alkynyl of 2-7 carbon atoms, alkoxy of 1-6 carbon atoms, or halogen; wherein each alkyl or alkenyl moiety of R₄₈ or R₄₉ may be optionally and independently substituted with hydroxyl, —CN, halogen, trifluoroalkyl of 1-6 carbon atoms, trifluoroalkoxy of 1-6 carbon atoms, —NO₂ or phenyl; and provided that at least one of R₄₈ or R₄₉ is hydroxyl;
R₅₀, R₅₁, R₅₂, R₅₃, and R₅₄ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein each alkyl or alkenyl moiety of R₅₁, R₅₂, R₅₃ or R₅₄ may be optionally and independently substituted with hydroxyl, —CN, halogen, trifluoroalkyl of 1-6 carbon atoms, trifluoroalkoxy of 1-6 carbon atoms, —NO₂ or phenyl; wherein the phenyl moiety of R₅₁ or R₅₂ may be optionally mono-, di-, or tri-substituted with a substituent, the same or different, selected from alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt thereof;
g. a compound of Formula VIII, having the structure: 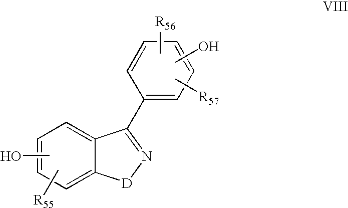
wherein,
R₅₅ and R₅₆ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, trifluoroalkyl of 1-6 carbon atoms, trifluoroalkoxy of 1-6 carbon atoms, alkylthio of 1-6 carbon atoms, or aryl of 6-10 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₇ is hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, alkylthio of 1-6 carbon atoms, trifluoroalkyl of 1-6 carbon atoms, or trifluoroalkoxy of 1-6 carbon atoms, aryl of 6-10 carbon atoms, —NO₂, —CN, —NR₅₈R₅₉ or —N(R₅₈)COR₅₉, —CHFCN, —CF₂CN, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₅₈, —CO₂R₅₈, —NO₂, —CONR₅₈R₅₉, —NR₅₈R₅₉ or —N(R₅₈)COR₅₉;
R₅₈ and R₅₉ are each, independently, hydrogen, alkyl of 1-6 carbon atoms, or aryl of 6-10 carbon atoms;
D is O, S, or NR₆₀;
R₆₀ is hydrogen, alkyl of 1-6 carbon atoms, aryl of 6-10 carbon atoms, —COR₅₈, —CO₂R₅₈, or —SO₂R₅₈;
h. a compound of Formula IX, having the structure: 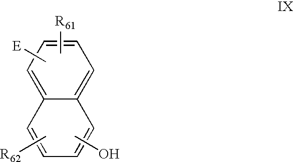
wherein, 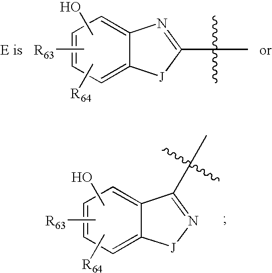
R₆₁, R₆₃, and R₆₄ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₂ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₆₅R₆₆, —N(R₆₅)COR₆₆, —CN, —CHFCN, —CF₂CN, alkynyl of 2-7 carbon atoms, or alkenyl of 2-7 carbon atoms; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₆₅, —CO₂R₆₅, —NO₂, —CONR₆₅R₆₆, —NR₆₅R₆₆ or —N(R₆₅)COR₆₆;
R₆₅ and R₆₆ are each, independently, hydrogen, alkyl of 1-6 carbon atoms, alkenyl of 2-7 carbon atoms, aryl of 6-10 carbon atoms, —CN, —CHFCN, or —CF₂CN; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₆₇, —CO₂R₆₈, —NO₂, CO NR₆₇R₆₈, —NR₆₇R₆₈ or —N(R₆₇)CO R₆₈;
R₆₇ and R₆₈ are each, independently, hydrogen, alkyl of 1-6 carbon atoms, or aryl of 6-10 carbon atoms;
J is O, S, or NR₆₉;
R₆₉ is hydrogen, alkyl of 1-6 carbon atoms, aryl of 6-10 carbon atoms, —COR₆₅, —CO₂R₆₅, or —SO₂R₆₅;
or a pharmaceutically acceptable salt thereof;
i. a compound of Formula X, having the structure: 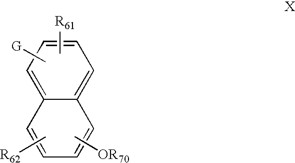
wherein, 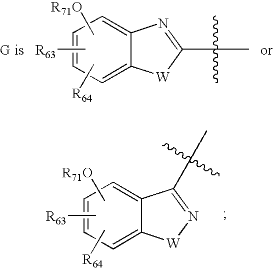
wherein
R₆₁, R₆₂, R₆₃, and R₆₄ are as defined above;
W is O, S, or NR₆₉;
R₆₉ is as defined above; and
R₇₀ and R₇₁ are alkyl of 1-6 carbon atoms;
or a pharmaceutically acceptable salt thereof;
j. a compound of Formula XI, having the structure: 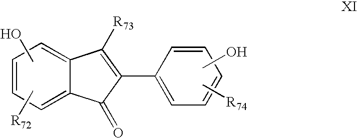
wherein,
R₇₂ is hydrogen, hydroxyl, halogen, trifluoroalkyl of 1-6 carbon atoms, alkyl of 1-6 carbon atoms, cycloalkyl of 3-8 carbon atoms, alkenyl of 2-7 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, —CN, —NO₂, —CHFCN, —CF₂CN, aryl of 6-10 carbon atoms, —NR₇₅R₇₆, —NCOR₇₅, —SR₇₅, —SOR₇₅, —SO₂R₇₅, or a 5 or 6-membered heterocyclic ring having 1 to 4 heteroatoms selected from O, N or S; wherein each alkyl or alkenyl moiety is optionally substituted with hydroxyl, —CN, halogen, trifluoroalkyl of 1-6 carbon atoms, trifluoroalkoxy of 1-6 carbon atoms, —COR₇₅, —CO₂R₇₅, —CONR₇₅R₇₆, or —NR₇₅R₇₆;
R₇₃ is hydrogen, hydroxyl, alkyl of 1-6 carbon atoms, halogen, phenyl substituted with R₁, alkylthio of 1-6 carbon atoms, thioalkyl of 1-6 carbon atoms, amino, aminoalkyl of 1-6 carbon atoms, alkylamino of 1-6 carbon atoms, alkoxy of 1-6 carbon atoms, or alkenyl of 2-7 carbon atoms;
R₇₄ is hydrogen, halogen, hydroxyl, alkyl of 1-6 carbon atoms, alkoxy of 1-6 carbon atoms, trifluoroalkyl of 1-6 carbon atoms, or trifluoroalkoxy of 1-6 carbon atoms;
R₇₅ and R₇₆ are each, independently, hydrogen, alkyl of 1-6 carbon atoms, or aryl of 6-10 carbon atoms;
or a pharmaceutically acceptable salt thereof;
k. a compound of Formula XII, having the structure: 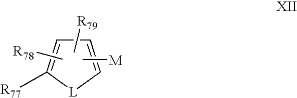
wherein,
R₇₇ is phenyl optionally substituted with 1-4 T groups;
R₇₈ is phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
R₇₉ is hydrogen, phenyl optionally substituted with 1-4 T groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;
L is O, —CH═CH—, or S;
T is —OH, —OR₈₀, halogen, —CN, —CO₂H, —CO₂R₈₀, alkyl of 1-6 carbon atoms, alkenyl of 2-7 carbon atoms, alkynyl of 2-7 carbon atoms, perfluoroalkyl of 1-6 carbon atoms, or —COR₈₀;
M is —CHO, —CN, —CO₂H, —CO₂R₈₀, —CONR₈OR₈₁, —NO₂, —CH═NR₈₀, —CH═NOH, or —CH═NOR₈₀;
R₈₀ and R₈₁ are each, independently, alkyl of 1-6 carbon atoms, alkenyl of 2-7 carbon atoms, alkynyl of 2-7 carbon atoms, or cycloalkyl of 3-8 carbon atoms;
with the proviso that at least one of R₇₈ or R₇₉ is phenyl or phenyl substituted with 1-4 T groups;
or a pharmaceutically acceptable salt thereof;
l. a compound of Formula XIII, having the structure: 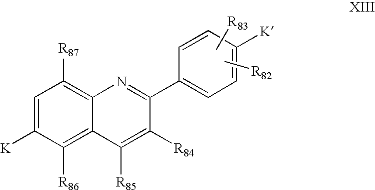
wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each, independently, H, halogen, C₁-C₆ alkyl, C₂-C₇ alkenyl, or C₁-C₆ alkoxy;
R₈₄ is H, halogen, or C₁-C₆ alkyl;
R₈₅ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, or heteroaryl;
R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₅, R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
wherein each alkynyl moiety of R₈₅, R₈₆ or R₈₇ may be optionally substituted with halogen, —CN, —CHO, acyl, trifluoroalkyl of 1-6 carbon atoms, trialkylsilyl, or optionally substituted phenyl;
wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R₈₅, R₈₆ and R₈₇ are H, C₁-C₆ alkyl, C₂-C₇ alkenyl, or C₁-C₆ alkoxy, then at least one of R₈₂ and R₈₃ is halogen, C₁-C₆ alkyl, C₂-C₇ alkenyl, or C₁-C₆ alkoxy; provided that at least one of R₈₅ and R₈₇ is other than H;
or a pharmaceutically acceptable salt thereof;
m. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each H;
R₈₄ is H, halogen, or C₁-C₆ alkyl;
R₈₅, R₈₆ and R₈₇ are each, independently, H, C₁-C₆ alkyl, C₂-C₇ alkenyl, or C₁-C₆ alkoxy,
provided that at least one of R₈₅, R₈₆ and R₈₇ is C₁-C₆ alkyl, C₂-C₇ alkenyl, or C₁-C₆ alkoxy;
wherein each alkyl or alkenyl moiety of R₈₅, R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
or a pharmaceutically acceptable salt thereof; or
n. a compound of Formula XIII, wherein:
K and K′ are each, independently, OH or OV;
V is alkyl, alkenyl, benzyl, acyl of 2-7 carbon atoms, aroyl, alkoxycarbonyl, sulfonyl or phosphoryl;
R₈₂ and R₈₃ are each, independently, H, halogen, C₁-C₆ alkyl, C₂-C₇ alkenyl, or C₁-C₆ alkoxy;
R₈₄ is H;
R₈₅ is H, aryl, or substituted aryl;
R₈₆ and R₈₇ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R₈₆ and R₈₇ is halogen, C₁-C₆ alkyl, C₂-C₇ alkenyl, C₂-C₇ alkynyl, C₃-C₇ cycloalkyl, C₁-C₆ alkoxy, —CN, —CHO, acyl of 2-7 carbon atoms, phenyl, aryl or heteroaryl;
wherein each alkyl or alkenyl moiety of R₈₆ or R₈₇ may be optionally substituted with halogen, OH, —CN, trifluoroalkyl of 1-6 carbon atoms, trifluoroalkoxy of 1-6 carbon atoms, —NO₂, or phenyl;
wherein each alkynyl moiety of R₈₆ or R₈₇ may be optionally substituted with halogen, —CN, —CHO, acyl of 2-7 carbon atoms, trifluoroalkyl of 1-6 carbon atoms, trialkylsilyl, or optionally substituted phenyl;
wherein the phenyl moiety of R₈₆ or R₈₇ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
or a N-oxide thereof or a pharmaceutically acceptable salt thereof or a prodrug thereof.
